(19) United States
(12) Patent Application Publication
(10) Pub. No.: US 2023/0220425 A1
(43) Pub. Date: Jul. 13, 2023

(54) PAM-REDUCED AND PAM-ABOLISHED CAS DERIVATIVES COMPOSITIONS AND USES THEREOF IN GENETIC MODULATION

(71) Applicant: TARGETGENE BIOTECHNOLOGIES LTD, Kibbutz Magal (IL)

(72) Inventors: Yoel Moshe SHIBOLETH, Kibbutz Magal (IL); Dan Michael WEINTHAL, Rehovot (IL); Devin Lee TRUDEAU, Rehovot (IL); Talya KUNIK, Ramat Gan (IL); Mati COHEN, Ganei Tikva (IL)

(21) Appl. No.: 17/772,842

(22) PCT Filed: Oct. 28, 2020
(86) PCT No.: PCT/IL2020/051122

§ 371 Date: Apr. 28, 2022

Related U.S. Application Data
(60) Provisional application No. 62/926,764, filed on Oct. 28, 2019.
(60) Provisional application No. 63/068,060, filed on Aug. 20, 2020.

(51) Int. Cl. C12N 15/90; C12N 9/22; C12N 15/11; C12N 15/10; A61K 38/46

(52) U.S. Cl. CPC C12N 15/907; C12N 9/22; C12N 15/11; C12N 15/102; A61K 38/465; C12N 2310/20; C12N 2800/80

(57) ABSTRACT

The invention provides highly effective and versatile CRISPR/Cas protein variants, compositions, methods and uses thereof in gene editing. More specifically, the invention relates to PAM-reduced or PAM-abolished Cas proteins and chimeras, complexes and conjugates thereof, genetic editing systems and to therapeutic and non-therapeutic methods and uses of the PAM-reduced or PAM-abolished Cas proteins.

The Sequence Listing in ASCII text file format of 2,693,418 bytes in size, created on Oct. 6, 2022, with the file name “2022-11-30SequenceListing_SHIBOLETH1_ST25,” filed in the U.S. Patent and Trademark Office on even date herewith, is hereby incorporated herein by reference.

### FIELD OF THE INVENTION

The invention relates to genetic editing systems and methods. More specifically, the invention provides highly effective and versatile CRISPR/Cas protein variants, compositions, methods and uses thereof in gene editing.

### BACKGROUND ART

References considered to be relevant as background to the presently disclosed subject matter are listed below:

[1] Anders, C., et al (2014). Nature; 513(7519):569-73.

[2] Kleinstiver, et al (2015) Nature Biotechnology; 33, 1293-1298.

[3] Hirano, S., et 1(2016) Molecular Cell; Volume 61, Issue 6, 886-894.

[4] Hu, J. H., et al (2018) Nature; 556, 57-63.

[5] Nishimasu H, et al. (2018) Science; 361(6408): 1259-1262.

[6] Kleinstiver, B. P., et al (2015) Nature; 523, 481-485.

[7] Ma, D., et al (2019) Nature Communications; 10, Article number: 560.

[8] Tsai, S. Q., et al (2014) Nature Biotechnology; 32, 569-576.

[9] Chatterjee, P., et al (2018) Science Advances; Vol. 4, no. 10, eaau0766.

[10] Liu, Y., et al (2015) Current Pharmaceutical Design; Volume 21, Issue 22.

[11] Liu, J. J., et al (2019) Nature; 566, pages 218-223

Acknowledgement of the above references herein is not to be inferred as meaning that these are in any way relevant to the patentability of the presently disclosed subject matter.

### BACKGROUND OF THE INVENTION

CRISPR-Cas endonucleases are RNA/protein complexes that specifically recognize target DNA sequences and cleave them. All known CRISPR-Cas proteins have a requirement for a “protospacer adjacent motif” (PAM), a flanking DNA sequence either 5′ or 3′ of the target sequence. Different Cas proteins have different PAM-binding domains and recognize different PAM nucleotide sequences. For example, the PAM of Streptococcus pyogenes Cas9 (spCas9) is 5′-NGG-3′, the PAM of Streptococcus canis Cas9 (scCas9) is 5′-NNG-3′, the PAM of Streptococcus aureus Cas9 (saCas9) is 5′-NNGRRN-3′, the PAM of Streptococcus thermophilus Cas9 1 (st1Cas9) is 5′-NNAGAAW-3′, the PAM of Streptococcus thermophilus Cas9 3 (st3Cas9) is 5′-NGGNG-3′, the PAM of Neisseria meningitides Cas9 (nmCas9) is 5′-NNNNGATT-3′, the PAM of CasX from deltaproteobacteria is 5′-TTCN-3′, the PAM of Cas12a from Francisella tularensis is 5′-TTN-3′, the PAM of Cas12a from Acidaminococcus sp. is 5′-TTTN-3′, the PAM of Cas12a from Lachnospiraceae is 5′-TTTN-3′, the PAM of Cas14a1 is 5′-TTTN-3′, the PAM of Cas14b5 is 5′-TTAT-3′, the PAM of CasF-1 is 5′-VTTR-3′, the PAM of CasF-2 is 5′-TBN-3′, and the PAM of CasF-3 is 5′-VTTN-3′ . When applying Cas-based genome editing and designing sgRNAs for targeting specific genes, one must always take into account the availability of a PAM sequence adjacent to the target, thus limiting choice of target cleavage location. For example, SpCas9 requires NGG, N being any nucleotide and G a guanine. Thus, the chance of a randomly selected sequence being targetable by Cas9 is 1/4×1/4=1/16.

In a dead or deactivated nuclease Cas (dCas) fused to a FokI-nuclease (dCas-FokI) configuration, the target-choice limitation becomes even more pronounced due to PAMs being necessary at both chimeric-nuclease sites, restricting the theoretical choice to a 1/16×1/16=1/256 plus the requirement for a specific distance between the monomers on the DNA, or in other words, less than 4 times per kb of DNA (actual numbers may vary due to nucleotide distribution not being truly random, but this does not change the general conclusion). Thus, it is even more advantageous to reduce or abolish the PAM restriction when using dCas-FokI-nuclease configuration, which now benefits from less-restricted or totally unrestricted choice of target sequence respectively, but also has higher specificity due to use of two specificity-conferring nucleic acids (SCNAs, also known as “single guide RNAs”, sgRNAs).

Thus, while CRISPR based systems suffer from low specificity and dCas-FokI suffer from severe sgRNA design limitations, a PAM-reduced chimera would be less limited and a PAM-free dual RNA system does not suffer from either of these impediments and is thus inherently versatile, useful and safe.

PAM sequences are recognized by the PAM binding-domain of Cas proteins. In Cas9 proteins, specificity is mediated by an “RXR” motif (Arginine, any amino acid, Arginine): a pair of Arginines (Arg) that are predicted to interact with the guanines of the NGG sequence via hydrogen bonds [1]. In principle, mutating these sites or adjacent sites may change the specificity of Cas9 proteins towards PAM sequences. Researchers have succeeded in altering the PAM binding domain (PAMBD) of Cas proteins, however, attempts at deleting this domain or replacing it with alternative domains of other proteins, have not been reported.

Kleinstiver et al targeted the RXR motif and made various mutations that conferred different PAM specificities [2]. However, a mutation of any or both of the Arginines to Q (Gln) failed to show significant cleavage. Restoration of cleavage activity of a protein containing only the second Arg mutation was possible by introducing an Arg two amino acids towards the C terminus, which was later found to adopt a similar structural configuration to the canonical RXR motif [3]. However, they were not successful in removing the requirement for a PAM nor were they successful in removing the requirement for the Arginines in the PAMBD.

Hu et al. used phage-based selections to evolve an expanded PAM SpCas9 variant (xCas9) that can recognize a broad range of PAM sequences including NG, GAA and GAT [4]. Their selections did not target the RXR motif and instead focused on adjacent structural regions. Nishimasu et al. identified a SpCas9 mutant having expanded PAM recognition, cleaving TGA, TGT, and TGC targets in addition to the native NGG site [5]. Notably, R1333 is retained and R1335 function is replaced by R1337.

Staphyloccus aureus Cas9 has a NNGRRN PAM, and this recognition is mediated in part by a “RX24R” motif. A SaCas9 variant with E782K/N968K/R1015H triple mutations (SaCas9-KKH) recognizes NNNRRT PAM [6], while retaining the R991. This variant was further engineered by Ma et al. who identified several variants with expanded recognition capability at NNVRRN, NNVACT, NNVATG, NNVATT, NNVGCT, NNVGTG, and NNVGTT PAM sequences [7]. “V42” variant had R991K mutation but introduced L988R. “V17” variant had R9911, but introduced L989R. Thus, the dual arginine configuration was retained.

Protein DNA interaction may be mediated by alternative domains such as non-sequence specific DNA binding domains. DNA binding proteins (DBPs) are a class of proteins that bind DNA, in either a sequence specific fashion or a non-sequence specific fashion. Non-sequence specific DNA binding proteins/domains (NSDBs) interact non-specifically with DNA base and backbone elements. The use of Non-sequence specific DNA binding domains in engineering novel mutants of Cas proteins for genome editing has never been published.

Therefore, there is a need for reduction and abolishment of Cas PAM requirements, for the provision of effective but specific gene editing systems. The present invention addresses these needs by providing highly specific, versatile, effective and safe gene editing systems that display reduced or abolished PAM restriction.

### SUMMARY OF THE INVENTION

In a first aspect, the invention relates to a clustered regularly interspaced short palindromic repeats (CRISPR)-Cas protein or cas protein derived domain having reduced or abolished Protospacer Adjacent Motif (PAM) constraint or any variant, mutant, fusion/chimeric protein, complex or conjugate thereof. In more specific embodiments, at least one of the PAM binding domain (PAMBD or PBD) and/or PAM recognition motif, and/or the HNH-nuclease domain, any fragment of said PBD, and/or PAM recognition motif, and/or of the HNH-nuclease domain, and at least one amino acid residue adjacent to the PBD, and/or to the PAM recognition motif, and/or to the HNH-nuclease domain of the Cas protein is deleted, replaced, mutated or substituted.

In yet a further aspect, the invention provides a nucleic acid guided genome modifier or effector chimeric or fusion protein or any complex or conjugate thereof. More specifically, nucleic acid guided genome modifier/effector chimeric or fusion protein of the invention comprises:

(a) at least one Cas protein or any Cas protein derived domain having reduced or abolished PAM constraint or any variant, or mutant thereof. It should be noted that in some optional embodiments, at least one of: the PBD of the Cas protein, and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced; and

(b) at least one nucleic acid modifier or effector component.

In yet another aspect, the invention provides a nucleic acid molecule comprising a nucleic acid sequence encoding at least one Cas protein or any Cas protein derived domain, having reduced or abolished PAM constraint or any fragment, variant, mutant, fusion protein or conjugate thereof. It should be noted that in some optional embodiments, at least one of the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

A further aspect of the invention relates to a nucleic acid guided genome modifier/effector system. In more specific embodiments, the system of the invention comprises the following components: The first component (a), of the system of the invention may be at least one Cas protein or Cas protein derived domain, having reduced or abolished PAM constraint or any fragment, variant, mutant, fusion/chimeric protein, complex or conjugate thereof. Alternatively, the system of the invention may comprise at least one nucleic acid sequence encoding this Cas protein or any part thereof (e.g., N- and or C-terminal fragments thereof), variant, mutant, fusion/chimeric protein or conjugate thereof. It should be noted that in some optional embodiments, at least one of: at least one of the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

The second component (b), of the system of the invention may be at least one target recognition element, or alternatively, any nucleic acid sequence encoding the target recognition element.

In yet some further aspect thereof, the invention relates to a host cell that is either genetically modified by, or comprising at least one of:

(a) at least one Cas protein or any Cas derived domain having reduced or abolished PAM constraint or any variant, mutant, fusion/chimeric protein, complex or conjugate thereof, or alternatively, at least one nucleic acid sequence encoding the Cas protein or any fragment or parts thereof (N- and/or C terminal fragments thereof), variant, mutant, fusion/chimeric protein or conjugate thereof. It should be noted that in some optional embodiments, at least one of: the PBD and/or PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or the PAM recognition motif, and/or to the HNH-nuclease domain of the Cas protein, is deleted, mutated, substituted or replaced.

(b) at least one target recognition element or any nucleic acid sequence encoding the target recognition element;

(c) at least one nucleic acid cassette or any vector or vehicle comprising the nucleic acid sequence of (a), the nucleic acid sequence of (b) or the nucleic acid sequence of (a) and (b); and

(d) at least one system comprising (a) and (b).

A further aspect of the invention relates to a composition comprising at least one of:

(a) at least one Cas protein or any Cas derived domain having reduced or abolished PAM constraint or any variant, mutant, fusion/chimeric protein, complex or conjugate thereof, or at least one nucleic acid sequence encoding the Cas protein or any fragment, part, variant, mutant, fusion/chimeric protein or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

(b) at least one target recognition element or any nucleic acid sequence encoding such target recognition element.

(c) at least one nucleic acid cassette or any vector or vehicle comprising the nucleic acid sequence of (a), the nucleic acid sequence of (b) or the nucleic acid sequence of (a) and (b).

(d) at least one system comprising (a) and (b), and

(e) at least one host cell modified by, and/or comprising at least one of: the nucleic acid cassette or any vector or vehicle of (c) and the at least one system of (d);

or any matrix, nano- or micro-particle comprising at least one of (a), (b), (c), (d) and (e). It should be noted that the composition of the invention may optionally further comprises at least one of pharmaceutically acceptable carrier/s, diluent/s, excipient/s and additive/s.

In yet a further aspect, the invention provides a method of modifying at least one target nucleic acid sequence of interest in at least one cell. More specifically, the method may comprise the steps of contacting the cell with:

First (a), at least one Cas protein having reduced or abolished PAM constraint or any variant, mutant, fusion/chimeric protein, complex or conjugate thereof, or at least one nucleic acid sequence encoding said Cas protein or any fragment, part, variant, mutant, fusion/chimeric protein, complex or conjugate thereof. It should be noted that in some optional embodiments at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced; and

Second (b), at least one target recognition element or any nucleic acid sequence encoding such target recognition element.

Alternatively, the method may contact the cell with (c), at least one nucleic acid cassette or any vector or vehicle comprising the nucleic acid sequence of (a), the nucleic acid sequence of (b) or the nucleic acid sequence of (a) and (b).

In yet some further embodiments (d), the cells may be contacted with at least one system or composition comprising (a) and (b).

A further aspect of the invention relates to a method of curing or treating, preventing, inhibiting, reducing, eliminating, protecting or delaying the onset of a pathologic disorder or condition in a subject in need thereof. More specifically, the method of the invention may comprise the steps of administering to the treated subject an effective amount of at least one of:

(a) at least one Cas protein or any Cas protein derived domain, having reduced or abolished PAM constraint or any fragment, variant, mutant, fusion/chimeric protein, complex or conjugate thereof, or alternatively or additionally, at least one nucleic acid sequence encoding the Cas protein or any fragment, part, variant, mutant, fusion/chimeric protein, complex or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding the target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b);

(e) at least one host cell modified by and/or comprising at least one of: (a), (b), (c) and (d); and

(f) at least one composition comprising at least one of (a), (b), (c), (d) and (e).

Still further, another aspect of the invention relates to an effective amount of at least one of:

(a) at least one Cas protein or any Cas protein derived domain having reduced or abolished PAM constraint or any fragment, variant, mutant, fusion/chimeric protein, complex or conjugate thereof, or at least one nucleic acid sequence encoding said Cas protein or any variant, mutant, fusion/chimeric protein or conjugate thereof. More specifically, in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding said target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b);

(e) at least one host cell modified by, and/or comprising at least one of: (a), (b), (c) and (d); and

(f) at least one composition comprising at least one of (a), (b), (c), (d) and (e); for use in methods of curing or treating, preventing, inhibiting, reducing, eliminating, protecting or delaying the onset of a pathologic disorder or condition in a subject in need thereof.

In yet some further aspects thereof, the invention provides an effective amount of at least one of:

(a) at least one Cas protein or any Cas protein derived domain having reduced or abolished PAM constraint or any fragment, variant, mutant, fusion/chimeric protein, complex or conjugate thereof, or at least one nucleic acid sequence encoding said Cas protein or any variant, mutant, fusion/chimeric protein or conjugate thereof. More specifically, in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding the target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b); and

(e) at least one composition comprising at least one of (a), (b), (c), and (d); for use in method of modifying at least one target nucleic acid sequence of interest in at least one cell.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D: Droplet digital (ddPCR) analysis of the EMX1 gene targeting in human cells

FIG. 1A: Graph showing positive control SpCas9 with a single EMX1 sgRNA. Calculated Non-homologous end joining (NHEJ) erroneous repair (ER) efficiency percentage of total accepted droplets.

FIG. 1B: Graph showing tested dead ScCas9-FokI combined with (or without) a pair of SCNAs (sgRNAs) situated at gaps of 15 or 27 bases apart. Calculated NHEJ-ER percentage of total accepted droplets.

FIG. 1C-1D: Graphs showing highly efficient human genome editing (upper panel (1C): correctly spaced 15 nt gap pair of SCNAs) and no editing detected (lower panel (1D): wrongly spaced 27 nt gap pair of SCNAs). Raw data for the calculation of FIG. 1B. Threshold crosshairs were placed identically for both treatments. Circled top left quarter droplets are erroneous repair products of NHEJ at the intended target site and are droplets containing a single template. Droplets containing more than one template of which one or more are WT and at least one is mutated, are present in the clearly visible “tail” to the right of these droplets included in the circle and in the top right panel.

FIG. 2: Schematic representation of a chimeric dCas-FokI construct bound to DNA target site

A protein comprising a Nuclear Localization Sequence (NLS), FokI nuclease monomer (FokI), nuclease-deficient Cas nucleoprotein (dCas), and a non-sequence-specific DNA-binding domain (NSDB), bound to a single guide RNA (sgRNA), is shown bound to a DNA target site that is complementary to the sgRNA. Two monomers of this protein are bound to DNA target sites separated by a double-stranded gap region (dsDNA), positioning the FokI domains for dimerization and cleavage.

FIG. 3: Comparison of NHEJ erroneous repair percentages in the human Myeloperoxidase (MPO) gene for dScCas9-FokI derivatives wherein two domains have been deleted

The figure shows comparison of NHEJ erroneous repair percentages in the targeted human Myeloperoxidase (MPO) gene assayed by ddPCR for different dScCas9-FokI derivatives at two timepoints (76 hrs [wavy fill] or 120 hrs [hatched fill] post transfection) in two separate biological experiments. All constructs in the figure have an NNG PAM, a combination of an SV40 and an SV40-derived bipartite Nuclear Localization Signal (NLS), a deletion of the scLoop and the RuvC+Rec1/2 domain (“Ancestor Rec”) ancestral mutations as described in Table 10C and in Example 2. Constructs were delivered by plasmid into Hek293 cells together with a plasmid encoding a pair of SCNAs (sgRNAs). Different constructs denoted by their “TG number” (as can be found also in table 10C) have different combinations of differently altered domains. Ancestral RuvC+Rec1/2 domain (“Ancestor Rec”) is as described in Example 16; Wild type (wt) HNH is the HNH domain of scCas9; HNH deletion is a protein wherein the wt HNH domain has been deleted.

FIG. 4: Comparison of NHEJ erroneous repair percentages in the human Myeloperoxidase (MPO) gene for dScCas9-FokI derivatives lacking both a native PAM-binding domain (PAMBD) and a native scLoop

The figure shows comparison of NHEJ erroneous repair percentages in the targeted human Myeloperoxidase (MPO) gene assayed by ddPCR for different dScCas9-FokI derivatives at two timepoints (76 hrs [black] or 120 hrs [white] post transfection) in two separate biological experiments. All constructs have had the native PAM binding domain [PAMBD] replaced with the domain indicated: Zinc Finger (ZF), LacI (Lac), SSo7D, HMGN or STO7 all as described in Example 2. All constructs have a mutated ScCas9 “scLoop”, an SV40-Nuclear Localization Signal (NLS) combined with a nucleoplasmin NLS, an HNH deletion and the ancestral RuvC+Rec1/2 mutations as described in Example 16. Constructs were delivered by plasmid into Hek293 cells together with a plasmid encoding a pair of SCNAs (sgRNAs). Different constructs denoted by their “TG number” (as can be found also in Table 10C) have different combinations of altered domains. Ancestral RuvC+Rec1/2 (“Ancestral mutations”) is as described in Example 16; Del HNH is a protein wherein the wt HNH of scCas9 has been deleted; wt FokI is the FokI-nuclease domain of FokI.

FIG. 5A-5B: Comparison of NHEJ erroneous repair percentages in the human EMX1 gene for dScCas9-FokI and dScCas9-FokI dHNH

The figure shows comparison of NHEJ erroneous repair percentages in the human Homeobox 1(EMX1) gene assayed by ddPCR for dScCas9-FokI and dScCas9-FokI dHNH (both with NNG PAM) with a pair of SCNAs (sgRNAs) at 2 different gaps compared to SpCas9 (NGG PAM) with a single sgRNA.

FIG. 5A: Graph showing tested dScCas9-FokI and dScCas9-FokI dHNH combined with (or without) a pair of SCNAs (sgRNAs) situated at gaps of 15 or 27 bases apart. Calculated percentage of total accepted droplets. Positive control spCas9 with a single EMX1 sgRNA. Calculated percentage of total accepted droplets. Results show highest activity for SpCas9 (single sgRNA), somewhat lower for dScCas9-Fok combined with a pair of SCNAs spaced 15 nucleotide (nt) apart, lower yet significant activity for dScCas9 dHNH-Fok combined with a pair of SCNAs spaced 15 nt apart and greatly reduced activity with a 27 nt gap.

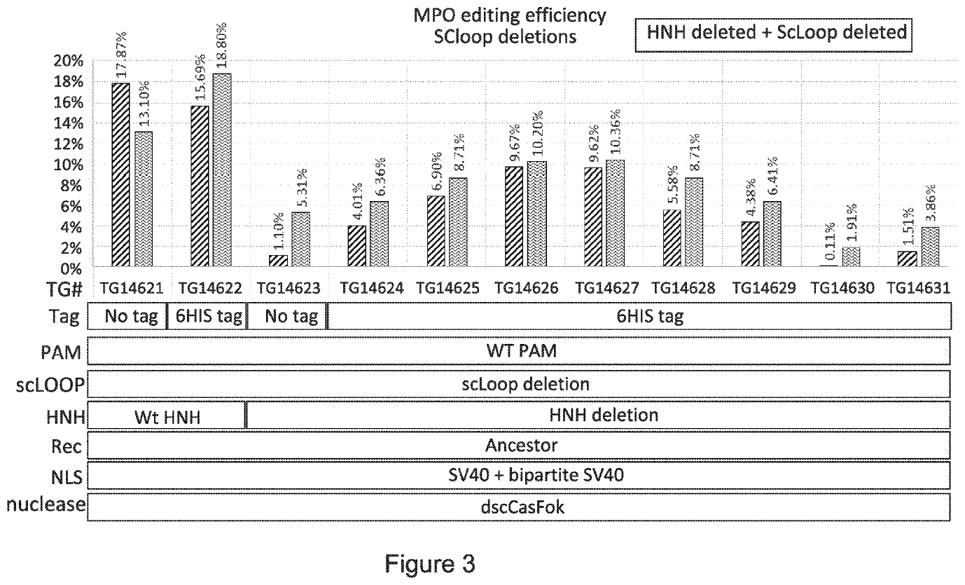

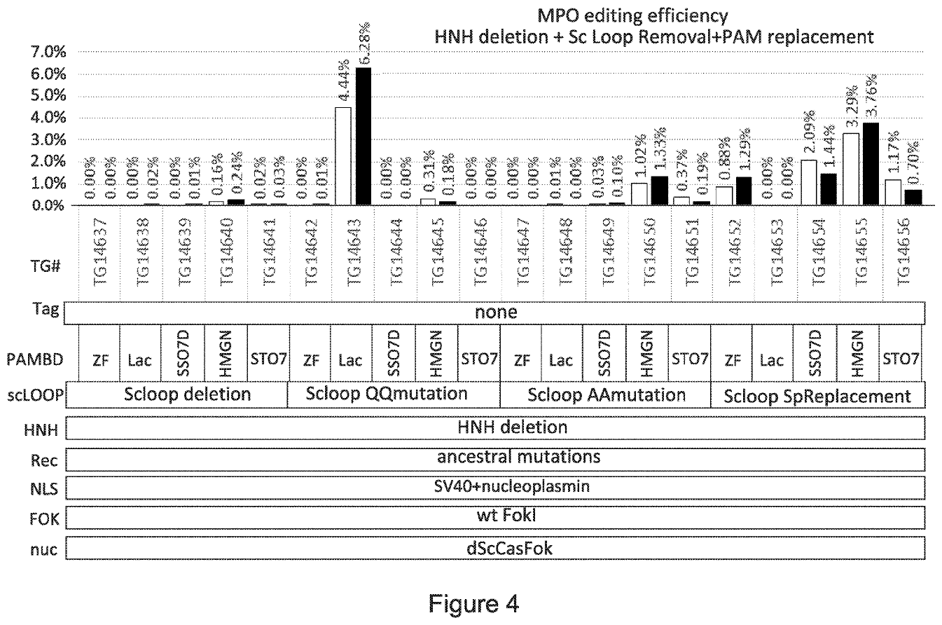

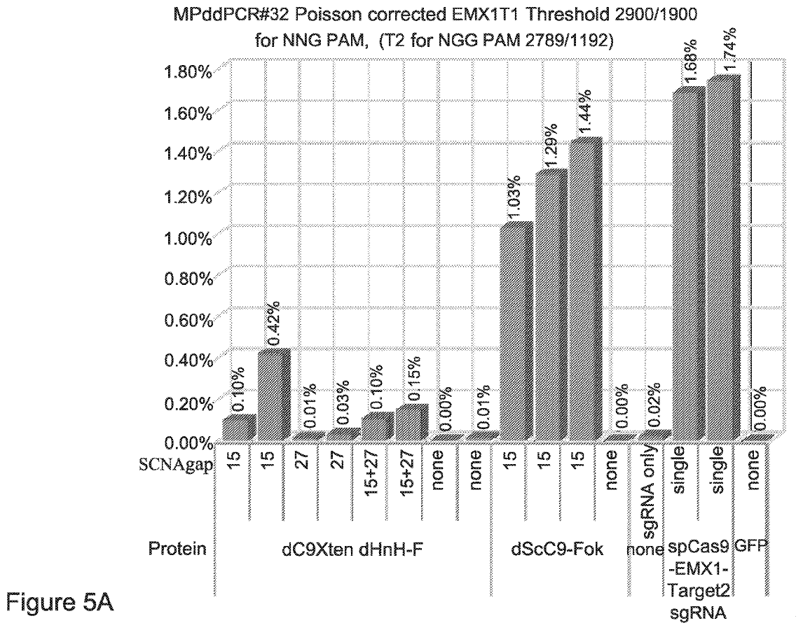

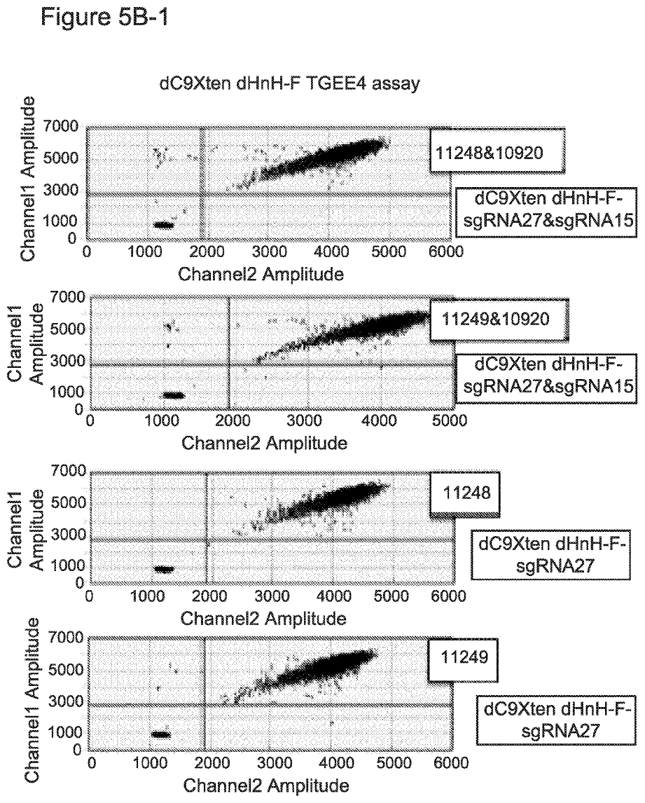

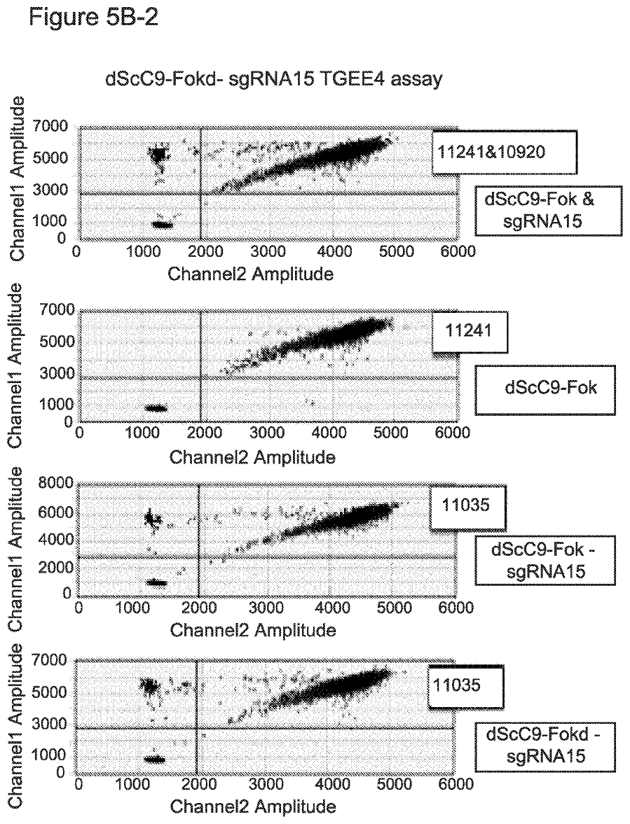

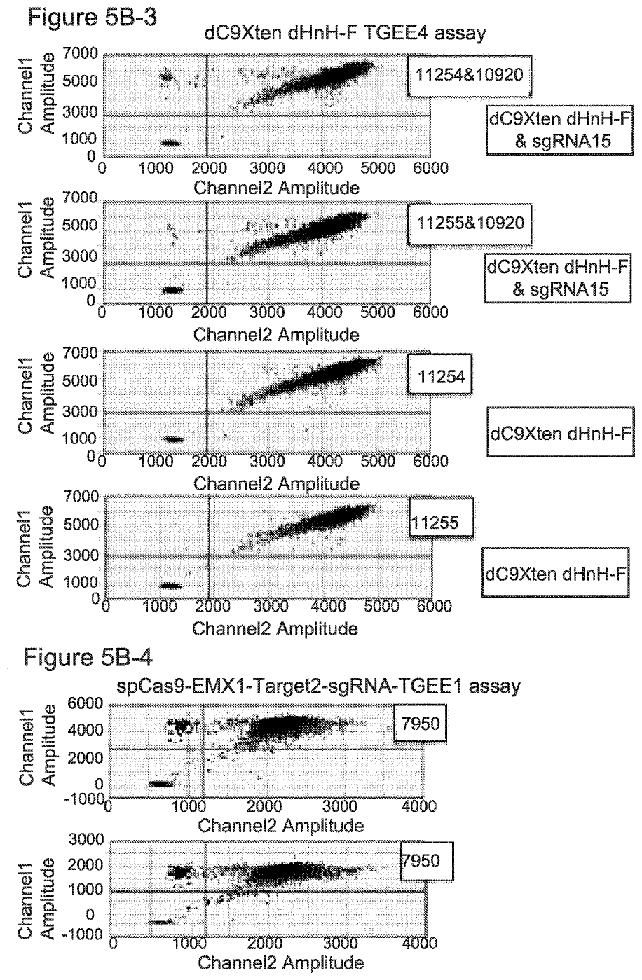

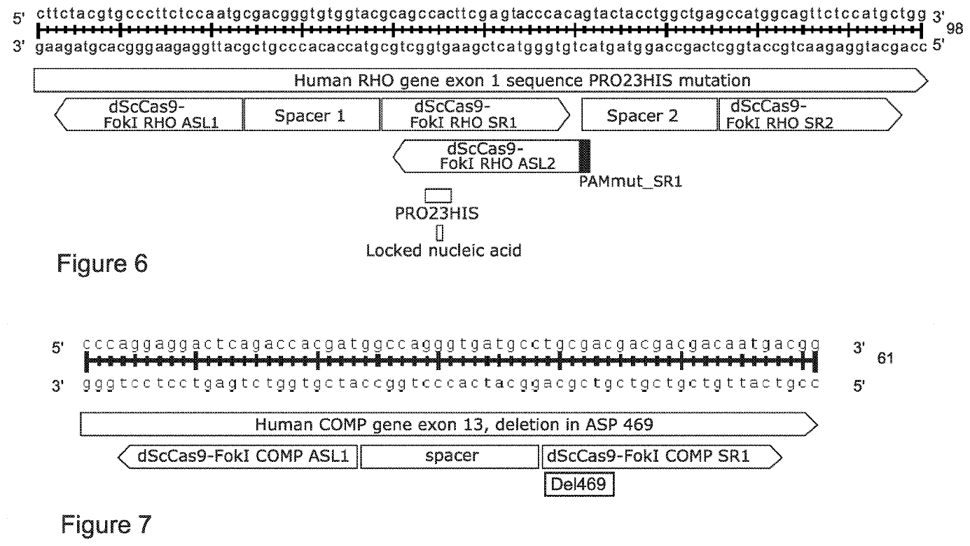

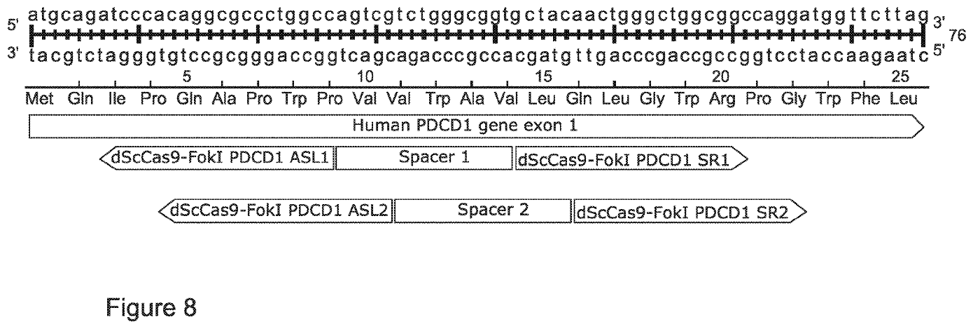

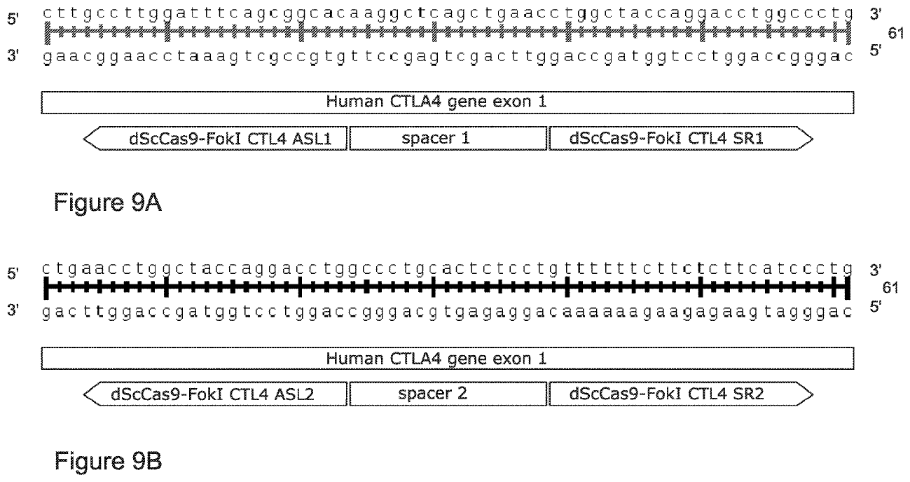

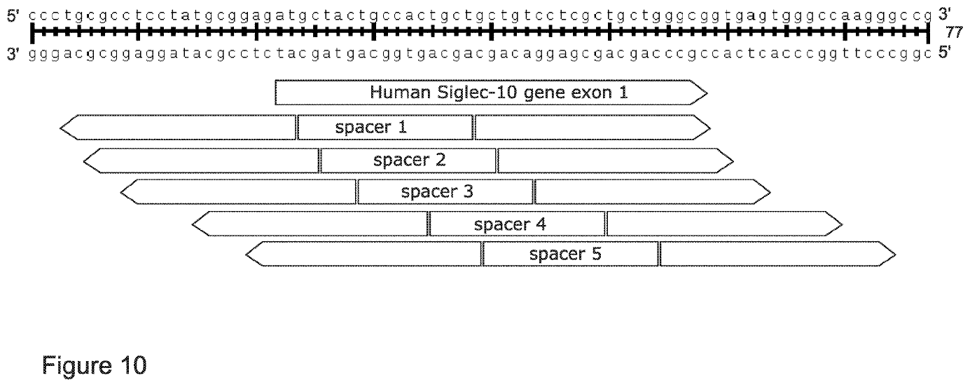

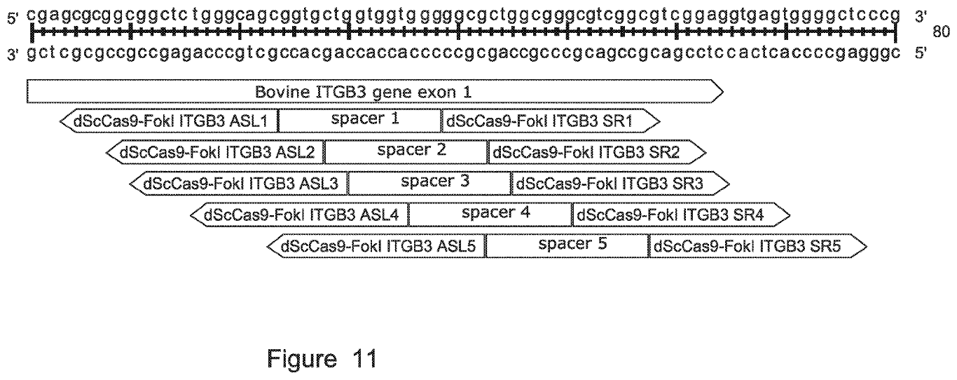

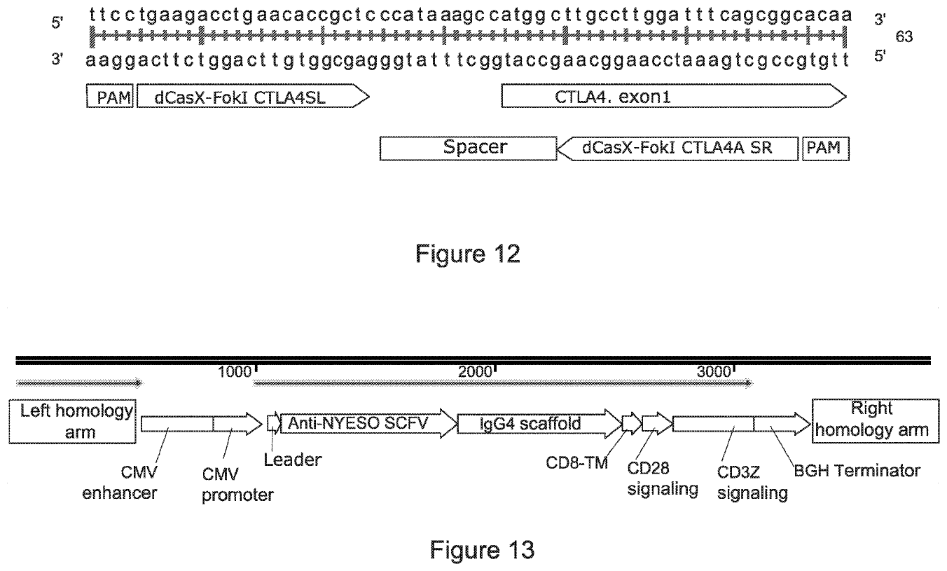

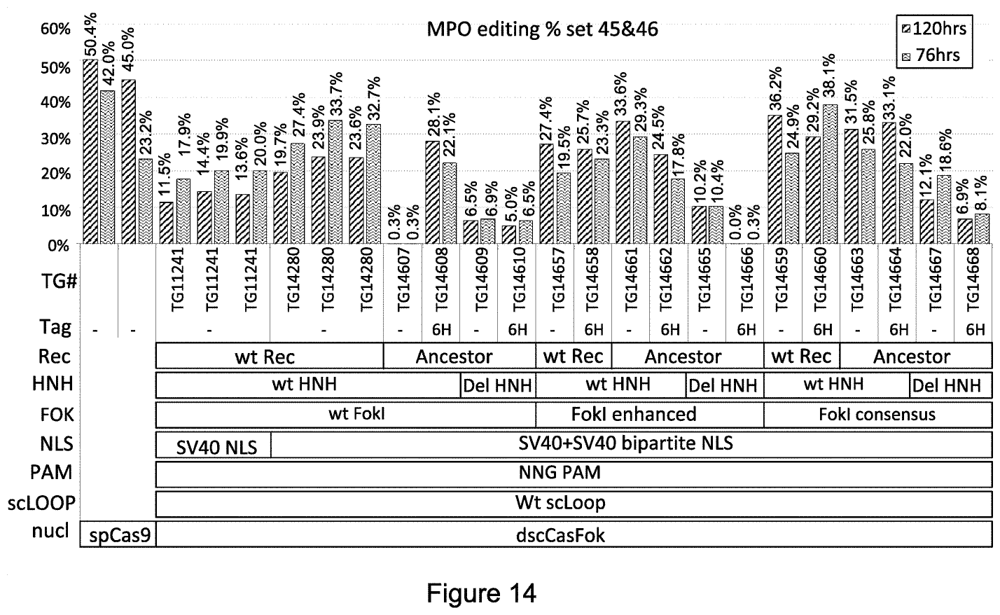

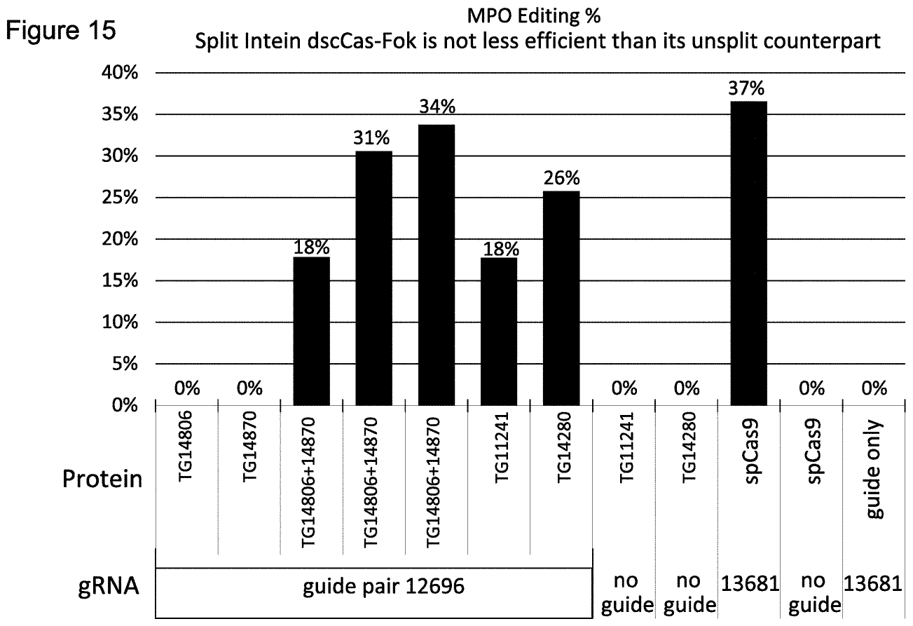

FIG. 5B: Graphs (5B-1, 5B-2, 5B-3, 5B-4) showing raw data for the calculation of FIG. 3A. Threshold crosshairs were placed identically for all treatments as marked in the figure except SpCas9 and which was analyzed with a second assay (NGG target not being available at the NNG target). Top left quarter droplets are erroneous repair products of NHEJ at the intended target site and are droplets containing a single template. Droplets containing more than one template of which one or more are WT are present in the clearly visible “tail” to the right of these droplets.

FIG. 6: Targeting the RHO gene by the editing system of the invention

The figure illustrates the target sequence within exon 1 of the RHO gene of SEQ ID NO. 214, targeted by the SCNA of SEQ ID NOs. 215, 216 of set 1, and of SEQ ID NOs. 217 and 218, of set 2. The 5′ to 3′ upper sequence in the figure is denoted by the nucleic acid sequence SEQ ID NO. 416, the 3′ to 5′ lower sequence in the figure is sequence in the figure is denoted by the nucleic acid sequence SEQ ID NO. 417.

FIG. 7: Targeting the COMP gene by the editing system of the invention

The figure illustrates the target sequence within exon 1 of the COMP gene of SEQ ID NO. 219, targeted by the SCNA of SEQ ID NOs. 220, 221 of set 1. The 5′ to 3′ upper sequence in the figure is denoted by the nucleic acid sequence SEQ ID NO. 418, the 3′ to 5′ lower sequence in the figure is denoted by the nucleic acid sequence SEQ ID NO. 419.

FIG. 8: Targeting the PDCD1 gene by the editing system of the invention

The figure illustrates the target sequence within exon 1 of the PDCD1 gene of SEQ ID NO. 222, targeted by the SCNA of SEQ ID NOs. 223, 224 of set 1, and of SEQ ID NOs. 225 and 226, of set 2. The 5′ to 3′ upper sequence in the figure is denoted by the nucleic acid sequence SEQ ID NO. 420, the 3′ to 5′ lower sequence in the figure is denoted by the nucleic acid sequence SEQ ID NO. 421, the amino acid sequence in the figure is denoted by SEQ ID NO. 422.

FIG. 9A-9B: Targeting the CTLA4 gene by the editing system of the invention

The figure illustrates the target sequence within exon 1 of the CTLA4 gene of SEQ ID NO. 227, targeted by the SCNA of SEQ ID NOs. 228, 229 of set 1 (FIG. 9A), and of SEQ ID NOs. 230 and 231, of set 2 (FIG. 9B). The 5′ to 3′ upper sequence in FIG. 9A is denoted by the nucleic acid sequence SEQ ID NO. 493, the 3′ to 5′ lower sequence in FIG. 9A is denoted by the nucleic acid sequence SEQ ID NO. 494, the 5′ to 3′ upper sequence in the FIG. 9B is denoted by the nucleic acid sequence SEQ ID NO. 495, and the 3′ to 5′ lower sequence in FIG. 9B is denoted by the nucleic acid sequence SEQ ID NO. 496.

FIG. 10: Targeting the SIGLEC10 gene by the editing system of the invention

The figure illustrates the target sequence within exon 1 of the SIGLEC10 gene of SEQ ID NO. 232, targeted by the SCNA of SEQ ID NOs. 233, 234 of set 1, and of SEQ ID NOs. 235 and 236 of set 2, and of SEQ ID NOs. 237 and 238, of set 3, and of SEQ ID NOs. 239 and 240 of set 4, and of SEQ ID NOs. 241 and 242 of set 5. The 5′ to 3′ upper sequence in the figure is denoted by the nucleic acid sequence SEQ ID NO. 497, the 3′ to 5′ lower sequence in the figure is denoted by the nucleic acid sequence SEQ ID NO. 498.

FIG. 11: Targeting the ITGB3 gene by the editing system of the invention

The figure illustrates the target sequence within exon 1 of the ITGB3 gene of SEQ ID NO. 243 targeted by the SCNA of SEQ ID NOs. 244, 245 of set 1, and of SEQ ID NOs. 246 and 247 of set 2, and of SEQ ID NOs. 248 and 249 of set 3, and of SEQ ID NOs. 250 and 251 of set 4, and of SEQ ID NOs. 252 and 253 of set 5. The 5′ to 3′ upper sequence in the figure is denoted by the nucleic acid sequence SEQ ID NO. 499, the 3′ to 5′ lower sequence in the figure is denoted by the nucleic acid sequence SEQ ID NO. 500.

FIG. 12: Targeting the CTLA4 gene by the editing system of the invention

The figure illustrates the target sequence within exon 1 of the CTLA4 gene of SEQ ID NO. 285 targeted by the SCNA of SEQ ID NOs. 286, 287. The 5′ to 3′ upper sequence in the figure is denoted by the nucleic acid sequence SEQ ID NO. 501, the 3′ to 5′ lower sequence in the figure is denoted by the nucleic acid sequence SEQ ID NO. 502.

FIG. 13: CAR-T expression cassette for PDCD-1 exon 1 integration

The figure illustrates the sequence map of the CAR-T cassette SEQ ID NO. 293 for integration within exon 1 of the PDCD-1 gene exon 1 shown in FIG. 8. The cassette is composed of a Left homology arm to the PDCD-1 locus, a CMV enhancer and promoter, an N-terminal leader, an anti-NY ESO scfv, IgG4 scaffold, CD8-TM, CD28 signaling domain, CD3zeta signaling domain, BGH terminator, and Right homology arm to the PDCD-1 locus.

FIG. 14: Comparison of NHEJ erroneous repair percentages in the human Myeloperoxidase (MPO) gene for dScCas9-FokI derivatives

The figure shows comparison of NHEJ erroneous repair percentages in the targeted human Myeloperoxidase (MPO) gene assayed by ddPCR for different dScCas9-FokI derivatives at two timepoints (76 hrs or 120 hrs post transfection) in two separate biological experiments. All constructs except where noted here have an NNG PAM, Wild-type ScCas9 “scLoop”, and a combination of an SV40 and an SV40-derived bipartite Nuclear Localization Signal (NLS). Nuclease (nucl) expressing constructs were delivered by plasmid into Hek293 cells together with a plasmid encoding a pair of SCNAs (sgRNAs) compared to SpCas9 (NGG PAM) with a single sgRNA. Different constructs denoted by their “TG number” (as can be found also in table 18) have different combinations of altered domains. Wt Rec is the dead Rec domain of dscCas9; ancestral Rec (Ancestor) is as described in Example 16; wt HNH is the HNH domain of scCas9; Del HNH is a protein wherein the wt HNH has been deleted as described in Example 2; wt FokI is the FokI-nuclease domain of FokI; FokI enhanced is as described in Example 16; Fold consensus is as described in Example 16. Tag is 6His-Tag (6H) or none (−).

FIG. 15: Split Intein delivery with a reduced PAM dual-RNA guided ribonucleoprotein.

Cells were transfected with double plasmid concentrations of one of each half Intein (TG14806 and TG14870) or with a combination of both together at a similar total concentration of plasmid. An sgRNA pair was delivered by plasmid 12696 targeting Myeloperoxidase (MPO) exon 1. Controls include TG11241, TG12480 and spCas9. ddPCR was done using assay TGEE6 with primers 3261 and 3266. Reference probe was 3289 and drop-off probe was 3291.

### DETAILED DESCRIPTION OF THE INVENTION

The present invention provides a nucleic acid guided genome effector or modifier protein having reduced or abolished Protospacer Adjacent Motif (PAM) restriction or constraint, as well as variants, mutants, fusion proteins and conjugates thereof. It should be noted that the effector or modifier protein of the invention may comprise at least one clustered regularly interspaced short palindromic repeats (CRISPR)-Cas protein or Cas protein derived domain. In some embodiments, the PAM binding domain/PAM recognition motif of the Cas protein of the invention, may be deleted or replaced. These improved Cas proteins or Cas derived domain disclosed by the invention enable reduction and abolishment of Cas PAM requirements, and thereby the provision of highly specific, versatile, effective but specific gene editing systems, compositions and methods.

Thus, in a first aspect, the invention relates to a CRISPR-Cas protein or cas protein derived domain having reduced or abolished Protospacer Adjacent Motif (PAM) constraint or any fragment, variant, mutant, fusion/chimeric protein, complex or conjugate thereof. In more specific embodiments, at least one of the PAM binding domain (PBD) and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

The protospacer adjacent motif (PAM), as used herein, is a short DNA sequence (usually 2-6 base pairs in length) that follows the DNA region targeted for cleavage by the CRISPR system, serving as a binding signal. The PAM is required for a Cas nuclease to cut and is generally found 3-4 nucleotides downstream from the cut site. The canonical PAM (of SpCas9) is the sequence 5′-NGG-3′ where “N” is any nucleobase followed by two guanine (“G”) nucleobases. This short DNA sequence, the PAM, is frequently used to mark proper target sites and discriminate between ‘self’ and ‘non-self’ potential target sequences.

The Cas proteins provided by the invention display reduced or abolished PAM restriction, constraint, requirement or limitation. More specifically, a Cas protein displaying a “PAM-reduced” or “PAM abolished” requirement, restriction, constraint, requirement or limitation as used herein, is considered as a Cas protein having either (a) a less stringent PAM requirement than that of the wild-type PAM requirement of the corresponding wild-type Cas protein; or (b) substitution of a PAM-requiring Cas protein by a less stringent Cas protein or portion thereof. It should be noted that the fewer nucleotides that are required for recognition, the less stringent the PAM, is. In some embodiments, PAM-reduced or abolished Cas protein may require three or less, two or less or one or less nucleotides in a PAM sequence adjacent to the target site. In certain embodiments, PAM-reduced or abolished Cas protein binds the target site recognized by the targeting elements, with no further requirements of specific nucleotides in sequences adjacent to the target site. In some embodiments a PAM -reduced Cas protein, may be a protein that display reduced restriction, constraint, requirement or limitation in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced, inhibited, decreased, eliminated, restriction, constraint, requirement or limitation of about 98% to about 100% as compared to that of the wild-type PAM requirement of the corresponding wild-type Cas protein. More specifically, a protein that display reduced restriction, constraint, requirement or limitation in aboutl%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% , 99.9%, 99.99%, 99.999%, 99.9999% or about 100%, as compared to that of the wild-type PAM requirement of the corresponding wild-type Cas protein. It should be further noted that a “PAM-free” system would have no PAM requirement. Thus, in some embodiments, the invention further provides Cas protein variants that display no PAM requirement, referred to herein as PAM-abolished or PAM-free Cas proteins.

As indicated above, the invention provides Cas protein-derived domains that display PAM-reduced or abolished PAM restriction, constraint, requirement or limitation. In some embodiments such domain may comprise any domain of the Cas protein that maintain the ability of such Cas protein to bind at least one target recognition element (e.g., gRNA) as will be discussed herein after, that guide and direct the Cas protein to a predetermined target nucleic acid sequence. In some embodiments, a cas-protein-derived domain applicable in the present invention may be any protein fragment of Cas comprising between about 50 to 500 amino acid residues or more, that display at least 50-100% homology or identity to at least 50 consecutive amino acid residues of the entire Cas-protein. In some more specific embodiments, the invention provides PAM abolished or reduced CRISPR-Cas proteins that may be any member of a clustered regularly interspaced short palindromic repeat (CRISPR) Class 2 or Class 1 system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target including outside of a bacterium.

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. Partial complementary may mean less than 100% complementarity, for example 80% complementarity,

In some specific embodiment, the PAM abolished or reduced CRISPR-Cas proteins of the invention may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. In more specific embodiments, the nucleic acid guided DNA binding protein nuclease may be CRISPR-associated endonuclease 9 (Cas9) system. The type II CRISPR—Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cast and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene.

Thus, according to such embodiments, the PAM abolished or reduced CRISPR-Cas proteins of the invention is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA” or as a specificity conferring nucleic acid (SCNA).

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR—Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

As discussed herein, in some embodiments, the PAM-reduced or abolished Cas protein of the invention may comprise at least one of, deletion, substitution, mutation or replacement of at least one of, the PAM binding domain (PBD), and/or the PAM recognition motif, or deletion, substitution, mutation or replacement in any fragments thereof, or in any amino acid residue thereof, or in any adjacent amino acid residue/s thereof. As used herein, a “PAM binding domain” or a “PAM binding motif” of the CRISPR-Cas protein of the present disclosure, refers to any amino acid residue or to any sequence, secondary structure and/or three dimensional tertiary structure (formed by either proximate or distant residues) that is involved or participates directly or indirectly in recognition and binding of the PAM in the target nucleic acid sequence. The “PAM binding domain” or a “PAM binding motif” may therefore comprise at least one amino acid residue, a linear peptide composed of two or more residues, any secondary or three dimensional tertiary structure formed by at least two amino acid residues located either in close proximity in a linear sequence or located at distant parts or domain of the protein. In some embodiments, the “PAM binding domain” or a “PAM binding motif” of the CRISPR-Cas protein of the present disclosure may involve residues derived from the N-terminal and/or the C-terminal parts of the proteins forming a structure that participates in PAM binding and recognition. The “PAM binding domain” or the “PAM binding motif” of the CRISPR-Cas protein of the present disclosure may comprise in some embodiments at least one of loop/s, alpha helix/helices, beta sheet/s and any combinations thereof. In some embodiments, the “PAM binding domain” or “PAM binding motif” of the Cas protein used by the present disclosure may comprise at least two loop structures. More specifically, such loops may include in some embodiments a loop referred to herein as the “PAM BD loop” that is comprised within the PAM binding domain of the Cas protein (derived from the C′ terminal part of the protein), and at least one additional structure derived from a distant part of the Cas protein (the N terminal part of the protein), for example, a loop structure referred to herein as the “ScLoop”. In some embodiments, the “PAM binding domain” in accordance with the present disclosure comprises the PAM BD loop. Such domain may comprise residues from about position 1108+/−10 amino acid residues, to about position 1375+/−10 amino acid residues. In some embodiments, the PAM binding domain of ScCas9 may comprise, or may be comprised within residues Glu1108 to Asp1375 of the ScCas9, as denoted by SEQ ID NO. 258. In yet some further specific embodiments, residues Glu1108 to Asp1375 of SEQ ID NO. 258, may comprise the amino acid sequence as denoted by SEQ ID NO. 503, and any variants and homologs thereof. Still further, in some embodiments this domain of Cas may comprise the “PAM binding domain”, that may comprise residues from about position 1228+/−10 amino acid residues, to about position 1343+/−10 amino acid residues. In yet some further embodiments, such sequence may be referred to herein as the “PAM binding domain”, “PAM PBD” “the whole PAM PBD”, “the entire PAM PBD”. In more specific embodiments, the PAM PBD may comprise the sequence of residues Glu1228 to Tyr1343 of the ScCas9 as denoted by SEQ ID NO. 258. In yet some further embodiments, the PAM PBD of ScCas9 may comprise the amino acid sequence as denoted by SEQ ID NO. 504, and any variants and homologs thereof. In yet some further embodiments, the PAM PBD of ScCas9 may comprise the PAM BD loop. In some embodiments, such PAM BD loop may comprise residues from about position 1330+/−10 amino acid residues, to about position 1342+/−10 amino acid residues. In certain embodiments, the PAM BD loop may comprise the sequence of residues Thr1330 to Arg1342 of the ScCas9 as denoted by SEQ ID NO. 258. In some specific embodiments, the PAM BD loop may comprise the sequence as denoted by SEQ ID NO. 505, and any variants and homologs thereof. Still further, in some embodiments the “PAM binding motif” of the invention may comprise a second loop, referred to herein as the “ScLoop”. In more specific embodiments such loop may comprise the amino acid residues from position 367+/−10 amino acid residues, to about position 376+/−10 amino acid residues. In certain embodiments, the “ScLoop” may comprise the sequence of residues Ile367 to Ala376 of the ScCas9 as denoted by SEQ ID NO. 258. In some specific embodiments, the PAM “ScLoop” may comprise the sequence as denoted by SEQ ID NO. 291, and any variants and homologs thereof.

Thus, in some embodiments, the CRISPR-Cas protein of the invention may be at least one of Cas9, CasX, Cas14a1, Cas14b5, CasF-1, CasF-2, CasF-3, an ancestral Cas9 and Cas12a.

In more specific embodiments, the CRISPR-Cas protein of the invention is at least one of Streptococcus canis Cas9 (ScCas9), Streptococcus pyogenes Cas9 (SpCas9), an ancestral Cas9, deltaproteobacteria CasX, Cas12a, Cas14a1, or Cas14b5. In some specific embodiments, at least one PAM-interacting Arginine and/or Lysine residue of the PBD of the Cas proteins is either deleted or replaced by at least one amino acid residue.

In some specific embodiments, the Cas9 of Streptococcus canis may be applicable as the PAM abolished or reduced CRISPR-Cas protein of the invention. In some embodiments, the Cas9 protein may be the protein sequence denoted by SEQ ID NO. 258. In some embodiments, the Cas protein used by the invention is the ScCas9, where the RXR motif of the PAM binding domain of said Cas is deleted or replaced.

Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS, specifically, the SpCas9 of protein id: AAK33936.1, may be applicable as the PAM abolished or reduced CRISPR-Cas protein of the invention. In some embodiments, the Cas9 protein may be the protein sequence as denoted by SEQ ID NO. 257. In yet some further specific embodiments, the RXR motif of the PAM binding domain of spCas9 is deleted or replaced, thereby providing the PAM-reduced or abolished Cas protein of the invention.

In yet some further embodiments, the Streptococcus aureus Cas9 (saCas9) as denoted by SEQ ID NO. 313 protein may be applicable as the PAM abolished or reduced CRISPR-Cas protein of the invention. In more specific embodiments, the RXR motif of the PAM binding domain of said Cas protein is deleted or replaced.

Still further, CasX may be used as the PAM abolished or reduced CRISPR-Cas proteins of the invention. More specifically, CasX was identified by metagenomic analysis of bacteria from groundwater and characterized as an RNA-guided DNA nuclease. It recognizes a 5′-TTCN PAM. It shares no similarity to other reported Cas endonucleases except for a RuvC domain located at the C-terminus. The above features of CasX correlate with those of type V Cas12; however, the size of CasX (-980 aa) is smaller than those of reported Cas12 (˜1200 aa). For example, sgRNA-bound Deltaproteobacteria CasX (DpbCasX) contains 20-nt guide segment and recognizes a TTCN PAM element resulting in dsDNA target cleavage with 10-nt staggered ends. Another example of CasX i.e. Planctomycetes CasX (P1mCasX). In some embodiments, the CasX protein may be the protein sequence as denoted by SEQ ID NO. 269. In some embodiments, wherein the Cas protein used for the PAM-abolished or reduced Cas protein of the invention is CasX, the PAM interacting Lysine of such Cas, is replaced.

In some embodiments, wherein the Cas protein used for the PAM-abolished or reduced Cas protein of the invention is Cas14a1, the PBD of such Cas protein includes residues 1-266, of the amino acid sequence as denoted by SEQ ID NO. 308.

In some embodiments, wherein the Cas protein used for the PAM-abolished or reduced Cas protein of the invention is Cas14b5, the PBD of such Cas protein includes residues 1-300, of the amino acid sequence as denoted by SEQ ID NO. 309.

In some embodiments, wherein the Cas protein used for the PAM-abolished or reduced Cas protein of the invention is Cas12a, the PBD of such Cas protein includes residues 663-761 and 601-642, of the amino acid sequence as denoted by SEQ ID NO. 310 (Cas12a from Francisella tularensis), or residues 536-577 and 599-718 of the amino acid sequence as denoted by SEQ ID NO. 311 (Cas12a from Acidaminococcus sp), or residues 527-568 and 588-678 of the amino acid sequence as denoted by SEQ ID NO. 312 (Cas12a from Lachnospiraceae bacterium).

In yet some further embodiments, the CRISPR-Cas protein used for the PAM-abolished or reduced Cas protein of the invention is or is derived from CasF-1. In some embodiments, this CasF-1 protein may be the protein sequence as denoted by SEQ ID NO. 358.

In yet some further embodiments, the CRISPR-Cas protein used for the PAM-abolished or reduced Cas protein of the invention is or is derived from CasF-2. In some embodiments, this CasF-1 protein may be the protein sequence as denoted by SEQ ID NO. 359.

In yet some further embodiments, the CRISPR-Cas protein used for the PAM-abolished or reduced Cas protein of the invention is or is derived from CasF-3. In some embodiments, this CasF-1 protein may be the protein sequence as denoted by SEQ ID NO. 360.

In yet some further embodiments, the CRISPR-Cas protein used for the PAM-abolished or reduced Cas protein of the invention is or is derived from an ancestral Cas9. In some embodiments, this ancestral Cas9 protein may be the protein sequence as denoted by SEQ ID NO. 268.

It should be noted that any CRISPR/Cas proteins may be used by the invention, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1 , Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJ010A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01 -2, Neisseria meningitides 053442, Neisseria meningitides alpha14, Neisseria meningitides Z2491 , Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

As indicated above, the PAM-binding domain (PBD) of the PAM-reduced or abolished Cas protein of the invention, may be deleted or replaced by at least one other amino acid residue. In some embodiments, the ScCas9 PAM binding domain comprises a loop comprising amino acid residues Thr1330 to Arg1342 of the amino acid sequence as denoted by SEQ ID NO: 258. In more specific embodiments, the PAM-reduced or abolished Cas protein of the invention may comprise the amino acid sequence as denoted by SEQ ID NO. 258, with a deletion or replacement of resides 1330 to 1342 thereof, or any fragment or at least one amino acid residue thereof.

In some further embodiments, the ScCas9 PAM binding domain comprises a helical bundle comprising amino acid residues Glu1228 to Tyr1343 of the amino acid sequence as denoted by SEQ ID NO: 258. In more specific embodiments, the PAM-reduced or abolished Cas protein of the invention may comprise the amino acid sequence as denoted by SEQ ID NO. 258, with a deletion or replacement of resides 1228 to 1343 thereof, or any fragment or at least one amino acid residue thereof.

In some further embodiments, the ScCas9 PAM binding domain comprises amino acid residues Glu1108 to Asp1375 of the amino acid sequence as denoted by SEQ ID NO: 258. In more specific embodiments, the PAM-reduced or abolished Cas protein of the invention may comprise the amino acid sequence as denoted by SEQ ID NO. 258, with a deletion or replacement of resides 1108 to 1375 thereof, or any fragment or at least one amino acid residue thereof.

In yet some further embodiments of the CRISPR-Cas protein of the invention, at least one amino acid residue adjacent to said PBD, may comprise a second loop (Sc loop). In yet some further embodiments, such Sc loop may comprise the amino acid residues Ile367 to Ala376 of ScCas9, as deoted by SEQ ID NO: 291. Specifically, residues Ile367 to Ala376 of ScCas9 that comprises the amino acid sequence as denoted by SEQ ID NO: 258. Thus, in some embodiments, the PAM reduced or abolished Cas protein of the invention may comprise a deletion or replacement of a Second loop (Sc loop) wherein said Sc loop comprises amino acid residues Ile367 to Ala376 of the amino acid sequence as denoted by SEQ ID NO: 258.

In yet some further embodiments, at least one amino acid residue adjacent to said PBD may be replaced and/or deleted. In some embodiments, such residues may comprise at least one of residue Lys1337 and residue Gln1338.

Thus, at least one of: residues Thr1330 to Arg1342, residues Glu1228 to Tyr1343, residues Glu1108 to Asp1375, residues Ile367 to Ala376 and residues Lys1337 and Gln1338, of ScCas9, as specified above, may be replaced or deleted.

In some embodiments, the PAM abolished or reduced ScCas9 of the invention may comprise an amino acid sequence as denoted by SEQ ID NO: 258, with a replacement or deletion of at least one of: residues Thr1330 to Arg1342, residues Glu1228 to Tyr1343, residues Glu1108 to Asp1375, residues Ile367 to Ala376, residue Lys1337 and residue Gln1338.

In yet some alternative embodiments, where the PAM-reduced or abolished Cas protein of the invention is the SpCas9, such PAM-reduced or abolished Cas has a deletion or replacement of the appropriate PAM binding domain, or parts thereof. In some specific embodiments, such SpCas may comprise the amino acid sequence as denoted by SEQ ID NO: 257. More specifically, in some embodiments, the PBD of such Cas comprises a loop comprising amino acid residues Thr1325 to Arg1335 of the amino acid sequence as denoted by SEQ ID NO: 257. Thus, in some embodiments, such PAM-reduced or abolished Cas may comprise the amino acid sequence as denoted by SEQ ID NO: 257, with a deletion or replacement of residues Thr1325 to Arg1335 thereof. In some embodiments, the PBD of such Cas comprises a helical bundle comprising amino acid residues Glu1219 to Tyr1336 of the amino acid sequence as denoted by SEQ ID NO: 257, thus, in some embodiments, such PAM-reduced or abolished Cas may comprise the amino acid sequence as denoted by SEQ ID NO: 257, with a deletion or replacement of residues Glu1219 to Tyr1336 thereof.

In some embodiments, the PBD of such Cas comprises amino acid residues Glu1099 to Gln1368 of the amino acid sequence as denoted by SEQ ID NO: 257, thus, in some embodiments, such PAM-reduced or abolished Cas may comprise the amino acid sequence as denoted by SEQ ID NO: 257, with a deletion or replacement of residues Glu1099 to Gln1368 thereof. The PAM abolished or reduced Cas9 protein of the invention comprises deletion and/or replacement of the PBD, or of any fragments thereof, or of any amino acid residues adjacent to the indicated PDB. As specified above, for ScCas9 for example, comprises in some embodiments of the invention, an amino acid sequence as denoted by SEQ ID NO. 258, with a replacement or deletion of at least one of: residues Thr1330 to Arg1342, residues Glu1228 to Tyr1343, residues Glu1108 to Asp1375, residues Ile367 to Ala376, residue Lys1337 and residue Gln1338. It should be understand however, that the present invention further encompasses PBD sequences that should be replaced or deleted in the PAM reduced or abolished Cas9 protein, that may include one, two three, four, five, six, seven, eight, nine, ten or more amino acid residues N′ and/or C′ of the indicated residues, specifically, one, two, three or more residues. For example, in some embodiments, the PAM abolished or reduced Cas9 protein of the invention comprises deletion and/or replacement of residues Thr1330 to Arg1342 of ScCas9. In such case, it should be understood that the invention encompasses PAM reduced or abolished ScCas9 protein with a deletion or replacement of residues 1330+/−one, two, three or more residues to 1342+/−one, two, three or more residues. More specifically, in some non-limiting embodiments, a deletion or replacement may comprise any one of residues 1327, 1328, 1329, 1330, 1331, 1332 or 1333, to any one of residues 1339, 1340, 1341, 1342, 1343, 1344 or 1345. Similarly, the deletion and/or replacement of at least one of residues Glu1228 to Tyr1343, residues Glu1108 to Asp1375, residues Ile367 to Ala376, further encompasses in some embodiments of the invention, replacement and/or deletion of residues Glu1228+/−one, two, three or more residues to Tyr1343+/−one, two, three or more residues, residues Glu1108+/−one, two, three or more residues to Asp1375+/−one, two, three or more residues, and/or residues Ile367+/−one, two, three or more residues to Ala376+/−one, two, three or more residues. It should be understood that such extension of +/−one, two, three or more residues apply for any PBD or any fragment thereof, of any of the Cas proteins disclosed by the invention.

Still further, as indicated above, in some other embodiments, the PAM abolished or reduced ScCas9 of the invention may comprise different deletions and/or replacements of the Sc loop, and/or Sc loop with one or more point mutations.

In some further embodiments, the PAM abolished or reduced ScCas9 of the invention may comprise an Sc loop with one or more point mutations. In some embodiments, at least one of the Arg residues of the Sc Loop are substituted. In yet some further embodiments, at least one of the Arg residues 370 and 372 of the PAM abolished or reduced ScCas9 of the invention is substituted. In yet some further embodiments, at least one or more of the Arg residues 370 and 372 of SEQ ID NO. 258 are substituted. In some specific embodiments, the PAM abolished or reduced ScCas9 of the invention may comprise two Gln residues that substitute Arg residues 370 and 372 of SEQ ID NO. 258. Such mutated Sc loop is designated herein as the QQ mutant. In yet some further embodiments, the PAM abolished or reduced ScCas9 of the invention may comprise two Ala residues that substitute Arg residues 370 and 372 of SEQ ID NO. 258. Such mutated Sc loop is designated herein as the AA mutant. In yet another embodiment, the PAM abolished or reduced ScCas9 of the invention may comprise a Sc loop with the loop replaced by corresponding residues from SpCas9 proteins in combination with ancestral mutations. In some embodiments, such PAM abolished or reduced ScCas9 may comprise Sc loop sequences derived from SpCas9, replacing the Sc loop (residues 367 to 376) containing region of ScCas9 that in some embodiments may comprise residues 318 to 497 of SEQ ID NO.258, with residues 318 to 487 of SEQ ID NO.257 (SpCas9). In yet some further embodiments the PAM abolished or reduced ScCas9 of the invention may comprise complete deletion of the Sc Loop in combination with ancestral mutations. In yet some further embodiments, the PAM abolished or reduced ScCas9 of the present disclosure may comprise in addition to the replacement, and/or deletion and/or mutations in the Sc loop described herein, any additional deletion, replacement and/or mutation/s in at least one additional domain, for example, as described herein after.

It should be understood that the PAM-reduced or abolished Cas protein of the invention comprises deletion or replacement of at least part of the PBD thereof, specifically, deletion or replacement of at least 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, 98% to about 100%, of the amino acid resides of the PBD thereof and/or any adjacent sequences. Specifically, deletion or replacement of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the amino acid resides of the PBD thereof and/or any adjacent sequences.

In yet some further embodiments, the PAM abolished or reduced CasX of the invention may comprise complete or partial deletion of the PAM binding domain that in some embodiments relates to residues 210-230 and/or residues 513-530 of CasX. In more specific embodiments, CasX comprises the amino acid sequence as denoted by SEQ ID NO. 269. In yet some further embodiments, the PAM abolished or reduced ancestral Cas9 of the invention may comprise complete or partial deletion of the PAM binding domain that in some embodiments relates to residues 1108 to 1375 of ancestral Cas9. In more specific embodiments, ancestral Cas9 comprises the amino acid sequence as denoted by SEQ ID NO. 268.

In yet some further embodiments, the PAM-reduced or abolished Cas protein of the present disclosure may comprise at least one of a deletion, substitution or replacement of the HNH-nuclease domain of said Cas protein or of any fragments and amino acid residues thereof. In some embodiments, the amino acid sequence of the ScCas9 comprise the amino acid sequence as denoted by SEQ ID NO. 506.

It yet some further embodiments, the PAM-reduced or abolished CRISPR-Cas protein of the invention may further comprise at least one Non-Specific DNA Binding Domain (NSBD). In yet some further embodiments, the NSBD may be added to said Cas (either to the N′ and/or C′ terminus thereof) and/or may replaces at least one of the PAM binding domain, and/or the PAM recognition motif, and/or the HNH-nuclease domain, and/or at least one adjacent amino acid residue thereof. In some embodiments, the NSBD lacks an RXR motif.

Non-sequence Specific DNA Binding domains (NSDBs), as used herein are a class of proteins that interact non-specifically with DNA base and backbone elements.

Thus, in some embodiments, the NSBD may be at least one Double-Stranded DNA binding domain or protein (dsDBP), and any variant and fragments thereof. In certain embodiments, the at least one dsDBP of the CRISPR-Cas protein of the invention, is at least one of: at least one Zinc finger (ZF), TAL effector (TALE), Non-specific RVD from AvrBS3 protein family (e.g., NS residues 12,13. Jens Boch, Science 326, Dec 2009), Helix-turn-helix (HTH), SRC Homology 3 (SH3) domain, chromatin-binding domain (CBD) protein and Sticky-C (StkC), domain or protein, and any variant and fragments thereof.

More specifically, Zinc fingers are small protein domains that coordinate one or more zinc ions. Different ZFs can bind to and recognize DNA, RNA or proteins. DNA recognition can occur via sequence-specific and non-specific interactions, which are controlled by amino acids in the ZF-DNA interface (Bulyk, Huang, Choo, & Church, 2001, PNAS, 98:7158-63). Fusion of sequence-specific zinc fingers to functional domains like nucleases has been used to engineer sequence-specific nucleases (Tzfira et al., 2012, Plant Biotechnol J, 10:373-89), and sequence specific transcription-activators (Beerli, Dreier, & Barbas, 2000, PNAS, 97:1495-500). It should be noted however that unlike Zinc finger nucleases where ZF confer specificity to a specific DNA sequence, in some embodiments of the invention, the ZF used provide only a non-specific DNA binding activity.

In some embodiments, the zinc finger applicable by the invention is a 24-residue zinc finger variant which has broad sequence specificity (non-stringent sequence requirement) and enhances non-specific binding to DNA (Chou et al, 2017, PLoS ONE, 12:e0175051). In yet some further specific embodiments, the Cys2His2 finger domains in testis zinc-finger protein may be applicable by the invention. In some particular and non-limiting embodiments, the zinc finger applicable by the invention may comprise the amino acid sequence as denoted by SEQ ID NO. 265, or any fragments, derivatives and variants thereof.

More specifically, RVDs from AvrBS3 protein family are small protein domains that can bind to and recognize specific DNA sequences. DNA recognition can occur via sequence-specific and non-specific interactions, which are controlled by amino acids in the RVD from AvrBS3 protein family-DNA interface (Moscou et al, 2009, Science, 326:1501).

In some embodiments, the RVD from AvrBS3 protein family recognizes all base pairs (Boch et al, 2009, Science, 326:1509-12). In some particular and non-limiting embodiments, the Non-specific RVD from AvrBS3 protein family applicable by the invention may comprise the amino acid sequence as denoted by SEQ ID NO. 404, or any fragments, derivatives and variants thereof. In yet some further embodiments, the Helix-turn-helix (HTH) domain may be used as DBP by the invention. More specifically, the helix-turn-helix domain, as used herein, is comprised of two helices that bind to and recognize DNA, separated by a short turn motif. They may be found in proteins involved in DNA transcription regulation and other activities. The helix-turn-helix domain can include two or more helices, as well as beta sheet domains. For example, the “winged helix-turn-helix” domain comprises a 3-helical bundle followed by a 3-stranded beta sheet.

In yet some further specific embodiments, the HTH applicable in the present disclosure may comprise Lac repressor (Lad) residues 1 to 46, known to fold independently, bind non-specifically, and facilitate diffusion along DNA (Kalodimos et al, 2004, Science 305:386-9). In some particular and non-limiting embodiments, Lac repressor residues 1 to 46, may comprise the amino acid sequence as denoted by SEQ ID NO. 259, or any fragments, derivatives and variants thereof.

Still further, in some further embodiments, at least one SH3 domain may be used as DBP by the invention. More specifically, the five-stranded beta-barrel is a protein motif composed of five beta-strands (also known as a “SRC homology 3 domain” or SH3 domain). In the HIV integrase, a five-stranded beta-barrel mediates non-specific binding to DNA. One beta-barrel motif described here comprises residues 219 to 270 of the HIV integrase protein (Eijkelenboom et al, 1999, Proteins 36:556-64). In some particular and non-limiting embodiments, HIV integrase residues 219 to 270, may comprise the amino acid sequence as denoted by SEQ ID NO. 260, or any fragments, derivatives and variants thereof.

In yet some further embodiments, a SRC Homology 3 (SH3) SH3 domain-like protein applicable in the present invention may comprise the Sso7D DBP from Sulfolobus solfataricus. More specifically, residues 1 to 64, of the Sso7D DBP, which also has been found to mediate non-specific DNA binding interactions (Kalichuk et al, 2016, Scientific Reports 6:37274), may be used as DBP in accordance with the invention. In some particular embodiments, residues 1 to 64 of Sso7D DNA-binding protein may comprise the amino acid sequence as denoted by SEQ ID NO. 261, or any fragments, derivatives and variants thereof.

In certain embodiments, the Sto7D DBP from Sulfolobus tokodaii, may be used as DBP by the present invention. In more specific embodiments, residues 1 to 64 of the Sto7D DBP, may be used, more specifically, residues 1 to 64 that comprise the amino acid sequence as denoted by SEQ ID NO. 262, or any fragments, derivatives and variants thereof.

Still further, in some embodiments, CBDs (chromatin-binding domains), may be used by the invention as DBPs. More specifically, Chromatin is a structure formed by the assembly of DNA and proteins. Chromatin-binding proteins interact with DNA in the context of chromatin and may be involved in forming and regulating the condensed structure, which can govern DNA accessibility to transcription, replication, and other functions. Non-limiting examples for CBDs applicable in the present disclosure include HMGs and StkCs.

In some further specific embodiments, HMGs (high mobility group proteins) may be used as DBPs by the present invention. High mobility group proteins are chromosomal proteins involved in DNA replication, recombination, repair, and transcription. These proteins can bind to and alter chromatin structure, and comprise three families HMGA, HMGB, and HMGN (Reeves, 2010, Biochim Biophys Acta, 1799(1-2):3). For example, the HMGB family are alpha helical protein domains, which can bind to the minor groove of DNA in a non-sequence specific manner, and can bend DNA. Transient interactions of HMGB with DNA may mediate stable interactions of transcription factors with their DNA targets (Agresti & Bianchi, 2003, Curr Opin Genet Dev, 13:170-8).

In some specific embodiments, the HMGB protein used herein may comprise residues 2 to 79 of human HMGB4. In further particular embodiments, such domain may comprise the amino acid sequence as denoted by SEQ ID NO. 264, or any fragments, derivatives and variants thereof.

In some specific embodiments, the HMGN protein used herein may comprise residues 1 to 100 of human HMGN. In further particular embodiments, such domain may comprise the amino acid sequence as denoted by SEQ ID NO. 482, or any fragments, derivatives and variants thereof.

In some specific embodiments, the HMGN protein used herein may comprise residues 1 to 100 of human HMGB1. In further particular embodiments, such domain may comprise the amino acid sequence as denoted by SEQ ID NO. 483, or any fragments, derivatives and variants thereof.

In some specific embodiments, the HMGN protein used herein may comprise residues 1 to 100 of human HMGB3. In further particular embodiments, such domain may comprise the amino acid sequence as denoted by SEQ ID NO. 484, or any fragments, derivatives and variants thereof. HMG proteins have been used to improve Cas9/Cpfl activity in human cells (US patent application 201762531222). While in Cas9/Cpfl the use of HMG domain is for chromatin unfolding and remodeling, in some embodiments of the present invention, HMG domains are used to bind DNA non-specifically as a replacement for PAM-BD.

Still further, in some embodiments, Sticky-C (StkC) may be used as DBPs by the present invention. More specifically, the C-terminal chromatin binding domain of Arabidopsis MBD7 methyl-CpG-binding domain, which allows MBD7 to bind to DNA independently of methylation state (Zemach et al., 2009, Exp Cell Res, 315:3554-62). When fused to other proteins, StkC can improve chromatin binding affinity, without compromising their ability to bind native target sites. In some particular embodiments, the StkC domain used by the present invention is residues 232-305 from MBD7. More specifically, in some embodiments, such domain comprises amino acid sequence as denoted by SEQ ID NO. 263.

Thus, in more specific embodiments, the invention provides at least one PAM-abolished or reduced Cas protein that comprises the following NBDSs that replace at least part of the PBDs thereof, specifically, the ZF domain or protein used for the CRISPR-Cas protein of the invention is at least one Cys₂His₂ zinc-finger domains (TZD) of testis zinc-finger protein, in yet some further embodiments, the HTH domain or protein comprise Lac repressor residues 1 to 46, still further, in some embodiments, the SH3 domain may comprise at least one of: residues 219 to 270 of the human immune deficiency virus (HIV) integrase protein, residues 1 to 64 of the Sso7D DNA-binding protein of Sulfolobus solfataricus and residues 1 to 64 of the Sto7D DNA-binding protein from Sulfolobus tokodaii. In certain embodiments, the StkC domain used for the CRISPR-Cas protein of the invention comprise residues 232-305 of Arabidopsis MBD7 methyl-CpG-binding domain. In yet some further embodiment, the CBD used for the CRISPR-Cas protein of the invention comprises at least one High Mobility Group (HMG) protein, in more specific embodiments, such HMG protein may be any one of HMGA, HMGB and HMGN. In yet some alternative embodiments, the PAM binding domain and/or at least one adjacent amino acid residue of the CRISPR-Cas protein of the invention may be replaced by at least one NSBD, that may be at least one single-strand binding proteins or domains (SSB).

Single-stranded DNA binding proteins (SSBs) are a class of proteins that bind ssDNA in a non-sequence specific fashion and stabilize and protect single strand DNAs in vivo.

In some embodiments, human SSB1 domain 1, may be used by the present invention. In yet some further embodiments, the SSB used in the invention may comprise SEQ ID NO 405, or any fragments, derivatives and variants thereof.

In some embodiments, the CRISPR-Cas protein of the invention may be a Cas mutant or variant. It should be understood that such mutation or variation is according to some embodiments of the invention, in addition to the deletion of the PBD of the Cas protein or replacement thereof with at least one NSBD as discussed above. “Mutant” or “variant” as used herein, refers to a Cas protein encoded by a sequence comprising at least one mutation, or in which at least a portion of the functionality of the sequence has been lost, or changed. As used herein, the term “mutation,” refers to any change in a nucleic acid sequence that may arise from at least one of, a deletion, addition, substitution, or rearrangement of at least one nucleotide in the mutated sequence. The mutation may also affect one or more properties of the proteins and/or steps that the sequence is involved in. For example, a change in a DNA sequence may lead to the synthesis of an altered mRNA and/or a protein that is active, partially active, inactive, or displaying at least one altered property, specifically, stability, bioavailability, solubility, size and the like.

Thus, in some specific embodiments, such mutant or variant may be a Cas protein having altered activity, stability, specificity, solubility, bioavailability, size or any other altered functional and/or structural property. In some embodiments, such Cas protein may be a Cas protein having reduced or abolished nucleolytic activity. In yet some further embodiments, such Cas protein may have a reduced size. In certain embodiments, the Cas mutant or variant of the invention may comprise at least one of: (a) at least one point mutation substituting aspartic acid residue at position 10 to alanine (D10A) and/or at least one point mutation substituting histidine residue 849 to alanine (H849A). This Cas mutant is devoid of nucleolytic activity and is also referred to herein as a dead Cas, or “dCas”. It should be however noted that any other additional or alternative mutation or substitution that results in a dead Cas is encompassed by the present disclosure. Still further, in some embodiments, the Cas of the present disclosure may comprise at least one of; (b) at least one deletion, substitution and/or replacement of at least one of: (i) the HNH-nuclease domain or any fragment thereof, and/or at least one amino acid residue thereof; (ii) the REC1/2 domain or any fragments thereof and/or at least one amino acid residue thereof; (iii) the FLEX domain, or any fragments thereof and/or at least one amino acid residue thereof; (iv) the RUVC domain or any fragments thereof and/or at least one amino acid residue thereof, and (v) any combinations of (i), (ii), (iii), and (iv); and (c) at least one mutation in at least one residue of at least one of (i) the HNH-nuclease domain or any fragment thereof; (ii) the REC1/2 domain or any fragments thereof; (iii) the FLEX domain, or any fragments thereof; (iv) the RUVC domain or any fragments thereof; and (v) and any combinations of (i), (ii), (iii), and (iv). Still further, in some embodiments, the PAM-reduced or abolished Cas protein of the invention may comprise a deletion in at least one of (i), (ii), (iii), and (iv) or any combinations thereof and additionally, at least one mutation in at least one amino acid residue comprised within the PBD of the ScCas9. Non-limiting embodiments for such mutations may comprise the QQ mutant that may comprise two Gln residues that substitute Arg residues 370 and 372 of the ScCas9 as denoted by SEQ ID NO. 258. In yet some further embodiments, the PAM abolished or reduced ScCas9 of the invention may comprise the AA mutant, that comprise two Ala residues that substitute Arg residues 370 and 372 of SEQ ID NO. 258. As shown by Examples 2 and 3, the PAM abolished or reduced ScCas9 of the present disclosure may comprise Sc loop QQ or AA mutations, or alternatively, Sc loop depletion or replacement thereof with at least one dsDBP or SSB as discussed above, combined with deletion of HNH, and replacement of the RuvC and Recl and Rec2 domains with ancestral versions.

More specifically, in some embodiments, the PAM-reduced or abolished Cas protein of the invention may be a defective Cas mutant that lacks any nucleolytic activity. More specifically, as discussed previously, Cas9 generates double strand breaks (DSBs) through the combined activity of two nuclease domains, RuvC and HNH. The exact amino acid residues within each nuclease domain that are critical for endonuclease activity are D10A for HNH and H840A for RuvC (in S. pyogenes Cas9) and DlOA for HNH and H849A for RuvC (in S. canis Cas9). It should be therefore noted that the invention further encompasses modified versions of the Cas9 enzyme containing only one active catalytic domain (called “Cas9 nickase”). Cas9 nickases still bind DNA based on gRNA specificity, but nickases are only capable of cutting one of the DNA strands, resulting in a “nick”, or single strand break, instead of a DSB.

In some embodiments, the PAM-reduced or abolished Cas protein of the invention may further comprise at least one substitution in residues corresponding to of D10A and H849A of ScCas9. Still further, as illustrated in Example 3, in an attempt to reduce the size of the PAM-reduced or abolished Cas protein of the invention, several variants having deletions of certain domain of Cas or fragments thereof, were generated. Thus, in some embodiments, the PAM-reduced or abolished Cas protein of the invention may further comprise a deletion of the HNH-nuclease domain or any fragment thereof of residues corresponding to, specifically, of residues 793-914 of ScCas9 (the HNH domain is denoted by SEQ ID NO. 506). In yet some further embodiments, the PAM-reduced or abolished Cas protein of the invention may further comprise a deletion of the REC2 domain or any fragments thereof corresponding to, specifically, of residues 180-308 of ScCas9. In some further embodiments, the PAM-reduced or abolished Cas protein of the invention may further comprise a deletion of the FLEX domain or any fragments thereof corresponding to, specifically, of residues 1012-1079 of ScCas. Still further, in some embodiments, the PAM-reduced or abolished Cas protein of the invention may further comprise a deletion of the RUVC domain or any fragments thereof of residues corresponding to, specifically, of residues 1-60, 728-784, and 918-1108 of ScCas. It should be understood that the different residues of the various domains that may be deleted or replaced in the PAM reduced or abolished Cas protein of the invention, specifically, at least one of HNH-nuclease domain, REC2 domain, RUVC domain, FLEX domain, as specified herein refer to ScCas9, specifically, the ScCas9 that comprises the amino acid sequence as denoted by SEQ ID NO. 258. However, the invention encompasses the use of any Cas protein, and specifically, any of the Cas proteins disclosed by the invention, and therefore, the corresponding HNH-nuclease domain, REC2 domain, RUVC domain, FLEX domain, that comprise amino acid sequences that correspond to those specified for ScCas herein above, may be deleted and/or replaced by in each of the PAM reduced or abolished Cas protein of the invention. It should be understood that in certain embodiments, the PAM-reduced or abolished Cas protein of the invention may comprise in addition to the deletion and/or replacement of at least part of the PBD thereof, any additional mutation, deletion or insertion, specifically, as discussed above, provided that such PAM-reduced or abolished Cas protein of the invention still retains, at least in part, the ability of binding and being guided by at least one target recognition element.

Thus, according to some embodiments, the CRISPR-Cas protein of the invention or any variant, mutant, fusion/chimeric protein, complex or conjugate thereof, is capable of binding at least one target recognition element. As used herein a “target recognition element” is a nucleic acid sequence (either RNA or DNA or a modified nucleic acid or a combination thereof) that directs the PAM abolished or reduced Cas protein of the invention or any chimera/fusion, conjugate or complex thereof to the desired target site in a target nucleic acid sequence. Thus, the target recognition element targets the nucleic acid-modifier or effector component attached/fused, complexed or conjugated to the PAM-reduced or abolished Cas protein of the invention (forming the chimeric protein, conjugate or complex of the invention) to the target site. In more specific embodiments, such at least one target recognition element may be or may comprise at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids, for example, DNA:RNA hybrid stem-loop, or combinations thereof.

In yet a further aspect, the invention provides a nucleic acid guided genome modifier or effector chimeric or fusion protein, or any modifier/effector chimera or conjugate thereof. More specifically, nucleic acid guided genome modifier/effector chimeric or fusion protein, or conjugate or complex of the invention comprises at least two essential elements:

The first component (a), is at least one Cas protein, or any Cas protein derived domain, having reduced or abolished PAM constraint or any fragment, variant, or mutant thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

The second component (b), is at least one nucleic acid modifier or effector component. As indicated above, these two components (a) and (b), are provided by the invention as a fusion or chimeric protein or alternatively, as a conjugate or complex, that display a nucleic acid guided effector/modifier function on a target nucleic acid sequence of interest.

As will be further elaborated herein after, the nucleic acid guided genome modifier or effector chimeric protein, complex or conjugate of the invention is capable of modifying (either physically, and/or functionally), a target nucleic acid sequence, for example, a target sequence in at least one of a chromosomal, mitochondrial DNA, or DNA of any cellular organelle, for example, chloroplast, amyloplast and chromoplast, or any other extra-chromosomal nucleic acid molecule (e.g., any genetic element such as plasmids and viral nucleic acid sequences). It should be further noted that the nucleic acid guided genome modifier or effector chimeric protein of the invention is capable of binding, or being guided or directed to the specific target sequence by a nucleic acid target recognition element that provides the specificity and binding capabilities of the modifier chimera of the invention to the target nucleic acid through base-pairing of the target recognition element and a target nucleic acid. In some embodiments, the modification on the target sequence may include, but is not limited to: mutation, deletion, insertion, replacement, binding, digestion, nicking, methylation, acetylation, ligation, recombination, helix unwinding, chemical modification, labeling, activation, and inactivation or any combinations thereof, as well as any editing activity (e.g., mutation, substitution, replacement, deletion or insertion of at least a part of the target sequence). Still further, the target nucleic acid functional modification may lead to, but is not limited to: changes in transcriptional activation, transcriptional inactivation, alternative splicing, chromatin rearrangement, pathogen inactivation, virus inactivation, change in cellular localization, compartmentalization of nucleic acid, changes in stability, and the like, or combinations thereof.

In some embodiments, the Cas protein used for the nucleic acid guided genome modifier or effector chimeric protein, complex or conjugate of the invention is at least one of Cas9, CasX, Cas12al, Cas14a1, CasF-1, CasF-2, CasF-3, an ancestral Cas and Cas14b5.

In more specific embodiments, the Cas protein used for the nucleic acid guided genome modifier or effector chimeric protein of the invention may be at least one of ScCas9, SpCas9, CasF-1, CasF-2, CasF-3, ancestral Cas9, Cas12a, Cas14a1, Cas14b5, and deltaproteobacteria CasX. In yet some further specific embodiments, at least one PAM interacting Arginine and/or lysine residue of the PBD of such Cas protein is deleted, substituted or replaced.

Still further, in some alternative embodiments, the Cas used as a component of the nucleic acid guided genome modifier or effector chimeric protein, complex or conjugate of the invention may be ScCas9. In more specific embodiments, the ScCas9 may comprise an amino acid sequence as denoted by SEQ ID NO. 258, with a replacement or deletion of at least one of: residues Thr1330 to Arg1342, residues Glu1228 to Tyr1343, residues Glu1108 to Asp1375, residues Ile367 to Ala376, at least one of residue Lys1337 and residue Gln1338. As indicated herein before in connection with other aspects of the invention, the particular residues defining the PBD of the Cas protein of the invention, specifically, the ScCas, may start at least one, two, three or more residues N′ or C′ to the specified starting residue, and/or end at least one, two, three or more residues C′ or N′ to the specified end residue. For example, in case of residues Thr1330 to Arg1342, the deleted or replaced sequence may comprise a sequence stating at any one of residues 1327, 1328, 1329, 1330, 1331, 1332 or 1333 of ScCas, and ends at any one of residues 1339, 1340, 1341, 1342, 1343, 1344 or 1345.

In yet some further embodiments, the PAM-reduced or abolished CRISPR-Cas protein of the invention may further comprise at least one Non-Specific DNA Binding Domain (NSBD). In yet some further embodiments, the NSBD may be added to said Cas (either to the N′ and/or C′ terminus thereof) and/or may replace at least one of the PAM binding domain, and/or the PAM recognition motif, and/or the HNH-nuclease domain, and/or at least one adjacent amino acid residue thereof. In more specific embodiments, such NSBD may be at least one dsDBP, and any variant and fragments thereof.

In some particular embodiments, the at least one dsDBP used to replace the PAM binding domain of the Cas protein used by the nucleic acid guided genome modifier or effector chimeric protein of the invention, may be at least one of: at least one ZF, HTH, SH3 domain, Non-specific RVD from AvrBS3 protein family, a CBD protein and StkC, domain or protein, and any variant and fragments thereof.

In certain embodiments, the ZF domain or protein may be at least one Cys₂His₂ TZD. In some further embodiments, the HTH domain or protein comprise Lac repressor (also referred to herein as Lad) residues 1 to 46. In some embodiments, the SH3 domain comprise at least one of: residues 219 to 270 of HIV integrase, protein residues 1 to 64 of the Sso7D DNA-binding protein of Sulfolobus solfataricus, and residues 1 to 64 of the Sto7D DNA-binding protein from Sulfolobus tokodaii. In certain embodiments, the StkC domain comprises residues 232-305 of Arabidopsis MBD7 methyl-CpG-binding domain. Still further, in some embodiments, the CBD comprises at least one High Mobility Group (HMG) protein. More specifically, such HMG protein may be any one of HMGA, HMGB and HMGN.

In more specific embodiments, the Cys₂His₂ of Testis zinc finger 3 (TZD) used herein may comprise the amino acid sequence as denoted by SEQ ID NO. 265. In yet some further embodiments, the Lac repressor residues 1 to 46, that may be applicable in the present invention, may comprise the amino acid sequence as denoted by SEQ ID NO. 259. In certain embodiments, the SH3 domain comprising at least one of: residues 219 to 270 of HIV integrase protein, that may be applicable in the present invention, may comprise the amino acid sequence as denoted by SEQ ID NO. 260. Still further, in some embodiments, residues 1 to 64 of the Sso7D DNA-binding protein of Sulfolobus solfataricus, that may be applicable in the present invention, may comprise the amino acid sequence as denoted by SEQ ID NO. 261. In some embodiments, residues 1 to 64 of the Sto7D DNA-binding protein from Sulfolobus tokodaii, that may be applicable in the present invention, may comprise the amino acid sequence as denoted by SEQ ID NO. 262. In some further embodiments, StkC domain that comprise residues 232-305 of Arabidopsis MBD7 methyl-CpG-binding domain and may be applicable in the present invention, may comprise the amino acid sequence as denoted by SEQ ID NO. 263. In yet some further embodiments, CBD that comprises at least one High Mobility Group (HMG) protein, and may be applicable in the present invention, may comprise the amino acid sequence as denoted by SEQ ID NO. 264.

In some alternative or additional embodiments, at least one SSB may be used to replace the PAM binding domain of the Cas protein of the nucleic acid guided genome modifier or effector chimeric protein of the present disclosure. In some specific embodiments, the SSB used may comprise the amino acid sequence as denoted by SEQ ID NO. 405.

In certain embodiments, the Cas protein used as one of the components of the nucleic acid guided genome modifier or effector chimeric protein of the invention may be a Cas mutant or variant. In some specific embodiments, such mutant or variant may be a Cas protein having altered activity, stability, specificity, solubility, size or any other altered functional and/or structural property. In some embodiments, such Cas protein may be a Cas protein having reduced or abolished nucleolytic activity. In yet some further embodiments, such Cas protein may have a reduced size. In more specific embodiments, such mutant or variant further comprises in addition to deletion, substitution, mutation or replacement of the PBD or parts thereof, at least one of: (a) at least one point mutation substituting aspartic acid residue at position 10 to alanine (D10A) and/or at least one point mutation substituting histidine residue 849 to alanine (H849A). Still further, the Cas may further or alternatively comprise at least one of (b) at least one deletion and/or substitution, and/or mutation and/or replacement of at least one of: (i) the HNH-nuclease domain or any fragment thereof and/or at least one amino acid residue thereof; (ii) the REC2 domain or any fragments thereof and/or at least one amino acid residue thereof; (iii) the FLEX domain or any fragments thereof and/or at least one amino acid residue thereof; (iv) the RUVC domain or any fragments thereof and/or at least one amino acid residue thereof. And (v), any combinations of (i), (ii), (iii), and (iv); and (c) at least one mutation in at least one residue of at least one of (i) the HNH-nuclease domain or any fragment thereof; (ii) the REC2 domain or any fragments thereof; (iii) the FLEX domain, or any fragments thereof; (iv) the RUVC domain or any fragments thereof; and (v) any combinations of (i), (ii), (iii), and (iv). Still further, in some embodiments, the PAM-reduced or abolished Cas protein of the invention may comprise a deletion in at least one of (i), (ii), (iii), and (iv) or any combinations thereof and additionally, at least one mutation in at least one amino acid residue comprised within the PBD of the ScCas9. Non-limiting embodiments for such mutations may comprise the QQ mutant that may comprise two Gln residues that substitute Arg residues 370 and 372 of the ScCas9 as denoted by SEQ ID NO. 258. In yet some further embodiments, the PAM abolished or reduced ScCas9 of the invention may comprise the AA mutant, that comprise two Ala residues that substitute Arg residues 370 and 372 of SEQ ID NO. 258. As shown by Examples 2 and 3, the PAM abolished or reduced ScCas9 of the present disclosure may comprise Sc loop QQ or AA mutations, or alternatively, Sc loop depletion or replacement thereof with at least one dsDBP or SSB as discussed above, combined with deletion of HNH, and replacement of the RuvC and Recl and Rec2 domains with ancestral versions.

As indicated above, in more specific embodiments, the PAM-reduced or abolished Cas protein used as one of the components of the nucleic acid guided genome modifier or effector chimeric protein of the invention, may be a defective CRISPR-Cas protein, specifically, dCas protein that is a Cas protein devoid of a nucleolytic activity. Such mutant, in addition to the deletion or replacement of the PBD or any parts thereof, also carries at least one mutation that abolishes, or at least reduces its nucleolytic activity.

Thus, in some alternative embodiments, PAM-reduced or abolished Cas protein of the invention or any chimera or conjugate thereof, may be a defective nuclease, or defective enzyme. A defective enzyme (e.g., a defective mutant, variant or fragment) may relate to an enzyme that displays an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100%, as compared to the wild type active nuclease. More specifically, an enzyme that displays an activity reduced in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% , 99.9%, 99.99%, 99.999%, 99.9999% or about 100%, as compared to the wild type active nuclease.

In some specific and non-limiting embodiments, the PAM-reduced or abolished Cas protein used for the effector/modifier chimera or conjugate of the invention, may be a defective Cas that carries at least one substitution, specifically, at least one of D10A and H849A.

In yet some further embodiments, the PAM-reduced or abolished Cas protein used for the effector/modifier chimera or conjugate of the invention, may be a Cas protein that has a deletion in the HNH-nuclease domain or in any fragment thereof. In some further embodiments, the PAM-reduced or abolished Cas protein used for the effector/modifier chimera or conjugate of the invention, may be a Cas protein that has a deletion in the REC2 domain or in any fragments thereof. In some further embodiments, the PAM-reduced or abolished Cas protein used for the effector/modifier chimera or conjugate of the invention, may be a Cas protein that has a deletion in the FLEX domain (for example, as denoted by SEQ ID NO. 292), or in any fragments thereof. In some further embodiments, the PAM-reduced or abolished Cas protein used for the effector/modifier chimera or conjugate of the invention, may be a Cas protein that has a deletion in the RUVC domain or in any fragments thereof.

In certain embodiments, the Cas protein used as one of the main components in the nucleic acid guided genome modifier or effector chimeric protein of the invention, is capable of binding at least one target recognition element. It should be understood that the nucleic acid guided genome modifier/effector chimeric protein, complex or conjugate of the invention is capable of binding or being associated with the target recognition element, for example, by any one of, affinity of non-covalent bonds such electrostatic interactions (salt bridges), dipolar interactions (hydrogen bonding, H-bonds), entropic effects (hydrophobic interactions) and dispersion forces (base stacking). As indicated above, the genome modifier/effector chimeric protein, complex or conjugate of the invention is a nucleic acid guided effector, that is capable of binding to at least one target recognition element that guides or directs the effector to the specific target nucleic acid sequence.

In more specific embodiments, such at least one target recognition element is at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids, for example, DNA:RNA hybrid stem-loop, or combinations thereof. In some further specific embodiments, the target recognition element of the invention is a guide RNA (gRNA), such gRNA in accordance with some embodiments may include split-gRNA (i.e. separate or annealed tracer and target-specific RNAs) and single-gRNA form.

The invention provides a nucleic acid guided genome effector/modifier chimeric/fusion protein, complex or conjugate that comprise the PAM abolished or reduced Cas protein of the invention and at least one nucleic acid modifier component. As indicated above, the second component of the nucleic acid guided genome modifier chimeric protein of the invention is a nucleic acid modifier or effector component. In some embodiments, such effector or modifier component may be a protein-based modifier, a nucleic acid-based modifier or any combinations thereof. In some embodiments, “the nucleic acid modifier or effector” component may be any component, element or specifically protein, polypeptide or nucleic acid sequence or oligonucleotide that upon direct or indirect interaction with a target nucleic acid sequence, modify or modulate the structure, function (e.g., expression), or stability thereof. Such modification may include the modification of at least one functional group, addition or deletion of at least one chemical group by modifying an existing functional group or introducing a new one such as methyl group. The modifications may include cleavage, methylation, demethylation, deamination and the like. Specific modifier component applicable in the present invention may include but are not limited to a protein-based modifier, for example, a nuclease, a methyltransferase, a methylated DNA binding factor, a transcription factor, transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, any combinations thereof or any fusion proteins comprising at least one of the modifier proteins disclosed by the invention.

As will be elaborated herein below, “activity” of the nucleic acids modifier or effector component in the nucleic acid guided genome modifier/effector chimeric protein, complex or conjugate of the invention, referred to herein may relate in some embodiments to any modification performed in any nucleic acid molecule or sequence, for example, any sequence encoding a product, or alternatively any non-coding sequences. Such modification in some embodiments may result (specifically in case performed on a coding sequence, or alternatively in a regulatory non-coding sequence), in modulation of the expression, stability or activity of the encoded product. Non-limiting examples for such modification may be nucleolytic distraction, methylation, demethylation, acetylation and the like. In some specific embodiments, such nucleic acid modifier protein may be a nuclease, and the activity referred to herein may be the nucleolytic activity of the nuclease. However, in some alternative embodiments, the invention further encompasses other activities that do not relate to nucleolytic activity.

“Modulation” as used herein means a perturbation of function and/or activity, stability and/or structure. In certain embodiments, modulation means an increase in gene expression. In certain embodiments, modulation means a decrease in gene expression. In yet some further embodiments, modulation may further include editing functions (specifically, deletion, insertion, mutations, substitutions or replacement) performed by the modifier or effector of the invention on a target nucleic acid sequence.

Thus, target nucleic acid modification performed by the modifier or effector component of the nucleic acid guided genome modifier/effector chimeric protein, complex or conjugate of the invention may include, but is not limited to: mutation, deletion, insertion, replacement, binding, digestion, nicking, methylation, acetylation, ligation, recombination, helix unwinding, chemical modification, labeling, activation, and inactivation or any combinations thereof. Target nucleic acid functional modification may lead to, but is not limited to: changes in transcriptional activation, transcriptional inactivation, alternative splicing, chromatin rearrangement, pathogen inactivation, virus inactivation, change in cellular localization, compartmentalization of nucleic acid, changes in stability, and the like, any editing activity (e.g., mutation, substitution, replacement, deletion or insertion of at least a part of the target sequence), or combinations thereof.

In yet some particular and non-limiting embodiments, the effector/modifier component of the nucleic acid guided genome modifier/effector chimeric protein, complex or conjugate of the invention may be any of the proteins indicated above, with the proviso that such effector is not a recombinase.

In some specific embodiments, the nucleic acid modifier or effector component of the nucleic acid guided genome modifier or effector chimeric protein of the invention, may be at least one nuclease. More specifically, as used herein, the term “nuclease” refers to an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. The nucleases belong just like phosphodiesterase, lipase and phosphatase to the esterases, a subgroup of the hydrolases. This subgroup includes the Exonucleases which are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly (A)-specific 3′ to 5′ exonuclease. Members of this family include Exodeoxyribonucleases producing 5′-phosphomonoes ters, Exoribonucleases producing 5′-phosphomonoesters, Exoribonucleases producing 3′-phosphomonoesters and Exonucleases active with either ribo-or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above.

In more specific embodiments, such nuclease may be a Type IIS restriction endonuclease or any fragment, variant, mutant, fusion protein or conjugate thereof.

A restriction enzyme is an embodiment for endonuclease that cleaves DNA into fragments at or near its specific recognition sites within the molecule. To cut DNA, most restriction enzymes make two incisions, through each sugar-phosphate backbone (i.e. each strand) of the DNA double helix. In some embodiments, Type IIS restriction enzymes recognize asymmetric DNA sequences and cleave outside of their recognition sequence, which can be removed, and can thus be used. Non-limiting examples of such restriction enzymes may include, but are not limited to FokI, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, HgaI, HphI, HpyAV, MboII, MlyI, MmeI, MnlI, NmeAIII, PleI, Sapl, SfaNI, and I-TEVI.

In some specific embodiments, the nuclease used as the effector/modifier component in the chimeric modifier of the invention may be at least one type IIS nuclease or any cleavage domains thereof. These may include cleavage domains from Type IIS restriction endonucleases: AarI, Acc36I, AceIII, AclWI, AcuI, AjuI, Alol, AlwI, Alw26I, AmaCSI, ApyPI, AquII, AquIII, AquIV, ArsI, AsuHPI, BaeI, BarI, Bbr7I, BbsI, BbvI, BbvII, Bbv16II, BccI, BccI, Bce83I, BceAI, BceSIII, BceSIV, BcefI, BcgI, BciVI, Bco5I, Bco116I, BcoDI, BcoKI, BfiI, BfuI, BfuAI, BinI, Bli736I, Bme585I, BmrI, BmsI, BmuI, BpiI, BpmI, BpuAI, BpuEI, BpuSI, BsaI, BsaXI, BsbI, Bsc91I, BscAI, BseKI, BseMI, BseMII, BseRI, BseXI, BseZI, BsgI, BslFI, BsmAI, BsmBI, BsmFI, Bso31I, BsoMAI, Bsp423I, BspCNI, BspD6I, BspIS4I, BspKT5I, BspLU11III, BspQI, BspST5I, BspTNI, BspTS514I, Bst6I, Bst12I, Bst19I, Bst71I, BstBS32I, BstFZ438I, BstGZ53I, BstH9I, BstMAI, BstOZ616I, Bst31TI, BstTS5I, BstV1I, BstV2I, BsuI, Bsu6I, Bsu537I, BtgZI, BtsI, BtsIMutI, BtsCI, Bvel, Bve1B23I, CatHI, CchII, CchIII, Cco14983III, CdpI, CjeI, CjeF38011III, CjeIAIII, CjeNII, CjeNIII, CjePI, CjeP659IV, CjeYHOO2IV, CjuII, CseI, CspCI, CstMI, DraRI, DrdIV, EacI, Eam1104I, EarI, EciI, Eco31I, Eco57I, FaqI, FauI, Fph2801I, GeoICI, GsuI, HgaI, Hin4I, Hin4II, HphI, Hpy99XXII, HpyAV, HpyC1I, Ksp632I, LguI, Lsp1109I, LweI, MaqI, MboII, Mcr10I, MlyI, MmeI, MnlI, NcuI, NgoAVII, NgoAVIII, NlaCI, NmeAIII, NmeA6CIII, PciSI, PcoI, PhaI, PlaDI, PheI, PpiI, PpsI, PspOMII, PspPRI, PsrI, RceI, RdeGBII, RlaII, RleAI, RpaI, RpaBI, RpaB5I, Rtr1953I, SapI, SchI, SdeAI, SdeOSI, SfaNI, SmuI, SspD5I, SstE37I, Sth132I, StsI, TaqII, TaqIII, TsoI, TspDTI, TspGWI, TstI, Tth111II, TthHB27I, UbaF9I, UbaF11I, UbaF12I, UbaF13I, UbaF14I, Vga43942II, VpaK32I, WviI.

Still further, in some embodiments, the Type IIS restriction endonuclease used as the modifier component in the nucleic acid guided genome modifier/effector chimeric protein, of the invention, may be FokI or any fragment, variant, mutant, fusion protein or conjugate thereof. The enzyme FokI (Fok-1), naturally found in Flavobacterium okeanokoites, is a bacterial type IIS restriction endonuclease consisting of an N-terminal DNA-binding domain and a non-specific DNA cleavage domain at the C-terminal. Once the protein is bound to duplex DNA via its DNA-binding domain at the 5′-GGATG-3′ recognition site, the DNA cleavage domain is activated through dimerization and cleaves, without further sequence specificity, the first strand 9 nucleotides downstream and the second strand 13 nucleotides upstream of the nearest nucleotide of the recognition site leaving a typical 4 base overhang. DNA cleavage is mediated through the non-specific cleavage domain which also includes the dimerization surface. The dimer interface is formed by the parallel helices a4 and a5 and two loops P1 and P2 of the cleavage domain. The Fold cleavage domain's molecular mass is 21.8 kDa, being composed of 194 amino acids. In some embodiments, FokI may comprise the amino acid sequence as denoted by SEQ ID NO:256, or any fragments, derivatives and variants thereof. In yet some further embodiments, a Fold variant useful in the present invention may comprise ancestral mutations. In some specific embodiments such FokI variant may comprise the amino acid sequence as denoted by SEQ ID NO. 439 (also referred to herein in the text and the figures as “ancestral Fold”, or as “consensus FokI”. In yet some further embodiments, a FokI variant may comprise the amino acid sequence as denoted by SEQ ID NO. 486 (also referred to herein as “enhanced Fold”). It should be appreciated that the present disclosure further encompasses any variations of the specified FokI variants.

As indicated herein, the present disclosure provides an effective nucleic acid guided genome modifier or effector chimeric protein, fusion protein, conjugate or complex having reduced or abolished PAM constraint or restriction. As shown by the present examples, this chimeric protein, fusion protein, conjugate or complex comprises at least one PAM reduced or abolished Cas protein that may be any Cas protein, that may be according to non-limiting embodiments of the present disclosure at least one of ScCas9, SpCas9, an ancestral Cas9, deltaproteobacteria CasX, Cas12a, CasF-1, CasF-2, CasF-3, Cas14a1, and Cas14b5. Still further, the nucleic acid guided genome modifier or effector chimeric protein, fusion protein, conjugate or complex of the present disclosure further comprises at least one nucleic acid modified component that may be any of the nucleic acid modifier proteins discussed in the present disclosure, for example, at least one FokI protein or any variants thereof, specifically any of the variants discussed by the present disclosure, for example, any FokI that comprise at least one ancestral or consensus mutation. Examples for optional Fold variants may include “consensus FokI”, “enhanced FokI” and the like, as further specified in Example 16. Particular relevant variants are disclosed for example, in FIG. 14 and Table 18. As to the Cas protein used in the nucleic acid guided genome modifier or effector chimeric protein, fusion protein, conjugate or complex of the present disclosure may be either a native Cas protein, for example, any of the Cas proteins disclosed by the invention, specifically, the ScCas, that displays a reduce PAM constraint or restriction, specifically, a PAM sequence comprising one restricting nucleotide (NNG). Still further, in some alternative or additional embodiments, the Cas protein used in the nucleic acid guided genome modifier or effector chimeric protein, fusion protein, conjugate or complex of the present disclosure, may comprise at least one deletion, replacement and/or substitution of at least one domain, fragment or at least one amino acid residue thereof. Non-limiting examples for such relevant domains or fragments that may be deleted, replaced or include at least one substitution or mutation may be the PAM binding domain (for example, the PAM BD loop) or any fragments or amino acid residues thereof, or any PAM recognition motif (for example, the Scloop) as disclosed by the present disclosure. Specific embodiments for such PAM recognition domains or sequences may include at least one of residues Thr1330 to Arg1342 (that is also referred to herein as the PAMBD loop), residues Ile367 to Ala376 (that is also referred to herein as the Scloop), residues Lys1337 and residue Gln1338 of ScCas, or any corresponding residues of other Cas homologs (e.g., any one of SpCas9, an ancestral Cas9, deltaproteobacteria CasX, Cas12a, CasF-1, CasF-2, CasF-3, Cas14a1, or Cas14b5). In some embodiments, the PAMBD loop may be deleted, either entirely, or partially (specifically, a PAM BD loop that comprise the amino acid sequence of residues 1330 to 1342 of scCas9). In yet some further embodiments, the PAMBD loop may be replaced either entirely, or partially with PAMBD loop of homologous Cas (e.g. the PAMBD loop of SpCas). In yet some further embodiments, the PAMBD loop may be replaced either entirely, or partially with at least one NSBD, as will be discussed herein after. Examples for such nucleic acid guided genome modifier or effector chimeric protein, fusion protein, conjugate or complex are disclosed for example by Example 2, and in Tables 10A and 10B. Examples for relevant NSBDs applicable in such variants include, but are not limited to any dsDBP, for example, at least one of: at least one ZF, HTH, SH3 domain, Non-specific RVD from AvrBS3 protein family, a CBD protein and StkC, domain or protein, and any variant and fragments thereof. Still further, in some alternative or additional embodiments, the Cas used or the nucleic acid guided genome modifier or effector chimeric protein, fusion protein, conjugate or complex of the present disclosure may comprise at least one deletion, replacement and/or at least one mutation in the Scloop. In some embodiments, the nucleic acid guided genome modifier or effector chimeric protein, fusion protein, conjugate or complex of the present disclosure comprise a Cas protein having a deletion of the entire Scloop (e.g., residues Ile367 to Ala376 of ScCas), or of any fragments thereof. Specific and non-limiting embodiments for such variants can be found in Example 2, FIGS. 3, 4 and Table 10C. In yet some further embodiments, the nucleic acid guided genome modifier or effector chimeric protein, fusion protein, conjugate or complex of the present disclosure comprise a replacement of the Scloop, or of at least a fragment thereof. In some embodiments, the Scloop may be replaced by at least one NSBD protein, specifically, any of the NSBDs disclosed above. In yet some further embodiments, the Cas protein of the nucleic acid guided genome modifier or effector chimeric protein, fusion protein, conjugate or complex of the present disclosure comprises an Scloop that comprise at least one mutation or substitution of at least one residues thereof. Examples for such Scloop substitutions or mutations include, but are not limited to the Scloop QQ mutation or AA mutation. Specific and non-limiting embodiments for such variants can be found in Example 2, FIG. 4 and Table 10C. Still further, in some alternative or additional embodiments, the Cas used or the nucleic acid guided genome modifier or effector chimeric protein, fusion protein, conjugate or complex of the present disclosure may comprise at least one deletion, replacement and/or at least one mutation in the HNH-nuclease domain or any fragment thereof. In some embodiments, the variants of the invention may comprise a deletion of the entire HNH domain, or of any fragments thereof. In yet some further embodiments, the nucleic acid guided genome modifier or effector chimeric protein, fusion protein, conjugate or complex may comprise a Cas having a replacement of the HNH domain with at least one HNH domain of a Cas homologues, or alternatively, with at least one linker. It should be noted that any of the linkers disclosed by the invention may be applicable in replacing the HNH domain. In yet some further embodiments, the nucleic acid guided genome modifier or effector chimeric protein, fusion protein, conjugate or complex may comprise a Cas having a replacement of the HNH domain or parts thereof with at least one NSBD protein, specifically, any of the NSBDs disclosed above. Non-limiting embodiments for such variants may be found in Examples 2, 3 and 16, in Tables 10A, 10b, 10c, 12 and 18, and FIGS. 3, 4, and 14. Still further, in some alternative or additional embodiments, the Cas used or the nucleic acid guided genome modifier or effector chimeric protein, fusion protein, conjugate or complex of the present disclosure may comprise at least one deletion, replacement and/or at least one mutation in at least one of, the REC2 domain or any fragments thereof, the FLEX domain, or any fragments thereof and/or the RUVC domain or any fragments thereof. In some embodiments, the nucleic acid guided genome modifier or effector chimeric protein, fusion protein, conjugate or complex of the present disclosure comprise ancestral mutation in at least one of, the REC2 domain and/or the RUVC domain. Non-limiting embodiments for such variants may be found in Examples 2, 3 and 16, in Tables 10A, 10b, 10c, 12 and 18, and in FIGS. 3, 4, and 14. Still further, in some alternative or additional embodiments, the Cas used for the nucleic acid guided genome modifier or effector chimeric protein, fusion protein, conjugate or complex of the present disclosure may comprise at least one NSDB that may replace any of the Cas fragments or domains as discussed above (e.g., the PAMBD loop, the Scloop, the HNH domain, and PAMBD). In yet some further embodiments, the NSBD may be added to the Cas protein or to any of the nucleic acid guided genome modifier or effector chimeric protein, fusion protein, conjugate or complex. Such NSBD may be added at the N′-terminus of the Cas protein or the nucleic acid guided genome modifier or effector chimeric protein, fusion protein, conjugate or complex, within the Cas Protein or the nucleic acid guided genome modifier or effector chimeric protein, fusion protein, conjugate or complex, and/or at the C′-terminus of the Cas protein or the nucleic acid guided genome modifier or effector chimeric protein, fusion protein, conjugate or complex of the present disclosure. Examples for relevant NSBDs applicable in such variants include, but are not limited to any dsDBP, for example, at least one of: at least one ZF, HTH, SH3 domain, Non-specific RVD from AvrBS3 protein family, a CBD protein and StkC, domain or protein, and any variant and fragments thereof. Non-limiting examples for such variants may be found in Example 13, Table 17, that present variant having an HMGN domain at the N-terminus of the variant.

In yet some further alternative and/or additional embodiments, any of the variants discussed herein may comprise additional linkers (either short, long, positively charged etc.), as discussed by the present disclosure. In yet some further embodiments, any of the variants of the invention may further comprise additional elements, such as NLS, as discussed in the present disclosure, e.g., bipartite SV40 NLS, nucleoplasmin NLS and the like. Still further, it should be understood that the nucleic acid guided genome modifier or effector chimeric protein, fusion protein, conjugate or complex of the present disclosure may comprise any of the modifications (e.g., deletions, replacements, substitutions) discussed above, or any combinations thereof, provided that said nucleic acid guided genome modifier or effector chimeric protein, fusion protein, conjugate or complex display a reduced or abolished PAM constraint, as disclosed herein.

Thus, in some particular and non-limiting embodiments, the nucleic acid guided genome modifier or effector chimeric protein, complex or conjugate provided by the invention may be a fusion protein or chimera composed of a Cas protein having reduced or abolished PAM constraint or restriction, fused to Fold, or any fragments or derivatives thereof. In some specific embodiments, the nucleic acid guided genome modifier or effector provided by the invention may be any of the following chimeric proteins: dScCas9-FokI-ZF1, dScCas9-FokI-ZF2, dScCas9-FokI-ZF3, dScCas9-FokI-ZF4, dScCas9-FokI-LAC, dScCas9-FokI-LAC2, dScCas9-FokI-LAC3, dScCas9-FokI-LAC4, dScCas9-FokI-HIVIN, dScCas9-FokI-HIVIN2, dScCas9-FokI-HIVIN3, dScCas9-FokI-HIVIN4, dScCas9-FokI-SS 07D, dScCas9-FokI-SSO7D2, dScCas9-FokI-SSO7D3, dScCas9-FokI-SSO7D4, dScCas9-FokI-STO7, dScCas9-FokI-STO72, dScCas9-FokI-STO73, dScCas9-FokI-STO74, dScCas9-FokI-StkC, dScCas9-FokI-StkC2, dScCas9-FokI-HMGB4, dScCas9-FokI-LoopDel, dScCas9-FokI-LoopQQ, dScCas9-FokI-LoopAA, the dScCasFok, ancestral RuvC+Rec, ScLoopA, SV40+nucleoplasminNLS, the dScCasFok, ancestral RuvC+Rec, ScLoopQQ, SV40+nucleoplasminNLS, the dScCasFok, ancestral RuvC+Rec, ScLoopAA, SV40+nucleoplasminNLS, Cas9dScCasFok-2NLS, dScCasFok-HNHA-2NLS, dScCasFok-HNHA-PAMBDwholeA-2NLS, dScCasFok-HNHA-PAMBDloopA-2NLS , dScCasFok-Zinc finger-PAMBD loop replacement longer linkers-2NLS, dScCasFok-HNHA-Zinc finger-PAMBD loop replacement-2NLS, dScCasFok-HNHA-Zinc finger-PAMBD loop replacement longer linkers-2NLS, dScCasFok-Lac repressor DBD-PAMBD whole replacement-2NLS, dScCasFok-HNHA-Lac repressor DBD DBD-PAMBD whole replacement-2NLS, dScCasFok-SSO7D-PAMBD whole replacement-2NLS, dScCasFok-SSO7D-PAMBD whole replacement-longer linkers-2NLS, dScCasFok-HNHA-SSO7D-PAMBD whole replacement-2NLS, dScCasFok-HNHA-SSO7D-PAMBD whole replacement-longer linkers-2NLS, dScCasFok-STO7D-PAMBD loop replacement-longer linker-2NLS, dCasFok-HNHA-STO7D-PAMBD loop replacement-longer linker-2NLS, dCasFok-HNHA-HIV integrase DBD-PAMBD replacement whole replacement-longer linker-2NLS, dCasFok-HNHA-HIV integrase DBD-PAMBD replacement-loop replacement-longer linker-2NLS, dScCasFok-HNHΔ with longer linkers-2NLS, dScCasFok-HNHΔ replaced with SSB-2NLS, dScCasFok-HNHΔ with larger deletion-2NLS, dScCasFok-HNHΔ with positively charged linker-2NLS, dScCasFok-HNHΔ with short positively charged linker-2NLS, dScCasFok-HNHΔ with H-NS linker-2NLS, dScCasFok SV40+bipartite SV40 NLS, dScCasFok SV40+bipartite SV40 NLS 6× His, dScCasFok ancestral RuvC+Rec1/2domain SV40 and bipartite SV40 NLS, dScCasFok HNHΔ SV40+bipartiteSV40 NLS, dScCasFok HNHA with longer linker SV40+bipartiteSV40 NLS, dScCasFok HNH replaced with SSB SV40+bipartiteSV40 NLS, dScCasFok HNHΔ with longer deletion SV40+bipartiteSV40 NLS, dScCasFok HNHΔ with longer positively charged linker SV40+bipartiteSV40 NLS, dScCasFok HNHΔ with shorter positively charged linker SV40+bipartiteSV40 NLS, dScCasFok HNH replaced with StkC SV40+bipartiteSV40 NLS, dScCasFok HNHΔ with HNS linker SV40+bipartiteSV40 NLS, dScCasFok ancestral RuvC+Rec1/2HNHΔ S V40+bipartiteS V40 NLS, dScCasFok ancestral RuvC+Rec1/2HNHΔ with longer linker SV40+bipartiteSV40 NLS, dScCasFok ancestral RuvC+Rec1/2HNH replaced with SSB SV40+bipartiteSV40 NLS, dScCasFok ancestral RuvC+Rec1/2HNHΔ with longer deletion SV40+bipartiteSV40 NLS, dScCasFok ancestral RuvC+Rec1/2HNHΔ with longer positively charged linker SV40+bipartiteSV40 NLS, dScCasFok ancestral RuvC+Rec1/2HNHΔ with shorter positively charged linker SV40+bipartiteSV40 NLS, dScCasFok ancestral RuvC+Rec1/2HNH replaced with StkC SV40+bipartiteSV40 NLS, dScCasFok ancestral RuvC+Rec1/2HNHΔ with HNS linker SV40+bipartiteSV40 NLS; dScCasFok,HNHA, whole PAMBD replaced with Sto7, longer linkers, SV40+nucleoplasmin NLS; dScCasFok,HNHA, PAMBD loop replaced with Sto7, longer linkers, SV40+nucleoplasmin NLS; dScCasFok,HNHA,PAMBD loop replaced with HMGN, SV40+nucleoplasmin NLS; dScCasFok,HNHA, whole PAMBD replaced with HMGN, SV40+nucleoplasmin NLS; dScCasFok,HNHA,PAMBD loop replaced with StkC, SV40+nucleoplasmin NLS; dScCasFok,HNHA,whole PAMBD replaced with StkC, SV40+nucleoplasmin NLS; dScCasFok, S V40+bipartiteS V40, ancestral mutations in RuvC+REC1/2 domain, Scloop deletion; dScCasFok,SV40+bipartiteSV40, ancestral mutations in RuvC+REC1/2 domain, Scloop deletion, 6His tag; dScCasFok,SV40+bipartiteSV40, ancestral mutations in RuvC+REC 1/2 domain, Scloop deletion, HNHdeletion; dScCasFok,SV40+bipartiteSV40, ancestral mutations in RuvC+REC1/2 domain, Scloop deletion, HNHdeletion, 6His tag; dScCasFok,SV40+bipartiteSV40, ancestral mutations in RuvC+REC1/2 domain, Scloop deletion, HNHdeletion, 6His tag; dScCasFok, SV40+bipartiteSV40, ancestral mutations in RuvC+REC1/2 domain, Scloop QQmutant, HNHdeletion, 6His tag; dScCasFok, SV40+bipartiteSV40, ancestral mutations in RuvC+REC 1/2 domain, Scloop AAmutant, HNHdeletion, 6His tag; dScCasFok, SV40+bipartiteSV40, ancestral mutations in RuvC+REC1/2 domain, Scloop Sp Replacement, HNHdeletion, 6His tag; dScCasFok, SV40+bipartiteSV40, ancestral mutations in RuvC+REC1/2 domain, Scloop deletion, HNHdeletion, 6His tag; dScCasFok, SV40+bipartiteSV40, ancestral mutations in RuvC+REC 1/2 domain, Scloop QQmutant, HNHdeletion, 6His tag; dScCasFok, SV40+bipartiteSV40, ancestral mutations in RuvC+REC1/2 domain, Scloop AAmutant, HNHdeletion, 6His tag; dScCasFok, SV40+nucleoplasmin, ancestral mutations in RuvC+REC1/2 domain, Scloop deletion, HNH deletion, PAM replaced with Zinc finger; dScCasFok, SV40+nucleoplasmin, ancestral mutations in RuvC+REC1/2 domain, Scloop deletion, HNH deletion, whole PAMBD replaced with LacI DNA binding domain; dScCasFok, SV40+nucleoplasmin, ancestral mutations in RuvC+REC1/2 domain, Scloop deletion, HNH deletion, whole PAMBD replaced with SSO7D; dScCasFok, SV40+nucleoplasmin, ancestral mutations in RuvC+REC1/2 domain, Scloop deletion, HNH deletion, PAMBD loop replaced with HMGN; dScCasFok, SV40+nucleoplasmin, ancestral mutations in RuvC+REC1/2 domain, Scloop deletion, HNH deletion, whole PAMBD replaced with STO7; dScCasFok, SV40+nucleoplasmin, ancestral mutations in RuvC+REC1/2 domain, Scloop QQmutation, HNH deletion, PAMBD loop replaced with Zinc finger; dScCasFok, SV40+nucleoplasmin, ancestral mutations in RuvC+REC1/2 domain, Scloop QQmutation HNH deletion, whole PAMBD replaced with Lad DNA binding domain; dScCasFok, SV40+nucleoplasmin, ancestral mutations in RuvC+REC1/2 domain, Scloop QQmutation, HNH deletion, whole PAMBD replaced with SSO7D; dScCasFok, SV40+nucleoplasmin, ancestral mutations in RuvC+REC1/2 domain, Scloop QQmutation, HNH deletion, PAMBD loop replaced with HMGN; dScCasFok, SV40+nucleoplasmin, ancestral mutations in RuvC+REC1/2 domain, Scloop QQmutation, HNH deletion, whole PAMBD replaced with STO7; dScCasFok, SV40+nucleoplasmin, ancestral mutations in RuvC+REC1/2 domain, Scloop AAmutation, HNH deletion, PAMBD loop replaced with Zinc finger; dScCasFok, SV40+nucleoplasmin, ancestral mutations in RuvC+REC1/2 domain, Scloop AAmutation HNH deletion, wholePAMBD replaced with LacI DNA binding domain; dScCasFok, SV40+nucleoplasmin, ancestral mutations in RuvC+REC1/2 domain, Scloop AAmutation, HNH deletion, whole PAMBD replaced with SSO7D; dScCasFok, SV40+nucleoplasmin, ancestral mutations in RuvC+REC1/2 domain, Scloop AAmutation, HNH deletion, PAMBD loop replaced with HMGN; dScCasFok, SV40+nucleoplasmin, ancestral mutations in RuvC+REC1/2 domain, Scloop AAmutation, HNH deletion, whole PAMBD replaced with STO7; dScCasFok, SV40+nucleoplasmin, ancestral mutations in RuvC+REC1/2 domain, Scloop SpReplacement, HNH deletion, PAMBD loop replaced with Zinc finger; dScCasFok, SV40+nucleoplasmin, ancestral mutations in RuvC+REC1/2 domain, Scloop SpReplacement HNH deletion, whole PAMBD replaced with LacI DNA binding domain; dScCasFok, SV40+nucleoplasmin, ancestral mutations in RuvC+REC1/2 domain, Scloop SpReplacement HNH deletion, whole PAMBD replaced with SSO7D; dScCasFok, SV40+nucleoplasmin, ancestral mutations in RuvC+REC1/2 domain, Scloop SpReplacement HNH deletion, PAMBD loop replaced with HMGN; dScCasFok, SV40+nucleoplasmin, ancestral mutations in RuvC+REC1/2 domain, Scloop SpReplacement HNH deletion, whole PAMBD replaced with STO7 or any variants or mutants thereof.

Still further, in some specific embodiments of the present disclosure, the nucleic acid guided genome modifier or effector provided by the invention may be dScCasFok, SV40 NLS. In yet some further embodiments, the nucleic acid guided genome modifier or effector provided by the invention may be the dScCasFok, SV40+SV40 bipartite NLS. Still further, in some embodiments, the nucleic acid guided genome modifier or effector provided by the invention may be the dCasFok, FokI consensus, SV40+bipartiteSV40. In certain embodiments, the nucleic acid guided genome modifier or effector provided by the invention may be the dCasFok, FokI consensus, SV40+bipartiteSV40, ancestral mutations in RuvC+REC1/2 domain, HNH deletion. In certain embodiments, the nucleic acid guided genome modifier or effector provided by the invention may be the dScCasFok, SV40+nucleoplasmin, ancestral mutations in RuvC+REC1/2 domain, Scloop QQmutation HNH deletion, whole PAMBD replaced with LacI DNA binding domain. Still Further, in certain embodiments, the nucleic acid guided genome modifier or effector provided by the invention may be the dScCasFok, SV40+nucleoplasmin, ancestral mutations in RuvC+REC1/2 domain, Scloop SpReplacement, HNH deletion, PAMBD loop replaced with Zinc finger. In yet some further embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, whole PAMBD replaced with SSO7D. In certain embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, PAMBD loop replaced with HMGN. In certain embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, whole PAMBD replaced with STO7.

In some embodiment, the dScCas9-FokI-ZF1, may comprise an amino acid sequence as denoted by SEQ ID NO. 24, dScCas9-FokI-ZF2, may comprise an amino acid sequence as denoted by as denoted by SEQ ID NO. 25, dScCas9-FokI-ZF3 may comprise an amino acid sequence as denoted by, as denoted by SEQ ID NO. 26, dScCas9-FokI-ZF4 may comprise an amino acid sequence as denoted by SEQ ID NO. 27, dScCas9-FokI-LAC may comprise an amino acid sequence as denoted by SEQ ID NO. 28, dScCas9-FokI-LAC2 may comprise an amino acid sequence as denoted by SEQ ID NO. 29, dScCas9-FokI-LAC3 may comprise an amino acid sequence as denoted by SEQ ID NO. 30, dScCas9-FokI-LAC4 may comprise an amino acid sequence as denoted by, as denoted by SEQ ID NO. 31, dScCas9-FokI-HIVIN may comprise an amino acid sequence as denoted by SEQ ID NO. 32, dScCas9-FokI-HIVIN2 may comprise an amino acid sequence as denoted by SEQ ID NO. 33, dScCas9-FokI-HIVIN3 may comprise an amino acid sequence as denoted by SEQ ID NO. 34, dScCas9-FokI-HIVIN4 may comprise an amino acid sequence as denoted by SEQ ID NO. 35, dScCas9-FokI-SSO7D may comprise an amino acid sequence as denoted by SEQ ID NO. 36, dScCas9-FokI-SSO7D2 may comprise an amino acid sequence as denoted by SEQ ID NO. 37, dScCas9-FokI-SSO7D3 may comprise an amino acid sequence as denoted by SEQ ID NO. 38, dScCas9-FokI-SSO7D4 may comprise an amino acid sequence as denoted by SEQ ID NO. 39, dScCas9-FokI-STO7 may comprise an amino acid sequence as denoted by SEQ ID NO. 40, dScCas9-FokI-ST072 may comprise an amino acid sequence as denoted by, as denoted by SEQ ID NO. 41, dScCas9-FokI-ST073 may comprise an amino acid sequence as denoted by SEQ ID NO. 42, dScCas9-FokI-ST074 may comprise an amino acid sequence as denoted by SEQ ID NO. 43, dScCas9-FokI-StkC may comprise an amino acid sequence as denoted by SEQ ID NO. 44, dScCas9-FokI-StkC2, as denoted by SEQ ID NO. 266 and dScCas9-FokI-HMGB4 as denoted by SEQ ID NO.267. Chimeras based on variants of Cas9 (truncated versions) may include the dScCas9-FokI.dHNH.dREC2, as denoted by SEQ ID NO. 14, the dScCas9-FokI.dHNH.dFLEX, as denoted by SEQ ID NO. 15, the dScCas9-FokI.dREC2.dFLEX, as denoted by SEQ ID NO. 16, dScCas9-FokI.dHNH.dREC2.dFLEX, as denoted by SEQ ID NO. 17, the dScCas9-FokI-LoopDel may comprise an amino acid sequence as denoted by SEQ ID NO. 345, the dScCas9-FokI-LoopQQ may comprise an amino acid sequence as denoted by SEQ ID NO. 346, the dScCas9-FokI-LoopAA may comprise an amino acid sequence as denoted by SEQ ID NO. 347, the dScCas9-FokI-ScLoop ASp may comprise an amino acid sequence as denoted by SEQ ID NO. 348, the dScCasFok, ancestral RuvC+Rec1/2, ScLoopA, SV40+nucleoplasminNLS may comprise an amino acid sequence as denoted by SEQ ID NO. 394, the dScCasFok, ancestral RuvC+Rec1/2, ScLoopQQ, SV40+nucleoplasminNLS may comprise an amino acid sequence as denoted by SEQ ID NO. 395, the dScCasFok, ancestral RuvC+Rec1/2, ScLoopAA, SV40+nucleoplasminNLS may comprise an amino acid sequence as denoted by SEQ ID NO. 396, the dScCasFok-2NLS may comprise an amino acid sequence as denoted by SEQ ID NO: 330, the dScCasFok-HNHA-2NLS may comprise an amino acid sequence as denoted by SEQ ID NO:331, the dScCasFok-HNHA-PAMBD whole A-2NLS may comprise an amino acid sequence as denoted by SEQ ID NO: 332, the dScCasFok-HNHA-PAMBDloopA-2NLS may comprise an amino acid sequence as denoted by SEQ ID NO: 333, the dScCasFok-Zinc finger-PAMBD loop replacement-longer linkers-2NLS may comprise an amino acid sequence as denoted by SEQ ID NO: 334, the dScCasFok-HNHA-Zinc finger-PAMBD loop replacement-2NLS may comprise an amino acid sequence as denoted by SEQ ID NO: 314, the dScCasFok-HNHA-Zinc finger-PAMBD loop replacement-longer linkers-2NLS may comprise an amino acid sequence as denoted by SEQ ID NO: 315, the dScCasFok-Lac repressor DBD-PAMBD whole replacement-2NLS may comprise an amino acid sequence as denoted by SEQ ID NO: 335, the dScCasFok-HNHA-Lac repressor DBD-PAMBD whole replacement-2NLS may comprise an amino acid sequence as denoted by SEQ ID NO: 316, the dScCasFok-SSO7D-PAMBD whole replacement-2NLS may comprise an amino acid sequence as denoted by SEQ ID NO: 336, the dScCasFok-SSO7D-PAMBD whole replacement-longer linkers-2NLS, may comprise an amino acid sequence as denoted by SEQ ID NO: 337, the dScCasFok-HNHA-5507D-PAMBD whole replacement-2NLS may comprise an amino acid sequence as denoted by SEQ ID NO: 317, the dScCasFok-HNHA-5507D-PAMBD whole replacement-longer linkers-2NLS may comprise an amino acid sequence as denoted by SEQ ID NO: 338, the dScCasFok-STO7D-PAMBD loop replacement-longer linker-2NLS may comprise an amino acid sequence as denoted by SEQ ID NO: 339, the dCasFok-HNHA-STO7D-PAMBD loop replacement-longer linker-2NLS may comprise an amino acid sequence as denoted by SEQ ID NO: 318, the dCasFok-HNHA-HIV integrase DBD-PAMBD replacement whole replacement-longer linker-2NLS may comprise an amino acid sequence as denoted by SEQ ID NO: 340, the dCasFok-HNHA-HIV integrase DBD-PAMBD replacement-loop replacement-longer linker-2NLS may comprise an amino acid sequence as denoted by SEQ ID NO: 341, the dScCasFok-HNHΔ with longer linkers-2NLS may comprise an amino acid sequence as denoted by SEQ ID NO: 342, the dScCasFok-HNHΔ replaced with SSB-2NLS may comprise an amino acid sequence as denoted by SEQ ID NO: 343, the dScCasFok-HNHΔ with larger deletion-2NLS may comprise an amino acid sequence as denoted by SEQ ID NO: 344, the dScCasFok-HNHΔ with positively charged linker-2NLS may comprise an amino acid sequence as denoted by SEQ ID NO: 349, the dScCasFok-HNHΔ with short positively charged linker-2NLS may comprise an amino acid sequence as denoted by SEQ ID NO: 350, the dScCasFok-HNHΔ with H-NS linker-2NLS may comprise an amino acid sequence as denoted by SEQ ID NO: 351, the dScCasFok SV40+bipartite SV40 NLS may comprise an amino acid sequence as denoted by SEQ ID NO: 375, the dScCasFok SV40+bipartite SV40 NLS 6× His may comprise an amino acid sequence as denoted by SEQ ID NO: 376, the dScCasFok ancestral RuvC+Rec1/2domain SV40 and bipartite SV40 NLS may comprise an amino acid sequence as denoted by SEQ ID NO: 377, the dScCasFok HNHA SV40+bipartiteSV40 NLS may comprise an amino acid sequence as denoted by SEQ ID NO: 378, the dScCasFok HNHΔ with longer linker SV40+bipartiteSV40 NLS may comprise an amino acid sequence as denoted by SEQ ID NO: 379, the dScCasFok, HNH replaced with SSB SV40+bipartiteSV40 NLS may comprise an amino acid sequence as denoted by SEQ ID NO: 380, the dScCasFok, HNHΔ with longer deletion SV40+bipartiteSV40 NLS may comprise an amino acid sequence as denoted by SEQ ID NO: 381, the dScCasFok HNHΔ with longer positively charged linker SV40+bipartiteSV40 NLS may comprise an amino acid sequence as denoted by SEQ ID NO: 382, the dScCasFok HNHΔ with shorter positively charged linker SV40+bipartiteSV40 NLS may comprise an amino acid sequence as denoted by SEQ ID NO: 383, the dScCasFok HNH replaced with StkC SV40+bipartiteSV40 NLS may comprise an amino acid sequence as denoted by SEQ ID NO: 384, the dScCasFok HNHΔ with HNS linker SV40+bipartiteSV40 NLS may comprise an amino acid sequence as denoted by SEQ ID NO: 385, the dScCasFok ancestral RuvC+Rec1/2HNHA, SV40+bipartiteSV40 NLS may comprise an amino acid sequence as denoted by SEQ ID NO: 386, the dScCasFok ancestral RuvC+Rec1/2HNHΔ with longer linker SV40+bipartiteSV40 NLS may comprise an amino acid sequence as denoted by SEQ ID NO: 387, the dScCasFok ancestral RuvC+Rec1/2HNH replaced with SSB SV40+bipartiteSV40 NLS may comprise an amino acid sequence as denoted by SEQ ID NO: 388, the dScCasFok ancestral RuvC+Rec1/2HNHΔ with longer deletion SV40+bipartiteSV40 NLS may comprise an amino acid sequence as denoted by SEQ ID NO: 389, the dScCasFok ancestral RuvC+Rec1/2HNHΔ with longer positively charged linker SV40+bipartite SV40 NLS may comprise an amino acid sequence as denoted by SEQ ID NO: 390, the dScCasFok ancestral RuvC+Rec1/2HNHΔ with shorter positively charged linker SV40+bipartiteSV40 NLS may comprise an amino acid sequence as denoted by SEQ ID NO: 391, the dScCasFok ancestral RuvC+Rec1/2HNH replaced with StkC SV40+bipartiteSV40 NLS may comprise an amino acid sequence as denoted by SEQ ID NO: 392, the dScCasFok ancestral RuvC+Rec1/2HNHΔ with HNS linker SV40+bipartiteSV40 NLS may comprise an amino acid sequence as denoted by SEQ ID NO: 393, dScCasFok,HNHA, whole PAMBD replaced with Sto7, longer linkers, SV40+nucleoplasmin NLS may comprise an amino acid sequence as denoted by SEQ ID NO. 433; dScCasFok,HNHA, PAMBD loop replaced with Sto7,1onger linkers, SV40+nucleoplasmin NLS may comprise an amino acid sequence as denoted by SEQ ID NO. 434; dScCasFok,HNHA,PAMBD loop replaced with HMGN, SV40+nucleoplasmin NLS may comprise an amino acid sequence as denoted by SEQ ID NO. 435; dScCasFok,HNHA, whole PAMBD replaced with HMGN , SV40+nucleoplasmin NLS may comprise an amino acid sequence as denoted by SEQ ID NO. 436; dScCasFok,HNHA,PAMBD loop replaced with StkC, SV40+nucleoplasmin NLS may comprise an amino acid sequence as denoted by SEQ ID NO. 437; dScCasFok,HNHA,whole PAMBD replaced with StkC, SV40+nucleoplasmin NLS may comprise an amino acid sequence as denoted by SEQ ID NO. 438; the dScCasFok,SV40+bipartiteSV40, ancestral mutations in RuvC+REC1/2 domain, Scloop deletion may comprise an amino acid sequence as denoted by SEQ ID NO. 450; the dScCasFok,SV40+bipartiteSV40, ancestral mutations in RuvC+REC1/2 domain, Scloop deletion, 6His tag may comprise an amino acid sequence as denoted by SEQ ID NO. 451; the dScCasFok,SV40+bipartiteSV40, ancestral mutations in RuvC+REC1/2 domain, Scloop deletion, HNHdeletion may comprise an amino acid sequence as denoted by SEQ ID NO. 452; the dScCasFok,SV40+bipartiteSV40, ancestral mutations in RuvC+REC1/2 domain, Scloop deletion, HNHdeletion, 6His tag may comprise an amino acid sequence as denoted by SEQ ID NO. 453; the dScCasFok,SV40+bipartiteSV40, ancestral mutations in RuvC+REC1/2 domain, Scloop deletion, HNHdeletion, 6His tag may comprise an amino acid sequence as denoted by SEQ ID NO. 454; the dScCasFok, SV40+bipartiteSV40, ancestral mutations in RuvC+REC1/2 domain, Scloop QQmutant, HNHdeletion, 6His tag may comprise an amino acid sequence as denoted by SEQ ID NO. 455; the dScCasFok, SV40+bipartiteSV40, ancestral mutations in RuvC+REC1/2 domain, Scloop AAmutant, HNHdeletion, 6His tag may comprise an amino acid sequence as denoted by SEQ ID NO. 456; the dScCasFok, SV40+bipartiteSV40, ancestral mutations in RuvC+REC1/2 domain, Scloop Sp Replacement, HNHdeletion, 6His tag may comprise an amino acid sequence as denoted by SEQ ID NO. 457; the dScCasFok, SV40+bipartiteSV40, ancestral mutations in RuvC+REC1/2 domain, Scloop deletion, HNHdeletion, 6His tag may comprise an amino acid sequence as denoted by SEQ ID NO. 458; the dScCasFok, SV40+bipartiteSV40, ancestral mutations in RuvC+REC1/2 domain, Scloop QQmutant, HNHdeletion, 6His tag may comprise an amino acid sequence as denoted by SEQ ID NO. 459; the dScCasFok, SV40+bipartiteSV40, ancestral mutations in RuvC+REC1/2 domain, Scloop AAmutant, HNHdeletion, 6His tag may comprise an amino acid sequence as denoted by SEQ ID NO. 460; the dScCasFok, SV40+nucleoplasmin, ancestral mutations in RuvC+REC1/2 domain, Scloop deletion, HNH deletion, PAMBD loop replaced with Zinc finger may comprise an amino acid sequence as denoted by SEQ ID NO. 461; the dScCasFok, SV40+nucleoplasmin, ancestral mutations in RuvC+REC1/2 domain, Scloop deletion, HNH deletion, whole PAMBD replaced with LacI DNA binding domain may comprise an amino acid sequence as denoted by SEQ ID NO. 462; the dScCasFok, SV40+nucleoplasmin, ancestral mutations in RuvC+REC1/2 domain, Scloop deletion, HNH deletion, whole PAMBD replaced with SSO7D may comprise an amino acid sequence as denoted by SEQ ID NO. 463; the dScCasFok, SV40+nucleoplasmin, ancestral mutations in RuvC+REC1/2 domain, Scloop deletion, HNH deletion, PAMBD loop replaced with HMGN may comprise an amino acid sequence as denoted by SEQ ID NO. 464; the dScCasFok, SV40+nucleoplasmin, ancestral mutations in RuvC+REC1/2 domain, Scloop deletion, HNH deletion, whole PAMBD replaced with STO7 may comprise an amino acid sequence as denoted by SEQ ID NO. 465; the dScCasFok, SV40+nucleoplasmin, ancestral mutations in RuvC+REC1/2 domain, Scloop QQmutation, HNH deletion, PAMBD loop replaced with Zinc finger may comprise an amino acid sequence as denoted by SEQ ID NO. 466; the dScCasFok, SV40+nucleoplasmin, ancestral mutations in RuvC+REC1/2 domain, Scloop QQmutation HNH deletion, whole PAMBD replaced with LacI DNA binding domain may comprise an amino acid sequence as denoted by SEQ ID NO. 467; the dScCasFok, SV40+nucleoplasmin, ancestral mutations in RuvC+REC1/2 domain, Scloop QQmutation, HNH deletion, whole PAMBD replaced with SSO7D may comprise an amino acid sequence as denoted by SEQ ID NO. 468; the dScCasFok, SV40+nucleoplasmin, ancestral mutations in RuvC+REC1/2 domain, Scloop QQmutation, HNH deletion, PAMBD loop replaced with HMGN may comprise an amino acid sequence as denoted by SEQ ID NO. 469; the dScCasFok, SV40+nucleoplasmin, ancestral mutations in RuvC+REC1/2 domain, Scloop QQmutation, HNH deletion, whole PAMBD replaced with STO7 may comprise an amino acid sequence as denoted by SEQ ID NO. 470; the dScCasFok, SV40+nucleoplasmin, ancestral mutations in RuvC+REC1/2 domain, Scloop AAmutation, HNH deletion, PAMBD loop replaced with Zinc finger may comprise an amino acid sequence as denoted by SEQ ID NO. 471; the dScCasFok, SV40+nucleoplasmin, ancestral mutations in RuvC+REC1/2 domain, Scloop AAmutation HNH deletion, whole PAMBD replaced with LacI DNA binding domain may comprise an amino acid sequence as denoted by SEQ ID NO. 472; the dScCasFok, SV40+nucleoplasmin, ancestral mutations in RuvC+REC1/2 domain, Scloop AAmutation, HNH deletion, whole PAMBD replaced with SSO7D may comprise an amino acid sequence as denoted by SEQ ID NO. 473; the dScCasFok, SV40+nucleoplasmin, ancestral mutations in RuvC+REC1/2 domain, Scloop AAmutation, HNH deletion, PAMBD loop replaced with HMGN may comprise an amino acid sequence as denoted by SEQ ID NO. 474; the dScCasFok, SV40+nucleoplasmin, ancestral mutations in RuvC+REC1/2 domain, Scloop AAmutation, HNH deletion, whole PAMBD replaced with STO7 may comprise an amino acid sequence as denoted by SEQ ID NO. 475; the dScCasFok, SV40+nucleoplasmin, ancestral mutations in RuvC+REC1/2 domain, Scloop SpReplacement, HNH deletion, PAMBD loop replaced with Zinc finger may comprise an amino acid sequence as denoted by SEQ ID NO. 476; the dScCasFok, SV40+nucleoplasmin, ancestral mutations in RuvC+REC1/2 domain, Scloop SpReplacement HNH deletion, whole PAMBD replaced with LacI DNA binding domain may comprise an amino acid sequence as denoted by SEQ ID NO. 477; the dScCasFok, SV40+nucleoplasmin, ancestral mutations in RuvC+REC1/2 domain, Scloop SpReplacement HNH deletion, whole PAMBD replaced with SSO7D may comprise an amino acid sequence as denoted by SEQ ID NO. 478; the dScCasFok, SV40+nucleoplasmin, ancestral mutations in RuvC+REC1/2 domain, Scloop SpReplacement HNH deletion, PAM replaced with HMGN may comprise an amino acid sequence as denoted by SEQ ID NO. 479; the dScCasFok, SV40+nucleoplasmin, ancestral mutations in RuvC+REC1/2 domain, Scloop SpReplacement HNH deletion, PAM replaced with STO7 may comprise an amino acid sequence as denoted by SEQ ID NO. 480. Still further, in some specific embodiments of the present disclosure, the nucleic acid guided genome modifier or effector provided by the invention may be the dScCasFok, SV40 NLS (referred to herein as TG11241). In some embodiments, such nucleic acid guided genome modifier or effector may comprise the amino acid sequence as denoted by SEQ ID NO. 2.

In some embodiments, the nucleic acid guided genome modifier or effector provided by the invention may be the dScCasFok, SV40+SV40 bipartite NLS (14280). In more specific embodiments such the nucleic acid guided genome modifier or effector may comprise the amino acid sequence as denoted by SEQ ID NO. 375.

In some embodiments, the nucleic acid guided genome modifier or effector provided by the invention may be the dCasFok, FokI consensus, SV40+bipartiteSV40 (14659). In more specific embodiments such the nucleic acid guided genome modifier or effector may comprise the amino acid sequence as denoted by SEQ ID NO. 444.

In some embodiments, the nucleic acid guided genome modifier or effector provided by the invention may be the dCasFok, FokI consensus, SV40+bipartiteSV40, ancestral mutations in RuvC+REC1/2 domain, HNH deletion (14667). In more specific embodiments such the nucleic acid guided genome modifier or effector may comprise the amino acid sequence as denoted by SEQ ID NO. 448.

In some embodiments, the nucleic acid guided genome modifier or effector provided by the invention may be the dScCasFok, SV40+nucleoplasmin, ancestral mutations in RuvC+REC1/2 domain, Scloop QQmutation HNH deletion, whole PAMBD replaced with LacI DNA binding domain (14643). In more specific embodiments such the nucleic acid guided genome modifier or effector may comprise the amino acid sequence as denoted by SEQ ID NO.467.

In some embodiments, the nucleic acid guided genome modifier or effector provided by the invention may be the dScCasFok, SV40+nucleoplasmin, ancestral mutations in RuvC+REC1/2 domain, Scloop SpReplacement, HNH deletion, PAMBD loop replaced with Zinc finger (14652). In more specific embodiments such the nucleic acid guided genome modifier or effector may comprise the amino acid sequence as denoted by SEQ ID NO. 476.

In some embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, whole PAMBD replaced with SSO7D (14654). In more specific embodiments such the nucleic acid guided genome modifier or effector may comprise the amino acid sequence as denoted by SEQ ID NO. 478.

In some embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, PAMBD loop replaced with HMGN (14655). In more specific embodiments such the nucleic acid guided genome modifier or effector may comprise the amino acid sequence as denoted by SEQ ID NO. 479.

In some embodiments, the nucleic acid guided genome modifier or effector provided by the invention may be the dScCasFok, SV40+nucleoplasmin, ancestral mutations in RuvC+REC1/2 domain, Scloop SpReplacement HNH deletion, whole PAMBD replaced with STO7 (14656). In more specific embodiments such the nucleic acid guided genome modifier or effector may comprise the amino acid sequence as denoted by SEQ ID NO. 480.

In yet some further embodiments, the nucleic acid guided genome modifier chimeric protein of the invention may comprise as the effector/modifier component, any other nuclease as discussed above. In some specific and non-limiting embodiments, such chimeras may include the dScCas9-CspCI that comprise in some embodiments the amino acid sequence as denoted by SEQ ID NO. 288, the dScCas9-BsgI, that comprise in some embodiments the amino acid sequence as denoted by SEQ ID NO. 289, the dScCas9-BbvI, that comprise in some embodiments the amino acid sequence as denoted by SEQ ID NO. 290.

In yet some embodiments, the invention further encompasses nucleic acid guided genome modifier chimeric protein that may comprise as the PAM-free or reduced Cas protein, any Cas protein, for example, CasX, dCas14 or ancestral Cas, fused to at least one effector/modifier that may be a nuclease such as FokI. Examples for such chimeras may include the dCasX-FokI, that comprise in some embodiments the amino acid sequence as denoted by SEQ ID NO. 45, the dCasX-FokI DNTSB, that comprise in some embodiments the amino acid sequence as denoted by SEQ ID NO. 46, the dCasX-FokI dTSL, that comprise in some embodiments the amino acid sequence as denoted by SEQ ID NO. 47, the dCasX-FokI dNTSB dTSL, that comprise in some embodiments the amino acid sequence as denoted by SEQ ID NO. 48, the dCasX-FokI (default linker), that comprise in some embodiments the amino acid sequence as denoted by SEQ ID NO. 210, the dCasX-FokI (longer linker), that comprise in some embodiments the amino acid sequence as denoted by SEQ ID NO. 211, the dCasX-FokI (shorter linker, small flexible amino acids and uncharged), that comprise in some embodiments the amino acid sequence as denoted by SEQ ID NO. 212, and the dCasX-FokI (linker composed of small uncharged amino acids, and contains nuclear localization tag), that comprise in some embodiments the amino acid sequence as denoted by SEQ ID NO. 213.

Still further, in some embodiments, the invention further encompasses variants of the nucleic acid guided genome modifier chimeric protein of the present disclosure that comprises at least one PAM-free or reduced CasX protein, as discussed above. In some specific embodiments, such variants may comprise at least one substitution in at least one amino acid residue. In some further embodiments, the PAM-free or reduced CasX-FokI chimeras of the present disclosure may comprise at least one substitution in at least one of K226, S521K, 5525 and/or G577K. In some embodiments, these substitutions may be included in each of the PAM-free or reduced CasX protein of the present disclosure, specifically, in at least one of the chimeras of SEQ ID NO. 45, 46, 47, 48, 210, 211, 212, and 213. In yet some specific embodiments, the PAM-free or reduced CasX-FokI chimeras of the present disclosure may comprise at least one substitution in at least one of K226, specifically, substituting lysine K226 to Alanine, specifically, K226A. Thus, in some embodiments, the chimeras of at least one of SEQ ID NO. 45, 46, 47, 48, 210, 211, 212, and 213, may comprise a K226A substitution. In yet some alternative embodiments, the K226 may be substituted to glutamin, specifically, K226Q. Thus, in some embodiments, the chimeras of at least one of SEQ ID NO. 45, 46, 47, 48, 210, 211, 212, and 213, may comprise a K226Q substitution. Still further, in some embodiments serine 521 may be substituted to lysine, specifically, S521K. Thus, in some embodiments, the chimeras of at least one of SEQ ID NO. 45, 46, 47, 48, 210, 211, 212, and 213, may comprise a S521K substitution. In yet some further embodiments serine 525 may be substituted to lysine, specifically, S525K. Thus, in some embodiments, the chimeras of at least one of SEQ ID NO. 45, 46, 47, 48, 210, 211, 212, and 213, may comprise a S525K substitution.

In yet some further embodiments, the chimeras of the invention may comprise substitution of glycine 577 to lysine, specifically G577K. Thus, in some embodiments, the chimeras of at least one of SEQ ID NO. 45, 46, 47, 48, 210, 211, 212, and 213, may comprise a G577K substitution.

In some embodiments, the nucleic acid guided genome modifier chimeric protein provided by the invention may comprise as the PAM-free or reduced Cas protein, the dCas14 protein. Non-limiting examples for such nucleic acid guided genome modifier chimeric protein may include the dCas14-FokI, that in some embodiments may comprise the amino acid sequence as denoted by SEQ ID NO. 271, the StkC-dCas14-FokI, that in some embodiments may comprise the amino acid sequence as denoted by SEQ ID NO. 272, the HIVINT-dCas14-FokI, that in some embodiments may comprise the amino acid sequence as denoted by SEQ ID NO. 273, the SS07D-dCas14-FokI, that in some embodiments may comprise the amino acid sequence as denoted by SEQ ID NO. 274, the HMGN-dCas14-FokI, that in some embodiments may comprise the amino acid sequence as denoted by SEQ ID NO. 275, the HMGN-dCas14-FokI-HMGB1, that in some embodiments may comprise the amino acid sequence as denoted by SEQ ID NO. 276, the HMGN-dCas14-FokI-HMGB3, that in some embodiments may comprise the amino acid sequence as denoted by SEQ ID NO. 277, the HMGN-dCas14-FokI-HMGB4, that in some embodiments may comprise the amino acid sequence as denoted by SEQ ID NO. 278, the dCas14-FokI-HMGB1, that in some embodiments may comprise the amino acid sequence as denoted by SEQ ID NO. 279, the dCas14-FokI-HMGB3, that in some embodiments may comprise the amino acid sequence as denoted by SEQ ID NO. 280 and the dCas14-FokI-HMGB4, that in some embodiments may comprise the amino acid sequence as denoted by SEQ ID NO. 281.

Non-limiting examples for such nucleic acid guided genome modifier chimeric protein may comprise the HMGN-dScCas-FokI, that in some embodiments may comprise the amino acid sequence as denoted by SEQ ID NO. 354, the HMGN-dScCas-FokI-HMGB1, that in some embodiments may comprise the amino acid sequence as denoted by SEQ ID NO. 355, the HMGN-dScCas-FokI-HMGB3, that in some embodiments may comprise the amino acid sequence as denoted by SEQ ID NO. 356, the HMGN-dScCas-FokI-HMGB4, that in some embodiments may comprise the amino acid sequence as denoted by SEQ ID NO. 357, the dScCas-FokI-HMGB1, that in some embodiments may comprise the amino acid sequence as denoted by SEQ ID NO. 320, the dScCas-FokI-HMGB3, that in some embodiments may comprise the amino acid sequence as denoted by SEQ ID NO. 321 and the dScCas-FokI-HMGB4, that in some embodiments may comprise the amino acid sequence as denoted by SEQ ID NO. 322.

Still further, in some embodiments, the nucleic acid guided genome modifier chimeric protein provided by the invention may comprise as the PAM-free or reduced Cas protein, the Ancestral dCas9 protein, non-limiting example for such chimera, using FokI as the effector/modifier component may be the Ancestral dCas9-FokI, that in some embodiments comprise the amino acid sequence as denoted by SEQ ID NO. 284.

In some embodiments, the nucleic acid guided genome modifier chimeric protein provided by the invention may comprise as the PAM-free or reduced Cas protein, at least one of the dCasFl-Fok protein, dCasF2-Fok protein and the dCasF3-Fok protein. In some specific embodiments, the PAM-free or reduced chimeras of the invention may be a fusion protein between a nuclease-deficient CasF-2 and FokI, designed by dCasF2-Fok. In some embodiments, dCasF2-Fok chimera of the invention may comprise the dCasF2 amino acid sequence as denoted by SEQ ID NO 361.

In some further embodiments, the dCasF2 of the dCasF2-Fok fusion protein may have all or part of its RuvC nuclease domain deleted. In certain embodiment, the RuvC domain includes dCasF-2 residues 389-410, residues 601-612, and residues 689-706 of the dCasF2 sequence as denoted by SEQ ID NO. 361. In some embodiments, the RuvC domain residues may be replaced by short linkers (Gly-Gly-Ser-Gly, as denoted by SEQ ID NO. 399). In some particular embodiments, the fusion deletion variants of dCasF2-Fok may comprise dCasF2-Fok with a deletion of residues 389-410 (designated herein as dell). In some specific embodiments, the dell chimera may comprise the amino acid sequence as denoted by SEQ ID NO: 362. In yet some further embodiments, the fusion deletion variants of dCasF2-Fok may comprise dCasF2-Fok with a deletion of residues 601-612 (designated herein as de12). In some specific embodiments, the dell chimera may comprise the amino acid sequence as denoted by SEQ ID NO: 364.

In yet some further embodiments, the fusion deletion variants of dCasF2-Fok may comprise dCasF2-Fok with a deletion of residues 689-706 (designated herein as de13). In some specific embodiments, the dell chimera may comprise the amino acid sequence as denoted by SEQ ID NO: 365. Still further, in some embodiments, the present disclosure provides dCasF2-FokI chimeras that comprise either an N′-terminal FokI (SEQ ID NO: 363), or a C′-terminal Fold (SEQ ID NO. 366).

As indicated above, the effector/modifier component of the nucleic acid guided genome modifier or effector chimeric protein, complex or conjugate of the invention may perform any functional or physical modification on a target nucleic acid sequence. Physical modification as used herein includes cleavage, addition, deletion, insertion, editing functions (e.g., substitutions, mutations, deletions, insertions), addition of a chemical group, labeling, and the like. Functional modifications include gene repression or activation.

Repression or activation is achieved when the PAM-reduced or abolished Cas protein of the invention or any chimeric effector or modifier thereof, that comprise at least one transcription activator or repressor as the modifier/effector component, is guided to the target (e.g., by the target recognition element, discussed herein after), for example, a promoter region of the desired gene or any other control or regulatory element. A non-limiting example for repressor useful in the invention as the effector/modifier component, may be the Krüppel associated box (KRAB) domain, which enhances repression of the targets (Gilbert et al., Cell 154:442-451 (2013)). Thus, in some embodiments, the guided effector/modifier-chimeric protein or conjugate of the invention may comprise the KRAB—MeCP2 fusion protein.

Still further, activation of a target sequence is achieved when a transcriptional activator is used as the effector/modifier component in the chimeric protein or conjugate of the invention. A non-limiting example for such activator may be the Herpes simplex virus protein vmw65, also known as VP16 (Gilbert et al., Cell 154:442-451 (2013)).

In some further embodiments, the nucleic acid guided genome modifier chimeric protein of the invention may further comprise additional elements, for example, at least one cellular localization domain such as Nuclear localization signal (NLS), at least one Mitochondrial leader sequence (MLS), for example, at least one Chloroplast leader sequence; and/or any sequences designed to transport or lead or localize a protein to a nucleic acid containing organelle, a cellular compartment or any subdivision of a cell.

According to some embodiments, a “cellular localization domain” which can localize the nucleic acid guided genome modifier chimeric protein of the invention or a system comprising the modifier/effector chimeric protein and at least one target recognition element, or any complex thereof, to a specific cellular or sub cellular localization in a living cell, may optionally be part of the modifier/effector component of the nucleic acid guided genome modifier chimeric protein of the invention. The cellular localization domain may be constructed by fusing the amino-acid sequence of one of these components to amino-acids incorporating a domain comprising a Nuclear localization signal (NLS); a Mitochondrial leader sequence (MLS); a Chloroplast leader sequence; and/or any sequences designed to transport or lead or localize a protein to a nucleic acid containing organelle, a cellular compartment or any subdivision of a cell. In some exemplary embodiments, the organism is eukaryotic and the cellular localization domain comprises a nuclear localization domain (NLS) which allows the protein access to the nucleus and the genomic DNA within. Still further, in some embodiment, the at least two components of the nucleic acid guided genome modifier chimeric proteins of the invention, specifically, the PAM-reduced or abolished Cas protein and the effector/modifier component may be fused together or alternatively, may be linked to form the chimeric protein or conjugate by at least one linker. In yet some further alternative or additional embodiments, at least one linker in accordance with the present disclosure may replace at least one fragment or domain of the Cas protein used for the nucleic acid guided genome modifier chimeric proteins of the present disclosure, for example, replacement of the HNH domain by the linkers disclosed herein Example 3, specifically in nucleic acid guided genome modifier chimeric proteins that comprise the amino acid sequence as denoted by any one of SEQ ID NO. 342 344, 349 350 and 351. In some embodiments, the linker may comprise any compound bridging or connecting at least one amino acid residue of each component of the nucleic acid guided genome modifier chimeric proteins of the present disclosure. In some further embodiments, such linker is any inorganic or organic molecule, any small molecule, any peptide (L- as well as D-aa residues), or any combinations thereof. Still further, in some embodiments, the term “linker” in the context of the invention concerns an amino acid sequence of about 1 to about 20 or more amino acid residues positioned between, and connecting the at least two components of the nucleic acid guided genome modifier chimeric proteins of the invention. It should be appreciated that the linker may be positioned in the central region of at least one of the components of the nucleic acid guided genome modifier chimeric proteins of the invention and/or in at least one of their termini, namely at the C-terminus and/or at the N-terminus thereof. For example, a linker in accordance with the invention may be of 1, 2, 3, 4, 5, 6, 7, 8, 9, 10, 11, 12, 13, 14, 15, 16, 17, 18, 19 or 20 or more amino acid residues long. In some embodiments the linker according to the present invention encompasses 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 1-2 or 1 amino acid residue/s. In other embodiments the linker encompasses 1, 2, 3, 4, 5, 6, 7, 8, 9, 10, 11, 12, 13, 14, 15, 16, 17, 18, 19, 20 or more amino acid residues. In some embodiments, the linker used in the present invention referred to herein as “short linker/s”, may comprise between about 1 to 5 amino acid residues, specifically, 1, 2, 3, 4, or 5 amino acid residues. In yet some further alternative embodiments, a linker used in the nucleic acid guided genome modifier chimeric proteins disclosed herein may be referred to herein as “long linker”, and may comprise about 5 to 20 or more amino acid residues, specifically, 5, 6, 7, 8, 9, 10, 11, 12, 13, 14, 15, 16, 17, 18, 19, 20. Specific embodiments refer to a linker composed of 12 amino acid residues. Particular and non-limiting embodiments for such linkers may include the linkers that comprise the amino acid sequences as denoted by any one of SEQ ID NO. 206, 207 (long), 306 (long), 208 (short) In yet some further embodiments, the linkers used by the nucleic acid guided genome modifier chimeric proteins of the present disclosure may comprise particular amino acid residues having a desired property, for example, positively charged amino acid residues, negatively charged amino acid residues, aromatic residues, and the like. In some embodiments, the linker used in the present disclosure may be a positively charged linker that comprise at least one Lys and/or Arg residues. Particular and non-limiting embodiments for such linker may include the linkers that comprise the amino acid sequences as denoted by any one of SEQ ID NO. 307 and 481, or alternatively the uncharged linker of SEQ ID NO. 209. Still further, the linkers used by the present disclosure may be derived from various polypeptides, for example, the H-NS nucleoid-associated protein. Non-limiting example for such linker may a linker comprising the amino acid sequence as dented by SEQ ID NO. 485. In some embodiments, the connection between the at least one linker and each of the components of the nucleic acid guided genome modifier chimeric proteins of the invention, may be formed by any means, such as covalent peptide bonds, disulfide bonds, chemical crosslinks, etc., or non-covalent associations, such as hydrogen bonding, van der Waal's contacts, electrostatic salt bridges, etc. In some embodiments, the linker is covalently linked or joined to the amino acid residues in its vicinity.

The invention provides nucleic acid guided genome modifier chimeric protein composed of two components as discussed above, the PAM-reduced or abolished Cas protein and the effector/modifier component. Non-limiting examples for such effector/modifier chimeras are disclosed by the invention, as specified above. However, it must be understood that the invention further encompasses any variant or any derivative of each of the chimeras provided herein, specifically, any variant, fragments, peptides or derivative of any of the chimeras disclosed by the amino acid sequences as denoted by any one of SEQ ID NO. 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492. More particular embodiments refer to the chimeras disclosed by the amino acid sequences as denoted by any one of SEQ ID NO. 2, 375, 444, 448, 467, 476, 478, 479 and 480, or any variants and derivatives thereof.

In some further embodiments, the nucleic acid guided genome modifier or effector provided by the invention may be divided into at least two polypeptides (or sequences encoding them) that can be reconstituted using inteins. Thus, in some embodiments, a fragment of the chimeras of the invention may encompass any N′-terminal and/or C′-terminal fragment or part of any of the chimeras disclosed in the present disclosure. In some embodiments, these fragments are attached or connected to an intein. As used herein, an intein is a segment of a protein that is able to excise itself and join the remaining portions (the exteins) with a peptide bond during protein splicing. Inteins have also been called protein introns, by analogy with (RNA) introns. They are intervening protein domains that can undergo a posttranslational autoprocessing termed protein splicing.

In some embodiments, the intein suitable for the invention may the N-terminal half of the DnaE intein from Nostoc punctiforme, and/or the C-terminal half of the DnaE intein from Nostoc punctiforme.

In some specific embodiments, the nucleic acid guided genome modifier or effector provided by the invention, specifically, the dScCas-FokI may be divided into at least two polypeptides (N′ and C′). In yet some further specific embodiments, these dScCas-FokI fragments, may comprise amino acid sequences as denoted by SEQ ID NO: 352 and 353.

It should be noted that “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that in some embodiments may undergo post-translational modifications, such as phosphorylation, acetylation, glycosylation, manosylation, amidation, carboxylation, sulfhydryl bond formation, cleavage and the like.

By “fragments or peptides” it is meant a fraction of the protein of the invention. A “fragment” of a molecule, such as any of the amino acid sequences of the present invention, is meant to refer to any amino acid subset. This may also include “variants” or “derivatives” thereof. A “peptide” is meant to refer to a particular amino acid subset having a functional, structural activity or function displayed by the protein disclosed by the invention.

It should be appreciated that the invention encompasses any variant or derivative of the CRISPR-Cas protein or chimeric protein of the invention and any polypeptides that are substantially identical or homologue. The term “derivative” is used to define amino acid sequences (polypeptide), with any insertions, deletions, substitutions and modifications to the amino acid sequences (polypeptide) that either do not alter the activity of the original polypeptides or alter it purposefully. In this connection, a derivative or fragment of the variant of the invention may be any derivative or fragment of the variant and/or mutated molecule, specifically as denoted by SEQ ID NO. 2, 10, 14, 15, 16, 17, 24-48, 56, 210-213, 256-281, 284,288-290, 308-318, 320-323, 330-366, and 375-396, 406, 407 and 426-480, 487-492, that do not reduce or alter the activity of the variant of the invention.

By the term “derivative” it is also referred to homologues, variants and analogues thereof. Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100% , specifically as compared to the entire sequence of the proteins of interest in accordance with the invention, for example, any of the proteins that comprise the amino acid sequence as denoted by SEQ ID NO. 2, 10, 14, 15, 16, 17, 24-48, 56, 210-213, 256-281, 284,288-290, 308-318, 320-323, 330-366, and 375-396, 406, 407 and 426-480. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 2, 10, 14, 15, 16, 17, 24-48, 56, 210-213, 256-281, 284, 288-290, 314-318, 320-323, 330-366, and 375-396, 406, 407 and 426-480, 487-492.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as well as “substituted,” “deleted”, “inserted”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M).

More specifically, amino acid “substitutions” are the result of replacing one amino acid with another amino acid having similar structural and/or chemical properties, i.e., conservative amino acid replacements Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention.

Still further, it should be understood that the nucleic acid modifier/effector component used for the nucleic acid guided genome modifier chimeric protein of the invention, may be either a protein-based component (in a chimeric or fusion protein as disclosed herein above), or in some alternative embodiments, a nucleic-acid based modifier. In some further specific embodiments, the nucleic acid modifier component of the chimeric protein of the invention may be a modifier based on at least one nucleic acid molecule having a catalytic activity, for example, a Ribozyme or DNAzyme, or any catalytic nucleic acid molecule or DNA machine (e.g., DNA tweezer), that may physically or functionally modify a target nucleic acid sequence.

In yet another aspect, the invention provides a nucleic acid molecule comprising a nucleic acid sequence encoding at least one Cas protein or any Cas protein derived domain, having reduced or abolished PAM constraint or any fragment, variant, mutant, fusion protein, complex or conjugate thereof. It should be noted that according to optional embodiments, at least one of the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

In more specific embodiments, the nucleic acid molecule of the invention may encode any of the CRISPR-Cas protein described herein, specifically, any of the Cas proteins having reduced or abolished PAM constraint or restriction, in accordance with the invention. In yet some further embodiments, the nucleic acid sequence of the invention may encode any of the nucleic acid guided genome modifier/effector chimeric protein, complex or conjugate described by the invention. Non-limiting examples for such chimeras may include, but are not limited to any one of the chimeras that comprise the amino acid sequence as denoted by any one of 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492. More particular embodiments refer to the chimeras disclosed by the amino acid sequences as denoted by any one of SEQ ID NO. 2, 375, 444, 448, 467, 476, 478, 479 and 480, or any variants and derivatives thereof. Still further, it should be understood that the invention further encompasses any nucleic acid sequence encoding any of the chimeras of the invention or any fragment, peptide variant or derivatives thereof, or any optimized version thereof. A non-limiting example for sequence encoding the dCas9-FokI chimeras optimized for plants, may refer to the nucleic acid sequence as denoted by SEQ ID NO. 58.

The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an ——OH, then and ——H, then an ——OH, then an ——H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

It should be noted that the nucleic acid molecules (or polynucleotides) according to the invention can be produced synthetically, or by recombinant DNA technology. Methods for producing nucleic acid molecules are well known in the art.

The nucleic acid molecule according to the invention may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides. In further embodiments the nucleic acid molecule according to the invention comprises 1,000-10,000 nucleotides, e.g., about 1,000, 2,000, 3,000, 4,000, 5,000, 6,000, 7,000, 8,000, 9,000 or 10,000 nucleotides. In yet further embodiments the nucleic acid molecule according to the invention comprises more than 10,000 nucleotides, for example, 20,000, 30,000, 40,000, 50,000, 60,000, 70,000, 80,000, 90,000 or 100,000 nucleotides.

The invention further encompasses in some embodiments thereof at least one nucleic acid cassette comprising the nucleic acid sequence of the invention, or any vector or vehicle thereof. More specifically, the nucleic acid molecules provided by the invention may be comprised in some embodiments, within nucleic acid cassettes. The term “nucleic acid cassette” refers to a polynucleotide sequence comprising at least one regulatory sequence operably linked to a sequence encoding a nucleic acid sequence of interest. All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence.

Still further, the nucleic acid molecules of the invention or any cassettes thereof may be comprised within vector/s. Vector/s, as used herein, are nucleic acid molecules of particular sequence that can be introduced into a host cell, thereby producing a transformed host cell or be transiently expressed in the cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, (as detailed below) useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, or they may be integrated into the target cell genome, through homologous recombination or random integration, e.g. retrovirus-derived vectors such as AAV, MMLV, HIV-1, ALV, etc.

As indicated above, in some embodiments, viral vectors may be applicable in the present invention. The term “viral vector” refers to a replication competent or replication-deficient viral particle which are capable of transferring nucleic acid molecules into a host.

In some embodiments such viral vectors may be used for transient expression of the components of the invention in the cell and may or may not be present in the cells ultimately delivered to the patient.

The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e. vectors constructed using complementary coding sequences from more than one viral subtype. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

More specifically, in some embodiments, the nucleic acid molecules suitable to methods of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (Ad11A and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells.

Production systems for rAAV vectors typically consist of a DNA-based vector containing a transgene expression cassette, which is flanked by inverted terminal repeats (payload). Construct sizes are limited to approximately 4.7-5.0 kb, which corresponds to the length of the wild-type AAV genome. In some embodiments it would thus be advantageous to have a payload smaller than this upper limit. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments AAV serotype 6 may be suitable for the methods of the invention. In yet some further embodiments, AAV serotype 8 may be suitable for the methods, systems, and the nucleic acid guided genome modifier chimeric protein of the invention.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the methods, systems, and the nucleic acid guided genome modifier chimeric protein of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene.

Still further, in some embodiments, SV40 may be used as a suitable vector by the methods, systems, and the nucleic acid guided genome modifier chimeric protein of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules of the invention that contains the nucleic acids sequence of interest into packaging cell lines and of collecting the viral particles that are generated by the packaging lines are well known in the art.

In some alternative embodiments, the vector may be a non-viral vector. More specifically, such vector may be in some embodiments any one of plasmid, minicircle and linear DNA, ssDNA (that are especially useful for donor integration at cleavage site) or RNA (useful to avoid long term expression and or integration) or a modified polynucleotide (mainly chemically protective modifications to protect RNA or DNA-RNA chimeras to enhance specificity and or stability). Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is sometimes less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA, RNA or RNP entrance into the targeted cells is facilitated. In more specific embodiments, the vector may be a naked DNA vector. More specifically, such vector may be for example, a plasmid, minicircle or linear DNA.

Naked DNA alone may facilitate transfer of a nucleic acid sequence (2-200 Kb or more) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or DoggyboneTM are double-stranded, linear DNA construct that solely encodes an payload expression cassette, comprising antigen, promoter, polyA tail and telomeric ends.

It should be appreciated that all DNA vectors disclosed herein, may be also applicable for the methods, systems and compositions of the invention.

Still further, it must be appreciated that the invention further provides any vectors or vehicles that comprise any of the nucleic acid molecules disclosed by the invention, as well as any host cell expressing the nucleic acid molecules disclosed by the invention.

It should be understood that any of the viral vectors disclosed herein may be relevant to any of the nucleic acid molecules discussed in other aspects of the invention, specifically to nucleic acid molecules encoding the SCNA (gRNA), the Donor or the protein components as described by the invention.

As indicated above, vectors may be provided directly to the subject cells thereby being contacted with the cell/s. In other words, the cells are contacted with vectors comprising the nucleic acid molecules of the invention that comprise the nucleic acid sequence of interest such that the vectors are taken up by the cells. Methods for contacting cells with nucleic acid vectors that are plasmids, such as electroporation, calcium chloride transfection, and lipofection (e.g. using Lipofectamin), are well known in the art. DNA can be introduced as naked nucleic acid, as nucleic acid complexed with an agent such as a liposome, nanoparticles or poloxamer, or can be delivered by viruses (e.g., adenovirus, AAV).

A further aspect of the invention relates to a nucleic acid guided genome modifier/effector system.

In more specific embodiments, the system of the invention comprises the following components:

The first component (a), of the system of the invention may be at least one Cas protein or Cas protein derived domain, having reduced or abolished PAM constraint or any fragment, variant, mutant, fusion/chimeric protein, complex or conjugate thereof. Alternatively, the system of the invention may comprise at least one nucleic acid sequence encoding this Cas protein or any variant, mutant, fusion/chimeric protein, complex or conjugate thereof. It should be noted that at least one of: the PBD of the Cas protein of the system of the invention, any fragment of said PBD, and at least one amino acid residue adjacent to the PBD, is deleted or replaced.

The second component (b), of the system of the invention may be at least one target recognition element, or alternatively, any nucleic acid sequence encoding the target recognition element.

According to some embodiments, the system disclosed herein is modular and components thereof can self-assemble within a target cell either in vivo or in vitro, allowing the supply of one type of Cas protein having abolished or reduced PAM-restriction or constraint, or any fusion protein thereof with the modifier or effector component, at a time with one or a multiplicity of target recognition element/s concomitantly. Furthermore, in some embodiments, the Cas protein having abolished or reduced PAM-restriction or any fusion protein thereof can be delivered to a desired cell(s) and expressed in vivo, awaiting the delivery of any appropriate target recognition element/s at a later time. In some embodiments, the Cas protein having abolished or reduced PAM-restriction or any fusion protein, complex or conjugate thereof and the target recognition element/s may be delivered simultaneously, or essentially simultaneously. Thus, the combination of the Cas protein having abolished or reduced PAM-restriction or any fusion protein, complex or conjugate thereof and the target recognition element/s, preferably within the desired target cell, may accomplish the induction of specific genomic double strand breaks (DSBs), or any other desired nucleic acid modification, in vivo.

In some embodiments, the Cas protein of the system of the invention may be at least one of Cas9, CasX, Cas14a1, Cas14b5, CasF, ancestral Cas and Cas12a or any variant, mutant, fusion/chimeric protein, complex or conjugate thereof. In yet some further embodiments, the chimeric or fusion protein, complex or conjugate of such Cas protein of the system of the invention, may further comprise at least one nucleic acid modifier component.

In more specific embodiments, the Cas protein used as a component in the system of the invention, may be at least one of ScCas9, SpCas9, an ancestral Cas9, deltaproteobacteria CasX, Cas12a, CasF-1, CasF-2, CasF-3, Cas14a1, or Cas14b5. According to such embodiments, at least one PAM interacting Arginine residue of the PBD of such Cas protein may be deleted or replaced.

In more specific embodiments, the Cas used for the systems of the invention may be ScCas9. In some embodiments, the ScCas9 may comprise an amino acid sequence as denoted by SEQ ID NO. 258, with a replacement or deletion of at least one of: residues Thr1330 to Arg1342, residues Glu1228 to Tyr1343, residues Glu1108 to Asp1375, residues Ile367 to Ala376 and residues Lys1337 and Gln1338. As indicated herein before in connection with other aspects of the invention, the particular residues defining the PBD of the Cas protein of the invention, specifically, the ScCas, may start at least one, two, three or more residues N′ or C′ to the specified starting residue, and/or end at least one, two, three or more residues C′ or N to the specified end residue.

For example, in case of residues Thr1330 to Arg1342, the deleted or replaced sequence may comprise a sequence stating at any one of residues 1327, 1328, 1329, 1330, 1331, 1332 or 1333 of ScCas, and ends at any one of residues 1339, 1340, 1341, 1342, 1343, 1344 or 1345.

In yet some further specific embodiments, the PAM-reduced or abolished CRISPR-Cas protein of the invention may further comprise at least one NSBD. In yet some further embodiments, the NSBD may be added to said Cas (either to the N′ and/or C′ terminus thereof) and/or may replace at least one of the PAM binding domain, and/or the PAM recognition motif, and/or the HNH-nuclease domain, and/or at least one adjacent amino acid residue thereof.

More specifically, such NSBD may be at least one dsDBP binding domain or protein, and any variant and fragments thereof.

In some specific embodiments, the at least one dsDBP that replaces the PAM binding domain of the Cas protein used by the systems of the invention and/or at least one adjacent amino acid residues thereof, may be at least one of: at least one ZF, HTH, SH3 domain, Non-specific RVD from AvrBS3 protein family, CBD protein and StkC, domain or protein, and any variant and fragments thereof.

In more specific embodiments, the Cys₂His₂ of Testis zinc finger 3 (TZD) used herein may comprise the amino acid sequence as denoted by SEQ ID NO. 265. In yet some further embodiments, the Lac repressor (Lad) residues 1 to 46, that may be applicable in the present invention, may comprise the amino acid sequence as denoted by SEQ ID NO. 259. In certain embodiments, the SH3 domain comprising at least one of: residues 219 to 270 of HIV integrase protein, that may be applicable in the present invention, may comprise the amino acid sequence as denoted by SEQ ID NO. 260. Still further, in some embodiments, residues 1 to 64 of the Sso7D DNA-binding protein of Sulfolobus solfataricus, that may be applicable in the present invention, may comprise the amino acid sequence as denoted by SEQ ID NO. 261. In some embodiments, residues 1 to 64 of the Sto7D DNA-binding protein from Sulfolobus tokodaii, that may be applicable in the present invention, may comprise the amino acid sequence as denoted by SEQ ID NO. 262. In some further embodiments, StkC domain that comprise residues 232-305 of Arabidopsis MBD7 methyl-CpG-binding domain and may be applicable in the present invention, may comprise the amino acid sequence as denoted by SEQ ID NO. 263. In yet some further embodiments, CBD that comprises at least one High Mobility Group (HMG) protein, and may be applicable in the present invention, may comprise the amino acid sequence as denoted by SEQ ID NO. 264.

Still further, in some embodiments, the PAM binding domain of the Cas protein used by the systems of the invention and/or at least one adjacent amino acid residues is replaced by at least one NSBD that may be in some embodiments, at least one SSB.

In yet some further embodiments, the Cas protein used as a component in the system of the invention may be a Cas mutant or variant. In some specific embodiments, such mutant or variant may be a Cas protein having altered activity, stability, specificity, solubility, size or any other altered functional and/or structural property. In some embodiments, such Cas protein may be a Cas protein having reduced or abolished nucleolytic activity. In yet some further embodiments, such Cas protein may have a reduced size. More specifically, such mutant or variant further comprises at least one of: (a) at least one point mutation substituting aspartic acid residue at position 10 to alanine (D10A) and/or at least one point mutation substituting histidine residue 849 to alanine (H849A); the Cas protein may further or alternatively comprise (b) at least one deletion, substitution, mutation and/or replacement of at least one of: (i) at least one of the HNH-nuclease domain or any fragment thereof and/or at least one amino acid residue thereof; (ii) the REC2 domain or any fragments thereof and/or at least one amino acid residue thereof; (iii) the FLEX domain or any fragments thereof and/or at least one amino acid residue thereof; (iv) the RUVC domain or any fragments thereof and/or at least one amino acid residue thereof, and (v) any combinations of (i), (ii), (iii), and (iv); and (c) at least one mutation in at least one residue of at least one of (i) the HNH-nuclease domain or any fragment thereof; (ii) the REC2 domain or any fragments thereof; (iii) the FLEX domain, or any fragments thereof; (iv) the RUVC domain or any fragments thereof; and (v) and any combinations of (i), (ii), (iii), and (iv). Still further, in some embodiments, the PAM-reduced or abolished Cas protein of the invention may comprise a deletion in at least one of (i), (ii), (iii), and (iv) or any combinations thereof and additionally, at least one mutation in at least one amino acid residue comprised within the PBD of the ScCas9. Non-limiting embodiments for such mutations may comprise the QQ mutant that may comprise two Gln residues that substitute Arg residues 370 and 372 of the ScCas9 as denoted by SEQ ID NO. 258. In yet some further embodiments, the PAM abolished or reduced ScCas9 of the invention may comprise the AA mutant, that comprise two Ala residues that substitute Arg residues 370 and 372 of SEQ ID NO. 258. As shown by examples 2 and 3, the PAM abolished or reduced ScCas9 of the present disclosure may comprise Sc loop QQ or AA mutations, or alternatively, Sc loop depletion or replacement thereof with at least one dsDBP or SSB as discussed above, combined with deletion of HNH, and replacement of the RuvC and Rec domains with ancestral versions.

In yet some further embodiments, in case a chimeric or conjugated Cas protein is used by the systems of the invention, the Cas protein is a mutated Cas. Such Cas mutant is in some embodiments a defective CRISPR-Cas protein devoid of a nucleolytic activity.

It should be appreciated that in more specific embodiments, the chimeric protein or conjugate used for the systems of the invention may be any of the Chimeric proteins defined by the invention, specifically, in any of the aspects disclosed herein.

In some particular embodiments, the systems of the invention may use any of the nucleic acid guided genome modifier chimeric protein of the invention, specifically, any chimera comprising the amino acid sequence as denoted by any one of 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492, or any variants, fragments or derivatives thereof as specified by the invention herein before. More particular embodiments refer to the chimeras disclosed by the amino acid sequences as denoted by any one of SEQ ID NO. 2, 375, 444, 448, 467, 476, 478, 479 and 480, or any variants and derivatives thereof.

Still further, in some embodiments, the Cas protein or any variant, mutant, fusion protein, complex or conjugate thereof, used by the systems of the invention is capable of binding at least one target recognition element. In some specific embodiments, such at least one target recognition element may be at least one nucleic acid target recognition element. More specifically, at least one of: a single strand RNA molecule, a double strand RNA molecule, a single strand DNA, a double strand DNA, a modified DNA molecule, a modified RNA molecule, a LNA, a PNA and any hybrid or combinations thereof.

As used herein a “target recognition element” is a nucleic acid sequence (either RNA or DNA or a modified nucleic acid or a combination thereof) that will direct the nucleic acid-modifier/effector component (e.g., protein that directly or indirectly modify the target sequence) of the chimeric protein or conjugate of the invention.

More specifically, in some embodiments, the target recognition element of the invention, that is also referred to herein as a specificity conferring nucleic acid (SCNA), or as guide nucleic acids (e.g., guide RNA), may comprise at least one of: a single-strand DNA, a single strand RNA, a double strand RNA, a modified DNA, a modified RNA, a locked-nucleic acid (LNA) and a peptide-nucleic acid (PNA), any hybrids thereof, or any combinations thereof. In some embodiments, the target recognition element or SCNA of the invention comprises a specificity-defining sequence configured to specifically interact with the target nucleic acid. The interaction between the target recognition element, or SCNA and the target nucleic acid is through base pairing, selected from the group consisting of a full double helix base pairing, a partial double helix base pairing, a full triple helix base pairing, a partial triple helix base pairing, and D-loops, R-loops or branched forms, formed by said base pairing.

In additional embodiments, the target recognition element or SCNA may comprise a recognition region, configured to associate/bind/attach with a PAM reduced/free Cas protein of the invention or any chimera, complex or conjugate thereof, specifically, the nucleic acid guided genome modifier/effector chimeric protein, complex or conjugate of the invention. In some embodiments, the recognition region comprises a modification selected from the group consisting of 5′-end modification, 3 ′-end modification, and internal modification. The modification may be selected from, but not limited to nucleotide modification, Biotin, Fluorescein, Amine-linkers, oligo-peptides, Aminoallyl, a dye molecule, fluorophores, Digoxygenin, Acrydite, Adenylation, Azide, NHS-Ester, Cholesteryl-TEG, Alkynes, Photocleavable Biotin, Thiol, Dithiol, Modified bases, phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxylnosine, 5-nitroindole, 2-O-methyl RNA bases, Iso-dC, Iso-dG, Flourine modified bases and Phosphorothioate bonds, and proteins covalently bound by their interaction with the specific nucleotide sequences. The proteins covalently bound by their interaction with the specific nucleotide sequences are selected from Agrobacterium VirD2 protein, Picomavirus VPg, Topoisomerase, PhiX174 phage A protein, PhiX A* protein and any variants thereof.

In some embodiments, the association/binding/attachment between the modification on the target recognition element or SCNA and the PAM reduced/free Cas protein of the invention or any chimera, complex or conjugate thereof, results from a non-covalent interaction of a binding-pair selected from: Biotin-Avidin; Biotin-Streptavidin; Biotin-modified forms of Avidin; Protein-protein interactions; protein-nucleic acid interactions; ligand-receptor interactions; ligand-substrate interactions; antibody-antigen interactions; single chain antibody-antigen; antibody or single chain antibody-hapten interactions; hormone-hormone binding protein; receptor-agonist; receptor-receptor antagonist; anti-Fluorescein single-chain variable fragment antibody (anti-FAM ScFV)—Fluorescein; anti-DIG single-chain variable fragment (scFv) immunoglobin (DIG-ScFv)—Digoxigenin (DIG); IgG-protein A; enzyme-enzyme cofactor; enzyme-enzyme inhibitor; single-strand DNA-VirE2; StickyC—dsDNA; RISC—RNA; viral coat protein-nucleic acid and Agrobacterium VirD2-VirD2 binding protein; and any variants thereof.

In some embodiments, binding/association between the target recognition element, or SCNA and the PAM reduced/free Cas protein of the invention or any chimera, complex or conjugate thereof is covalently created in vivo. In some embodiments, the covalent association of the PAM reduced/free Cas protein of the invention or any chimera, complex or conjugate thereof and the target recognition element or SCNA results from a biological interaction of Agrobacterium VirD2-Right border sequence or any variants thereof, and is created in a bacterium comprising Agrobacterium.

In some embodiments, the recognition region comprises a nucleotide motif capable of interacting/attaching/binding with the PAM reduced/free Cas protein of the invention or any chimera, complex or conjugate thereof. In some embodiments, the interaction pair is selected from: Zinc finger protein-Zinc finger motif; restriction enzyme recognition domain-restriction enzyme recognition sequence; DNA binding domain of transcription factor-DNA motif; repressor-operator; Leucine zipper -promoter; Helix loop helix-E box domain; RNA binding motifs comprising Arginine-Rich Motif domains, αβ protein domains, RNA Recognition Motif (RRM) domains, MS2 coat protein-MS2 RNA binding hairpin, K-Homology Domains, Double Stranded RNA Binding Motifs, RNA-binding Zinc Fingers, and RNA-Targeting Enzymes-cognate specific RNA sequence; HIV-rev protein-Stem IIB of the HIV rev response element (RRE); Bovine immunodeficiency virus (BIV) Tat main binding domain-loop 1 of the BIV trans-acting response element (TAR) sequence; Phage lambda, phi21, and P22 Nproteins-The boxB loop hairpins in the N-utilization (nut) sites in their respective RNAs.

Still further, in case of a nucleic acid-modifier that is a protein such as a nuclease, the target recognition element may be a nucleic acid guide that targets the nuclease to a specific target position within a target nucleic acid sequence (e.g., SCNA, gRNA). The recognition of the target by the target recognition element is facilitated in some embodiments by base-pairing interactions. These target recognition elements are specifically relevant in case of guided nucleases. In some embodiments, for nucleases displaying a nucleolytic activity, directing the nuclease to a specific predetermined target site in the target nucleic acid may result in cleaving the phosphodiester bonds between monomers of nucleic acids (e.g., DNA and/or RNA) that may lead in some embodiments to specific modifications thereof, such as mutations, deletions, frame-shifts, insertion of a Donor nucleic acid, or replacement of the target or a portion thereof with an alternative Donor nucleic acid.

In yet some alternative embodiments, where the modifier used performs a modulation other than nucleolytic activity, directing the modifier to the target site may result in targeted modulation (e.g., activation or repression, methylation or demethylation and the like) of the target nucleic acid sequence targeted by the target recognition element. It should be noted that a target recognition element may comprise between about 3 nucleotides to about 100 nucleotides, specifically, 3, 4, 5, 6, 7, 8, 9, 10,15, 20, 25, 30, 35, 40, 45, 50, 55, 60, 65, 70, 75, 80, 85, 90, 95 100 or more. More specifically between about 10 nucleotides to 70 nucleotides or more.

It should be understood that in some embodiments, for modifying the target nucleic acid sequence by the nucleic acid guided genome modifier chimeric/fusion protein complex or conjugate of the systems of the invention, at least two target recognition elements may be used to guide at least two PAM reduced or abolished Cas protein and fusion proteins thereof to a single target site within the target nucleic acid sequence. According to such embodiments, each of the respective target recognition elements (e.g., SCNAs or gRNAs), may be directed to a target sequence that is located at a distance of between about 5 to 50 nucleotides from the target sequence/s recognized by at least one other targeting recognition element/s. In some embodiments, the distance may be any one of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more. In some embodiments, the target recognition elements used in the systems of the invention may be designed for targeting target sequences that are located at a distance of about 10 to 30 nucleotides from each other, specifically, 10, 11, 12, 13, 14, 15, 16, 17, 18, 19, 20, 21, 22, 23, 24, 25, 26, 27, 28, 29, 30, nucleotides, more specifically, 15 nucleotides or more, or 27 nucleotides or more. In some embodiments, the target recognition elements used in the systems of the invention may be designed for targeting target sequences that are located at a distance of about 15 nucleotides.

In some embodiments, the target recognition element/s, disclosed herein, that are also referred to herein as the programming oligonucleotides or SCNAs, have an infinite repertoire of sequences, thus conceivably achieving extreme sequence specificity in high complexity genomes. Moreover, as many programming oligonucleotides can be supplied concomitantly with a single protein effector moiety, e.g., the PAM-reduced or abolished Cas protein of the invention or any nucleic acid guided genome modifier or effector chimeric protein, complex or conjugate thereof, it is possible to modify more than one target at the same time, providing additional advantages over methods known in the art. This can be useful, for example, for rapidly knocking out a multiplicity of genes, or for inserting several different traits in different locations, or for tagging several different locations with one donor nucleotide tag.

Designing and preparing synthetic target recognition element/s is relatively simple, rapid and relatively inexpensive. It is also possible, in some embodiments of this invention, to produce target recognition elements in-vivo, circumventing the necessity to deliver chemically synthesized target recognition elements to a cell. Furthermore, these elements can be designed to base pair to almost any desired target sequence, and thus, can direct the molecular complex to almost any target sequence. Moreover, several sequences may be targeted in the same cell concomitantly. For example, in editing functions which require more than one cleavage site, such as deletion or replacement of specific stretches of nucleic acid, by simply providing different target recognition elements and one effector moiety, specifically, the Cas protein having abolished or reduced PAM-restriction or any fusion protein thereof, in accordance with the invention. The systems of the invention may comprise the nucleic acid guided effector/modifier and the at least one target recognition element, either in a mature form, for example, as a protein moiety and as a target recognition element (e.g., gRNA, split-gRNA), or as a Ribonucleoprotein (RNP). However, the invention further encompasses the option of a system comprising nucleic acid sequences encoding each of these components, specifically, nucleic acid sequence encoding the nucleic acid guided effector/modifier of the invention, and nucleic acid sequence encoding the at least one target recognition element. Thus, in some embodiments, the systems of the invention may comprise at least one nucleic acid sequence encoding said Cas protein or any variant, mutant, fusion/chimeric protein thereof and at least one nucleic acid sequence encoding at least one target recognition element, specifically, at least one gRNA. In some embodiments, the nucleic acid sequence encoding the Cas protein of the invention or any chimeric or fusion protein thereof, and the nucleic acid sequence encoding the target recognition element may be comprised in one or more nucleic acid cassette or any vector or vehicle.

In yet some further aspect thereof, the invention relates to at least one host cell, that is either genetically modified by, or that comprises at least one of:

(a), at least one Cas protein or any Cas derived domain having reduced or abolished PAM constraint or any fragment, variant, mutant, fusion/chimeric protein, complex or conjugate thereof, or alternatively, at least one nucleic acid sequence encoding the Cas protein or any variant, mutant, fusion/chimeric protein or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

(b) at least one target recognition element or any nucleic acid sequence encoding the target recognition element;

(c) at least one nucleic acid cassette or any vector or vehicle comprising at least one of the nucleic acid sequence of (a), the nucleic acid sequence of (b) or the nucleic acid sequence of (a) and (b); and

(d) at least one system comprising (a) and (b).

In yet some further embodiments, the CRISPR-Cas protein comprised within the host cell of the invention may be any of the Cas proteins as defined by the invention. It should be noted that in some further embodiments, the invention provide host cells comprising any of the fusion/chimeric protein or conjugates of the Cas protein, specifically, the nucleic acid guided genome modifier chimeric protein, complex or conjugate as defined by the invention. In some specific embodiments, the host cell of the invention may comprise any of the nucleic acid guided effector/modifier disclosed by the invention, for example, any of the modifiers comprising the amino acid sequence as denoted by any one of SEQ ID NO. 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492. More particular embodiments refer to the chimeras disclosed by the amino acid sequences as denoted by any one of SEQ ID NO. 2, 375, 444, 448, 467, 476, 478, 479 and 480, or any variants and derivatives thereof.

In yet some further embodiments, the host cell of the invention may comprise any of the nucleic acid molecules defined by the invention. Still further, the host cell of the invention may comprise any of the systems disclosed by the invention, as specified herein.

In some further embodiments, the host cells of the invention may comprise at least one target recognition element that may be at least one of: a single strand RNA molecule, a double strand RNA molecule, a ssDNA, a dsDNA, a modified DNA molecule, a modified RNA molecule, a LNA, a PNA and any hybrid or combinations thereof.

The term “host cell” includes a cell into which a heterologous (e.g., exogenous) nucleic acid or protein (e.g., PAM-reduced or abolished Cas protein or the nucleic acid guided genome modifier/effector chimeric protein, complex or conjugate of the invention) or Ribonucleoprotein (RNP) thereof (e.g., the system of the invention), has been introduced. Persons of skill upon reading this disclosure will understand that such terms refer not only to the particular subject cell, but also is used to refer to the progeny of such a cell. Because certain modifications may occur in succeeding generations due to either mutation or environmental influences, developmental maturation, or due to the intended action of the invention, such progeny may not, in fact, be identical to the parent cell, but are still included within the scope of the term “host cell”. In some embodiments, the host cells provided by the invention are transduced or transfected by the nucleic acid sequences provided by the invention that encode the PAM abolished or reduced CRISPR-Cas proteins of the invention, any chimeric proteins thereof, and systems. This may refer in some embodiments, to cells that underwent a transfection procedure, meaning the introduction of a nucleic acid, e.g., an expression vector, or a replicating vector, into recipient cells by nucleic acid-mediated gene transfer. Alternatively, or in combination with nucleic acids encoding components of the invention, all or part of the components may be delivered as an RNA, as a protein, or as a preassembled RNP. Transfection of eukaryotic cells may be either transient or stable, and is accomplished by various ways known in the art.

For example, transfection of eukaryotic cells may be chemical, e.g. via a cationic polymer (such as DEAE-dextran, polyethyleneimine, dendrimer, polybrene, calcium), calcium phosphate (e.g. phosphate, lipofectin, DOTAP, lipofectamine, CTAB/DOPE, DOTMA) or via a cationic lipid. Transfection of eukaryotic cells may also be physical, e.g. via a direct injection (for example, by Micro-needle, AFM tip, Gene Gun,), via biolistic particle delivery (for example, phototransfection, Magnetofection), or via electroporation (i.e., Lonza Nucleofector), laser-irradiation, sonoporation or a magnetic nanoparticle. Transfection of eukaryotic cells may also be biological (i.e., use of Agrobacterium in plants).

The term “host cells” as used herein refers to any cell known to a skilled person wherein the functional fragments or peptides thereof or any nucleic acid molecule or combination thereof according to the invention may be introduced. For example, a host cell may be any prokaryotic or eukaryotic cell of a unicellular or multi-cellular organism. More specifically, eukaryotic host cell/s in accordance with the invention may include, but is not limited to a yeast, fungi, a plant, an insect cell, an invertebrate cell, vertebrate cell, mammalian cell and the like. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

The “host cell” as used herein refers also to cells which can be transformed or transfected with naked DNA, any plasmid or expression vectors constructed using recombinant DNA techniques. A drug resistance or other selectable marker carried on the transforming or transfecting plasmid is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the phenotype for survival.

Eukaryotic cells may be mammalian cells, plant cells, fungi or cells of any organism. As used herein, the term “eukaryotic cell” refers to any cell type known to a person skilled in the art which is suitable for genetic manipulation. It should be noted that the term “eukaryotic cells” as used herein, further encompasses the autologous cells or allogeneic cells used by the methods of the invention via adoptive transfer, as discussed herein after in connection with other aspects of the invention. Thus eukaryote cells as herein defined may be derived from animals, plants and fungi, for example, but not limited to, insect cells, yeast cells or mammalian cells.

It should be further understood that the term “Cell”, is defined here as to comprise any type of cell, prokaryotic or a eukaryotic cell, isolated or not, cultured or not, differentiated or not, and comprising also higher level organizations of cells such as tissues, organs, calli, organisms or parts thereof. Exemplary cells include, but are not limited to: vertebrate cells, mammalian cells, human cells, plant cells, animal cells, invertebrate cells, nematodal cells, insect cells, stem cells, and the like.

Still further, it should be appreciated that the present disclosed further encompasses any cell or population of host cells as defined above, that are genetically modified or edited by the PAM-reduced or abolished Cas protein of the present disclosure or any chimera, conjugate, complex and system thereof. The invention further encompasses any genetically modified organism, either eukaryotic or prokaryotic that has been modified by the PAM-reduced or abolished Cas protein of the present disclosure, or to any tissue, cell (e.g., gamete cells, embryonic cells and the like) or organ derived from such genetically modified organism.

A further aspect of the invention relates to a composition comprising at least one of:

(a) at least one Cas protein or any Cas derived domain having reduced or abolished PAM constraint or any variant, mutant, fusion/chimeric protein, complex or conjugate thereof, or at least one nucleic acid sequence encoding the Cas protein or any fragment, variant, mutant, fusion/chimeric protein or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

(b) at least one target recognition element or any nucleic acid sequence encoding such target recognition element.

(c) at least one nucleic acid cassette or any vector or vehicle comprising at least one of the nucleic acid sequence of (a), the nucleic acid sequence of (b) or the nucleic acid sequence of (a) and (b).

(d) at least one system comprising (a) and (b), and

(e) at least one host cell modified by, and/or comprising at least one of: the nucleic acid cassette or any vector or vehicle of (c) and the at least one system of (d);or any matrix, nano- or micro-particle comprising at least one of (a), (b), (c), (d) and (e). It should be noted that the composition of the invention may optionally further comprises at least one of pharmaceutically acceptable carrier/s, diluent/s, excipient/s and additive/s.

In some specific embodiments, the composition may comprise any of the CRISPR-Cas protein as defined by the invention, any of the fusion/chimeric protein, complex or conjugate thereof, specifically, the nucleic acid guided genome modifier/effector chimeric protein, complex or conjugate disclosed by the invention, and/or any of the nucleic acid molecules of the invention, and/or any of the systems disclosed by the invention, and/or any of the host cells is defined by the invention.

In some embodiments, the compositions of the invention may comprise as an active ingredient, any of the PAM-reduced or abolished Cas protein of the invention, any of the nucleic acid guided genome modifier/effector chimeric protein, complex or conjugate of the invention, any systems thereof, any encoding nucleic acid sequence or any host cell comprising the same. In some particular embodiments, the composition of the invention may comprise any of the chimeras disclosed by the invention, specifically, any chimera comprising the amino acid sequence of any one of SEQ ID NO. 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492.

More particular embodiments refer to the chimeras disclosed by the amino acid sequences as denoted by any one of SEQ ID NO. 2, 375, 444, 448, 467, 476, 478, 479 and 480, or any variants and derivatives thereof.. The term “effective amount” relates to the amount of an active agent present in a composition, specifically, the PAM abolished or reduced CRISPR-Cas proteins of the invention, any chimeric proteins thereof, RNPs thereof, systems thereof, nucleic acid sequence encoding said CRISPR-Cas proteins or any vectors thereof, host cell/s transformed or transfected by said nucleic acid sequences, as provided by the invention and described herein, or cells comprising any of the nucleic acid guided genome modifier/effector chimeric protein, complex or conjugate of the invention as disclosed herein, that is needed to provide a desired level of active agent in the bloodstream or at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

An “effective amount” of the PAM abolished or reduced CRISPR-Cas proteins of the invention, any chimeric proteins thereof, systems, nucleic acid, host cell of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

In yet some further embodiments, the composition of the invention may optionally further comprise at least one of pharmaceutically acceptable carrier/s, excipient/s, additive/s diluent/s and adjuvant/s .

The pharmaceutical compositions of the invention can be administered and dosed by the methods of the invention, in accordance with good medical practice, systemically, for example by parenteral intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically the protein, nucleic acid, host cell of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question.

Still further, pharmaceutical preparations are compositions that include the protein, nucleic acid, host cell of the invention present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid or liquid such as tablets, capsules, powders, granules, ointments, solutions, suppositories, injections, inhalants, gels, microspheres, and aerosols. As such, administration of the protein, nucleic acid, host cell of the invention can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

Still further, the composition/s of the invention and any components thereof may be applied as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month. The application of the PAM abolished or reduced CRISPR-Cas proteins, any chimeric proteins thereof, systems thereof, nucleic acid sequence encoding said CRISPR-Cas proteins or any vectors thereof, host cell/s transformed or transfected by said nucleic acid sequence, in accordance with the invention or of any component thereof, or the effects thereof, may last up to the lifetime of the patient, a day, two days, three days, four days, five days, six days, a week, two weeks, three weeks, four weeks, a month, two months three months or even more. More specifically, for one, two, three, four, five, six, seven, eight, nine, ten, eleven, twelve months of more or for several years.

In yet a further aspect, the invention provides a method of modifying at least one target nucleic acid sequence of interest in at least one cell or in a biochemical reaction. More specifically, the method may comprise the steps of contacting the cell or the biochemical reaction with:

First (a), at least one Cas protein having reduced or abolished PAM constraint or any fragment, variant, mutant, fusion/chimeric protein, complex or conjugate thereof, or at least one nucleic acid sequence encoding said Cas protein or any variant, mutant, fusion/chimeric protein, complex or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced; and

Second (b), at least one target recognition element or any nucleic acid sequence encoding such target recognition element.

Alternatively, the method may involve contacting the cell or the biochemical reaction with (c), at least one nucleic acid cassette or any vector or vehicle comprising the nucleic acid sequence of (a), the nucleic acid sequence of (b) or the nucleic acid sequence of (a) and (b).

In yet some further embodiments (d), the cells or the biochemical reaction may be contacted with at least one system or composition comprising (a) and (b).

The invention provides methods for modifying at least one target nucleic acid sequence of interest in a target cell or in a biochemical reaction. It should be understood that as used herein, modifications performed in the target nucleic acid sequence encompass either physical modifications or functional modifications as discussed herein. Thus, in some embodiments, modifications include but are not limited to cleavage, deletion, insertion, replacement, binding, digestion, nicking, methylation, acetylation, ligation, recombination, helix unwinding, chemical modification, labeling, activation, and inactivation or any combinations thereof, as well as any editing activity (e.g., mutation, substitution, replacement, deletion or insertion of at least a part of the target sequence). Still further, functional modifications of the target sequence may lead to, but is not limited to: changes in transcriptional activation, transcriptional inactivation, alternative splicing, chromatin rearrangement, pathogen inactivation, virus inactivation, change in cellular localization, compartmentalization of nucleic acid, changes in stability, and the like, or combinations thereof.

As noted above, modifications of a target sequence by the methods of the invention, may include editing of the target sequence. It should be therefore understood that in some embodiments, both directed non-homologous-end-joining (NHEJ) and assisted homologous recombination (HR) may be utilized by the methods of the invention specifically and in a programmable manner to achieve one or more of the following:

(a) Mutating a DNA sequence by cleaving inside it, creating a double strand break (DSB), to be somewhat degraded by the endogenous nucleases and re-ligated by the endogenous NHEJ DNA repair mechanism to create either an in-frame deletion and/or a frame-shift mutation of the DNA.

In NHEJ one or more nucleotides may also be added in the DSB in a yet uncharacterized endogenous mechanism, essentially achieving the same effect of frame shifting or mutation.

(b) Deleting a stretch of DNA sequence by cleaving two sequences flanking it, to be re-ligated by the endogenous NHEJ DNA repair mechanism, or by assisted HR by cleaving in or near the sequence to be deleted and supplying a donor DNA which is subsequently recombined into the target, and which contains sequences flanking the sequence to be deleted in the target.

(c) Inserting a donor nucleic acid into a DSB by cleaving a target nucleic acid and supplying a Donor DNA to be either ligated directly into the gap by the NHEJ mechanism, or preferably, supplying a donor that has homology to the ends of the gap to be recombined and ligated into the gap by assisted HR.

(d) Replace a target nucleic acid sequence by cleaving both sequences flanking it, and supplying a donor nucleic acid to be inserted, to be ligated within the target flanking sequence either by NHEJ, or preferably, recombined and ligated by HR, by adding sequences similar to the target nucleic acid, or those flanking it, at the termini of the donor. “Donor nucleic acid” is defined here as any nucleic acid supplied to an organism or receptacle to be inserted or recombined wholly or partially into the target sequence either by DNA repair mechanisms, homologous recombination (HR), or by non-homologous end-joining (NHEJ).

As indicated above, the method of the invention involves contacting the target cell or the biochemical reaction with the PAM-reduced or abolished Cas protein of the invention or any fusion protein of conjugate thereof, specifically, any of the nucleic acid guided genome modifier/effector chimeric protein, complex or conjugate of the invention and any systems thereof. The term “contacting” as used herein, means to bring, put, incubate or mix together. More specifically, in the context of the present invention, the term “contacting” includes all measures or steps, which allow the protein or nucleic acid molecules, vectors, vehicles, compositions or systems of the invention such that they are in direct or indirect contact with the target cell/s, the genetic material of the cell or the nucleic acid sequence of interest in said biochemical reaction. To induce the desired modification of the target nucleic acid sequence as discussed herein, either in vitro or in vivo, the nucleic acid molecules, proteins, RNAs, RNPs or combinations thereof of the invention may be provided to and/or contacted with the target cells for about several minutes to about 24 hours, e.g., 5 minutes, 10 minutes, 15 minutes, 20 minutes, 25 minutes, 30 minutes, 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1 .5 days, every 2 days, every 3 days, or any other frequency from about every day to about every four days. The nucleic acid proteins, RNAs, RNPs molecules of the invention or any combinations thereof, may be provided to the target cells one or more times, e.g. one time, twice, three times, or more than three times, and the cells allowed to incubate with the nucleic acid molecules for some amount of time following each contacting event e.g. 16-24 hours.

In some embodiments, the CRISPR-Cas protein used by the methods of the invention may be any of the Cas proteins as defined as defined by the invention. In yet some further embodiments, any of the fusion/chimeric protein or conjugate of such Cas proteins that may be used by the methods of the invention, may be the nucleic acid guided genome modifier chimeric protein, complex or conjugate according to any one as defined by the invention. More specifically, any of the modifiers/effectors of the invention that comprise the amino acid sequence of any one of SEQ ID NO. 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492, or any variants or derivatives thereof. More particular embodiments refer to the chimeras disclosed by the amino acid sequences as denoted by any one of SEQ ID NO. 2, 375, 444, 448, 467, 476, 478, 479 and 480, or any variants and derivatives thereof. In yet some further embodiments, the methods of the invention may use any of the nucleic acid molecules disclosed by the invention. Still further, the method of the invention may use any of the systems disclosed by the invention.

In some embodiments, the method of the invention may be applicable for any cell of at least one organism of the biological kingdom Animalia. In some embodiments, the method of the invention may be applicable for any cell of at least one organism of the biological kingdom Animalia. In more specific embodiments, such cell may be derived from any unicellular or multicellular invertebrate or vertebrate. More specifically, cells derived from invertebrates, are cells derived from an organism of the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida—Segmented worms like earthworm and the Phylum Echinodermata—Echinoderms. Still further, in some embodiments, the methods of the invention may be applicable for a cell derived from any vertebrate organism, specifically, an organism derived from any of the vertebrates groups that include Fish, Amphibians, Reptiles, Birds and Mammals (e.g., Marsupials, Primates, Rodents and Cetaceans),In some particular embodiments, the methods of the invention may be particularly applicable for modifying a target nucleic acid sequence of interest in a cell of a mammal (specifically, at least one of a human, Cattle, rodent, domestic pig (swine, hog), sheep, horse, goat, alpaca, lama and Camels), an avian, an insect, a fish, an amphibian, a reptile, a crustacean, a crab, a lobster, a snail, a clam, an octopus, a starfish, a sea-urchin, jellyfish, and worms.

The methods of the invention provide targeted modification (either physical or functional as discussed above) of a target nucleic acid sequence of interest, in a target cell.

The term “target nucleic acid sequence of interest” as used herein refers to a gene or fragments thereof, or any coding or non-coding or regulatory sequence in chromosomal DNA, or in DNA of any organelle of a eukaryotic cell, for example, mitochondria, chloroplast, amyloplast and chromoplast, any non-chromosomal and/or exogenous nucleic acid sequence (e.g., plasmid/s, viruses and/or other genetic elements), or any fragment thereof to be targeted for mutation, deletion, insertion, replacement, repression, activation, or any other modulations as specified above. It should be appreciated that in some embodiments, the target sequence may be a predetermined target sequence.

Still further, it should be understood that “Gene” as used herein, may be a natural (e.g., genomic) or synthetic gene comprising transcriptional and/or translational regulatory sequences and/or a coding region and/or non-translated sequences (e.g., introns, 5′- and 3 ′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3 ′-untranslated sequences linked thereto. A gene may also be an amplified nucleic acid molecule produced in vitro comprising all or a part of the coding region and/or 5′- or 3 ′-untranslated sequences linked thereto.

Still further, in some embodiments, the target nucleic acid sequence is a DNA sequence. In some embodiments, the target DNA is genomic DNA. It should be understood that in some embodiments, the term “genomic DNA” encompasses chromosomal DNA, and also mitochondrial genome. Thus, in some embodiments, the target nucleic acid sequence is an extra-chromosomal nucleic acid sequence. In some specific embodiments, the extra-chromosomal target nucleic acid sequence resides in an organelle such as mitochondria, chloroplast, amyloplast, chromoplast, and any non-chromosomal and/or exogenous nucleic acid sequence (e.g., plasmid/s, viruses and/or other genetic elements). In some embodiments, the target nucleic acid sequence is a viral nucleic acid sequence. In some embodiments, the target nucleic acid sequence is a synthetic nucleic acid sequence.

In some further embodiments, the target nucleic acid sequence of interest modified in a target cell or in a biochemical reaction by the methods of the invention may be, or may be comprised within at least one of: at least one gene encoding at least one tumor associated antigen (TAA), at least one gene encoding at least one immune checkpoint receptor proteins or ligand, at least one gene encoding a protein involved in at least one congenital disorder, at least one gene encoding at least one gene encoding receptors for at least one viral antigen, at least one gene associated with at least one inborn error of metabolism (IEM) disorder, Immunoglobulin locus, T cell receptor (TCR) locus, safe harbor site/s (SHS), and any coding sequence or non-coding sequence involved with at least one pathologic disorder.

As indicated above, the target nucleic acid sequence may be any sequence encoding a tumor associated antigen. Thus, in some embodiments, the target genes targeted by the methods of the invention may be genes associated with or encoding at least one TAA. Tumor or cancer associated antigen (TAA), as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present invention, include, but are not limited to, RAGE-1, tyrosinase, MAGE-1, MAGE-2, NY-ESO-1, Melan-A/MART-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph. and TRP-1. Still further, TAA may be recognized by CD8+T cells as well as CD4+T cells. Non limiting examples of TAA recognized by CD8+T cells may be CSNK1A1, GAS7, HAUS3, PLEKHM2, PPP1R3B, MATN2, CDK2, SRPX (P55L), WDR46 (T227I), AHNAK (S4460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PAS5A WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+T cells may be ERBB2IP (E805G), CIRH1A (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (S1002I). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph.

Cancer antigen and tumor antigen are used interchangeably herein. The antigens may be related to cancers that include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer). In some specific embodiments, a TAA targeted by the methods of the invention may be the SIGLEC10 gene.

In some further embodiments, the methods of the invention may target regulatory sequences, such sequences may include any non-coding sequence. In some embodiments, the target sequence targeted by the methods of the invention is the Empty Spiracles Homeobox 1 (EMX1) gene, that encodes a member of the EMX family of transcription factors. The EMX1 gene, along with its family members, are expressed in the developing cerebrum and plays a role in specification of positional identity, the proliferation of neural stem cells, differentiation of layer-specific neuronal phenotypes and commitment to aneuronalor glial cell fate. The human EMX1 gene has a sequence as provided by NCBI Accession Number NC_000002.12, range 72910949-72936691 (NCBI gene ID 2016),In yet some further embodiments, the human EMX1 protein is denoted by NCBI Accession Number NP 004088.2.

According to some embodiments, the target nucleic acid sequence is a gene or any fragment thereof or any non-coding sequence involved in a genetic trait, and the modification results in changes in the transcription or translation of a genetic element, by a technical procedure that may include permanently replacing, knocking-out, temporarily or permanently enhancing, shutting-off, knocking-down, and frameshifting. In some embodiments, the genetic trait is modified by editing the genetic element sequence itself, its regulatory sequences, genes regulating the gene of interest or their regulatory sequences in a regulatory chain of events.

In some embodiments, the target sequence may be a target gene or any other coding and/or non-coding sequence involved in a congenital disorder. In some particular embodiments, such gene may be a gene involved in Autosomal dominant Retinitis Pigmentosa (adRP). In some further specific embodiments, the target gene may be the rhodopsin (RHO) gene. The RHO gene (also known as long Wavelength Sensitive opsin, L opsin, LWS opsin, MGC:21585, MGC:25387, Noerg 1, Opn2, Ops, opsin 2, Red Opsin, Rod Opsin, RP4), encodes a photoreceptor required for image-forming vision at low light intensity and for photoreceptor cell viability after birth. The protein encoded by this gene is found in rod cells that can sense light and initiate the phototransduction cascade in rod photoreceptors. The encoded protein binds to 11-cis retinal and is activated when light hits the retinal molecule. Defects in this gene are a cause of congenital stationary night blindness. The human RHO gene has a sequence as provided by NCBI Accession Number: NC_000003.12 (range 129528639-129535344) (Gene ID 6010). In yet some further embodiments, the human RHO protein is denoted by NCBI Accession Number NP_000530.1.

In some other embodiment, the target gene may be the Myeloperoxidase (MPO) gene. Myeloperoxidase (MPO), as used herein, is the most toxic enzyme found in the azurophilic granules of neutrophils. MPO utilizes H202 to generate hypochlorous acid (HClO) and other reactive moieties, which kill pathogens during infections. The MPO gene is located on the long arm segment q12-24 of chromosome 17 and the primary transcriptional product of this gene consists of 11 introns and 12 exons. Alternative splicing of the MPO mRNA gives two transcripts of 3.6 and 2.9 kB. The primary translation product is an 80 kDa precursor protein that undergoes a series of modifications including cleavage of a signal peptide, N-linked glycosylation, and limited deglycosylation, to form the catalytically inactive MPO precursor (apoproMPO). In the next step, MPO gains catalytic activity by incorporation of an iron-heme molecule into the catalytic centrum. Heme is covalently attached by two ester bonds and, unique for heme containing enzymes, a third sulfonium linkage, that uniquely orients one heme molecule into the enzyme pocket. The unique configuration of the heme moiety confers MPO with very high oxidative potential, enabling chlorination at physiological pH. Cleavage of proMPO leads to a 59 kDa a-subunit and a 13.5 kDa (3-subunit that are covalently attached through the heme moiety. A disulfide bridge joins the two heavy-light protomers in mature 150 kDa MPO. MPO expression levels depend upon allelic polymorphisms in the promoter region. Neutrophils are the main source of MPO where it accounts for 5% of the dry weight of the cell, making MPO the most abundant protein in neutrophils. MPO is transcribed only in promyelocytes during neutrophil differentiation in the bone marrow.

It should be further appreciated that in the context of the present disclosure, unless specifically indicated “MPO” encompasses both the MPO gene and the MPO protein. The human MPO gene has a sequence as provided by Accession Number: NC_000017.11. In yet some further specific embodiment, such sequence may comprise the nucleic acid sequence as denoted by SEQ ID NO: 373. In yet some further embodiments, the human MPO protein is denoted by Accession Number: NP_000241. Still further in some embodiments, the MPO protein may comprise the amino acid sequence as denoted by SEQ ID NO: 374. Still further in some other specific embodiment, the invention further provides the mouse MPO encoding sequence as denoted by Accession Number NC_000077, that may in some embodiments comprise the nucleic acid sequence as denoted by SEQ ID NO: 397. In yet some further embodiments, the mouse MPO protein is denoted by Accession Number: NP_034954. Still further in some embodiments, the mouse MPO protein may comprise the amino acid sequence as denoted by SEQ ID NO: 398.

MPO plays a role in suppressing the adaptive immune response. Mechanistically, MPO released from neutrophils inhibits LPS-induced DC activation as measured by decreased IL-12 production and CD86 expression consequently, limiting T cell proliferation and proinflammatory cytokine production. In contrast, a pathogenic role for MPO in driving autoimmune inflammation was also demonstrated. More specifically, increased MPO levels and activity have been observed in many inflammatory conditions and autoimmune diseases including multiple sclerosis (MS) and rheumatoid arthritis (RA). MPO plays a role in modulation of vasculature functioning, associated with chronic vascular diseases such as atherosclerosis. In the extracellular matrix (ECM), MPO works as a nitric oxide(NO)-scavenger consuming NO that leads to impaired endothelial relaxation. MPO and its oxidative species present in the atherothrombotic tissue, promotes lipid peroxidation, conversion of LDL to a highly-uptake atherogenic form, selectively modulates Apolipoprotein A-I (apoA-I) generating dysfunctional HDL particles more susceptible to degradation and impairs the ability of apoA-I to promote cholesterol efflux. Moreover, elevated systemic levels of MPO and its oxidation products are associated with increased cardiovascular risk. As indicated above, MPO has been implicated in variety of pathologic conditions, and thereof targeting the MPO gene provides a specific therapeutic tool for treating and preventing disorders or conditions caused thereby.

In yet some further embodiments, the congenital disorder may be Pseudoachondroplasia (PSACH). In some specific embodiments, the gene targeted by the methods of the invention may be the Cartilage Oligomeric Matrix Protein (COMP) gene. The protein encoded by this gene is a noncollagenous extracellular matrix (ECM) protein. It consists of five identical glycoprotein subunits, each with EGF-like and calcium-binding (thrombospondin-like) domains. Oligomerization results from formation of a five-stranded coiled coil and disulfides. Binding to other ECM proteins such as collagen appears to depend on divalent cations. Contraction or expansion of a 5 aa aspartate repeat and other mutations can cause pseudochondroplasia (PSACH) and multiple epiphyseal dysplasia (MED). The human COMP gene has a sequence as provided by NCBI Accession Number NC_000019.10 (18782773..18791305, complement) (Gene ID 1311). In yet some further embodiments, the human COMP protein is denoted by NCBI Accession Number NP_000086.2.

Still further in some embodiments, immune checkpoint receptor proteins or ligands (such as those targeted by checkpoint inhibitors in cancer checkpoint therapy and which can block inhibitory checkpoints, restoring immune system function) and any genes encoding such receptors or ligands may be targeted by the methods of the invention. More specifically, the methods of the invention may target immune checkpoint receptor proteins or ligands that include PD-1/PD-L1 and CTLA-4/B7-1/B7-2. In some specific embodiments, the target gene is a gene encoding the PDCD1 gene. Programmed Cell Death 1 PDCD1, also known as PD-1 and CD279, encodes a cell surface membrane protein of the immunoglobulin superfamily that has a role in regulating the immune system's response to the cells of the human body by down-regulating the immune system and promoting self-tolerance by suppressing T cell inflammatory activity, thus functioning as an immune checkpoint. The human PDCD1gene has a sequence as provided by NCBI Accession Number: NC_000002.12 (241849881..241858908, complement) (GENE ID 5133). In yet some further embodiments, the human PDCD1 protein is denoted by NCBI Accession Number: NP_005009.2.

In yet some further embodiments, the target gene may be the CTLA4 gene that encodes CTLA4. CTLA4 or CTLA-4 (cytotoxic T-lymphocyte-associated protein 4), also known as CD152 (cluster of differentiation 152), is a protein receptor that functions as an immune checkpoint and downregulates immune responses. CTLA4 is constitutively expressed in regulatory T cells but only upregulated in conventional T cells after activation. The human CTLA4gene has a sequence as provided by NCBI Accession Number: NC_000002.12 (203867771..203873965) (GENE ID 1493). In yet some further embodiments, the human CTLA4 protein is denoted by NCBI Accession Number: NP_001032720.1.

Still further, in some embodiments, the target nucleic acid sequence of the invention may be any gene encoding, or a sequence involved in the expression of immunological receptors, specifically, T cell receptors (TCR), B cell receptors (BCR) and antibodies. According to such embodiments, the target sequence targeted by the methods of the invention may be located at the immunoglobulin locus, specifically, any one of the Immunoglobulin heavy chain locus, Immunoglobulin lc chain locus, Immunoglobulin 2 chain locus, TCRP chain locus, TCRa chain locus, TCRy chain and the TCR6 chain locus.

In yet some further embodiments, the target sequence may be a sequence enabling insertion of a desired nucleic acid sequence. In more specific embodiments, the target sequence may be within GSHs. Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism. GSHs are thus ideal sites for transgene insertion whose use can empower functional genetics studies in basic research and therapeutic applications in human gene therapy. Non-limiting examples for SHS sites applicable in the present invention include the human AAVS1 site on chromosome 19q, and the human ROSA26, and CCR5 sites. In should be appreciated that in some embodiments of the invention, GSHs may be useful as target sequences, particularly in cases where it is desired to express an exogenous nucleic acid sequence of interest in a specific cell. Such encoding sequence may be inserted in GSH sites. Non-limiting examples for such exogenous nucleic acid sequences that can be inserted in GSHs, may be any sequence encoding a receptor or chimeric receptor, for example, any chimeric antigen receptor (CAR).

In yet some further embodiments, the target nucleic acid sequence may be a gene encoding viral receptors, for example, the Integrin Subunit Beta 3 (ITGB3) gene. The ITGB3 protein product is the integrin beta chain beta 3. Integrins are integral cell-surface proteins composed of an alpha chain and a beta chain. A given chain may combine with multiple partners resulting in different integrins. Integrin beta 3 is found along with the alpha Ilb chain in platelets. The human ITGB3 gene has a sequence as provided by NCBI Accession Number: NC_000017.11 (47253827..47313743) (GENE ID 3690). In yet some further embodiments, the human ITGB3 protein is denoted by NCBI Accession Number: NP_000203.2.

Still further, in some embodiments, the methods of the invention may be applicable for modifying at least one target nucleic acid sequence of interest in any cell of at least one organism of the biological kingdom Plantae.

It should be understood that the modification of a target nucleic acid sequence in a cell may be performed by the methods of the invention either in vitro/ex vivo or in vivo. “In vitro” is defined herein as an artificial environment outside the membranes of a whole or partial, differentiated or undifferentiated, living organism, organ, tissue, callus or cell. In some embodiments, the term in-vitro is not inside a viable cell.

“In vivo” is defined herein as inside a whole or partial, differentiated or undifferentiated, organism, organ, tissue, callus or cell.

It should be noted that in some embodiments, the method of the invention may be applicable for modification of at least one target nucleic acid sequence of interest in at least one cell may be performed in at least one organism of at least one of: the biological kingdom Plantae and the biological kingdom Animalia. Thus, as indicated above, the invention provides in vitro or ex vivo methods for performing a targeted modification in a gene in a cell or any parts thereof or in a tissue, or alternatively, in vivo methods for performing the desired manipulation in an organism, as disclosed by the invention.

As indicated above, the invention provides methods of manipulating a nucleic acid sequence of interest in a biological reaction or in a cell either in vitro/ex vivo, or in vivo in a target organism. The invention thus provides in some embodiments thereof non-therapeutic, and well as therapeutic methods based on manipulations and modifications of nucleic acid sequences of interest in a treated subject, and therefore relates to gene therapy. The non-therapeutic applications of such methods may encompass cosmetic, diagnostic and agricultural uses. The term “gene therapy” as herein defined, refers to the correction, modulation or ablation of at least one target gene. This term further encompasses insertion of a gene of interest into a target locus (e.g., chimeric receptors, such as CAR, TCRs, BCRs, or antibodies), or replacement of an endogenous gene with at least one nucleic acid sequence of interest. The method of the invention is also suitable for the treatment of diseases caused by the failure of a single gene, or of multiple genes (also referred to as polygenic or chromosomal), and is applicable in cases were specific mutations resulting in a defective gene or gene are identified or not.

The method of the invention is thus suitable for the treatment of diseases caused by the failure of a single gene, or of multiple genes (also referred to as polygenic or chromosomal), provided that the specific mutations resulting in a defective gene or gene are identified. Theoretically, if the dysfunctional gene is replaced with the corresponding healthy one, or alternatively, is knocked out or modulated, a cure can be achieved.

Thus, a further aspect of the invention relates to a method of curing or treating, preventing, inhibiting, reducing, eliminating, protecting or delaying the onset of a pathologic disorder or condition in a subject in need thereof. More specifically, the method of the invention may comprise the steps of administering to the treated subject an effective amount of at least one of:

(a) at least one Cas protein or any Cas protein derived domain, having reduced or abolished PAM constraint or any fragment, variant, mutant, fusion/chimeric protein, complex or conjugate thereof, or alternatively or additionally, at least one nucleic acid sequence encoding the Cas protein or any variant, mutant, fusion/chimeric protein, complex or conjugate thereof. It should be noted that in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding the target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b);

(e) at least one host cell modified by, and/or comprising at least one of: (a), (b), (c) and (d); and

(f) at least one composition comprising at least one of (a), (b), (c), (d) and (e).

In some embodiments, the methods of the invention may use any of the CRISPR-Cas proteins defined by the invention. In yet some further embodiments, the methods of the invention may use any of the fusion/chimeric protein, complex or conjugate of such Cas protein, specifically, any of the nucleic acid guided genome modifier or effector chimeric protein, complex or conjugate defined by the invention. In more specific embodiments, any of the guided genome modifier/effector chimeric proteins of the invention, specifically the proteins comprising the amino acid sequence as denoted by any one of SEQ ID NO. 2, 14, 15, 16, 17, 24-44, 45-48, 56, 210-213, 271-281, 284 and 288-290, 314-318, 320-323, 330-357, 362-366, and 375-396, 406, 407, 426-438, 440-480, 487-492, may be applicable in the methods of the invention. More particular embodiments refer to the chimeras disclosed by the amino acid sequences as denoted by any one of SEQ ID NO. 2, 375, 444, 448, 467, 476, 478, 479 and 480, or any variants and derivatives thereof. In yet some further embodiments, the methods of the invention may use any of the nucleic acid molecules defined by the invention as disclosed herein. Still further, the methods of the invention may use any of the systems of the invention. Still further, in some embodiments, any of the host cells defined by the invention may be used for the methods of the invention.

In some embodiments, the methods of the invention may be used for treating any subject of the biological kingdom Animalia or of the biological kingdom Plantae.

Subject suitable for the invention are Eukaryotic organisms, specifically, any unicellular or multicellular organisms of the Animalia and Plantae biological kingdoms.

In more specific embodiments, the methods of the invention may be applicable for any subject of the biological kingdom Animalia. It should be understood that an organism of the Animalia kingdom in accordance with the invention includes any invertebrate or vertebrate organism.

More specifically, Invertebrates are animals that neither possess nor develop a vertebral column (commonly known as a backbone or spine), derived from the notochord. This includes all animals apart from the subphylum Vertebrata. More specifically, invertebrates include the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida-Segmented worms like earthworm and the Phylum Echinodermata—Echinoderms Familiar examples of invertebrates include insects; crabs, lobsters and their kin; snails, clams, octopuses and their kin; starfish, sea-urchins and their kin; jellyfish and worms.

In some embodiments the invention may be applicable for any organism of the phylum arthropod that are invertebrate animals having an exoskeleton (external skeleton), a segmented body, and paired jointed appendages. Arthropods form the phylum Euarthropoda, which includes insects, arachnids, myriapods, and crustaceans.

Insects or Insecta are hexapod invertebrates and the largest group within the arthropod phylum. Definitions and circumscriptions vary; usually, insects comprise a class within the Arthropoda. As used here, the term Insecta is synonymous with Ectognatha. Insects have a chitinous exoskeleton, a three-part body (head, thorax and abdomen), three pairs of jointed legs, compound eyes and one pair of antennae. Insects are the most diverse group of animals; they include more than a million described species and represent more than half of all known living organisms. Insects can be divided into two groups historically treated as subclasses: wingless insects, known as Apterygota, and winged insects, known as Pterygota. The Apterygota consist of the primitively wingless order of the silverfish (Zygentoma). Archaeognatha make up the Monocondylia based on the shape of their mandibles, while Zygentoma and Pterygota are grouped together as Dicondylia. The Zygentoma themselves possibly are not monophyletic, with the family Lepidotrichidae being a sister group to the Dicondylia (Pterygota and the remaining Zygentoma). Paleoptera and Neoptera are the winged orders of insects differentiated by the presence of hardened body parts called sclerites, and in the Neoptera, muscles that allow their wings to fold flatly over the abdomen. Neoptera can further be divided into incomplete metamorphosis-based (Polyneoptera and Paraneoptera) and complete metamorphosis-based groups. It should be noted that the present invention is applicable for any of the insects of any of the groups and species disclosed herein. Still further, many insects are considered pests by humans Insects commonly regarded as pests include those that are parasitic (e.g. lice, bed bugs), transmit diseases (mosquitoes, flies), damage structures (termites), or destroy agricultural goods (locusts, weevils). Insects considered pests of some sort occur among all major living orders with the exception of Ephemeroptera (mayflies), Odonata, Plecoptera (stoneflies), Embioptera (webspinners), Trichoptera (caddisflies), Neuroptera (in the broad sense), and Mecoptera (also, the tiny groups Zoraptera, Grylloblattodea, and Mantophasmatodea). Of particular interest of this group is the Mosquito. More specifically, in some embodiments, the invention may be suitable for insects such as mosquito for example. Mosquitoes are a group of about 3500 species of small insects that are a type of fly (order Diptera). Within that order they constitute the family Culicidae. Superficially, mosquitoes resemble crane flies (family Tipulidae) and chironomid flies (family Chironomidae). It should be appreciated that in some embodiments, the term mosquito, as used herein includes all genera encompassed by the subfamilies Anophelinae and Culicinae. In yet some further embodiments, mosquito as used herein include, but is not limited to any mosquito of the following genera, Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, and Wyeomyia. Females of most species are ectoparasites, whose tube-like mouthparts (called a proboscis) pierce the hosts' skin to consume blood. Though the loss of blood is seldom of any importance to the victim, the saliva of the mosquito often causes an irritating rash that is a serious nuisance. Much more serious though, are the roles of many species of mosquitoes as vectors of diseases. In passing from host to host, some transmit extremely harmful infections such as malaria, yellow fever, Chikungunya, West Nile virus, dengue fever, filariasis, Zika virus and other arboviruses, rendering it the deadliest animal family in the world.

Of particular interest in some embodiments of the present invention is the bee. Bees are flying insects closely related to wasps and ants, known for their role in pollination and, in the case of the best-known bee species, the western honeybee, for producing honey and beeswax. Bees are a monophyletic lineage within the superfamily Apoidea and are presently considered a Glade, called Anthophila. There are nearly 20,000 known species of bees in seven recognized biological families, specifically, Andrenidae, Apidae, Colletidae, Halictidae, Megachilidae, Melittidae, Stenotritidae. Some species including honeybees, bumblebees, and stingless bees live socially in colonies. It should be understood that the present invention encompasses any of the bee species of any of the bee families indicated herein.

Still further, the invention may be useful for crustacean organisms. Crustaceans, as used herein, form a large, diverse arthropod taxon which includes crabs, lobsters, crayfish, shrimp, krill, woodlice, and barnacles, that are all encompassed by the present invention. The crustacean group is usually considered as a paraphyletic group and comprises all animals in the Pancrustacea Glade other than hexapods. Some crustaceans are more closely related to insects and other hexapods than they are to certain other crustaceans. In some embodiments, such crustaceans may be shrimp. The term shrimp is used to refer to decapod crustaceans and covers any of the groups with elongated bodies and a primarily swimming mode of locomotion i.e. Caridea and Dendrobranchiata.

In yet some further embodiments, the invention may be useful for organisms of the subphylum Chelicerata that is one of the major subdivisions of the phylum Arthropoda and includes the sea spiders, arachnids, and several extinct lineages. In more specific embodiments, the invention may be useful for organisms of the Arachnida that are a class including spiders (the largest order in the class), scorpions, Acari (ticks, mites), harvestmen, and solifuges.

Still further, in some embodiments, the methods of the invention may be applicable for a vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans).

Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish.

Still further, in some embodiments, the subject of the invention may be any one of a human or non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the subject of the invention may be a mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the invention may be applicable for any organism of the order primates. More specifically, primates are divided into two distinct suborders, the first is the strepsirrhines that includes lemurs, galagos, and lorisids. The second is haplorhines—that includes tarsier, monkey, and ape clades, the last of these including humans. In yet some further embodiments, the invention may be applicable for any organism of the subfamily Homininae, that includes the hylobatidae (gibbons) and the hominidae that includes ponqunae (orangutans) and homininae [gorillini (gorilla) and hominini ((panina(chimpanzees) and hominina (humans))].

In some specific embodiment, the methods of the invention may be applicable for a mammal that may be at least one of a Cattle, domestic pig (swine, hog), sheep, horse, goat, alpaca, lama and Camels.

In some embodiments, the invention may be applicable for subject of the Order Artiodactyla, including members of the family Suidae, subfamily SuMae and Genus Sus, and members of the family Bovidae, subfamily Bovinae including ungulates. More specifically, domestic cattle, bison, African buffalo, the water buffalo, the yak. Of particular interest in the present invention are domestic cattle being the most widespread species of the genus Bos and are most commonly classified collectively as Bos taurus.

More specifically, the subject the invention as well as the methods disclosed herein above offer great economic advantage for any industrial or agricultural use of animals, specifically, livestock. Thus, in some specific embodiments, the invention may be applicable for mammalian livestock, specifically those used for meat, milk and leather industries. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the subject of the invention may be Cattle, colloquially cows, that are the most common type of large domesticated ungulates, that belong to the Bovidae family

Still further, the Bovidae are the biological family of cloven-hoofed, ruminant mammals that includes bison, African buffalo, water buffalo, antelopes, wildebeest, impala, gazelles, sheep, goats, muskoxen. The biological subfamily Bovinae includes a diverse group of ten genera of medium to large-sized ungulates, including domestic cattle, bison, African buffalo, the water buffalo, the yak, and the four-horned and spiral-horned antelopes. Of particular interest in the present invention may be the domestic cattle are the most widespread species of the genus Bos and are most commonly classified collectively as Bos taurus. More specifically, Bos is the genus of wild and domestic cattle. Bos can be divided into four subgenera: Bos, Bibos, Novibos, and Poephagus. Subgenus Bos includes Bos primigenius (cattle, including aurochs), Bos primigenius primigenius (aurochs), Bos primigenius taurus (taurine cattle, domesticated) and Bos primigenius indicus (zebu, domesticated).

In yet some further embodiments, rodents may be of particular relevance since it represents the most popular and commonly accepted animal model in research.

Thus, in some further embodiment, the methods of the invention may be applicable for a mammal such as a rodent. Rodents are mammals of the order Rodentia, which are characterized by a single pair of continuously growing incisors in each of the upper and lower jaws. Rodents are the largest group of mammals. Non-limiting examples for such rodents that are applicable in the present invention, appear in the following list of rodents, arranged alphabetically by suborder and family Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides; extinct)], the kangaroo mice and rats (family Heteromyidae) [kangaroo mouse (genus Microdipodops), kangaroo rat (genus Dipodomys), pocket mouse (several genera)], the pocket gopher family (Geomyidae) [pocket gopher (multiple genera)]. Suborder Hystricomorpha, includes the agouti family (Dasyproctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [classic rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinomyidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodontomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomyid family (Nesomyidae), African pouched rat (genera Beamys, Cricetomys, and Saccostomus)], the Oriental dormouse family (Platacanthomyidae)[Asian tree mouse (genera Platacanthomys and Typhlomys)], the spalacid family (Spalacidae) [bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Tamias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)]. In yet some further embodiments, the subject of the invention may be a mouse. A mouse, plural mice, is a small rodent characteristically having a pointed snout, small rounded ears, a body-length scaly tail and a high breeding rate. The best known mouse species is the common house mouse (Mus musculus). Species of mice are mostly found in Rodentia and are present throughout the order. Typical mice are found in the genus Mus.

In yet some further embodiments, the organism applicable in the methods of the invention, may be avian organisms. In yet some further specific embodiments, the invention may be suitable for birds. More specifically, domesticated and undomesticated birds are also suitable organisms for the invention.

Therefore, in certain embodiments, the avian organism of the invention may be any one of a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. The term “avian” relates to any species derived from birds characterized by feathers, toothless beaked jaws, the laying of hard-shelled eggs, a high metabolic rate, a four-chambered heart, and a lightweight but strong skeleton. The term “hen” includes all females of the avian species.

In yet some further embodiments, the methods of the invention may be applicable for treating mammalian subjects, specifically, human subjects.

In yet some further embodiments, the methods of the invention may be applicable for treating any pathologic disorder in a subject, specifically, a mammalian subject, specifically, any one of a proliferative disorder, a congenital disorder, an immune-related condition, an inflammatory condition, a metabolic disorder, a disorder caused by a pathogen, an autoimmune disorder, a disorder associated with the expression of a coding or non-coding sequence and an IEM disorder.

More specifically, in some embodiments, the methods of the invention may be applicable for treating proliferative disorders. Proliferative disorders, such as cancer, may also be classified as genetic disorders or conditions, as they may result from a defect in a single or multiple genes. Some non-limiting examples of cancers that are classified as genetic disorders or conditions are FAP (familial adenomatous polyposis) or HNPCC (hereditary non-polyposis colon cancer) and breast or ovarian cancers that are associated with inherited mutations in either of the tumor suppressor BRCA1 or BRCA2 genes. The latter examples may be classified as polygenic (or chromosomal) genetic disorders. Approximately five to ten percent of cancers are entirely hereditary. Thus, proliferative disorders may also be treated by the methods of the invention.

More specifically, as used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors. Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. In yet some further embodiments, the methods of the invention may be applicable for any of the proliferative disorders discussed herein. In more specific and non-limiting embodiments, the methods of the invention may be specifically applicable for at least one of non-small cell lung cancer (NSCLC) melanoma, renal cell cancer, ovarian carcinoma and breast carcinoma.

In some specific embodiments, the methods of the invention may be applicable for treating and curing congenital disorders. A congenital disorder is any one of monogenic or chromosomal or multifactorial.

As indicated above, the invention provides methods for curing genetic disorders. Specifically, by replacing, mutating, deleting or inserting a sequence into a mal functioning or mutated gene or fragment/s thereof that are associated with the genetic condition using the methods of the invention. A genetic disorder or condition as herein defined is a disease caused by an abnormality in the DNA sequence of an individual. Abnormalities as used herein refer to a small mutation in a single gene. A genetic disorder or condition may be a heritable disorder and as such may be present from before birth. Other genetic disorders or conditions are caused by misregulation of a gene or new mutations or changes to the DNA.

Based on their genetic contribution, human genetic disorders or conditions can be classified as monogenic (i.e. which involve mutations in a single gene), chromosomal (also referred to as polygenic), or multifactorial genetic diseases. Monogenic diseases are caused by alterations in a single gene.

A hereditary disease may result unexpectedly when two healthy carriers of a defective recessive gene reproduce but can also happen when the defective gene is dominant.

The term “mutation” as herein defined refers to a change in the nucleotide sequence of the genome of an organism. This term further encompasses a desired outcome of NHEJ-erroneous repair. Mutations result from unrepaired damage to DNA or to RNA genomes (typically caused by radiation or chemical mutagens), from errors in the process of replication, or from the insertion or deletion of segments of DNA by mobile genetic elements. Mutations may or may not produce observable (phenotypic) changes in the characteristics of an organism. Mutation can result in several different types of change in the DNA sequence; these changes may have no effect, alter the product of a gene, or prevent the gene from functioning properly or completely. There are generally three types of mutations, namely single base substitutions, insertions and deletions and mutations defined as “chromosomal mutations”.

The term “single base substitutions” as herein defined refers to a single nucleotide base which is replaced by another. These single base changes are also called point mutations. There are two types of base substitutions, namely, “transition” and “transversion”. When a purine base (i.e. Adenosine or Thymine) replaces a purine base or a pyrimidine base (Cytosine, Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows:

In missense mutations the new base alters a codon, resulting in a different amino acid being incorporated into the protein chain. As a non-limiting example, the disease sickle cell anemia is a result of a single base substitution that is a missense mutation. In sickle cell anemia, the 17th nucleotide of the gene for the beta chain of haemoglobin (haem) is mutated from an ‘a’ to a ‘t’. This changes the codon from ‘gag’ to ‘gtg’, resulting in the 6th amino acid of the chain being changed from glutamic acid to Valine. This alteration to the beta globin gene alters the quaternary structure of haemoglobin, which has a profound influence on the physiology and wellbeing of the individual. In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly. Nonsense mutations are the molecular basis for between 15% to 30% of all inherited diseases. Some non-limiting examples include Cystic fibrosis, haemophilia, retinitis pigmentosa and Duchenne muscular dystrophy.

In silent mutations no change in the final protein product occurs and thus the mutation can only be detected by sequencing the gene. Most amino acids that make up a protein are encoded by several different codons (see genetic code). So, if for example, the third base in the ‘cag’ codon is changed to an ‘a’ to give ‘caa’, a glutamine (Q) would still be incorporated into the protein product, because the mutated codon still codes for the same amino acid. These types of mutations are ‘silent’ and have no detrimental effect.

Mutation may also arise from insertions of nucleic acids into the DNA or from duplication or deletions of nucleic acids therefrom. As herein defined, the term “insertions and deletions” refers to extra base pairs that are added or deleted from the DNA of a gene, respectively. The number of bases can range from a few to thousands. Insertions and deletions of one or two bases or multiples of one or two bases cause, inter alia, frame shift mutations (i.e. these mutations shift the reading frame of the gene). These can have devastating effects because the mRNA is translated in new groups of three nucleotides and the protein being produced may be useless.

Insertions and deletions of three or multiples of three bases may be less serious because they preserve the open reading frame. However, a number of trinucleotide repeat diseases exist including, for example, Huntington's disease and fragile X syndrome.

In Huntington's disease, for example, the repeated trinucleotide is ‘cag’. This adds a string of glutamines to the Huntington protein. The abnormal protein produced interferes with synaptic transmission in parts of the brain leading to involuntary movements and loss of motor control. Genetic disorders (or conditions, diseases) that may be cured by the methods of the invention may be further classified as “recessive” and “dominant” as well as autosomal and X-linked (relating to the chromosome the gene is on).

The term “Autosomal dominant disorder” as referred to herein encompasses genetic disorders or diseases, in which only one mutated copy of the gene is required for a person to be affected. Each affected person usually has one affected parent. Some non-limiting examples of autosomal dominant genetic diseases are Huntington's disease, Neurofibromatosis 1, and Marfan syndrome.

The term “autosomal recessive disorder” as referred to herein, encompasses genetic diseases, in which two copies of the gene should be mutated for a person to be affected. An affected person usually has unaffected parents who each carry a single copy of the mutated gene (and are referred to as carriers). Some non-limiting examples of autosomal recessive disorders include Cystic fibrosis, sickle cell anemia, Tay-Sachs disease, spinal muscular atrophy, Sickle-cell disease (SCD) and phenylketonuria (PKU) which is an autosomal recessive metabolic genetic disorder.

The term “X-linked dominant” as herein defined refers to disorders that are caused by mutations in genes on the X chromosome. Males are more frequently affected than females, and the chance of passing on an X-linked dominant disorder differs between men and women. Some X-linked dominant conditions include, but are not limited to Aicardi Syndrome, and Hypophosphatemia. X-linked disorders may also be classified as “recessive X-linked”. Recessive X-linked disorders as herein defined are also caused by mutations in genes on the X chromosome. Males are more frequently affected than females, and the chance of passing on the disorder differs between men and women. Some non-limiting examples of recessive X-linked disorders are Hemophilia A, Duchenne muscular dystrophy, Color blindness, Muscular dystrophy, Androgenetic alopecia and G-6-PD (Glucose-6-phosphate dehydrogenase) deficiency.

Genetic disorders may also be Y-linked. The term “Y-linked disorders” as herein defined refers to genetic diseases that are caused by mutations on the Y chromosome. Only males can get them, and all of the sons of an affected father are affected.

Genetic disorders may also be classified as “Mitochondrial”. The term “Mitochondrial diseases” as herein defined refers to maternal inheritance, and only applies to genes in mitochondrial DNA. Because only egg cells contribute mitochondria to the developing embryo, only females can pass on mitochondrial conditions to their children. A non-limiting example of a mitochondrial genetic disease is Leber's Hereditary Optic Neuropathy (LHON).

In further embodiments, the genetic disorder may be a multifactorial genetic disease. Examples of multifactorial genetic diseases include, but are not limited to breast and ovarian cancers that are associated with the BRCA1 or BRCA2 gene, Alzheimer's disease, some forms of colon cancer, e.g. familial adenomatous polyposis (FAP) or hereditary non-polyposis colon cancer (HNPCC) as well as hypothyroidism.

Currently around 4,000 genetic disorders or conditions are known, with more being discovered. Most disorders or conditions are quite rare and affect one person in every several thousands or millions. Interestingly, Cystic fibrosis is one of the most common genetic disorders; around 5% of the population of the United States carry at least one copy of the defective gene.

The method of the invention may also be used for the treatment of orphan diseases. The term “orphan disease” as herein defined refers to a rare disease, which affects a small percentage of the population. Most rare diseases are genetic, and thus are present throughout the person's entire life, even if symptoms do not immediately appear. Many rare diseases appear early in life, and about 30 percent of children with rare diseases will die before reaching their fifth birthday. A disease may be considered rare in one part of the world, or in a particular group of people, but still be common in another. A rare disease was defined in the Orphan Drug Act of 1983 as one that afflicts fewer than 200,000 people in a nation. According to the National Institute of Health, some non-limiting examples of orphan diseases are Cystic fibrosis, Ataxia telangiectasia and Tay-Sachs, to name but few.

In some embodiments, the genetic disorder or condition encompassed by the invention is a monogenic genetic disease, which may be, but is not limited to Duchenne muscular dystrophy, Cystic Fibrosis, Tay—Sachs disease (also known as GM2 gangliosidosis or hexosaminidase A deficiency), Ataxia-Telangiectasia (A-T), Sickle-cell disease (SCD), or sickle-cell anemia (SCA or anemia), Lesch—Nyhan syndrome (LNS, also known as Nyhan's syndrome, Amyotrophic Lateral Sclerosis, Cystinosis, Kelley-Seegmiller syndrome and Juvenile gout), color blindness, Haemochromatosis (or haemosiderosis), Haemophilia, Phenylketonuria (PKU), Phenylalanine Hydroxylase Deficiency disease, Polycystic kidney disease (PKD or PCKD, also known as polycystic kidney syndrome), Alpha-galactosidase A deficiency, Fabry disease, Anderson-Fabry disease, Angiokeratoma Corporis Diffusum, CADASIL (cerebral autosomal dominant arteriopathy with subcortical infarcts and leukoencephalopathy), Cerebral arteriopathy with subcortical infarcts and leukoencephalopathy, Cerebral autosomal dominant ateriopathy with subcortical infarcts and leukoencephalopathy, Carboxylase Deficiency, Multiple (Late-Onset), Cerebroside Lipidosis syndrome, Gaucher's disease, Choreoathetosis self-mutilation hyperuricemia syndrome, Classic Galactosemia, Galactosemia, Crohn's disease, also known as Crohn syndrome and regional enteritis, Incontinentia Pigmenti (also known as “Bloch-Siemens syndrome,” “Bloch-Sulzberger disease,” “Bloch-Sulzberger syndrome” “melanoblastosis cutis,” and “naevus pigmentosus systematicus”), galactosemia Microcephaly, alpha-1 antitrypsin deficiency (Alpha-1), Adenosine deaminase (ADA) deficiency, Severe Combined Immunodeficiency (SCID), neurofibromatosis type 1 (NF1), Wiskott—Aldrich syndrome, Stargardt macular degeneration, Fanconi' s anemia, Spinal muscular atrophy (SMA) and Leber's congenital amaurosis (LCA).

In other embodiments non-hereditary diseases such as autoimmune diseases are particularly applicable for curing via knockout or downregulation of the auto-antigen by using the method or system of the invention.

In some specific embodiments, the methods of the invention may be applicable for treatment and/or curing of RP.

Retinitis pigmentosa (RP) is an inherited dystrophic or degenerative disease of the retina with a prevalence of roughly one in 4,000. Typically, the disease progresses from the midperiphery of the retina into the central retina and, in many cases, into the macula and fovea. Clinical features include night blindness starting in adolescence, followed by progressive loss of peripheral vision, referred to as “tunnel vision”, culminating in legal blindness or complete blindness in adulthood. Characteristic retinal findings on examination include bone-spicule formations and attenuated blood vessels, reduced visual fields, reduced and/or abnormal electroretinograms (ERGs), changes in structure imaged by optical coherence tomography (OCT), and subjective changes in visual function. However, features and findings are highly variable among patients, even among patients within the same family Currently, there are no effective treatments for RP.

The invention is applicable for all modes of inheritance are encountered, specifically, dominant, recessive, autosomal, X-linked, and even mitochondrial. adRP accounts for 25%-30% of the cases. It is assumed that each patient has a monogenic form of disease (or digenic in rare cases) but many different genes account for disease in RP patients as a group.

Finding genes and mutations causing adRP: for autosomal dominant diseases, the problems are compounded by the need to detect a single, heterozygous mutation in a diploid organism, the proverbial needle-in a-hay-stack. Clinical evaluation, NGS, segregation testing and linkage analysis are performed. It should be noted that the prevalence of adRP is around 1:15,000.

An accurate diagnosis of retinitis pigmentosa relies on the documentation of the progressive loss photoreceptor cell function, confirmed by a combination of visual field and visual acuity tests, fundus and optical coherence imagery, and electroretinography (ERG). The patient's family history is also considered due to the mode of inheritance. Clinical findings include night blindness or nyctalopia, Tunnel vision (due to loss of peripheral vision), Latticework vision, Photopsia (blinking/shimmering lights), Photophobia (Aversion to glare), Development of bone spicules in the fundus, Slow adjustment from dark to light environments and vice versa, Blurring of vision, Poor color separation, Loss of central vision and Eventual blindness.

In yet some further embodiments, the methods of the invention may be applicable for treating and curing PSACH. Pseudoachondroplasia (PSACH) is a skeletal dysplasia characterized by disproportionate short stature, small hands and feet, abnormal joints and early onset osteoarthritis. PSACH is caused by mutations in thrombospondin 5 (TSP-5, also known as cartilage oligomeric matrix protein or COMP), a pentameric extracellular matrix protein primarily expressed in chondrocytes and musculoskeletal tissues. The thrombospondin gene family is composed of matricellular proteins that associate with the extracellular matrix (ECM) and regulate processes in the matrix. Mutations in COMP interfere with calcium-binding, protein conformation and export to the extracellular matrix, resulting in inappropriate intracellular COMP retention. This accumulation of misfolded protein is cytotoxic and triggers premature death of chondrocytes during linear bone growth, leading to shortened long bones. Both in vitro and in vivo models have been employed to study the molecular processes underlying development of the PSACH pathology.

While PSACH is a rare disorder with an estimated birth prevalence of approximately 1/30,000 (www.orpha.net), the exact birth prevalence is not known since PSACH newborns are indistinguishable from other babies at birth.

PSACH is an autosomal dominant disorder that occurs as a (de novo) new event in 70-80% of families with the remaining cases being inherited from an affected parent.

The diagnosis of pseudoachondroplasia can be made on the basis of clinical findings and radiographic features. Identification of a heterozygous pathogenic variant in COMP on molecular genetic testing establishes the diagnosis if clinical features are inconclusive.

Pseudoachondroplasia is one of the most common skeletal dysplasias affecting all racial groups. However, no precise incidence figures are currently available.

Clinical findings include: Normal length at birth, Normal facies, Waddling gait, recognized at the onset of walking, Decline in growth rate to below the standard growth curve by approximately age two years, leading to moderately severe disproportionate short-limb short stature, Moderate brachydactyly, Ligamentous laxity and joint hyperextensibility, particularly in the hands, knees, and ankles, Mild myopathy reported for some individuals, Restricted extension at the elbows and hips, Valgus, varus, or windswept deformity of the lower limbs, Mild scoliosis, Lumbar lordosis (˜50% of affected individuals), Joint pain during childhood, particularly in the large joints of the lower extremities; may be the presenting symptom in mildly affected individuals.

Radiographic features include: Delayed epiphyseal ossification with irregular epiphyses and metaphyses of the long bones (consistent), Small capital femoral epiphyses, short femoral necks, and irregular, flared metaphyseal borders; small pelvis and poorly modeled acetabulae with irregular margins that may be sclerotic, especially in older individuals, Significant brachydactyly; short metacarpals and phalanges that show small or cone shaped epiphyses and irregular metaphyses; small, irregular carpal bones, Anterior beaking or tonguing of the vertebral bodies on lateral view. This distinctive appearance of the vertebrae normalizes with age, emphasizing the importance of obtaining in childhood the radiographs to be used in diagnosis.

In some specific embodiments, the methods of the invention may be applicable for treating and curing a MPO-related condition. In some embodiments, the MPO-related condition may be an immune-related disorder. An “Immune-related disorder” or “Immune-mediated disorder”, as used herein encompasses any condition that is associated with the immune system of a subject, more specifically through inhibition or the activation of the immune system, or that can be treated, prevented or ameliorated by reducing degradation of a certain component of the immune response in a subject, such as the adaptive or innate immune response. An immune-related disorder may include infectious condition (e.g., viral infections), metabolic disorders, auto-immune disorders, vasculitis, inflammation and proliferative disorders, specifically, cancer. In some embodiments, the immune-related disorder may be an autoimmune disease. In accordance with some embodiments, the methods of the invention are applicable in treating autoimmune disorders. An autoimmune disease is a condition arising from an abnormal immune response to a normal body part. Examples of an autoimmune disorder include Rheumatoid arthritis (RA), Multiple sclerosis (MS), Systemic lupus erythematosus (lupus), Type 1 diabetes, Psoriasis/psoriatic arthritis, Inflammatory bowel disease including Crohn's disease and Ulcerative colitis, and Vasculitis.

In some specific embodiments, the methods of the invention may be particularly applicable for autoimmune disorder such as multiple sclerosis (MS), Anti-neutrophil cytoplasmic antibodies (ANCAs)—related disorder, and systemic lupus erythematosus (SLE).

In some embodiments, the methods of the invention may be applicable for the treatment of MS and any related conditions or symptoms associated therewith. The term “Multiple Sclerosis” (MS) as herein defined is a chronic inflammatory neurodegenerative disease of the central nervous system that destroys myelin, oligodendrocytes and axons. MS is the most common neurological disease among young adults, typically appearing between the ages of 20 and 40. The symptoms of MS vary, from the appearance of visual disturbance such as visual loss in one eye, double vision to muscle weakness fatigue, pain, numbness, stiffness and unsteadiness, loss of coordination and other symptoms such as tremors, dizziness, slurred speech, trouble swallowing, and emotional disturbances. As the disease progresses patients may lose their ambulation capabilities, may encounter cognitive decline, loss of self-managing of everyday activities and may become severely disabled and dependent.

MS symptoms develop because immune system elements attack the brain's cells, specifically, glia and /or neurons, and damage the protective myelin sheath of axons. The areas in which these attacks occur are called lesions that disrupt the transmission of messages through the brain. Multiple sclerosis is classified into four types, characterized by disease progression: (1) Relapsing-remitting MS (RRMS), which is characterized by relapse (attacks of symptom flare-ups) followed by remission (periods of stabilization and possible recovery; while in some remissions there is full recovery, in other remissions there is partial or no recovery). Symptoms of RRMS may vary from mild to severe, and relapses may last for days or months. More than 80 percent of people who have MS begin with relapsing-remitting cycles; (2) Secondary-progressive MS (SPMS) develops in people who have relapsing-remitting MS. In SPMS, relapses may occur, but there is no remission (stabilization) for a meaningful period of time and the disability progressively worsens; (3) Primary-progressive MS (PPMS), which progresses slowly and steadily from its onset and accounts for less than 20 percent of MS cases. There are no periods of remission, and symptoms generally do not decrease in intensity; and (4) Progressive-relapsing MS (PRMS). In this type of MS, people experience both steadily worsening symptoms and attacks during periods of remission. It should be understood that the method of the invention may be applicable for any type, stage or condition of the MS patient. Treatment using the methods of the invention may result in some embodiments in alleviation of any symptoms, and/or in prolonging the remission period between attacks.

In yet some further embodiments, the methods of the invention may be applicable for the treatment of SLE and any related conditions or symptoms associated therewith. More specifically, Systemic lupus erythematosus (SLE), also known simply as lupus, is an autoimmune disease. Symptoms vary between people and may be mild to severe. Common symptoms include painful and swollen joints, fever, chest pain, hair loss, mouth ulcers, swollen lymph nodes, feeling tired, and a red rash which is most commonly on the face. The disease is characterized by periods of illness, called flares, and periods of remission during which there are few symptoms.

The cause of SLE is not clear, however, is thought to involve genetics together with environmental factors. There are a number of other types of lupus erythematosus including discoid lupus erythematosus, neonatal lupus, and subacute cutaneous lupus erythematosus. It should be appreciated that the invention encompasses each of these types.

Still further, in some embodiments, the methods of the invention may be relevant for other auto immune disorders. For example, for the treatment of ANCA-associated disorders. Anti-neutrophil cytoplasmic antibodies (ANCAs), as used herein, include the perinuclear anti-neutrophil cytoplasmic antibodies (P-ANCA) that target mostly the MPO or EGPA, and are therefore also known as MPO-ANCA, Cytoplasmic anti-neutrophil cytoplasmic antibodies (c-ANCAs), that mostly target the proteinase 3 (PR3) protein and therefore are also known as PR3-ANCA, which is mostly associated with GPA, and atypical ANCA (a-ANCA), also known as x-ANCA, and are a group of autoantibodies, mainly of the IgG type, directed against antigens in the cytoplasm of neutrophil granulocytes (the most common type of white blood cell) and monocytes. p-ANCA is also associated with several medical conditions, it is fairly specific, but not sensitive for ulcerative colitis; a majority of primary sclerosing cholangitis; focal necrotizing and crescentic glomerulonephritis; and rheumatoid arthritis.

In some embodiments, the methods of the invention may be applicable for any ANCA-related or associated disorders. More specifically, such disorders include, but are not limited to ANCA-associated vasculitides (AAV), ANCA-associated glomerulonephritis (AAGN), crescentic glomerulonephritis (NCGN), and Rapidly progressive glomerulonephritis (RPGN).

In some further embodiments, the methods of the invention may be applicable for treating immune-related disorder such as an inflammatory disorder. In accordance with some embodiments, the methods of the invention are applicable in treating an inflammatory disorder. The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause. In some embodiments, an inflammatory disease that may be applicable for the methods of the invention may be any one of atherosclerosis, Rheumatoid arthritis (RA) and inflammatory bowel disease (IBD).

In yet some further embodiments, the MPO-related condition may be a neurodegenerative disorder. In some specific embodiments, the methods of the invention are applicable in treating a neurodegenerative disorder. In some embodiments, the neurodegenerative disorder may further involve inflammatory and/or vascular causes. Neurodegeneration is the umbrella term for the progressive loss of structure or function of neurons, including synaptic dysfunction and death of neurons. Many neurodegenerative diseases including Alzheimer's and Parkinson's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention.

In more specific embodiments, the methods of the invention may be applicable for treating a neurodegenerative disorder such as Alzheimer's disease or Parkinson's disease.

Alzheimer's disease (AD), as used herein refers to a disorder that involves deterioration of memory and other cognitive domains that in general leads to death within 3 to 9 years after diagnosis. The principal risk factor for Alzheimer's disease is age. The incidence of the disease doubles every 5 years after 65 years of age. Up to 5% of people with the disease have early onset AD (also known as younger-onset), that may appear at 40 or 50 years of age. Many molecular lesions have been detected in Alzheimer' s disease, but the overarching theme to emerge from the data is that an accumulation of misfolded proteins in the aging brain results in oxidative and inflammatory damage, which in turn leads to energy failure and synaptic dysfunction. More specifically, accumulation of Aβ within has been shown in structurally damaged mitochondria isolated from the brains of patients with Alzheimer' s disease.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

There is no single linear known chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range up to 20 years, depending on age and other health conditions.

The most common early symptom of AD is difficulty remembering newly learned information because AD changes typically begin in the part of the brain that affects learning and memory. As AD advances through the brain it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

The National Institute of Neurological and Communicative Disorders and Stroke (NINCDS) and the Alzheimer's Disease and Related Disorders Association (ADRDA, now known as the Alzheimer's Association) established the most commonly used NINCDS-ADRDA Alzheimer's Criteria for diagnosis in 1984, extensively updated in 2007. These criteria require that the presence of cognitive impairment, and a suspected dementia syndrome, be confirmed by neuropsychological testing for a clinical diagnosis of possible or probable AD. A histopathologic confirmation including a microscopic examination of brain tissue is required for a definitive diagnosis. Good statistical reliability and validity have been shown between the diagnostic criteria and definitive histopathological confirmation. Eight cognitive domains are most commonly impaired in AD: memory, language, perceptual skills, attention, constructive abilities, orientation, problem solving and functional abilities. These domains are equivalent to the NINCDS-ADRDA Alzheimer's Criteria as listed in the Diagnostic and Statistical Manual of Mental Disorders (DSM-IV-TR) published by the American Psychiatric Association. Beside symptomatic treatments to temporarily slow the worsening of dementia symptoms, AD has no current cure, and the current treatments cannot stop AD from progressing. It should be understood that the methods of the invention may be applicable for any stage, condition or symptom associated with AD, of any of the MPO-related conditions discussed herein.

In some embodiments, the target sequence targeted by the gene editing systems provided by the invention may be any sequence encoding receptors for antigen derived from a pathogen specifically, viral, bacterial, fungal, parasitic pathogen and the like. Thus, in some embodiments, the therapeutic methods of the invention may be applicable for any condition caused by at least one pathogen. More specifically, any immune-related disorder or condition that may be a pathologic condition caused by any of the pathogens disclosed by the invention, for example, an infectious disease caused by a pathogenic agent, specifically, a viral, bacterial, fungal, parasitic pathogen and the like. Pathogenic agents include prokaryotic microorganisms, lower eukaryotic microorganisms, complex eukaryotic organisms, viruses, fungi, prions, parasites, yeasts, toxins and venoms. Still further, in some embodiments, the methods of the invention may be applicable for disorders caused by a viral pathogen. A viral pathogen, as used herein, may be in some embodiments, of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages). In yet some more specific embodiments, the methods of the invention may be applicable for a viral disorder such as a Foot and Mouth Disease.

In yet some other specific embodiments, the methods and composition of the invention may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present invention provides compositions and methods for use in the treatment, prevention, amelioration or delay the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal.

It should be appreciated that an infectious disease as used herein also encompasses any pathologic condition caused by toxins and venoms.

In yet some particular and non-limiting embodiments, the methods of the invention may be applicable for congenital disorders, for example any one of adRP or PSACH. In yet some further embodiments, the methods of the invention may be applicable for any proliferative disorder, specifically for at least one of non-small cell lung cancer (NSCLC) melanoma, renal cell cancer, ovarian carcinoma and breast carcinoma. Still further, in some specific embodiments, the methods of the invention may be applicable for any disorder caused by a pathogen, for example, a viral disorder. In yet some more specific embodiments, the methods of the invention may be applicable for a viral disorder such as a Foot and Mouth Disease.

It should be appreciated that the methods of the invention enable in vivo editing of a target nucleic acid sequence of interest in cells of the treated subjects, by administering to the treated subject the PAM abolished or reduced CRISPR-Cas proteins of the invention, any chimeric proteins thereof, complex, conjugate, systems and/or any nucleic acid molecules encoding the Cas proteins of the invention or any chimeras thereof. However, in some alternative embodiments, the desired editing of the target nucleic acid sequence, may be performed ex vivo. In such option, the editing, or genetic manipulation of the nucleic acid sequence of interest is performed in cells of an autologous or allogeneic source, that are then administered to the subject.

Thus, in some embodiments, the methods of the invention may involve the step of administering to the treated subject an effective amount of a cell that comprises the PAM-reduced or abolished Cas-protein of the invention and any fusion protein modifier or effector thereof, or a cell that has been modified by the modifier of the invention and any fusion protein modifier or effector thereof. In some embodiments, such cell has been ex vivo modified using the systems of the invention. Thus, in some embodiments thereof, the methods of the invention may comprise the step of administering to the treated subject a therapeutically effective amount of at least one cell as defined by the invention or of any composition comprising any of the cells disclosed by the invention.

Still further, in some embodiments, the cells may be of an autologous or allogeneic source.

In some embodiments, the “host cells” provided herein, specifically, the cells transduced or transfected with or comprising the PAM-reduced or abolished Cas protein or the nucleic acid guided genome modifier/effector chimeric protein, complex or conjugate of the invention, and any systems thereof, and/or the encoding nucleic acid molecules provided by the invention, may be cells of an autologous source. The term “autologous” when relating to the source of cells, refers to cells derived or transferred from the same subject that is to be treated by the method of the invention.

In yet some further embodiments, the cells transduced or transfected with the PAM abolished or reduced CRISPR-Cas proteins of the invention, any chimeric proteins thereof, systems and/or nucleic acid molecules of the invention used by the methods of the invention may be cells of an allogeneic source, or even of a syngeneic source.

The term “allogeneic” when relating to the source of cells, refers to cells derived or transferred from a different subject, referred to herein as a donor, of the same species. The term “syngeneic” when relating to the source of cells, refers to cells derived or transferred from a genetically identical, or sufficiently identical and immunologically compatible subject (e.g., an identical twin). The methods of the invention may be useful for mutating, deleting, inserting or replacing a target nucleic acid sequence of interest (e.g., a coding or non-coding sequence) or any fragment thereof in a eukaryotic cell, with a replacement sequence provided by the invention, using recombination or NHEJ. There are several types of eukaryotic cells that may be used by the methods of the invention. By way of example, eukaryotic cells may be, but are not limited to, stem cells, e.g. hematopoietic stem cells (HSCs), embryonic stem cells, totipotent stem cells, pluripotent stem cells or induced pluripotent stem cells, multipotent progenitor cells and plant cells.

Stem cells are generally known for their three unique characteristics: (i) they have the unique ability to renew themselves continuously; (ii) they have the ability to differentiate into somatic cell types; and (iii) they have the ability to limit their own population into a small number. In mammals, there are two broad types of stem cells, namely embryonic stem cells (ESCs), and adult stem cells. Stem cells may be autologous or heterologous to the subject. In order to avoid rejection of the cells by the subject's immune system, autologous stem cells are usually preferred.

Thus, in some embodiments, the eukaryotic cells according to the invention may be embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers. hESCs may be also derived from the inner cell mass (ICM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos.

In further embodiments, the eukaryotic cells according to the invention are totipotent stem cells. Totipotent stem cells are versatile stem cells, and have the potential to give rise to any and all human cells, such as brain, liver, blood or heart cells or to an entire functional organism (e.g. the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the eukaryotic cells according to the invention are pluripotent stem cells. Similar to totipotent stem cells, a pluripotent stem cell refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells.

In still further embodiments, the eukaryotic cells that may be applicable for therapeutic methods according to the invention, are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells but cannot into other types of cells. Another example is the mesenchymal stem cell, which can differentiate into osteoblasts, chondrocytes, and adipocytes. Multipotent progenitor cells may be obtained by any method known to a person skilled in the art.

In yet further embodiments, the eukaryotic cells according to the invention are induced pluripotent stem cells. Induced pluripotent stem cells, commonly abbreviated as iPS cells are a type of pluripotent stem cell artificially derived from a non-pluripotent cell, typically an adult somatic cell, even a patient's own. Such cells can be induced to become pluripotent stem cells with apparently all the properties of hESCs. Induction requires only the delivery of four transcription factors found in embryos to reverse years of life as an adult cell back to an embryo-like cell. For example, iPS cells could be used for autologous transplantation in a patient with a rare disease. The mutation or mutations responsible for the patient's disease state could be corrected ex vivo in the iPS cells obtained from the patient as performed by the methods of the invention and the cells may be then implanted back into the patient (i.e. autologous transplantation).

It should be understood that any of the cells disclosed herein may be used by the methods of the invention for ex vivo therapy as disclosed above.

As described herein above, the invention provides in some aspects thereof therapeutic and prophylactic methods.

It is to be understood that the terms “treat”, “treating”, “treatment” or forms thereof, as used herein, mean curing, preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention. As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals By “mammalian subject” is meant any mammal for which the proposed therapy is desired, including human, livestock, equine, canine, and feline subjects, most specifically humans.

Still further, the methods of the invention may be also applicable for curing, preventing, ameliorating and treating a pathologic disorder in any vertebrate or invertebrate organisms as disclosed by the invention. In yet some further embodiments, the invention may be further applicable for treating pathologic disorders in plants, specifically, any of the plants disclosed by the invention.

As described above, the PAM reduced or abolished Cas protein of the invention, any chimeras or systems thereof, and the encoding nucleic acid sequences of the invention may be administered by the methods of the invention either ex vivo, by introduction thereof into cells that are being transplanted or transferred to the treated subject, or alternatively in vivo, where the PAM reduced or abolished Cas protein of the invention or any vector or composition thereof are directly administered to the subject.

It should be therefore understood that the number of administrations of treatment to a subject may vary. Introducing the genetically modified or transiently expressing cells that comprise the genetic manipulation caused by the PAM reduced or abolished Cas protein of the invention, into the subject may be a one-time event; but in certain situations, such treatment may elicit improvement for a limited period of time and require an on-going series of repeated treatments. In other situations, multiple administrations of the genetically modified or transiently expressing cells may be required before an effect is observed. The exact protocols depend upon the disease or condition, the stage of the disease and parameters of the individual subject being treated.

As mentioned above, the invention concerns any eukaryotic organism and as such may be also applicable for members of the biological kingdom Plantae.

In more specific embodiments, the PAM-reduced or abolished Cas protein or the nucleic acid guided genome modifier/effector chimeric protein, complex or conjugate of the invention and any systems, compositions and methods thereof, may be applicable for any plant. In more specific embodiments, such plant may be a dioecious plant or monoecious plant.

More specifically, in some embodiments the organism of the biological kingdom Plantae may be a dioecious plant, specifically, a plant presenting biparental reproduction. In some specific embodiments, the plant manipulated by the methods and systems of the invention may be of the family Cannabaceae, specifically, any one of Cannabis (hemp, marijuana) and Humulus (hops). In more specific embodiments, the plant of the family Cannabaceae may be Cannabis (hemp, marijuana). In yet some further embodiments, the plant of the family Cannabaceae may be Humulus (hops).

In some embodiments, any plants are applicable in the present invention, for example, any model plants such as, Arabidopsis, Tobacco, Solanum licopersicum, Solanum tuberosum.

In yet some further embodiments, Canola, Cereals (Corn wheat, Barley), rice, sugarcane, Beet, Cotton, Banana, Cassava, sweet potato, lentils, chickpea, peas, Soy, nuts, peanuts, Lemna, Apple, may be applicable in the present invention.

A non-comprehensive list of useful annual and perennial, domesticated or wild, monocotyledonous or dicotyledonous land plant or Algae — (i.e unicellular or multicellular algae including diatoms, microalgae, ulva, nori, gracilaria), applicable in accordance with the invention may include but are not limited to crops, ornamentals, herbs (i.e., labiacea such as sage, basil and mint, or lemon grass, chives), grasses (i.e., lawn and biofuel grasses and animal feed grasses), cereals (i.e., rice, wheat, rye, oats, corn), legumes (i.e. soy, beans, lentils, chick peas, peas, peanuts), leafy vegetables (i.e. kale, bok-choi, cress, lettuce, spinach, cabbage), Amaranthacea (i.e. sugar beet, beet, quinoa, spinach), Compositea (i.e. sunflower, lettuce, aster), Malvaceae (i.e. cotton, cacao, okra, hibiscus), cucurbits (i.e., cucumber, squash, melon, watermelon), Solanaceous species (i.e tobacco, potato, tomato, petunia and pepper), Umbellifera (i.e. carrot, celery, dill, parsley, cumin), Crucifera (i.e., oilseed rape, mustard, brassicas, cauliflower, radish), Sesame, the monocot Aspargales (i.e. onion, garlic, leek, asparagus, vanilla, lilies, tulips, narcissus), Myrtacea (i.e., Eucalyptus, pomegranate, guava), Subtropical fruit trees (i.e. Avocado, Mango, Litchi, papaya), Citrus (i.e. orange, lemon, grapefruit), Rosacea (i.e. apple, cherry, plum, almond, roses), berry-plants (i.e. grapes, mulberries, blueberries, raspberry, strawberry), nut trees (i.e. macademia, hazelnut, pecan, walnut, chestnuts, brazil nut, cashew), banana and plantain, palms (i.e., oil-palm, coconut and dates), evergreen, coniferous or deciduous trees, woody species.

Still further, plants useful for food, beverage (i.e. passion fruit, citrus, Paulinia, Humulus), biofuel (i.e. Ricinus, maize, soy, oil-palm, Jatropha, Switchgrass) biopesticide (i.e. pyrethrum, neem tree), ornament (i.e. cut, gardened or potted flower species such as lilies, roses, carnations, Poinsettia, petunia, cactuses, daffodils, shrubs, climbing plants, junipers), fibers (i.e. cotton, flax, agave, cannabis), construction, paper and cardboard, pigments, latex (i.e. Hevea), alcohol (i.e. grape, rye, sugarcane, cereals, fruit), oil (i.e. soy, peanut, sesame, maize, canola, rape, olive, oil-palm, argan, nuts), sugar (i.e. maize, sugarcane, sugar-beet, maple), fruit and vegetable, tea, coffee, cacao, olives, spices (i.e. ginger, cinnamon, curry, fenugreek, cumin, pepper, cardamom), chemical extraction, phytochemicals, antioxidants (i.e. plants producing phenolics, carotenoids, anthocyanins, and tocopherols), non-sugar sweeteners (i.e. Stevia), medicinal or bioactive compound producing plants (i.e. poppy, alkaloid producing species, cannabis, willow, foxglove, Cinchona (quinine) and Artemisia (antimalarial)), lawns, research model plants (i.e. Arabidopsis, tobacco), cosmetically useful plants (i.e. argan, aloe, jojoba, lavender, chamomile, tea-tree, geranium), industrially useful plants, industrial feedstock plants, animal (incl mammal, fish and insect) feed and fodder plants, bio-amelioration plants, fertilization, breeding stock, as encompassed by the invention.

In yet some further embodiments, nonfood products made from plants include essential oils, natural dyes, pigments, waxes, resins, tannins, alkaloids, amber and cork. Products derived from plants include soaps, shampoos, perfumes, cosmetics, paint, varnish, turpentine, rubber, latex, lubricants, linoleum, plastics, inks, and gums.

In some embodiments, the methods and systems of the invention may be applicable for any plant parts, specifically, leaves, shoots, seedlings, fronds, cane, seeds, fruit, nuts, berries, flowers, trunks, branches, bark, roots, corms, rhizomes bulbs and stems, latexes and exudates. Other plants that are pests (i.e. Orobanchaceae, Cuscuta) or weeds (broad-leaf weeds such as Convolvulus, Datura and monocot grasses such as crab grass, Cyperus).

Still further, another aspect of the invention relates to an effective amount of at least one of:

(a) at least one Cas protein or any Cas protein derived domain having reduced or abolished PAM constraint or any fragment, variant, mutant, fusion/chimeric protein, complex or conjugate thereof, or at least one nucleic acid sequence encoding said Cas protein or any variant, mutant, fusion/chimeric protein or conjugate thereof. More specifically, in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding said target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b);

(e) at least one host cell modified by, and/or comprising at least one of: (a), (b),(c) and (d); and

(f) at least one composition comprising at least one of (a), (b), (c), (d) and (e); for use in methods of curing or treating, preventing, inhibiting, reducing, eliminating, protecting or delaying the onset of a pathologic disorder or condition in a subject in need thereof.

In yet some further aspects thereof, the invention provides an effective amount of at least one of: (a) at least one Cas protein or any Cas protein derived domain having reduced or abolished PAM constraint or any variant, mutant, fusion/chimeric protein, complex or conjugate thereof, or at least one nucleic acid sequence encoding said Cas protein or any fragment, variant, mutant, fusion/chimeric protein or conjugate thereof. More specifically, in some optional embodiments, at least one of: the PBD and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced;

(b) at least one target recognition element or any nucleic acid sequence encoding the target recognition element;

(c) at least one nucleic acid cassette or any vector or vehicle comprising the nucleic acid sequence of (a), the nucleic acid sequence of (b) or the nucleic acid sequence of (a) and (b);

(d) at least one system comprising (a) and (b); and

(e) at least one composition comprising at least one of (a), (b), (c), and (d); for use in method of modifying at least one target nucleic acid sequence of interest in at least one cell or in a biochemical reaction.

In some embodiments, any of the CRISPR-Cas protein as defined herein, any of the fusion/chimeric protein or conjugate thereof, specifically, any of the nucleic acid guided genome modifier/effector chimeric protein, complex or conjugate according to the invention, any of the nucleic acid molecules as defined by the invention, any of the systems defined by the invention, any of the host cells of the invention and any of the compositions disclosed by the invention, are provided herein for use in methods of curing or treating, preventing, inhibiting, reducing, eliminating, protecting or delaying the onset of a pathologic disorder or condition in a subject in need thereof, and for use in methods for modifying at least one target nucleic acid sequence of interest in at least one cell.

It should be appreciated that according to some embodiments, the invention further provides transgenic organism/s or knock out organism/s, having a predetermined genetic modification formed by the method described herein. In some embodiments, the organism is any plant or an animal disclosed by the invention.

It should be further understood that the invention further encompasses any kit comprising any of the systems, compositions and cells of the invention or any combinations thereof with any additional therapeutic agent.

A kit as used herein may comprise the compositions described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10 %.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should be understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Experimental Procedures

Cells

HEK-293 cells [Embryonic Kidney; Human (Homo sapiens), ATCC].

Constructs

Plasmids for the expression of the modified Cas constructs were constructed by Gibson assembly of a DNA fragment (ordered from GeneArt) into a KanR PUC57-based backbone under the CMV promotor and the BGH terminator (Construct number and description in Table 1 below; protein sequence are presented in sequence listing).

EMX1 target 1 sgRNA constructs were ordered (GeneArt) in pairs (for the dCas-FokI system (Example 1), and for the dScCas-FokI system (Example 3) one sgRNA under H1 promoter and the other under the human U6 promoter. Different sgRNA distances (15 base and 27 base “gaps”) were designed. sgRNAs expressed from plasmid SEQ ID: 294 have a 15 bp gap, sgRNAs expressed from plasmid SEQ ID: 1 have a 27 bp gap.

Another double sgRNA cassette was ordered with a spacer of 15 bp, forming plasmid number 10917, that comprises the nucleic acid sequence as denoted by SEQ ID NO. 23.

TABLE 1
Plasmids

| | | | Bacterial | Guide |
|---|---|---|---|---|
| Number | Name | Plasmid Description | stock # | description |
| 10916 | dScCas9-FokI | Plasmid ordered from | 10918 | Guide pair |
| SEQ ID | EMX1sgRNA27T1- | GeneArt. dC9F - | | with a 27 base |
| NO. 1 | NNG | deactivated ScCas9 | | gap |
| | | PAM = NNG with FokI | | |
| | | under CMV-Promoter and | | |
| | | BGH terminator. With U6 | | |
| | | and H1 promoters and | | |
| | | sgRNAs for EMX1 | | |
| | | distance(is not 15) distance | | |
| | | is 27 target 1 | | |
| 11035 | dScCas9-FokI - | Digestion of 10916 by MluI | 11035 | Guide pair |
| SEQ ID | EMX1sgRNA15T1- | and Gibson assembly with | | with a 15 base |
| NO. 294 | NNG | 1017 AgeI to replace | | gap |
| | | SCNAs region | | |
| 11235 | dScCas9- | Digestion of 10918 by MluI | 11241 | No guide RNA |
| SEQ ID | FokI-NNG | to omit sgRNA, resulting in | | |
| NO. 270 | | dScCas9-FokI -NNG | | |
| | | confirmed by seq as dCAs | | |
| | | (no HSSBs) no sgRNA | | |
| | | confirmed by seq seq - | | |
| | | 190507NN005 | | |
| 10917 | DemxT1v2 | Plasmid ordered from | 10920 | Guide pair |
| SEQ ID | EMX1sgRNA15T1- | GeneArt. U6 and H1 | | with a 15 base |
| NO. 23 | NNG | promoters and sgRNAs for | | gap |
| | | EMX1 distance 15 target 1 | | |
| 11248 | dScCas9-FokI | Deleting the HnH domain - | | Guide pair |
| SEQ ID | dHNH- | PCR on 10916 and cloning | | with a 27 base |
| NO. 9 | F-NNG EMX1 T1 | to 10918 dScCas9-FokI -F- | | gap |
| | sgRNA 27 | NNG. 3157/3207, | | |
| | | 3206/3158 to 10918 | | |
| | | EcoRI/BamHI/HindIII. | | |
| | | N50p64-78 | | |
| 11254 | dScCas9-FokI | 11248 digested by MluI To | | No guide RNA |
| SEQ ID | dHNH- | omit the sgRNA cassette | | |
| NO. 13 | F-NNG | colony - 1 N50p79-80 | | |

Cloning

Cloning of dScCas9—FokI (Example 1):

dScCas9-FokI plasmid construct 10916 (as denoted by SEQ ID NO. 1) containing CMV promoter, dScCas9-FokI, BGH-Terminator and a double sgRNA cassette containing the 27 spacer EMX1 Targetl sgRNAs was ordered from Geneart. This cassette encodes the dScCas9-FokI Protein that comprise the amino acid sequence as denoted by SEQ ID NO. 2. The sgRNA cassette for this cassette is EMX1sgRNA27T1-NNG (the nucleic acid sequence is denoted by SEQ ID NO. 3). Another double sgRNA cassette was ordered with a spacer of 15 bp, forming plasmid number 10917 EMX1sgRNA15T1-NNG (the nucleic acid sequence is denoted by SEQ ID NO. 4).

Cloning of dScCas9-FokI size-reduction (Example 3) construct 11248 (seq 9)

The following sequence containing CMV promoter, dHNH-dScCas9-Fok, BGH-Terminator and a double sgRNA cassette containing the 27 spacer EMX1 Targetl sgRNAs was constructed as follow: PCR on 10916 with primers 3157/3207,3206/3158 (Table 2) and Gibson assembly cloning to 10918 (Ecoli stock of plasmid 10916) dC9Xten-F-NNG digested by EcoRI/BamHI/HindIII.

TABLE 2
Primers list of dHNH

| Primer | | SEQ |
|---|---|---|
| name | Primer sequence | ID NO: |
| 3157 | ATTAGTTCATAGCCCATATATGGAGTTC | 5 |
| 3158 | TACGCGTAGTTGTGGGTTTGTC | 6 |
| 3206 | TCCAGAGAGAGAAAGAAGCGcGGCGGACT | 7 |
| | GTCTGAAGCTGAT | |
| 3207 | ATCAGCTTCAGACAGTCCGCCgCGCTTCTT | 8 |
| | TCTCTCTCTGGA | |

Reverse Transfection

Reverse transfection to HEK-293 cells was done using TransIT® LT1 transfection reagent (Mirus Bio) diluted in serum-free medium Opti-MEM® I (Gibco). Transfection reagent:DNA ratio of 5 pl per 1 μg DNA was used. For reverse transfection in a well in 96 wells plate: 100 ng plasmid DNA was used in 3 ul total volume (33.3 ng/u1). Transfection reagent was first diluted (1:19) in Opti-MEM® I. Then, 3 ul DNA (33.3 ng/u1) was added to the diluted transfect reagent, mixed gently and incubated 15-30 min in room temperature. Then, HEK-293 cells (3.6×10⁴) were gently added on the top of the TransIT®-DNA complexes and mixed as is customary. Cells were incubated 72 h in 37° C. in a CO2 incubator (HERACELL 150i, Thermo Scientific). If co-transfection was done, plasmids were equally mixed in advance to final DNA concentration of 100 ng/3 ul. (for example: if two plasmids were used, 50 ng from each plasmid was mixed in 3 ul final volume 33.3 ng/ul DNA, if three plasmids were used, 33.3 ng from each was mixed in 3 ul final volume 33.3 ng/ul DNA).

Genomic DNA Preparation

Genomic DNA (gDNA) from HEK-293 cells was extracted 72 hours post transfection, using the Quick-DNA™ 96 Kit (Zymo Research). Medium was removed using vacuum and glass Pasteur pipette. Then, 200 μl Genomic Lysis Buffer were added and using mechanic pipetation up and down, cells were directly lysed in the 96 plate well. The concentration of gDNA was determined using NanoDrop 2000 (Thermo Scientific).

Droplet Digital PCR Design and Execution.

QX200™ Droplet Digital™ PCR system (BIORAD) was used for the mutations analysis in the target site on the gDNA.

Assay designed, TGEE3 or TGEE4 for mutation detection in EMX1 gene -Target1:

Detailed reaction particulars and primers can be found in Table 3 (Tables 3a, 3b, 3c and 3d). As TGEE3 and TGEE4 are assays to analyze mutation in adjacent sites, same primers were used for the amplification of the target site were in both assays. In addition, the same reference probe was used for both assays. The drop-off probes were different and specific for each site.

Primers 3042-Forward and 3049-Reverse (as denoted by SEQ ID NO. 18 and SEQ ID NO. 19, respectively), were designed to amplify one specific 317 bp fragment in Tm=55° C. The primers that were designed as reference and drop off probes were first tested in a simple PCR reaction to amplify a specific fragment when using with the 3049-Reverse primer in Tm=55° C. Then, the reference probe was ordered from IDT with FAM™ modification in the 5′ end and with Iowa Black® Quencher in the 3′ end. The drop off probe was ordered from IDT with HEX™ modification in the 5′ end and with Iowa Black® Quencher in the 3′ end with 2 locked nucleic acid (LNA) bases inside the target site. Tm of reference and drop off probes were designed to be higher in 3-10° C. than 55° C.

TABLE 3a
Assay execution:

| | |
|---|---|
| EMX1-Target1-TGEE3 | for 1 reaction |
| orTGEE4assay | |
| 2XddPCR Supermix for probes | 11 μl |
| 20X NHEJ TGEE-3 or TGEE4 | 1 μl |
| EMX1-T1 | |
| HindIII diluted 2-5 U/μl | 1 μl |
| 150 ng gDNA | 9 μl |

TABLE 3b

| | |
|---|---|
| 20X NHEJ TGEE-3 or TGEE4 | μl for 100 reactions |
| EMX1-T1 | |
| 18 uM primer 3042-EMX1-T1- | 18 μl |
| ddPCR-F1 | |
| 18 uM primer 3049-EMX1-T1- | 18 μl |
| ddPCR-R2 | |
| 100 uM 3088-EMX1-T1-TGEE3- | 5 μl |
| reference probe | |
| 100 uM 3089-EMX1-T1-TGEE3- | 5 μl |
| drop off probe (HEX/2XLNAs) or | |
| 13175-EMX1-T1-TGEE4-drop | |
| off probe (HEX/2XLNAs) | |
| DDW | 54 μl |

TABLE 3c

| | |
|---|---|
| HindIII diluted 2-5 U/μl | μl for 100 |
| | reactions |
| 10X buffer CutSmart NEB | 10 μl |
| HindIII NEB | 25 μl |
| DDW | 65 μl |

TABLE 3d

| Primers and probes | for 1 reaction |
|---|---|
| 3042-EMX1-T1-ddPCR-F1 | gaaaccatgcccattc as denoted by SEQ ID NO: 18 |
| 3049-EMX1-T1-ddPCR-R2 | agagaagggtggttttc as denoted by SEQ ID NO: 19 |
| 3088-EMX1-T1-TGEE3- | /56-FAM/CT CTG TAT G/ZEN/G AAA AGA GCA |
| reference probe | T/3IABkFQ/ as denoted by SEQ ID NO: 20 |
| 3089-EMX1-T1-TGEE3-drop off | /5HEX/tcgtaga+g+tcccatgtc/3IABkFQ as denoted by |
| probe | SEQ ID NO: 21 |
| 3175-EMX1-T1-TGEE4-drop off | /5HEX/ATGTCTGC+C+GGCTTCC/3IABkFQ |
| probe | as denoted by SEQ ID NO: 22 |

It should be noted that the probes used herein, specifically, the probes of SEQ ID Nos. 20-22, that comprise an “+n nucleotide”, for example, +C or +G (n can be a, c, g, or t), refers to a “N”, where there is a methylene bridge bond linking the 2′ oxygen to the 4′ carbon of the RNA pentose ring. This bond causes higher structural rigidity and increased melting temperature of the oligonucleotide.

Data Analysis

QX200™ Droplet Reader and QuantaSoft™ software (BIORAD) were used for Data analyzing. For each assay and each experiment, a threshold was determined in relation to all experiment treatments, controls and no DNA control sample.

Thresholds were analyzed separately for each experiment and for each assay, as presented by Table 4:

TABLE 4
ddPCR Analysis thresholds

| Experiment | Assay TGEE3 | Assay TGEE4 |
|---|---|---|
| 1 (#13) | | FAM1840/HEX2410 |
| 2 (#16) | FAM 1500/HEX1430 | FAM1500/HEX1770 |
| 3 (#32) | | FAM 2900/HEX 1900 or |
| | | FAM2020/HEX1570 |

Poisson correction was done according to manufacturer's instructions (Droplet Digital PCR Applications Guide, BioRad, p7-8). Briefly, a Poisson correction factor is inferred by modeling a Poisson distribution from the fraction of empty cells. Explicitly, the Poisson correction factor is the infinite sum of the probability of a cell containing 1 DNA molecule only, plus two times the probability of two DNA molecules, plus three times the probability of three DNA molecules, and so on. This correction factor is multiplied with the observed number of hits to find the true number of DNA molecules.

Delivery—Protoplasts

Bioassay setup: Arabidopsis protoplast preparation is based on Wu et. al. (Wu et al, Plant methods 16, 2009):

Plant material: Arabidopsis grown under 16 hr day optimal light (150 μE·s⁻¹) @ 22° C. Leaves: 3-5 week old plants (W ˜2cm L ˜5cm).

Work Solutions: Enzyme solution. 1%Cellulase, 0.25% Macerozyme, 0.4M Mannitol, 10 mM CaCl₂, 20 mM KCl, 0.1% BSA, 20 mM MES pH5.7. Heat 50-55° C. 10 minutes to inactivate proteases and then filter. Use fresh. 10 ml/7-10 peeled leaves (1-5 gr)/dish.

Modified W5 solution. 154 mM NaCl, 125 mM CaCl₂, 5 mM KCl, 5 mM Glucose, 2 mM MES pH5.7. Wash twice with 25 ml/plate, +twice 3 ml for transfection wash+/ml resuspension

Modified MMg solution. (Resuspension solution) 0.4M Mannitol, 15 mM MgCl₂, 4 mM MES pH5.7.

Modified TEAMP transfection buffer (PEG solution). 40% PEG MW 4000, 0.1M CaCl₂, 0.2M Mannitol (freshly prepared!) volume=1:1 of 200 μl protoplasts in MMg +volume of DNA BSA. 1% BSA

Arabidopsis Protoplasts

1. Preheat water bath to 50-55° C., cool swing-out centrifuge, chill W5 and MMg, and cut tips.

2. Prepare fresh BSA coated plates (tentative: 1.25ml 1%BSA/well in water, incubate on bench till ready)

3. Make fresh enzyme solution 10 ml/treatment.

4. Pick 7-10 leaves, must not be wet. 10 leaves should yield ˜4-5 transformations.

5. Tape upper epidermis with Time-tape, lower with Magic tape. Easier to peel if petiole is stuck to time-tape only.

6. 0.22 μm-filter 10 ml fresh enzyme solution into each petri dish

7. Peel and discard Magic tape. Transfer Time-tape side to petri dish

8. Gently shake on platform shaker 40 rpm 20-60 min in light until protoplast release (check empirically)

9. Centrifuge in 50 ml tubes 100×g 3 min in swing-out rotor

10. Wash twice with 25 ml cold W5

11. Ice 30 min, count during this time in hemocytometer using light microscope

12. Centrifuge and resuspend in MMg to 2-5×10⁵ cells/ml (about 1 ml).

Transfection:

1. Make fresh PEG sol for transfection in 2 ml tube

2. Pour off BSA from 6-well plates and dry

3. Mix ˜5×10⁴ protoplasts (2×10⁴−1×10⁵) in 0.2 ml MMg with a mixture of Donor plasmid DNA (where relevant), Protein Moiety expressing plasmid DNA and SCNAs ssDNA to a total of 30-40 μg at RT° in 15 ml round-bottom (snap-cap) tubes. Alternatively, Donor DNA and Protein-moiety expressing DNA are constructed and delivered on a single plasmid.

4. Add equal volume (0.2 ml protoplasts +midiprep vol.) of fresh PEG sol

5. Incubate RT° 5 min

6. Wash by slowly adding 3 ml W5, lml at a time, and mixing

7. Centrifuge 100×g in swing-out 1 min

8. Repeat wash and pellet

9. Resuspend in lml W5

10. Pour into BSA-coated plates

11. Grow protoplasts under 16 hr day optimal light (150 μE·m⁻²·s⁻¹)@ 22° C., replacing media as needed.

Arabidopsis Floral Dip Protocol

5% sucrose solution containing 0.01-0.05% (vol/vol) Silwet L-77 and resuspended Agrobacterium cells.

In vitro activity assay (for Example 6)

Materials used:

Protein expression:

For protein expression, TNT® SP6 High-Yield Wheat Germ Protein E L3261 (Promega) was used as described in Table 5.

TABLE 5
In vitro protein expression
Reaction volume - 50 μl

| | | |
|---|---|---|
| TnT SP6 High-Yield Wheat | 30 | μl |
| Wheat Germ Master Mix | | |
| DNA (purified PCR product) | 8 | μl |
| DDW | 12 | μl |
| Total | 50 | μl |

TNT Template DNA should be about:

(3.7 Kb-2-3 μg DNA required) therefore 4 μg of PCR product for dScCAS9FokI (or equivalent other RNA guided nucleases)

sgRNA:

Alt-R® CRISPR-Cas9 sgRNA, 2 nmol (IDT), or equivalent

DNA substrate for the reaction:

Digest DNA in buffer 3.1 with BsaI (NEB). 100 ng/μl final concentration (10 μg/100 μl).

Take 2 μl (3 Kb plasmid) for the final reaction.

The in-vitro activity assay:

1. Make a mix by Table 6.

TABLE 6
In vitro nuclease assay
Experiment reaction (based on NEB cat. M0386S):

| | | Adjusted to | | | |
|---|---|---|---|---|---|
| | NEB | SP6 Promega TnT | | | |
| | protocol for | Volume | | | |
| | Cas9 | | 1X | | X |
| Nuclease-free water | 20 μl | | 15 | μl | |
| NEBuffer 3.1 | 3 μl | | 3 | μl | |
| 300 nM sgRNA | 3 μl (30 nM | | 3 | μl | |
| | final) | | | | |
| 1 μM Cas9 Nuclease, | 1 μl (~30 | TnT- | 6 | μl | |
| S. pyogenes (M0386S) | nM final) | Expressed | | | |
| NEB as a control | | RNA- | | | |
| | | guided | | | |
| | | nuclease | | | |
| Reaction volume | 27 μl | | 27 | μl | |
| Pre-incubate for 10 minutes at 25° C. | | | | | |
| 30 nM substrate DNA | 3 μl (3 nM | | 1-2 μl | | |
| | final) | | plasmid | | |
| | | | DNA | | |
| Total reaction volume | 30 μl | | 30 | μl | |

2. Mix thoroughly and pulse-spin in a microfuge.

3. Incubate at 37° C. for 15 minutes.

4. Add 1 μl of Proteinase K to each sample, Mix thoroughly and pulse-spin in a microfuge.

5. Incubate at room temperature for 10 minutes.

6. Proceed with fragment analysis.

Further considerations:

Calculation of target DNA amounts:

Due to detection limits of EtBr stained electrophoresis gels, use the following amounts of target DNA for the assay: for a sensitive assay, use an 80 bp target. Per assay use about 5 ng of biotinylated end-labeled DNA (biotin to be added via a biotinylated primer (IDT), detection via blotting and reacting the membrane with an Avidin-AP (or Avidin-peroxidase) conjugate and detection via colorimetric (or chemiluminescent assay)).

Alternatively, for simpler EtBr detection use about 200 ng unmodified DNA for a 3 kb target plasmid. This assay should be able to detect about 10% cleavage or more.

Amount of enzyme to be used

Optimally, 1 μM from the TnT protein expression reaction (bpi or less depending on TnT expression level). Practically, determined empirically in a dilution series.

Detection of digested target plasmid:

1% Agarose gel if DNA is 3600 bp-Et-Br detection

To ease gel analysis DNA may be digested with BsaI resulting in a restriction pattern shown in Table 7.

TABLE 7
In-vitro nuclease assay analysis - expected digestion results

| | | *Orientation | *Orientation |
|---|---|---|---|
| | | 1 | 2 |
| | | Enzyme + | Enzyme + |
| | BsaI | BsaI | BsaI |
| bp | 3235 | 1797 | 1700 |
| bp | | 1453 | 1528 |

*Orientation is determined by the insertion direction of the target PCR insert

Methods for Off-Target Detection Generated by the Nucleases of the Invention

Off-target mutations may cause genomic instability and disrupt the functionality of otherwise normal genes. Therefore, it is important to be able to detect the presence of off-target cleavage. Methods for off-target detection fall into two categories, biased and un-biased. Biased methods designed to detect mutations at predicted potential off-target sites whereas unbiased methods will ideally locate this kind of mutations anywhere in the genome.

All biased methods are PCR based assays and share the same initial steps. First, prediction of off-target sites will be made using different In Silico CRISPR\Cas9 design tools or other relevant bioinformatics methods. Next, primers are designed for those specific sites and amplified by PCR in order to validate the cleavage. From this step forward, other than the obvious sequencing of the PCR products, there are several other methods to detect those anticipate off-target mutations, such as High-resolution melting analysis (HRMA), Mismatch cleavage with T7 or Surveyor endonuclease and mobility assay by PAGE

All biased methods are cheap, simple and fast but suffer from an inability to detect off-target mutations that occur at frequencies <1%, and suffer from the fact that there is a good portion of the off-target sites that cannot be predicted by the design tools available today. Furthermore these methods are not practical for large scale screening. However, when a specific oncogene or important regulatory element has been even weakly predicted to be under risk, such targeted PCR based assays should be employed.

Among the many unbiased methods existing, listed here are few of the more efficient and more relevant to the examples of the invention:

1. LAM-HTGTS—Linear Amplification-Mediated High-Throughput Genome-Wide Translocation Sequencing

LAM PCR can detect unknown DNA sequence that is in proximity to a known one. LAM-HTGTS is based on the translocation between known DNA added to the cells (refer to as ‘bait’) and the unknown fragments of DNA where the off-target DSB have occur (refer to as ‘prey’). LAM-PCR oriented toward the ‘bait’ known DNA will also amplify the ‘prey’ sequence thus allowing us to locate and identify off target cleavage in the genome.

2. BLESS—Direct In Situ Breaks Labeling, Enrichment on Streptavidin, and Next-Generation Sequencing

As the name suggest, in this method one labels directly the DSB using Biotinylated pinheads' linkers, enriches them on streptavidin and eventually analyzes the fragments using NGS and PCR with linker-specific primers.

3. GUIDE-Seq-Genome-Wide Unbiased Identification of DSBs Enabled by Sequencing

In this method the identification of the DSBs occurs via blunt double-stranded oligodeoxynucleotide (dsODN) that integrate with blunt DSBs in the genome (caused by RNA guided nuclease) through end joining processes such as NHEJ. The dsODN integration sites then amplified using NGS in order to locate the off-target DSBs.

4. Digenome-Seq—In Vitro Nuclease-Digested Genomes (Digenomes).

The genome is digested in vitro, using the guided nuclease of the invention, into smaller fragments with identical 5′ ends (sequence reads). After applying whole genome sequencing (WGS) on those fragments they will vertically align at cleavage sites, while uncut sequences will be aligned in a staggered pattern. Hence, off-target DSBs can be identified.

5. Whole Genome Sequencing

High throughput sequencing of the entire genome and comparison to a reference sequence. The entire genome is screened for off-target mutations therefore it is very accurate but also very expensive. This method has serious drawbacks in discerning SNPs and sequencing errors from bona fide off-target mutations and in the limited number of genomes that can be sequenced.

### Example 1

### PAM-Reduced Cas-Based Nucleoprotein: dScCas9-FokI

Here, an example of a reduced-PAM nuclease-dead ScCas9-FokI nuclease fusion (dScCas9-FokI) is provided, thereby establishing the feasibility of creating effective and highly specific gene editing tools. FokI-Cas fusions have been shown for SpCas9 [8] but not for ScCas9. This alteration is highly beneficial due to a significantly larger choice of target sites caused by the removal of the requirement for NGG sequences flanking the target site. As illustrated by Table 8 below, very few potential target sites can be found bioinformatically in the two example genes with dSpCas9-FokI, while between 5.7- and 8.6-fold more targets can be targeted with dScCas9-FokI.

TABLE 8

| | | Sites in Programmed |
|---|---|---|
| Cas9 | Sites in EMX1 | Cell Death 1 (PDCD1) |
| PAM | gene (49.9 kb) | gene (9 kb) |
| SpCas9 | 4438 | 1057 |
| 5′-NGG-3′ | | |
| ScCas9 | 13596 | 2729 |
| 5′-NNG-3′ | | |
| dCas9-Fok | Sites in EMX1 | Sites in PDCD1 |
| PAM | gene (49.9 kb) | gene (9 kb) |
| SpCas9 | Only 416 | Only 160 |
| 5′-NGG-3′ | | |
| ScCas9 | 3561 | 904 |
| 5′-NNG-3′ | | |

dScCas9 (dead Streptococcus canis Cas9) has previously been inactivated by introduction of two mutations in both its HNH and RuvC nuclease domains, D1OA and H849A [9].

The PAM recognition domain of ScCas9 has been identified through its homology with SpCas9 by Chatterjee et al. PAM binding specificity is mediated by an “RXR” motif (Arginine, any amino acid, Arginine), in a similar manner to spCas9 [1]: both these proteins have a pair of Arginines (Arg) that are predicted to interact with the PAM DNA via hydrogen bonds. The native PAM requirement of ScCas9 is NNG while that of SpCas9 is NGG.

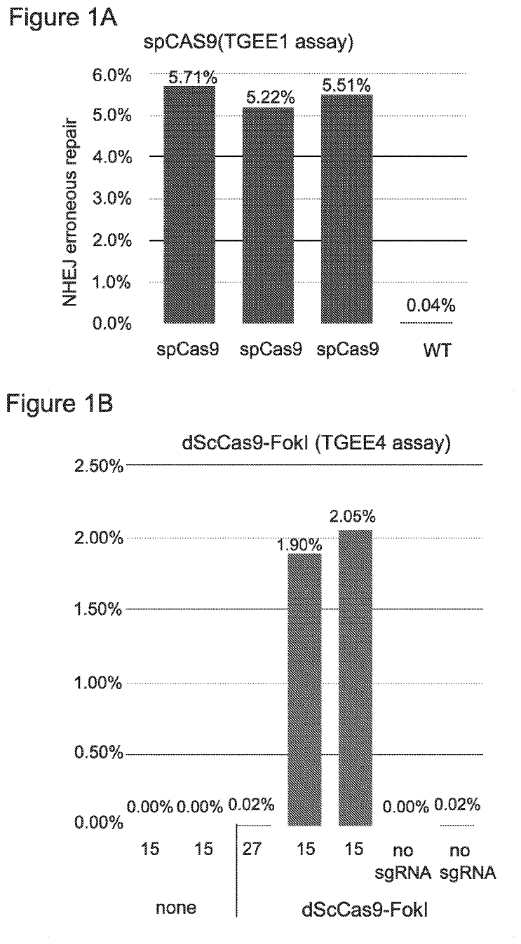

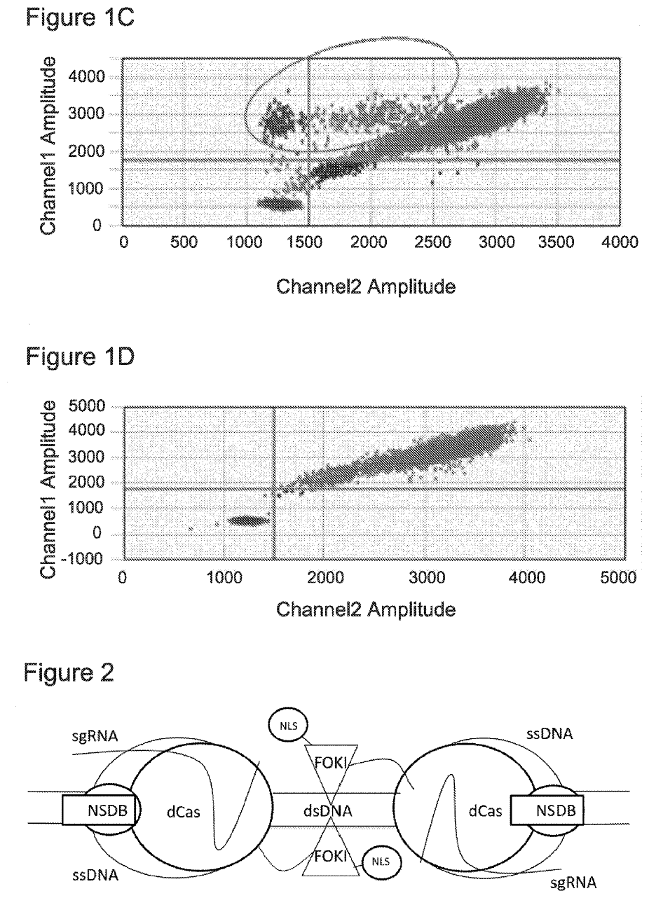

It was tested whether dScCas9-FokI can be used as a reduced-PAM obligatory-dimer genome editing system. Two SCNA (sgRNA) gaps were tested of 15 or 27 nucleotide bases. The expected best gap was 26 bases and thus 27 was not predicted to be optimal. Specifically, ddPCR analysis of the EMX1 gene targeting was tested in human cells. Different protein-SCNA (sgRNA) combinations were transfected into HEK293 cells and harvested at 72 hours post transfection. ddPCR analysis was made using BioRad software counting only single-template droplets which were HEX-negative FAM positive. This was followed by Poisson correction for inclusion of the expected number of 2,3,4 etc template containing droplets (see “tail” in FIG. 1C and 1D) as calculated from the empty/full droplet ratio multiplied by the percentage of blue droplets with a 95% confidence level. It was found that this construct is slightly less efficient than wild-type SpCas9 (see FIGS. 1A and 1B). Thus, this construct is highly useful and is a significant improvement upon both ScCas9 which is theoretically about 1 million times less precise, and upon dSpCas9-Fok which is impeded by a double NGG requirement.

### Example 2

### PAM-Abolished Cas-Based Nucleoproteins

To further improve the gene editing tools provided by the invention, the inventors next designed PAM-abolished Cas-based nucleoprotein. As illustrated in FIG. 2, dCas-FokI fusion protein comprising a Nuclear Localization Sequence (NLS), FokI nuclease monomer (FokI), nuclease-deficient Cas nucleoprotein (dCas), and a non-sequence-specific DNA-binding domain (NSDB), bound to a single guide RNA (sgRNA), was designed. Two monomers of this fusion protein bind DNA target sites separated by a double-stranded gap region (dsDNA), positioning the Fold domains for dimerization and cleavage, as schematically represented in FIG. 2.

More specifically, to reduce or abolish PAM recognition of the Cas nuclease, the inventors designed Cas variants where the PAM Binding Domain (PAM BD), including a loop containing amino acid residues Thr1330 to Arg1342 (corresponding to SpCas9 Lys1325-Arg1335) which include PAM interacting Arginine residues, is removed. While Chatterjee et al deleted Lys1337+Gln1338 (KQ) adjacent to these Arginine residues to revert the sequence to a sequence similar to SpCas9 (this deletion rendered the PAM more specific, from NNG to NGG), they did not delete the entire PAM recognition motif or the essential Arginine residues therefore retaining the requirement for at least one Guanine in the PAM.

Additional or alternative constructs encoding Cas variants with a deletion combining this loop, with a second loop (“Sc loop”) structurally adjacent (topologically but not linearly adjacent in the amino-acid sequence) to the PAM binding region and on the opposite side of the DNA helix, were also prepared. This deletion includes residues Ile 367 to Ala 376 (also performed by Chatterjee et al.). Deletion of this region further confines PAM requirement in ScCas9 from NNG to NAG while combining this mutation with KQ and Sc loop deletion further confines the specificity of ScCas9 to NGG.

Different variants of the Sc loop were designed and constructed in order to alter PAM specificity (ordered as gene synthesis constructs from various suppliers). Variants included deletions of the Sc loop, Sc loop with one or more point mutations including mutations of Arg residues 370 and 372 of ScCas9 (the ScCas9 amino acid sequence is as denoted by SEQ ID NO: 258), and Sc loop with sequences derived from homologous Cas9 variants. More specifically, the designed variants include dScCas9-FokI-LoopDel (complete deletion of the Sc Loop), as denoted by SEQ ID NO. 345; dScCas9-FokI-LoopQQ (Sc Loop with the two Arg residues mutated to Gln residues), as denoted by SEQ ID NO. 346; dScCas9-FokI-LoopAA (Sc Loop with the two Arg residues mutated to Ala residues), as denoted by SEQ ID NO. 347; dScCasFok, ScLoopASp, SV40+nucleoplasminNLS Cas9, as denoted by SEQ ID NO. 348 (Sc loop with the loop replaced by the corresponding region from SpCas9); the dScCasFok, ancestral RuvC+Rec1/2, ScLoopA, SV40+nucleoplasminNLS as denoted by SEQ ID NO: 394; the dScCasFok, ancestral RuvC+Rec1/2, ScLoopQQ, SV40+nucleoplasminNLS as denoted by SEQ ID NO: 395; the dScCasFok, ancestral RuvC+Rec1/2, ScLoopAA, SV40+nucleoplasminNLS as denoted by SEQ ID NO: 396. These modifications may also be combined with dScCas9-FokI mutations described in other examples.

Moreover, Sc loop deletions and replacements may be done in combination with mutations that may enhance stability/expression/activity, which may include ancestral mutations (see Example 16). Sc Loop deletion/mutation constructs were designed and synthesized, editing efficiency was tested, and the results are shown in Table 9. More specifically, SCNAs targeting the human MPO gene exon 1 were encoded by plasmid GeneMsgRNA15E1. MPO gene NHEJ erroneous repair was tested in Hek293 cells, harvested 72 hours after transfection with plasmids encoding the protein and the SCNAs. It was observed in one experiment (see for example ddPCR#76, Table 9), that while Sc Loop deletions and mutations had undetectable activity, addition of ancestral mutations in the REC domain improved the activity to roughly half of that of normal dScCasFok. In another experiment, all variants had detectable activity (see ddPCR#81, Table 9). Moreover, replacement of the ScLoop with the corresponding region from SpCas9 also resulted in a functional dScCasFok variant. This shows that it is possible to remove the Sc loop and retain high activity.

TABLE 9
Gene editing efficiencies of Sc loop replacement constructs

| | Construct | editing % | editing % |
|---|---|---|---|
| Description | (SEQ ID NO) | (ddPCR#76) | (ddPCR#81) |
| SpCas9, SV40 + nucleoplasmin NLS | 7665 (257) | 49.73% | 42.99% |
| dScCasFok, SV40 NLS | 11241 (2) | 17.77% | 21.24% |
| dScCasFok, ScLoopΔ, SV40 + nucleoplasminNLS | 14478 (345) | 0.00% | 7.01% |
| dScCasFok, ScLoopQQ, SV40 + nucleoplasminNLS | 14479 (346) | 0.01% | 8.86% |
| dScCasFok, ScLoopAA, SV40 + nucleoplasminNLS | 14480 (347) | 0.00% | 6.10% |
| dScCasFok, ScLoopΔSp , SV40 + nucleoplasminNLS | 14481 (348) | 8.43% | 7.59% |
| dScCasFok, ancestral RuvC + Rec1/2, ScLoopΔ, | 14483 (394) | 10.50% | 8.08% |
| SV40 + nucleoplasminNLS | | | |
| dScCasFok, ancestral RuvC + Rec1/2, ScLoopQQ, | 14484 (395) | 6.11% | 5.34% |
| SV40 + nucleoplasminNLS | | | |
| dScCasFok, ancestral RuvC + Rec1/2, ScLoopAA, | 14485 (396) | 6.41% | 6.22% |
| SV40 + nucleoplasminNLS | | | |

Editing efficiency was quantified by ddPCR (ddPCR#76 and #81 results are shown). NLS = Nuclear localization sequence.

In order to enable non-PAM-specific target DNA binding by the nucleoprotein complex, the PAM binding domain of Cas-based nucleoproteins is replaced by a Non-Sequence Specific DNA Binding Domain (NSDB).

More specifically, examples for constructs provided by the invention include dScCas9-FokI with PAM BD replaced with one or more NSDBs that include ZFs, TALEs, HTHs, SH3s, HMGs, or StkCs.

The PAM restrictions or constraints of these variants may be removed or reduced thereby widening the possibility to use these fusion proteins for genome editing in larger eukaryotic genomes without restriction of target site use.

The following PAM-abolished chimeras were constructed (ordered as gene synthesis constructs from various suppliers): dScCas9-FokI-ZF1, as denoted by SEQ ID NO. 24, dScCas9-FokI-ZF2, as denoted by SEQ ID NO. 25, dScCas9-FokI-ZF3, as denoted by SEQ ID NO. 26, dScCas9-FokI-ZF4, as denoted by SEQ ID NO. 27, dScCas9-FokI-LAC, as denoted by SEQ ID NO. 28, dScCas9-FokI-LAC2, as denoted by SEQ ID NO. 29, dScCas9-FokI-LAC3, as denoted by SEQ ID NO. 30, dScCas9-FokI-LAC4, as denoted by SEQ ID NO. 31, dScCas9-FokI-HIVIN, as denoted by SEQ ID NO. 32, dScCas9-FokI-HIVIN2, as denoted by SEQ ID NO. 33, dScCas9-FokI-HIVIN3, as denoted by SEQ ID NO. 34, dScCas9-FokI-HIVIN4, as denoted by SEQ ID NO. 35, dScCas9-FokI-SSO7D, as denoted by SEQ ID NO. 36, dScCas9-FokI-SSO7D2, as denoted by SEQ ID NO. 37, dScCas9-FokI-SSO7D3, as denoted by SEQ ID NO. 38, dScCas9-FokI-SSO7D4, as denoted by SEQ ID NO. 39, dScCas9-FokI-STO7, as denoted by SEQ ID NO. 40, dScCas9-FokI-STO72, as denoted by SEQ ID NO. 41, dScCas9-FokI-ST073, as denoted by SEQ ID NO. 42, dScCas9-FokI-ST074, as denoted by SEQ ID NO. 43, dScCas9-FokI-StkC, as denoted by SEQ ID NO. 44, dScCas9-FokI-StkC2, as denoted by SEQ ID NO. 266 and dScCas9-FokI-HMGB4 as denoted by SEQ ID NO.267.

PAM-abolished chimeras may also be combined with dScCas9-FokI mutations described in other examples. One important combination may be combination with HNH deletions (HNHA) described in EXAMPLE 3, which may allow additional space in the protein due to removal of the large HNH domain.

To evaluate ability of dCasFok variants to function with PAM BD replacements, constructs were tested in HEK293 cells for NHEJ erroneous repair using digital droplet PCR, using guide RNAs encoded by plasmid GeneMsgRNA15E1 (SEQ ID NO. 325) which targets human myeloperoxidase (MPO) gene exon 1, with ddPCR probe PM0Ex1-TGEE6 (SEQ ID NO. 327) and primers PM0Ex1 F2 FWD (SEQ ID NO. 328) and PMOEx2 R1 REV (SEQ ID NO. 329). The gene editing efficiencies of these contracts are shown in Table 10. SCNAs targeting the human MPO gene exon 1 were encoded by plasmid GeneMsgRNA15E1. MPO gene NHEJ erroneous repair was tested in Hek293 cells, harvested 72 hours after transfection with plasmids encoding the protein and the SCNAs. Activity of constructs was tested against a variant of dScCasFok with two nuclear localization sequences (SV40 (SEQ ID NO. 400) and nucleoplasmin (SEQ ID NO. 401)) at the N and C-termini, respectively.

TABLE 10A
Gene editing efficiencies of PAM BD replacement
constructs compared to dscCas9-Fok.

| | Construct | |
|---|---|---|
| | (SEQ ID #) | editing % |
| dScCasFok, 2NLS variant | 12863 | 19.59 |
| | (330) | |
| dScCasFok, 2NLS variant | 13185 | 13.69 |
| | (330) | |
| dScCasFok, HNHΔ, 2NLS variant | 13190 | 1.62 |
| | (331) | |
| dScCasFok, HNHΔ, PAMBD whole Δ, 2NLS variant | 13191 | 0.00 |
| | (332) | |
| dScCasFok, HNHΔ, PAMBD loop Δ, 2NLS variant | 13192 | 0.03 |
| | (333) | |
| dScCasFok, Zinc finger PAMBD loop replacement longer | 12869 | 0.00 |
| linkers, 2NLS variant | (334) | |
| dScCasFok, HNHΔ, Zinc finger PAMBD loop replacement, | 13194 | 0.07 |
| 2NLS variant | (314) | |
| dScCasFok, HNHΔ, Zinc finger PAMBD loop replacement | 13196 | 0.29 |
| longer linkers, 2NLS variant | (315) | |
| dScCasFok, Lac repressor DBD PAMBD whole replacement, | 12870 | 0.00 |
| 2NLS variant | (335) | |
| dScCasFok, HNHΔ, Lac Repressor DBD PAMBD whole | 13197 | 0.11 |
| replacement, 2NLS variant | (316) | |
| dScCasFok, SSO7D PAMBD whole replacement, 2NLS variant | 12878 | 0.00 |
| | (336) | |
| dScCasFok, SSO7D PAMBD whole replacement longer | 12880 | 0.00 |
| linkers, 2NLS variant | (337) | |
| dScCasFok, HNHΔ, SSO7D PAMBD whole replacement, | 13205 | 0.19 |
| 2NLS variant | (317) | |
| dScCasFok, HNHΔ, SSO7D PAMBD whole replacement | 13207 | 0.00 |
| longer linkers, 2NLS variant | (338) | |
| dScCasFok, STO7D PAMBD loop replacement longer linker, | 12885 | 0.00 |
| 2NLS variant | (339) | |
| dCasFok, HNHΔ, STO7D PAMBD loop replacement longer | 13212 | 0.04 |
| linker, 2NLS variant | (318) | |
| dCasFok, HNHΔ, HIV integrase DBD PAMBD replacement | 13203 | 0.00 |
| whole replacement longer linker, 2NLS variant | (340) | |
| dCasFok, HNHΔ, HIV integrase DBD PAMBD replacement | 13204 | 0.00 |
| loop replacement longer linker, 2NLS variant | (341) | |

Editing efficiency was quantified by ddPCR (ddPCR#64 results are shown). NLS = Nuclear localization sequence.

Nuclease activity on this endogenous gene in these human cells was successfully detected with five different constructs: (1) dScCas9-FokI, HNHA, Zinc finger PAMBD loop replacement, 2NLS (SEQ ID NO. 314); (2) dScCas9-FokI, HNHA, Zinc finger PAMBD loop replacement longer linkers, 2NLS (SEQ ID NO. 315); (3) dScCas9-FokI, HNHA, Lac repressor DBD PAMBD replacement whole replacement, 2NLS (SEQ ID NO. 316); (4) dScCas9-FokI, HNHA, SSO7D PAMBD replacement whole replacement, 2NLS (SEQ ID NO. 317); (5) dScCas9-FokI, HNHA, STO7D PAMBD replacement loop replacement longer linker, 2NLS (SEQ ID NO. 318) (Table 10A). Other PAM BD replacements were not found to have detectable activity.

Notably, dScCas9-FokI variants with deletions of the entire PAM binding loop or PAM binding domain were not found to have activity, demonstrating the requirement of replacing the PAM binding region with an appropriate NSDB. In many cases PAMBD replacements had different activities when different linker lengths were used, suggesting that proper orientation of the NSDB relative to the rest of the protein is essential to proper function. These results demonstrate the difficulty in abolishing the PAM binding regions in Cas-based nucleoproteins.

Two constructs with PAMBD replaced with Non-specific RVD from AvrBS3 protein family were further designed: dScCas9-FokI, HNHA, AvrBS3 PAMBD loop replacement, 2NLS (SEQ ID NO: 406), and dScCas9-FokI, HNHA, AvrBS3 PAMBD domain replacement, 2NLS (SEQ ID NO: 407). It should be noted that PAMBD loop as referred to herein comprises the amino acid sequence of residues 1330 to 1342, and the “PAMBD domain”, “entire PAMBD” OR “whole PAMBD” comprises the amino acid residues 1228 to 1343 of the ScCas9 (of SEQ ID NO. 258).

In addition, six constructs were further designed with PAMBD replaced with Sto7 (SEQ ID NO:433, 434), HMGN (SEQ ID NO:435,436), and StkC (SEQ ID NO:437,438). As shown in Table 10B below, replacement of whole PAMBD with Sto7 that had longer linkers resulted in a dScCasFok with gene editing efficiency of 0.45% (SEQ ID NO: 433). Replacement of PAMBD loop with HMGN resulted in a dScCasFok with gene editing efficiency of 0.54% (SEQ ID NO: 435). Replacement of either PAMBD loop or entire PAMBD with StkC resulted in a dScCasFok with gene editing efficiency of 0.18% (SEQ ID NOs:437, 438). These results illustrate the general concept that replacing the PAM with non-specific DNA binding domains can result in functional dScCasFok variants.

TABLE 10B
Gene editing efficiencies of PAM BD replacement constructs

| | Construct | editing % |
|---|---|---|
| Description | (SEQ ID NO) | (ddPCR#81) |
| SpCas9, SV40 + nucleoplasmin NLS | 7665 (257) | 42.99% |
| dScCasFok, SV40 NLS | 11241 (2) | 21.24% |
| dScCasFok, HNHΔ, whole PAMBD replaced with | 14268 (433) | 0.45% |
| Sto7, longer linkers, , SV40 + nucleoplasmin NLS | | |
| dScCasFok, HNHΔ, PAMBD loop replaced with | 14269 (434) | 0.02% |
| Sto7, longer linkers, SV40 + nucleoplasmin NLS | | |
| dScCasFok, HNHΔ, PAMBD loop replaced with | 14275 (435) | 0.54% |
| HMGN, SV40 + nucleoplasmin NLS | | |
| dScCasFok, HNHΔ, whole PAMBD replaced with | 14411 (436) | 0.01% |
| HMGN, SV40 + nucleoplasmin NLS | | |
| dScCasFok, HNHΔ, PAMBD loop replaced with | 14276 (437) | 0.18% |
| StkC, SV40 + nucleoplasmin NLS | | |
| dScCasFok, HNHΔ, whole PAMBD replaced with | 14277 (438) | 0.18% |
| StkC, SV40 + nucleoplasmin NLS | | |

Editing efficiency was quantified by ddPCR (ddPCR# #81 results are shown). NLS = Nuclear localization sequence.

The best PAM replacement constructs may be combined with the best HNH deletions (Table 11), the best Sc loop deletions/mutations (Table 9), and ancestral mutations (Table 18), in order to create PAM-replaced dCasFok variants without HNH or Sc loop, and stabilized by ancestral mutations. Candidate constructs were constructed and tested by ddPCR and the results are shown in Table 10C, and FIGS. 3 and 4. More specifically, the NHEJ erroneous repair percentages in the targeted human Myeloperoxidase (MPO) gene, was assayed by ddPCR for different dScCas9-FokI derivatives at two timepoints (76 hrs or 120 hrs post transfection) in two separate biological experiments. All constructs in FIG. 3, have an NNG PAM, a combination of an SV40 and an SV40-derived bipartite Nuclear Localization Signal (NLS), a deletion of the scLoop and the Rec domain ancestral mutations. The constructs presented in FIG. 4 have a mutated ScCas9 “scLoop”, an SV40-Nuclear Localization Signal (NLS) combined with a nucleoplasmin NLS, an HNH deletion and the ancestral Rec2 mutation.

Notably, many HNH-deleted, Sc loop-deleted/replaced, PAMBD replacement constructs were active in the ancestral mutation background. The variants with >1% gene editing activity included SEQ ID NO: 467, 474, 478, 479, 480, and included whole PAMBD or PAMBD loop replaced with Lac DNA binding domain, HMGN, SSO7D, and STO7, validating the approach of using non-specific DNA binding domains to replace the PAM domain

TABLE 10C
Gene editing efficiencies of PAM BD replacement constructs.

| | SEQ ID | TG | editing % |
|---|---|---|---|
| Description | NO | number | 120 hrs |
| Control | | | |
| Cas9 | 257 | 7665 | 50.38% |
| Cas9 | 257 | 7665 | 44.99% |
| dScCasFok, SV40 NLS | 2 | 11241 | 11.49% |
| dScCasFok, SV40 NLS | 2 | 11241 | 14.39% |
| dScCasFok, SV40 NLS | 2 | 11241 | 13.64% |
| dScCasFok, SV40 + SV40 bipartite NLS | 375 | 14280 | 19.66% |
| dScCasFok, SV40 + SV40 bipartite NLS | 375 | 14280 | 23.89% |
| dScCasFok, SV40 + SV40 bipartite NLS | 375 | 14280 | 23.59% |
| Sc Loop deletions | | | |
| dScCasFok, SV40 + bipartiteSV40, ancestral mutations | 450 | 14621 | 17.87% |
| in RuvC + REC1/2 domain, Scloop deletion | | | |
| dScCasFok, SV40 + bipartiteSV40, ancestral mutations | 451 | 14622 | 15.69% |
| in RuvC + REC1/2 domain, Scloop deletion, 6His tag | | | |
| dScCasFok, SV40 + bipartiteSV40, ancestral mutations | 452 | 14623 | 1.10% |
| in RuvC + REC1/2 domain, Scloop deletion, | | | |
| HNHdeletion | | | |
| dScCasFok, SV40 + bipartiteSV40, ancestral mutations | 453 | 14624 | 4.01% |
| in RuvC + REC1/2 domain, Scloop deletion, | | | |
| HNHdeletion, 6His tag | | | |
| dScCasFok, SV40 + bipartiteSV40, ancestral mutations | 454 | 14625 | 6.90% |
| in RuvC + REC1/2 domain, Scloop deletion, | | | |
| HNHdeletion, 6His tag | | | |
| dScCasFok, SV40 + bipartiteSV40, ancestral mutations | 455 | 14626 | 9.67% |
| in RuvC + REC1/2 domain, Scloop QQmutant, | | | |
| HNHdeletion, 6His tag | | | |
| dScCasFok, SV40 + bipartiteSV40, ancestral mutations | 456 | 14627 | 9.62% |
| in RuvC + REC1/2 domain, Scloop AAmutant, | | | |
| HNHdeletion, 6His tag | | | |
| dScCasFok, SV40 + bipartiteSV40, ancestral mutations | 457 | 14628 | 5.58% |
| in RuvC + REC1/2 domain, Scloop Sp Replacement, | | | |
| HNHdeletion, 6His tag | | | |
| dScCasFok, SV40 + bipartiteSV40, ancestral mutations | 458 | 14629 | 4.38% |
| in RuvC + REC1/2 domain, Scloop deletion, | | | |
| HNHdeletion, 6His tag | | | |
| dScCasFok, SV40 + bipartiteSV40, ancestral mutations | 459 | 14630 | 0.11% |
| in RuvC + REC1/2 domain, Scloop QQmutant, | | | |
| HNHdeletion, 6His tag | | | |
| dScCasFok, SV40 + bipartiteSV40, ancestral mutations | 460 | 14631 | 1.51% |
| in RuvC + REC1/2 domain, Scloop AAmutant, | | | |
| HNHdeletion, 6His tag | | | |
| HNH deletion + PAM replacement + Sc Loop | | | |
| deletion/mutation | | | |
| dScCasFok, SV40 + nucleoplasmin, ancestral mutations | 461 | 14637 | 0.00% |
| in RuvC + REC1/2 domain, Scloop deletion, HNH | | | |
| deletion, PAMBD loop replaced with Zinc finger | | | |
| dScCasFok, SV40 + nucleoplasmin, ancestral mutations | 462 | 14638 | 0.00% |
| in RuvC + REC1/2 domain, Scloop deletion, HNH | | | |
| deletion, whole PAMBD replaced with LacI DNA | | | |
| binding domain | | | |
| dScCasFok, SV40 + nucleoplasmin, ancestral mutations | 463 | 14639 | 0.00% |
| in RuvC + REC1/2 domain, Scloop deletion, HNH | | | |
| deletion, whole PAMBD replaced with SSO7D | | | |
| dScCasFok, SV40 + nucleoplasmin, ancestral mutations | 464 | 14640 | 0.16% |
| in RuvC + REC1/2 domain, Scloop deletion, HNH | | | |
| deletion, PAMBD loop replaced with HMGN | | | |
| dScCasFok, SV40 + nucleoplasmin, ancestral mutations | 465 | 14641 | 0.02% |
| in RuvC + REC1/2 domain, Scloop deletion, HNH | | | |
| deletion, whole PAMBD replaced with STO7 | | | |
| dScCasFok, SV40 + nucleoplasmin, ancestral mutations | 466 | 14642 | 0.00% |
| in RuvC + REC1/2 domain, Scloop QQmutation, HNH | | | |
| deletion, PAMBD loop replaced with Zinc finger | | | |
| dScCasFok, SV40 + nucleoplasmin, ancestral mutations | 467 | 14643 | 4.44% |
| in RuvC + REC1/2 domain, Scloop QQmutation HNH | | | |
| deletion, whole PAMBD replaced with LacI DNA | | | |
| binding domain | | | |
| dScCasFok, SV40 + nucleoplasmin, ancestral mutations | 468 | 14644 | 0.00% |
| in RuvC + REC1/2 domain, Scloop QQmutation, HNH | | | |
| deletion, whole PAMBD replaced with SSO7D | | | |
| dScCasFok, SV40 + nucleoplasmin, ancestral mutations | 469 | 14645 | 0.31% |
| in RuvC + REC1/2 domain, Scloop QQmutation, HNH | | | |
| deletion, PAMBD loop replaced with HMGN | | | |
| dScCasFok, SV40 + nucleoplasmin, ancestral mutations | 470 | 14646 | 0.00% |
| in RuvC + REC1/2 domain, Scloop QQmutation, HNH | | | |
| deletion, whole PAMBD replaced with STO7 | | | |
| dScCasFok, SV40 + nucleoplasmin, ancestral mutations | 471 | 14647 | 0.00% |
| in RuvC + REC1/2 domain, Scloop AAmutation, HNH | | | |
| deletion, PAMBD loop replaced with Zinc finger | | | |
| dScCasFok, SV40 + nucleoplasmin, ancestral mutations | 472 | 14648 | 0.01% |
| in RuvC + REC1/2 domain, Scloop AAmutation HNH | | | |
| deletion, whole PAMBD replaced with LacI DNA | | | |
| binding domain | | | |
| dScCasFok, SV40 + nucleoplasmin, ancestral mutations | 473 | 14649 | 0.03% |
| in RuvC + REC1/2 domain, Scloop AAmutation, HNH | | | |
| deletion, whole PAMBD replaced with SSO7D | | | |
| dScCasFok, SV40 + nucleoplasmin, ancestral mutations | 474 | 14650 | 1.02% |
| in RuvC + REC1/2 domain, Scloop AAmutation, HNH | | | |
| deletion, PAMBD loop replaced with HMGN | | | |
| dScCasFok, SV40 + nucleoplasmin, ancestral mutations | 475 | 14651 | 0.37% |
| in RuvC + REC1/2 domain, Scloop AAmutation, HNH | | | |
| deletion, whole PAMBD replaced with STO7 | | | |
| dScCasFok, SV40 + nucleoplasmin, ancestral mutations | 476 | 14652 | 0.88% |
| in RuvC + REC1/2 domain, Scloop SpReplacement, | | | |
| HNH deletion, PAMBD loop replaced with Zinc finger | | | |
| dScCasFok, SV40 + nucleoplasmin, ancestral mutations | 477 | 14653 | 0.00% |
| in RuvC + REC1/2 domain, Scloop SpReplacement | | | |
| HNH deletion, whole PAMBD replaced with LacI | | | |
| DNA binding domain | | | |
| dScCasFok, SV40 + nucleoplasmin, ancestral mutations | 478 | 14654 | 2.09% |
| in RuvC + REC1/2 domain, Scloop SpReplacement | | | |
| HNH deletion, whole PAMBD replaced with SSO7D | | | |
| dScCasFok, SV40 + nucleoplasmin, ancestral mutations | 479 | 14655 | 3.29% |
| in RuvC + REC1/2 domain, Scloop SpReplacement | | | |
| HNH deletion, PAMBD loop replaced with HMGN | | | |
| dScCasFok, SV40 + nucleoplasmin, ancestral mutations | 480 | 14656 | 1.17% |
| in RuvC + REC1/2 domain, Scloop SpReplacement | | | |
| HNH deletion, whole PAMBD replaced with STO7 | | | |

Editing efficiency was quantified by ddPCR (ddPCR#86 results are shown). NLS = Nuclear localization sequence.

PAM-independence of all constructs described here is tested using an assay such as the one described in Example 6.

### Example 3

### dCas-FokI Variants Optimized through Deletions

To reduce the size of the Cas variants of the invention and fusion proteins thereof, different regions of the Cas nucleoprotein are next deleted. Reduction of the protein size is beneficial for, among other reasons, to allow the construct to be placed in a recombinant Adeno Associated Virus (AAV) vector. Such vectors are considered highly efficient and safer than other viral delivery options. Moreover, AAVs can be targeted in vivo to specific tissues [10]. The maximum AAV packaging capacity is about 4.5 kb, too small for dSpCas9-FokI or dScCas9-FokI. A working example of a 4.437 kb HNH deletion variant is next constructed by the invention. Specifically, an HNH-nuclease deleted ScCas9-FokI (dHNH-dScCas9-FokI) is provided herein.

The HNH is one of the two nucleases of Cas9 proteins. To create “dead” version of this protein, substitutions in the catalytic residues in HNH and RUVC nuclease domains have been shown, i.e. for ScCas9 the mutations shown were D 10A and H849A (Chatterjee et al., 2018, Science Advances, Vol. 4, no. 10, eaau0766). As no nuclease activity is required for Cas when substituted with a FokI nuclease domain, the HNH nuclease domain is redundant and has been previously similarly removed from SpCas9 in a transcriptional-activator domain dCas9 fusion protein. Constructs comprising the nucleic acid sequence as denoted by SEQ ID NO. 9 (Plasmid 11248 with guide), and SEQ ID NO. 13 (Plasmid 11254 with no guide), were prepared as described in experimental procedures. These constructs encode the dScCas9-FokI dHNH chimera that comprise the amino acid sequence as denoted by SEQ ID NO. 10 (with and with no guide, respectively).

EMX1 gene NHEJ erroneous repair was tested in Hek293 cells, harvested 72 hours after transfection with plasmids encoding the protein and the SCNAs. Constructs with deleted HNH domain (dHNH) had significant genome editing activity with an SCNA gap of 15 (SEQ ID NO. 12) and greatly reduced activity with a gap of 27 (SEQ ID NO. 11). A ddPCR analysis was made using BioRad software counting only single-template droplets which were HEX-negative FAM positive. This was followed by Poisson correction for inclusion of the expected number of 2,3,4 etc template containing droplets (see “tails” in FIG. 5B) as calculated from the empty/full droplet ratio multiplied by the percentage of single template NHEJ erroneous repair droplets (top left quarters in plots of FIG. 5B) with a 95% confidence level. A mixture of 15- and 27-gap SCNAs gave intermediate activity as could be expected from competition between the protein and RNA components, see FIG. 5A. However, compared to dScCas9-Fok this construct had lower activity. To further improve the activity of the size-reduced chimera, additional constructs encoding variety of Cas9 chimeras with different deletions, were designed. These constructs encode the following size-reduced dScCas9-Fok chimeras: dScCas9-FokI.dHNH.dREC2 as denoted by SEQ ID NO: 14, dScCas9-FokI.dHNH.dFLEX as denoted by SEQ ID NO: 15, dScCas9-FokI.dREC2.dFLEX as denoted by SEQ ID NO. 16 and dScCas9-FokI.dHNH.dREC2.dFLEX as denoted by SEQ ID NO. 17.

Without being bound by the theory, it was hypothesized that HNH domain may also be replaced by peptides of various sequences. These peptides include shorter (about 1-5 residues) or longer (about 6-20 residues or longer) linkers and linkers with positively charged amino acids (such as Lys and Arg residues), deletions of different sizes (either larger or smaller), and linkers derived from the H-NS nucleoid-associated protein (AAVKSGTKAKRAQRP, as denoted by SEQ ID NO: 485). Constructs were designed and synthesized, editing efficiency was tested, and the results are shown in Table 11. SCNAs targeting the human MPO gene exon 1 were encoded by plasmid GeneMsgRNA15E1. MPO gene NHEJ erroneous repair was tested in Hek293 cells, harvested 72 hours after transfection with plasmids encoding the protein and the SCNAs. It appears that the proposed HNH replacements had detectable editing activity, and that this activity varied between different replacements. Highest activity was observed by replacing HNH domain with a longer linker (“dScCasFok, HNHΔ with longer linkers, 2NLS”, SEQ ID NO: 342), which edited 0.82% of cells.

TABLE 11
Gene editing efficiencies of HNH domain replacement constructs.

| | Construct | editing |
|---|---|---|
| Description | (SEQ ID NO) | % |
| dScCasFok, HNHΔ with longer linkers (GGSAGGTGGSGG, | 14001 (342) | 0.82% |
| SEQ ID NO: 306), 2NLS | | |
| dScCasFok, HNHΔ replaced with SSB, 2NLS | 14002 (343) | 0.20% |
| dScCasFok, HNHΔ with larger deletion (18 additional | 14003 (344) | 0.34% |
| residues), 2NLS | | |
| dScCasFok, HNHΔ with positively charged linker | 14004 (349) | 0.77% |
| (GGKARGKGGSGG, SEQ ID NO: 307), 2NLS | | |
| dScCasFok, HNHΔ with short positively charged linker | 14005 (350) | 0.48% |
| (GKSKG, SEQ ID NO: 481), 2NLS | | |
| dScCasFok, HNHΔ with H-NS linker | 14007 (351) | 0.04% |
| (AAVKSGTKAKRAQRP, SEQ ID NO: 485), 2NLS | | |

Editing efficiency was quantified by ddPCR (ddPCR#73 results are shown). NLS = Nuclear localization sequence.

The HNH domain may also be replaced by domains that are smaller or have different properties. These domains may comprise single-stranded DNA binding protein (SSB) which binds single-stranded DNA, sticky C which binds chromatin. Moreover, HNH domain deletions and replacements may be done in combination with mutations that may enhance stability/expression/activity, which may include ancestral mutations (see Example 16). Constructs were designed and synthesized, editing efficiency was tested, and the results are shown in Table 12. SCNAs targeting the human MPO gene exon 1 were encoded by plasmid GeneMsgRNA15E1. MPO gene NHEJ erroneous repair was tested in Hek293 cells, harvested 72 hours after transfection with plasmids encoding the protein and the SCNAs. As a positive control, a variant of dScCasFok (SEQ ID NO. 375) was used with two nuclear localization sequences, SV40 at the N-terminus (SEQ ID NO. 400), and a bipartite SV40 (SEQ ID NO. 402) at the C-terminus. Another control was a dScCasFok variant with a C-terminal 6× His tag (SEQ ID NO. 403).

It was found that the proposed HNH replacements had detectable editing activity, and that this activity varied between different replacements. Interestingly, ancestral mutations in the REC domain of Cas9 increased the activity of HNH domain deletions. For example the “dScCasFok, HNHΔ with longer linker, SV40+bipartiteSV40 NLS” (SEQ ID NO: 379) had an average editing efficiency of 1.27%, but when ancestral mutations were incorporated in the RuvC and Rec1/2 domains its activity increased to an average editing efficiency of 8.07% (SEQ ID NO: 387) (Table 12). These results show that it is possible to remove the HNH domain and retain high activity by proper choice of linker length and composition. Moreover, ancestral mutations may be important for such modifications, possibly through their effects on the stability, expression, or activity of the dCasFok protein.

TABLE 12
Gene editing efficiencies of HNH domain replacement constructs. ND = not done

| | Construct | editing % | editing % |
|---|---|---|---|
| Description | (SEQ ID NO) | (ddPCR76) | (ddPCR81) |
| dScCasFok, SV40 + bipartite SV40 NLS | 14280 (375) | 29.08% | 26.26% |
| dScCasFok, SV40 + bipartite SV40 NLS, | 14412 (376) | 30.81% | 26.08% |
| 6xHis | | | |
| dScCasFok, ancestral RuvC + Rec1/2domain, | 14314 (377) | 25.51% | 21.25% |
| SV40 and bipartite SV40 NLS | | | |
| dScCasFok, HNHΔ, SV40 + bipartiteSV40 | 14297 (378) | 2.88% | 2.96% |
| NLS | | | |
| dScCasFok, HNHΔ with longer linker, | 14298 (379) | 2.54% | 0% |
| SV40 + bipartiteSV40 NLS | | | |
| dScCasFok, HNH replaced with SSB, | 14299 (380) | 0.00% | ND |
| SV40 + bipartiteSV40 NLS | | | |
| dScCasFok, HNHΔ with longer deletion | 14300 (381) | 1.58% | ND |
| SV40 + bipartiteSV40 NLS | | | |
| dScCasFok, HNHΔ with longer positively | 14301 (382) | 4.05% | 3.98% |
| charged linker, SV40 + bipartiteSV40 NLS | | | |
| dScCasFok, HNHΔ with shorter positively | 14302 (383) | 3.01% | 3.06% |
| charged linker, SV40 + bipartiteSV40 NLS | | | |
| dScCasFok, HNH replaced with StkC, | 14303 (384) | 1.51% | ND |
| SV40 + bipartiteSV40 NLS | | | |
| dScCasFok, HNHΔ with HNS linker, | 14304 (385) | 1.69% | ND |
| SV40 + bipartiteSV40 NLS | | | |
| dScCasFok, ancestral RuvC + Rec1/2, HNHΔ, | 14315 (386) | 5.49% | 4.09% |
| SV40 + bipartiteSV40 NLS | | | |
| dScCasFok, ancestral RuvC + Rec1/2, HNHΔ | 14316 (387) | 9.59% | 6.54% |
| with longer linker, SV40 + bipartiteSV40 | | | |
| NLS | | | |
| dScCasFok, ancestral RuvC + Rec1/2, HNH | 14317 (388) | 4.78% | ND |
| replaced with SSB, SV40 + bipartiteSV40 | | | |
| NLS | | | |
| dScCasFok, ancestral RuvC + Rec1/2, HNHΔ | 14318 (389) | 4.84% | ND |
| with longer deletion SV40 + bipartiteSV40 | | | |
| NLS | | | |
| dScCasFok, ancestral RuvC + Rec1/2, HNHΔ | 14319 (390) | 4.10% | 4.69% |
| with longer positively charged linker, | | | |
| SV40 + bipartiteSV40 NLS | | | |
| dScCasFok, ancestral RuvC + Rec1/2, HNHΔ | 14320 (391) | 3.89% | 4.61% |
| with shorter positively charged linker, | | | |
| SV40 + bipartiteSV40 NLS | | | |
| dScCasFok, ancestral RuvC + Rec1/2, HNH | 14321 (392) | 1.79% | ND |
| replaced with StkC, SV40 + bipartiteSV40 | | | |
| NLS | | | |
| dScCasFok, ancestral RuvC + Rec1/2, HNHΔ | 14322 (393) | 4.53% | ND |
| with HNS linker, SV40 + bipartiteSV40 NLS | | | |

Editing efficiency was quantified by ddPCR (ddPCR#76 and #81 results are shown). NLS = Nuclear localization sequence.

Thus, from the results gathered from several tables above and below it may be seen that several optimal combinations—having increased activity—may be assembled into a single optimal construct, i.e. one containing a combination of dscCas9-Fok with both a ‘SV40NLS+bipartite SV4ONLS’ and ‘HMGN’, or in the delta-HNH (HNHA) background one with a ‘longer linker’, a ‘SV4ONLS+bipartite SV4ONLS’, and an ‘ancestral RuvC+REC1/2’ (SEQ ID NO. 387, table 12).This specific combination (SEQ ID NO. 387), for example, shows how several deleterious mutations in one background, when combined, can synergistically enhance performance in another.

Modifications that improve activity may be combined to create improved dScCasFok variants with high activity. These modifications may be combined with PAMBD replacement modifications (see Example 2). Several variants were designed:

dScCasFok, N-terminal HMGN, ancestral RuvC+Rec1/2, HNHΔ with longer linker, SV40 and bipartite SV40 NLS (SEQ ID NO. 426); dScCasFok, ancestral RuvC+Rec1/2, HNHΔ with longer linker, PAMBD loop replaced with Zinc Finger +longer linker, SV40 and bipartite SV40 NLS (SEQ ID NO. 427); dScCasFok, ancestral RuvC+Rec1/2, HNHΔ with longer linker, PAMBD replaced with Lac repressor DBD, SV40 and bipartite SV40 NLS (SEQ ID NO. 428); dScCasFok, ancestral RuvC+Rec1/2, HNHΔ with longer linker, PAMBD replaced with SSO7D, SV40 and bipartite SV40 NLS (SEQ ID NO. 429); dScCasFok, N-terminal HMGN, ancestral RuvC+Rec1/2, HNHΔ with longer linker, PAMBD loop replaced with Zinc Finger, SV40 and bipartite SV40 NLS (SEQ ID NO. 430); dScCasFok, N-terminal HMGN, ancestral RuvC+Rec1/2, HNHΔ with longer linker, PAMBD replaced with Lac repressor DBD, SV40 and bipartite SV40 NLS (SEQ ID NO. 431) and dScCasFok, N-terminal HMGN, ancestral RuvC+Rec1/2, HNHΔ with longer linker, PAMBD replaced with SSO7D, SV40 and bipartite SV40 NLS (SEQ ID NO. 432).

### Example 4

### dCas-FokI Division Using a Split-Intein System

dScCasFok may also be divided into two polypeptides (or sequences encoding them) that can be reconstituted using inteins which are intervening protein domains that can undergo a posttranslational autoprocessing termed protein splicing. This partition has been shown to work for SpCas9 (Truong et al, 2015, Nucleic Acids Res, 43:6450-8). A split-intein system was designed for dScCas9Fok that significantly reduces the size of the individual coding nucleotide sequences that need to be packed in systems like AAV. The N-terminal half of the DnaE intein from Nostoc punctiforme was fused to residues 1-809 of dScCasFok to make dScCasFok N-terminal intein (SEQ ID NO:353), and the C-terminal half of the DnaE intein from Nostoc punctiforme was fused to residues 810-1601 of dScCasFok to make dScCasFok C-terminal intein (SEQ ID NO:352). SV40-based NLS sequences were added to the N- and C-termini of each construct. Constructs were designed and synthesized, editing efficiency was tested, and the results are shown in Table 13. SCNAs targeting the human MPO gene exon 1 were encoded by plasmid GeneMsgRNA15E1. MPO gene NHEJ erroneous repair was tested in Hek293 cells, harvested 96 hours after transfection with plasmids encoding the proteins and the SCNAs.

TABLE 13
Gene editing efficiencies of dScCasFok split-intein constructs.

| Description | Construct (SEQ ID) | editing % |
|---|---|---|
| SpCas9 | 13681 (257) | 37 |
| dScCasFok, 1 NLS | 11241 (2) | 18 |
| dScCasFok, 2 NLS | 14280 (375) | 26 |
| Guide only | — | 0 |
| dScCasFok N-terminal intein, 2NLS variant | 14870 (353) | 0 |
| dScCasFok C-terminal intein, 2NLS variant | 14806 (352) | 0 |
| dScCasFok C and N-terminal inteins, 2NLS variants | 14806, 14870 | 34 |
| | (352 + 353) | |

Editing efficiency was quantified by ddPCR (ddPCR#96 results are shown). NLS = Nuclear localization sequence.

From these results, that are further presented by FIG. 15, it can be seen that splitting dscCasFok into two polypeptides, following their expression and reconstitution in human cells, is thus a viable strategy for delivery of large proteins such as dscCas-Fok. Gene editing efficiency of the reconstituted protein from inteins (34%) was observed to be higher than the entire dScCasFok (26%), possibly due to concentration or differences in nuclear import efficiency. Moreover, neither intein fragment had activity on its own showing the necessity of the intact protein being reconstituted from the fragments.

### Example 5

### Plant Optimized Chimeric Editing Systems

Induction of predetermined chromosomal double strand breaks (DSBs) in living cells or whole plants of Arabidopsis

The enzyme Phytoene Desaturase (PDS) is involved in the conversion of phytoene ζ-carotene in carotenoid biosynthesis. Disruption of Arabidopsis phytoene desaturase results in albino and dwarf phenotypes. This phenotype is explained by impaired chlorophyll, carotenoid, and gibberellin biosynthesis. Thus, a mutation in this gene is phenotypically detectable.

To establish and develop effective editing systems in plants, the inventors next adapted the gene editing systems of the invention for plant cells using plant promoters, SCNAs and plant target genes. More specifically, the inventors induce a chromosomal double-strand break (DSB) in the PDS gene in order to create a point mutation through a frameshift, thus knocking out the function of the gene endogenously by utilizing the NHEJ pathway using the programmable molecular machine.

In addition, the inventors specifically induce a chromosomal double-strand break (DSB) in the PDS gene in order to create an Insertion of a mCherry Donor sequence into an endogenous PDS sequence to knock out PDS by assisted homologous recombination using the programmable molecular machine. Tobacco protoplasts have been similarly used with ZFNs to insert a reporter gene into a specific location in a chromosome (Wright et al, 2005, Plant J, 44:693-705), however, in contrast, an endogenous Arabidopsis gene and not a transgene sequence is targeted by the present invention. Nucleic acids in Human or animal cells may similarly be targeted. In the examples shown here the protein moiety is expressed by way of plant promoters and thus relevant for plants.

Delivery of the editing systems of the invention to plants is performed by Agrobacterium transformation via floral dip, detection is by identifying white seedlings.

In another embodiment Plasmid Delivery is by PEG to protoplasts.

To target genomic DNA in-vivo to knock out PDS in plants and/or to insert the reporter gene into the targeted PDS gene, a visual assay was designed where seedling offspring of the targeted plants are white and/or express the reporter fluorophore or alternatively an Arabidopsis protoplast based assay whereby plasmid delivery is mediated by PEG and the detection is done by extracting and examining the genomic DNA of the PDS gene flanking the mutation/deletion/insertion site. In the former example the delivery method used is Agrobacterium mediated T-DNA transfer through floral-dip. In this bioassay the inventors deliver the system to the Arabidopsis flower with Agrobacterium carrying T-DNA. In the latter example the inventors use an Arabidopsis protoplast-based bioassay. In this bioassay the inventors deliver to these protoplasts a plasmid. In either assays the inventors deliver a T-DNA or plasmid respectively, expressing the molecular machine in-vivo and co-delivered also with a T-DNA or a plasmid encoding a pair of sgRNAs respectively, to target TCCAGATGAAAGTGC (as denoted by SEQ ID NO. 49) site on exon 14 to disrupt PDS activity. The PAM target sequence is NNG on both sides flanking the sgRNAs flanking this target site.

The sgRNAs for this example are arranged on opposing target strands, PAM-out.

The use of a pair of sgRNAs, for example, the right and the left target sgRNAs (as denoted by SEQ ID NOs.50 and 51, respectively), with an obligatory-dimerizing nuclease should provide greatly enhanced specificity and reduce off-target cleavage in the plant and thus reduce off-target mutations in its offspring.

Several methods of delivery of the sgRNA and the protein to plant cells can be used. One such method which is DNA-free and thus not susceptible to unwanted DNA integration is use of Ribonucleic-acid-protein complex (RNP) whereby the sgRNA can be supplied as synthetic RNA together with the protein (RNP) and be transfected to protoplasts (Murovec et al., Front Plant Sci. 2018; 9: 1594.).

More commonly, DNA delivery can be used avoiding the difficulties posed by regeneration of protoplasts.

For plasmid or agrobacterium delivery DNA encoding the following sgRNAs regulated by a pair of tail-to-tail PolIII plant promoters is used.

More specifically, the NekP-AtU61P cassette is used, where N denotes the sgRNA sequence, and guttagagetagaaatagcaagttaaaataaggetagtecguatcaacttgaaaaagtggcaccgagteggtgc (of SEQ ID NO. 52) denotes the Right Cas9 sgRNA scaffold and gcaccgacteggtgccactuttcaagttgataacggactagccuatutaacttgetatuctagactaaaac (of SEQ ID NO. 53) denotes the Left Cas9 sgRNA scaffold which is the same sequence but in opposite orientation to avoid promoter interference, thereby creating the sequence:

Ggatccatttaaattctagaggcgcgccaaaaaaagcaccgactcggtgccactttttcaagttgataacggactagccttattttaactt
gctatttctagctctaaaacNNNNNNNNNNNNNNNNNNNNaatcgctatgtcgactctatcattatataaactaagctgcta
tatatcacctgatcgatgtgggacttttgatcactccagaaatctcaaaattccggcagaacaattttgaatctcgatccgtagaaacgag
acggtcattgttttagttccaccacgattatatttgaaatttacgtgagtgtgagtgagacttgcataagaaaataaaatctttagttgggaa
aaaattcaataatataaatgggcttgagaaggaagcgagggataggcctttttctaaaataggcccatttaagctattaacaatcttcaa
aagtaccacagcgcttaggtaaagaaagcagctgagtttatatatggttagagacgaagtagtgattNNNNNNNNNNNNNN
NNNNNN [symbol] tttttt
ttaattaatagggataacagggtaatta, as denoted by SEQ ID NO. 54.

To target the PDS gene, the NekP-AtU61P that contain PDS specific sgRNA, is used

ggatccatttaaattctagaggcgcgccaaaaaaagcaccgactcggtgccactttttcaagttgataacggactagccttattttaactt
gctatttctagctctaaaacTGTCCCATTAGTTCACAACCaatcgctatgtcgactctatcattatataaactaagctgctata
tatcacctgatcgatgtgggacttttgatcactccagaaatctcaaaattccggcagaacaattttgaatctcgatccgtagaaacgaga
cggtcattgttttagttccaccacgattatatttgaaatttacgtgagtgtgagtgagacttgcataagaaaataaaatctttagttgggaaa
aaattcaataatataaatgggcttgagaaggaagcgagggataggcctttttctaaaataggcccatttaagctattaacaatcttcaaa
agtaccacagcgcttaggtaaagaaagcagctgagtttatatatggttagagacgaagtagtgattCTCCAAAAACCCGTTT
TGAT [symbol] ttttttttaa
ttaatagggataacagggtaatta, as denoted by SEQ ID NO. 55.

In addition, DNA expression construct comprising the nucleic acid sequence as denoted by SEQ ID NO. 60, that encodes the dScCas9-FokI protein of SEQ ID NO. 56, is used. This DNA expression cassette is composed of 2X35SP from pSAT6, as denoted by SEQ ID NO. 57, the Plant optimized dScCAS9-FokI, as denoted by SEQ ID NO. 58, and the 35ST from pSAT6, as denoted by SEQ ID NO. 59.

Donor DNA sequence comprising PD-mCherry-S encoding ORF of SEQ ID NO. 61, is used for NHEJ repair pathway, and DONOR PD-MCHERRY-S is used for homologous recombination repair pathway of SEQ ID NO. 62. The mCherry sequence is flanked by PDS sequences at the desired target site

SEQ ID NO. 55 and 60 encoding to SCNAs and Nuclease expression cassettes are ordered from GeneArt in pMA Km plasmid, where cassettes are flanked by I-SceI and pI-PspI respectively. The Nuclease cassette contains also a MluI site: pI-PspI-MluI-[Cassette]-pI-PspI. Each cassette was inserted to pPZP-RCS (SEQ ID NO. 295) into corresponding restriction site. The mCherry donor DNA cassettes (SEQ ID NO. 61 and 62) were ordered similarly but with MluI flanking the cassettes (MluI-Cassette-MluI).

SEQ ID NO. 61 was cloned into Ascl site of pPZP-RCS and introduced with the SCNA-Nuclease T-DNA by co-inoculation. The other donor DNA cassette (SEQ ID NO. 62) was introduced into the SCNA-Nuclease construct into MluI site.

For experiments where the PDS is knocked out (NHEJ), DNA from pooled protoplasts is analysed by PCR and restriction fragment analysis of the PCR product. PCR is conducted with the primers Primer2F and Primer2R, as denoted by SEQ ID NO. 63 and SEQ ID NO. 64, respectively.

Abolishment of cleavage with Hpyl88III in at least a portion of the amplified DNA indicates at least some successful gene targeting and directed mutation of the genomic template. Digestion of WT PCR product shows two bands of 112 and 200 bp while a targeted product shows different bands, typically single band sized dictated by the mutation occurred in the cell.

In experiments where a reporter gene is inserted using homologous recombination, a Donor DNA encoding mCherry is fused in frame to the endogenous PDS gene for homologous recombination repair mechanism, or simple mCherry for NHEJ mechanism. In both repair pathways, a successful targeting event results in a mRNA encodes a disrupted PDS fused to a full mCherry immediately followed by a STOP codon (“PD-mCherry”). Protoplasts suspended in W5 solution are screened for mCherry activity three days after transfection using an automated flow-cytometer (FACS) machine. PDS-modified protoplasts are detected by FACS analysis, where an insertion of mCherry donor DNA is detectable by mCherry fluorescence using a 561 nm excitation wavelength and detection of 590-630 nm emission. Threshold and compensation factors will be set to exclude any false positives.

Further characterization in the experiments is achieved by regenerating protoplasts on suitable media and examining their subsequent phenotypic character, where bleached plants or calli indicate successful gene-targeting.

In experiments using Agrobacterium transformation, the resulted product is not a single modified cell but a seed. The seeds are germinated and a targeted plant show white cotyledons and/or mCherry fluorescence.

It should be noted that representative population of cells treated under the conditions of the current example were taken for analysis using a method for off-target detection cited in the methods section. Due to use of dual RNA targeting, less off-target cleavage compared to Cas9 is expected.

### Example 6

### Gap Optimization and PAM Bias/Independence Detection

In order to test activity of different nucleases and different sgRNA combinations on different targets, an in-vitro assay that tests digestion kinetics is used. This test allows (A) PAM requirement elucidation of different proteins, (B) optimization of SCNA (sgRNA) gaps by using the same sgRNAs on targets whereby the SCNA-binding site on the target is placed at variable distances such as 15 nt apart, and nearer or further apart, and (C) use of different RNA-guided nucleases and optimization of their protein sequence.

Protein expression (TNT® SP6 High-Yield Wheat Germ Protein E L3261), is performed as described in Experimental procedures for Example 6 (Table 5).

Template DNA for TNT protein expression system is a PCR template prepared by performing PCR on 10916 (original dscCAS9-Fok construct, SEQ ID NO. 1) with primers 3241.SP6P SV4ONLS F, as denoted by SEQ ID NO. 65 and 3242.CASR, as denoted by SEQ ID NO. 66. The 10916 plasmid (of SEQ ID NO. 1) for human expression of dScCas9-FokI, is used here as an example for a template for amplifying the PCR product used in SP6 in-vitro transcription and translation of an RNA guided nuclease.

The sgRNA sequences 3243EMX115R, as denoted by SEQ ID NO. 67 and 3244EMX115L, as denoted by SEQ ID NO. 68, are used.

To create a plasmid with a suitable digestion pattern, a PCR product derived from part of the Human EMX1 gene was cloned as a target site into pGEM-T Easy plasmid vector (Promega). For the reduced-PAM (NNG) dScCas9-FokI the following insert (Expected size 235 bp) is prepared by PCR on Human gDNA with primers 3042 and 3240, as denoted by SEQ ID Nos. 67 and 68, respectively.

Construction of Different PAM Target Sequences

To create target plasmids with different PAM sites, the inventors amplify a product derived from part of the Human EMX1 gene as a target site into pGEM-T Easy plasmid vector (Promega). In this example the gap between the sgRNAs on the target DNA is set at 15 bp. As a 15 nt gap was found to be efficient in human cells with dScCas9-FokI, this example is used for this in-vitro assay also. To test different gaps an artificial PCR template can be produced adding or removing nucleotides in the gap between the sgRNA binding sites, this template also amplified by the primers below as needed. The inserts are variable according to the desired PAM sequence encoded the primers on both ends. PCR on human gDNA with the listed primer combinations (i.e. 1R with 1L etc.) should yield 64 products of 61 bp (for the 15 nt gap).

Right primers containing 64 PAM combinations, as denoted by SEQ ID NOs. 71-134, and presented by Table 14, left primers containing 64 PAM combinations, as denoted by SEQ ID NOs. 135-198, and presented by Table 15.

TABLE 14
Right primers

| primer | sequence | SEQ ID NO. |
|---|---|---|
| 1R | TTTggtggaggagtgcaggctct | 71 |
| 2R | ATTggtggaggagtgcaggctct | 72 |
| 3R | CTTggtggaggagtgcaggctct | 73 |
| 4R | GTTggtggaggagtgcaggctct | 74 |
| 5R | TATggtggaggagtgcaggctct | 75 |
| 6R | AATggtggaggagtgcaggctct | 76 |
| 7R | CATggtggaggagtgcaggctct | 77 |
| 8R | GATggtggaggagtgcaggctct | 78 |
| 9R | TCTggtggaggagtgcaggctct | 79 |
| 10R | ACTggtggaggagtgcaggctct | 80 |
| 11R | CCTggtggaggagtgcaggctct | 81 |
| 12R | GCTggtggaggagtgcaggctct | 82 |
| 13R | TGTggtggaggagtgcaggctct | 83 |
| 14R | AGTggtggaggagtgcaggctct | 84 |
| 15R | CGTggtggaggagtgcaggctct | 85 |
| 16R | GGTggtggaggagtgcaggctct | 86 |
| 17R | TTAggtggaggagtgcaggctct | 87 |
| 18R | ATAggtggaggagtgcaggctct | 88 |
| 19R | CTAggtggaggagtgcaggctct | 89 |
| 20R | GTAggtggaggagtgcaggctct | 90 |
| 21R | TAAggtggaggagtgcaggctct | 91 |
| 22R | AAAggtggaggagtgcaggctct | 92 |
| 23R | CAAggtggaggagtgcaggctct | 93 |
| 24R | GAAggtggaggagtgcaggctct | 94 |
| 25R | TCAggtggaggagtgcaggctct | 95 |
| 26R | ACAggtggaggagtgcaggctct | 96 |
| 27R | CCAggtggaggagtgcaggctct | 97 |
| 28R | GCAggtggaggagtgcaggctct | 98 |
| 29R | TCAggtggaggagtgcaggctct | 99 |
| 30R | ACAggtggaggagtgcaggctct | 100 |
| 31R | CCAggtggaggagtgcaggctct | 101 |
| 32R | GCAggtggaggagtgcaggctct | 102 |
| 33R | TTCggtggaggagtgcaggctct | 103 |
| 34R | ATCggtggaggagtgcaggctct | 104 |
| 35R | CTCggtggaggagtgcaggctct | 105 |
| 36R | GTCggtggaggagtgcaggctct | 106 |
| 37R | TACggtggaggagtgcaggctct | 107 |
| 38R | AACggtggaggagtgcaggctct | 108 |
| 39R | CACggtggaggagtgcaggctct | 109 |
| 40R | GACggtggaggagtgcaggctct | 110 |
| 41R | TCCggtggaggagtgcaggctct | 111 |
| 42R | ACCggtggaggagtgcaggctct | 112 |
| 43R | CCCggtggaggagtgcaggctct | 113 |
| 44R | GCCggtggaggagtgcaggctct | 114 |
| 45R | TGCggtggaggagtgcaggctct | 115 |
| 46R | AGCggtggaggagtgcaggctct | 116 |
| 47R | CGCggtggaggagtgcaggctct | 117 |
| 48R | GGCggtggaggagtgcaggctct | 118 |
| 49R | TTGggtggaggagtgcaggctct | 119 |
| 50R | ATGggtggaggagtgcaggctct | 120 |
| 51R | CTGggtggaggagtgcaggctct | 121 |
| 52R | GTGggtggaggagtgcaggctct | 122 |
| 53R | TAGggtggaggagtgcaggctct | 123 |
| 54R | AAGggtggaggagtgcaggctct | 124 |
| 55R | GACggtggaggagtgcaggctct | 125 |
| 56R | GACggtggaggagtgcaggctct | 126 |
| 57R | TCCggtggaggagtgcaggctct | 127 |
| 58R | ACCggtggaggagtgcaggctct | 128 |
| 59R | CCCggtggaggagtgcaggctct | 129 |
| 60R | GCCggtggaggagtgcaggctct | 130 |
| 61R | TCCggtggaggagtgcaggctct | 131 |
| 62R | ACCggtggaggagtgcaggctct | 132 |
| 63R | CCCggtggaggagtgcaggctct | 133 |
| 64R | GCCggtggaggagtgcaggctct | 134 |

TABLE 15
Left Primers

| primer | sequence | SEQ ID NO. |
|---|---|---|
| 1L | TTTtagaaactcgtagagtccca | 135 |
| 2L | ATTtagaaactcgtagagtccca | 136 |
| 3L | CTTtagaaactcgtagagtccca | 137 |
| 4L | GTTtagaaactcgtagagtccca | 138 |
| 5L | TATtagaaactcgtagagtccca | 139 |
| 6L | AATtagaaactcgtagagtccca | 140 |
| 7L | CATtagaaactcgtagagtccca | 141 |
| 8L | GATtagaaactcgtagagtccca | 142 |
| 9L | TCTtagaaactcgtagagtccca | 143 |
| 10L | ACTtagaaactcgtagagtccca | 144 |
| 11L | CCTtagaaactegtagagtccca | 145 |
| 12L | GCTtagaaactcgtagagtccca | 146 |
| 13L | TGTtagaaactcgtagagtccca | 147 |
| 14L | AGTtagaaactcgtagagtccca | 148 |
| 15L | CGTtagaaactcgtagagtccca | 149 |
| 16L | GGTtagaaactcgtagagtccca | 150 |
| 17L | TTAtagaaactcgtagagtccca | 151 |
| 18L | ATAtagaaactcgtagagtccca | 152 |
| 19L | CTAtagaaactcgtagagtccca | 153 |
| 20L | GTAtagaaactcgtagagtccca | 154 |
| 21L | TAAtagaaactcgtagagtccca | 155 |
| 22L | AAAtagaaactcgtagagtccca | 156 |
| 23L | CAAtagaaactcgtagagtccca | 157 |
| 24L | GAAtagaaactcgtagagtccca | 158 |
| 25L | TCAtagaaactcgtagagtccca | 159 |
| 26L | ACAtagaaactcgtagagtccca | 160 |
| 27L | CCAtagaaactcgtagagtccca | 161 |
| 28L | GCAtagaaactcgtagagtccca | 162 |
| 29L | TGAtagaaactcgtagagtccca | 163 |
| 3OL | AGAtagaaactcgtagagtccca | 164 |
| 31L | CGAtagaaactcgtagagtccca | 165 |
| 32L | GGAtagaaactcgtagagtccca | 166 |
| 33L | TTCtagaaactcgtagagtccca | 167 |
| 34L | ATCtagaaactcgtagagtccca | 168 |
| 35L | CTCtagaaactcgtagagtccca | 169 |
| 36L | GTCtagaaactcgtagagtccca | 170 |
| 37L | TACtagaaactcgtagagtccca | 171 |
| 38L | AACtagaaactcgtagagtccca | 172 |
| 39L | CACtagaaactcgtagagtccca | 173 |
| 40L | GACtagaaactcgtagagtccca | 174 |
| 41L | TCCtagaaactcgtagagtccca | 175 |
| 42L | ACCtagaaactcgtagagtccca | 176 |
| 43L | CCCtagaaactcgtagagtccca | 177 |
| 44L | GCCtagaaactcgtagagtccca | 178 |
| 45L | TGCtagaaactcgtagagtccca | 179 |
| 46L | AGCtagaaactcgtagagtccca | 180 |
| 47L | CGCtagaaactcgtagagtccca | 181 |
| 48L | GGCtagaaactcgtagagtccca | 182 |
| 49L | TTGtagaaactcgtagagtccca | 183 |
| 50L | ATGtagaaactcgtagagtccca | 184 |
| 51L | CTGtagaaactcgtagagtccca | 185 |
| 52L | GTGtagaaactcgtagagtccca | 186 |
| 53L | TAGtagaaactcgtagagtccca | 187 |
| 54L | AAGtagaaactcgtagagtccca | 188 |
| 55L | CAGtagaaactcgtagagtccca | 189 |
| 56L | GAGtagaaactcgtagagtccca | 190 |
| 57L | TCGtagaaactcgtagagtccca | 191 |
| 58L | ACGtagaaactcgtagagtccca | 192 |
| 59L | CCGtagaaactcgtagagtccca | 193 |
| 60L | GCGtagaaactcgtagagtccca | 194 |
| 61L | TGGtagaaactcgtagagtccca | 195 |
| 62L | AGGtagaaactcgtagagtccca | 196 |
| 63L | CGGtagaaactcgtagagtccca | 197 |
| 64L | GGGtagaaactcgtagagtccca | 198 |

Construction of Target Plasmid for Gap Analysis

To create target plasmids with different gaps an artificial PCR template can be produced adding or removing nucleotides in the gap between the sgRNA binding sites (shown below), this template also amplified by the primers 3042 and 3240, as denoted by SEQ ID Nos 69 and 70, respectively. The templates shown below are for NNG PAM but will be modified by the PCR primer combination chosen. The inserts are thus variable according to the desired PAM sequence encoded by the primers on both ends and the desired gap. PCR should yield products of varying lengths; template N14 results in a gap of 14 bp and an insert length 60. Similarly, N15, N16, N17, N25, N26 and N27, respectively have gaps of 15, 16, 17, 25, 26 or 27 bp and an insert size of 61, 62, 63, 71, 72 or 73 bp.

Spacer gap region is bold.

N14:
ctctagaaactcgtagagtcccatgtctgcggcttccagagcctgcactcctccaccttg, as
denoted by SEQ ID NO. 199,
N15:
ctctagaaactcgtagagtcccatgtctgccggcttccagagcctgcactcctccaccttg, as
denoted by SEQ ID NO. 200,
N16:
ctctagaaactcgtagagtcccatgtctgcacggcttccagagcctgcactcctccaccttg, as
denoted by SEQ ID NO. 201,
N17:
ctctagaaactcgtagagtcccatgtctgcaccggcttccagagcctgcactcctccaccttg, as
denoted by SEQ ID NO. 202,
N25:
ctctagaaactcgtagagtcccatgtctgccactgcagtgaggcttccagagcctgcactcctccaccttg,
as denoted by SEQ ID NO. 203,
N26:
ctctagaaactcgtagagtcccatgtctgccactgtcagtgaggcttccagagcctgcactcctccaccttg,
as denoted by SEQ ID NO. 204,
N27:
ctctagaaactcgtagagtcccatgtctgccactgtacagtgaggcttccagagcctgcactcctccaccttg,
as denoted by SEQ ID NO. 205.

These short target sequences are synthesized and cloned into pGEM-T easy cloning system.

Use of different RNA-guided nucleases and optimization of their protein sequence

To test the proteins of Example 1 and Example 2 the inventors designed assays for detection of RNA-guided nuclease activity as described, using a library of different in-vitro expressed proteins and a subset of gRNAs on a constant target plasmid will allow rapid scanning of properties of the proteins such as use of different PAM replacement domains, amino-acid substitutions, and different linkers. Following results in-vitro, in-vivo experiments can then confirm these results. It should be noted that representative population of cells treated under the conditions of the current example were taken for analysis using a method for off-target detection cited in the methods section. Due to use of dual RNA targeting, less off-target cleavage compared to Cas9 is expected.

### Example 7

### The Gene Editing System of the Invention for Use in Gene Therapy of Congenital Disorders

To further evaluate the feasibility of using the gene editing systems of the invention for gene therapy, several genes involved in congenital disorders are next examined

The gene editing systems of the invention for treating Autosomal dominant Retinitis Pigmentosa (adRP) Retinitis pigmentosa (RP) is an inherited dystrophic or degenerative disease of the retina with a prevalence of roughly one in 4,000.

There are currently twenty five genes reported to be involved in adRP. Mutations in the RHO gene (Rhodopsin) account for 30% of adRP, and almost all the mutations are missense mutations that lead to replacement of an amino acid reside with another. A specific mutation of Pro23His seems to be the most prevalent (13% of all adRP).

The inventors therefore next adapt the gene editing systems of the invention for knocking out the mutant allele of the RHO gene and/or replacing a mutated RHO gene in cells of patients suffering from adRP, with a wild type rhodopsin gene.

AAV is used as a vector and is constructed to comprise a cassette comprising a Donor DNA encoding the a part of Homo sapiens rhodopsin (RHO), whose whole mRNA is of 2.77 kb size (NM_000539, SEQ ID NO. 254, that encodes the amino acid sequence as denoted by SEQ ID NO. 255), as well as the nuclease of the gene editing system of the invention. Alternatively, the donor DNA and nuclease and gRNA may be delivered on separate AAV vectors or together or separately by other means of delivery (i.e. synthetically synthesized modified gRNAs, components encoded by nucleic acids or delivered as RNPs). Potentially a repair-donor-DNA for homologous recombination (HDR) can be significantly shorter allowing both repair-donor and the gene editing system of the invention expressing cassette to be co-delivered. Expression of site-specific nucleases such as the gene editing system of the invention should greatly enhance HDR. Stem/progenitor cell approaches exhibit enormous potential for RP treatment using strategies mainly aimed at the rescue and replacement of photoreceptors and RPE (retinal pigmented epithelium). The sources of stem/progenitor cells are classified into two broad categories: (a) Ocular-derived progenitor cells, such as retinal progenitor cells (RPCs), as well as (b) non-ocular-derived stem cells, including embryonic stem cells (ESCs), induced pluripotent stem cells (iPSCs), and mesenchymal stromal cells (MSCs).

Challenges still remaining are related to the proliferation and/or differentiation of the stem cells into target cells in vitro. Additional factors to consider are limited likelihood of long-term graft survival and host functional restoration in vivo.

The inventors use a genetic mouse/rat model of the P23H (proline to histidine) mutation. A mini-pig model of this mutation is also available. The pig eye is similar to the human eye in physiology, anatomy and metabolism.

Preclinical outcome measurements:

1. Full field ERG (Electroretinography).

2. Rodent behavioral analysis — Morris water maze, optokinetic

3. SD-OCT-OCT is an interferometer-based imaging technology providing cross-sectional images of tissues transparent to infrared illumination.

4. Digital imaging

As indicated above, the gene editing system of the invention is very well suited for gene therapy of adRP.

The main delivery method for RP is AAV which has a size limit of 4.7 kb. Thus, dScCas9-FokI optimized for small size by deletions described in Example 3 is used. Having dual guide RNAs confers high specificity of the gene editing system of the invention, potentially abolishing off-target mutations of competing genome editing methods. A major challenge is being able to differentiate single nucleotide mutations in the patient's DNA. This may possibly be addressed in two methods, each with its advantages.

1) Targeting only the mutant allele (i.e. Pro23His mutation in the Rhodopsin gene) to knock it out by using Locked Nucleic Acid (LNA) or similar RNA modifications in the SCNA which may be designed to be effective in such differentiation, are highly stable, and are suitable for systemic use (Di Martino MT, Gulla A, Gallo Cantafio ME, Altomare E, Amodio N, Leone E, et al. (2014) In Vitro and In Vivo Activity of a Novel Locked Nucleic Acid (LNA)-Inhibitor-miR-221 against Multiple Myeloma Cells. PLoS ONE 9(2): e89659). Thus, knockout of the dominant mutant allele will ostensibly leave sufficient WT recessive allele product to alleviate disease progression.

2) Targeting both alleles with the gene editing platform of the invention and concomitantly providing a fractional promoterless WT-like Donor (replacement) DNA for Homologous Recombination. Thus, the Pro23His mutation in the Rhodopsin gene can be corrected in the mutant allele without affecting the WT one and without increasing WT Rhodopsin levels which may be toxic in itself.

Gene/cell-therapy clinical trials for RP have a relatively short follow up period for safety (12-24 months). If safety results look promising, it is worth going into the possibly longer term follow up for efficacy trials.

FIG. 6 illustrates the target sequence within exon 1 of the RHO gene of SEQ ID NO. 214, targeted by the SCNA of SEQ ID NOs. 215, 216 of set 1, and of SEQ ID NOs. 217 and 218, of set 2. To increase specificity one or more locked nucleic acids (LNAs) may be included at one or more sites, possibly including base 7 in SEQ ID NO. 216 or base 16 in SEQ ID NO. 217. Gene targeting efficiency in human cells and mouse model may be evaluated by DNA purification and NGS. For human cells, primers SEQ ID NO. 304 and SEQ ID NO. 305 are used for PCR, followed by high-throughput DNA sequencing. For human treatments, the gene targeting efficiency may be evaluated through gain of vision or stop of degradation.

The gene editing systems of the invention for treating Pseudoachondroplasia (PSACH)

Pseudoachondroplasia (PSACH) is a skeletal dysplasia characterized by disproportionate short stature, small hands and feet, abnormal joints and early onset osteoarthritis. PSACH is caused by mutations in the gene encoding thrombospondin 5 (TSP-5, also known as cartilage oligomeric matrix protein or COMP), a pentameric extracellular matrix protein primarily expressed in chondrocytes and musculoskeletal tissues.

PSACH results from a dominant-negative effect of COMP mutations that lead to intracellular retention of mis-assembled pentameric COMP composed of a mix of both mutant and wild-type subunits. Over 100 mutations in COMP have been identified. Approximately 30% of cases result from deletion of one of five sequential aspartic acid residues at position 469-473 and is denoted as the D469del mutation. One mutation in which aspartic acid residue 469 is deleted, D469del, accounts for approximately 30% of PSACH cases.

Mutant COMP protein is misfolded, which led to a working model of the PSACH disease pathology in which mutations in COMP lead to accumulation of mature COMP in the rER, leading to excessive ER stress and ultimately premature chondrocyte death.

Currently there are no investigational therapies for pseudoachondroplasia, and therefore there is a clear unmet need for effective therapeutic systems applicable for this disease.

The inventors therefore evaluate the applicability of the gene editing systems of the invention for treating PSACH. The following preclinical model of Transgenic D469del-COMP mice with type II collagen/tetracycline-inducible promoter system, is therefore used.

More specifically, robust expression of the D469del-COMP mutant in a transgenic mouse is achieved using a tetracycline-inducible expression system. A transgenic mouse is generated that contained two expression cassettes; a cassette where the type II collagen promoter drives chondrocyte-specific expression of the rtTA protein (of the Tet-On expression system) and a cassette in which the sequence encoding D469del-COMP mutant is under transcriptional control of activated rtTA protein. High expression levels of mutant COMP occur in chondrocytes only when the rtTA protein is activated by the presence of doxycycline. Doxycycline is administered from conception through postnatal life. This D469del-COMP mouse recapitulates critical cellular and clinical features of PSACH including (1) retention of COMP and other extracellular matrix proteins, (2) the presence of intracellular matrix in the rER cisternae, (3) increased chondrocyte death, (4) limb shortening and (5) postnatal onset of dwarfing phenotype.

The inventors therefore next provide gene editing systems in accordance with the invention, adapted or the COMP gene, as denoted by SEQ ID NO. 219 (exon 13 of the COMP gene), using the SCNAs of sett as denoted by SEQ ID NO. 220 and 221. As shown in FIG. 7, Exon 13 is targeted. Exon 13 contains the deleted ASP469 residues in patients suffering from then PSACH disease.

Two weeks after treatment is done, a biopsy (cell sample) is taken from 3 different location from skeletal areas expected to pass treatment. DNA is purified and sent for NGS analysis to determine targeting efficiency.

The PCR primers for amplification before NGS analysis are SEQ ID NO. 296 and SEQ ID NO. 297. Representative population of cells treated under the conditions of the current example are taken for analysis using a method for off-target detection cited in the methods section. Due to use of dual RNA targeting, less off-target cleavage compared to Cas9 is expected.

### Example 8

The Gene Editing System of the Invention for Use on Immune Checkpoint Receptor Proteins or Ligand Genes, and Applications Thereof in Cancer Therapy

Targeting PD-1 and PDL-1

Use of monoclonal antibodies (MoAbs) targeting PD-1 or PDL-1 have been approved for many cancer indications. Programmed cell death protein 1 (PD-1) is expressed on the immune cell, while its ligand, PDL-1 may be expressed on the tumor cell. Thus, in an ex-vivo cell therapy approach (either of autologous or allogeneic origin), T-cells are collected either from a patient or from at least one compatible donor, and gene-edited to knockout PD-1. In some applications, the knockout of PD-1, may be combined with chimeric antigen receptor (CAR) insertion into the PD-1 cut site, and re-infused to the patient. These cells proliferate in the patient's bone marrow and attack the tumor cells.

The inventors first establish a system for treating non-small cell lung cancer (NSCLC). To ensure the usefulness of the CARs in the animal models expression of the cancer, specific antigens, recognized by the CARs, are examined This is done on the variety of NSCLC cell-lines and significant expression of the antigens is a first filter for selecting the working cell-lines. For the genetic mouse model, spontaneously developing tumors are extracted and examined for the expression of the antigens. Pre-clinical and clinical studies of CAR-T activity in NSCLC involve targeting cancer specific antigen NY-ESO-1/LAGE-1 (Robbins et al, 2011, J Clin Oncol, 29:917-24; Zhao et al, 2005, J Immunol, 174:4415-23; Purbhoo et al, 2006, J Immunol, 176:7308).

Mouse models for NSCLC involve both genetically modified mice that develop lung cancer and xenografts of human cancer cell-lines. The main limitations in the choice of model are: 1) the tumors must express the specific antigen targeted by the CAR, and 2) the T-cells used must match the model.

For the genetically modified mouse model the CCSP-rtTA TetO-KrasG12D is used on the background of TRP53-/-& Ink4A-/-. In this model, the specific mutated Kras gene is activated by doxycyclin giving rise to tumors within 1 month (Fisher et al, 2001, Genes Dev, 15:3249-62). In a preliminary study, the tumors from 5 of these mice are examined for the expression of the cancer-specific antigens for which CARs are available. In addition, as the CARs are developed against human proteins, it is ensured that they also recognize the mouse counterparts.

A variety of human NSCLC tumor cell-lines exist for use in xenograft mouse models (i.e. H1299, A549). Several of these cell-lines are obtained and confirmed for the expression of the tumor antigens targeted by the CARs. Three cell-lines expressing the tumor antigens are examined for their ability to produce tumors in immune deficient mice. Two are selected for the pre-clinical study.

In all studies, modified T-cells are compared with non-modified T-cell controls (Kalaitsidou et al, 2015, Immunotherapy, 7:487-97). Mice are either induced to produce tumors (genetically modified model) or injected subcutaneously in the flanks with the human cell-lines. Once tumors appear at a volume of ˜200mm³, the mice are injected with the modified or non-modified CAR-T cells. Mouse T-cells are used for the genetically modified model and human T-cells for the xenograft studies. Briefly, the experimental design involves 3 different CAR-T cell titers with at least 5 mice per experimental group. An additional control group involves modified and non-modified T-cells lacking the relevant CAR and a non receiving T-cell group (tumor only). The establishment of the mouse models at the CRO entails the following:

a. for the genetic mouse model, the time frame, tumors size and spread and statistical variance are examined following the induction of tumorigenesis;

b. for the xenograft studies, four selected cell-lines are examined for their ability to produce tumors in immune-compromised mice. Two cell-lines presenting the best characteristics in robust and timely tumor generation are chosen. The process also determines the time-frame, measurable variables, and statistical variance to derive the needed experimental group size;

c. the general reactivity of the mice in the two models to injection of the experimental PD null CAR-T cells is examined in non-tumor bearing mice.

The genetic mouse model is performed by introducing the PD-1-null CAR-T cells into the mice after the tumors reach a predetermined size (as determined by the previous step). Several doses of the experimental mouse T cells are used. Controls include PD-1-null T cells that do not express any CAR and cells expressing non-specific CARs.

The xenograft models are performed by engrafting the selected cells under the skin of immune deficient mice. After the tumors reach a predetermined size, several doses of the experimental human T cells are introduced. Controls are similar to the other model. Efficacy is determined by reduction of tumor volume, weight, survival of the mice, and immunohistopathological examination of extracted tumor. In this histo-examination the structure of the tumor, the number and characteristics of the infiltrating cells are tested as compared to the various controls. Robust statistical analysis may be employed to validate the data.

SCNA and HDR Design

Knockout of PD-1 is carried out by deletion of the first exon of the PDCD-1 gene and replacement by a CAR-T targeting NY-ESO-1/LAGE-1.

The chimeric antigen receptor cassette consists of: a constitutive human promoter, a fusion gene of an anti-NY-ESO-1/LAGE-1 scFv, IgG4 scaffold domain, human CD8 transmembrane domain, human CD28 signaling domain, and a human CD3zeta signaling domain as has been described for lentiviral vectors (Maus et al., 2016, Molecular Therapy-Oncolytics, 3:1-9). HDR is facilitated by 500 bp homology arms to the PDCD-1 exon 1. The DNA sequence is shown in SEQ ID NO. 293 and a sequence map is shown in FIG. 13.

The anti-NY-ESO-1 scFv is derived from a high-affinity antibody with 2nM affinity to NY-ESO-1 (Stewart-Jones et al., 2009, PNAS, 106:5784-8). scFv was constructed by fusing the variable regions of the light and heavy chains with a long glycine-rich linker. CMV promoter and BGH Terminator are used.

Two possible sites for dScCas9-FokI in the first exon of the PDCD1 gene, are denoted by SEQ ID NO. 222, using the SCNAs of sett as denoted by SEQ ID NO. 223 and 224, or using the SCNAs of set2 as denoted by SEQ ID NO. 225 and 226, also shown in FIG. 8. PD-1 gene NHEJ erroneous repair was tested in Hek293 cells, harvested 96 hours after transfection with plasmids encoding the protein and the SCNAs. ddPCR probe PDF4-TGEE40 (SEQ ID NO. 423) and primers PD F6 (SEQ ID NO. 424) and PD R4 (SEQ ID NO. 425). These results show the ability of dScCasFok to edit the PD-1 exon at high efficiency.

TABLE 16
Gene editing efficiencies of dScCasFok targeting the PD-1 exon 1.

| | Construct | Guide RNA | Editing % | Editing % |
|---|---|---|---|---|
| Description | (SEQ ID NO) | (SEQ ID NO) | (ddPCR76) | (ddPCR79) |
| SpCas9, | 7665 (257) | 14106 (411) | 11.1% | 10.8% |
| SV40 + nucleoplasmin NLS | | | | |
| dScCasFok, SV40 NLS | 11241 (2) | 14078 (412) | 0.8% | 1.4% |
| dScCasFok, SV40 NLS | 11241 (2) | 14080 (413) | 1.8% | 4.0% |
| dScCasFok, SV40 NLS | 11241 (2) | 14082 (408) | 29.5% | 29.0% |
| dScCasFok, SV40 NLS | 11241 (2) | 14084 (409) | 0.7% | 1.4% |
| dScCasFok, SV40 NLS | 11241 (2) | 14086 (410) | 0.4% | 0.7% |
| dScCasFok, SV40 NLS | 11241 (2) | 14088 (414) | 1.1% | 1.2% |
| dScCasFok, SV40 NLS | 11241 (2) | 14090 (415) | 5.6% | 0.7% |

Editing efficiency was quantified by ddPCR (ddPCR#76 and #79 results are shown). NLS = Nuclear localization sequence.

### Example 9

### dCasF-Fok Variants

a) for PD-1 knock-out

CasF is a family of Cas proteins found in the Biggiephage Glade, which are huge bacteriophages (Pausch et al (2020), Science, 369:333-7). Pausch et al investigated three CasF variants, CasF-1 (SEQ ID NO: 358), CasF-2 (SEQ ID NO: 359), and CasF-3 (SEQ ID NO: 360). These Cas proteins appear to use a single RuvC domain for both pre-crRNA processing and cleavage of both strands of DNA (Cas9, for comparison, uses accessory proteins for pre-crRNA processing, and an HNH domain for cleavage of the non-template strand of DNA). The RuvC nuclease activity may be inactivated with a D394A mutation in CasF-2 and a D371 mutation in CasF-1. CasF are distant homologues of Cas14a and Cas14b.

The PAM of CasF-1 is 5′-VTTR-3′, the PAM of CasF-2 is 5′-TBN-3′, and the PAM of CasF-3 is 5′-VTTN-3′. CasF-2 is preferable to use in a dCasF-Fok system due to the reduced PAM requirement. dCasF2Fok variants were designed with FokI at either the C-terminus (SEQ ID NO: 363) or N-terminus (SEQ ID NO: 366).

Several target sites with TBN PAM are possible in Exon 2 of PD-1, both in PAM-out and PAM-in configurations. The dual guide RNAs may be expressed using H1 and U6 promoters. Three PAM-out guides (SEQ ID NO: 367, 368, and 369) and three PAM-in guides (SEQ ID NO: 370, 371, 372) were designed.

b) for MPO gene

In a further example, dCasF2-Fok (a fusion protein between a nuclease-deficient CasF-2 (dCasF-2, SEQ ID NO: 361) and FokI), may have all or part of its RuvC nuclease domain deleted. The RuvC domain includes dCasF-2 residues 389-410, residues 601-612, and residues 689-706 of SEQ ID NO: 361. RuvC domain residues may be replaced by short linkers (Gly-Gly-Ser-Gly, as denoted by SEQ ID NO: 399). Three deletion variants of dCasF2-Fok were designed: dCasF2-Fok RuvC del 1 which has deletion in dCasF-2 residues 389-410 (SEQ ID NO 362), dCasF2-Fok RuvC del 2 which has deletion in dCasF-2 residues 601-612 (SEQ ID NO 364), and dCasF2-Fok RuvC del 3 which has deletion in dCasF-2 residues 689-706 (SEQ ID NO 365). Variants are tested on the MPO exon 1 using the same ddPCR primers and probe as for dScCas9-Fok and variants thereof. The plasmid for expression of MPO exon 1 guides (in PAM-out configuration) is shown in SEQ ID NO 319.

### Example 10

### dCas-FokI Variants with Optimized Linker Sequences

The dCas9-FokI used herein are subjected to optimization using different linker sequences. The “linker peptide” connecting the “Linking domain” attached to the guide RNA and the “Nuclease domain” that cleaves the DNA may be changed to alter its properties. These changes may include length and amino acid composition (including amino acid charge, size, hydrophobicity, secondary structure affinity).

The following examples of linkers of SEQ ID NOs. 206, 207, 208 and 209 that link dCasX to FokI in the chimeras of SEQ ID NOs. 210, 211, 212 and 213, respectively, are shown to illustrate possible changes to the linker peptide. The efficiency of the different chimeras linked by each of the linking domain is evaluated.

### Example 11

### Targeting CTLA-4

Use of MoAbs against immune checkpoint receptor proteins or ligands has shown greater toxicity when targeting cytotoxic T-lymphocyte-associated protein 4 (CTLA-4) presumably as it is a general systemic immune system checkpoint, whereas PD-1/PDL-1 seems to be more local/tumor specific. As designed here, ex-vivo modified CTLA-4 knockout lymphocytes would not only generate less autoimmune toxicity than MoAbs, as only a portion would contain the knockouts, but could be combined with a PD-1 antibody as well as with any additional cancer therapy. While the combination of both antibodies (anti PD1 and anti CTLA-4) has been approved only in melanoma, there is promising early data in NSCLC. The third indication would be advanced renal cell cancer, where the combination of PD-1 and CTLA-4 inhibition (by MoAbs) is an approved indication. Thus, as in melanoma, ex-vivo modified CTLA-4 knockout lymphocytes may generate less autoimmune toxicity than MoAbs, as only a portion of the cells would contain the knockouts, and thus could be combined with a PD-1 antibody.

The inventors therefore next evaluate the feasibility of using the gene editing system of the invention for targeting CTLA4, in lymphocytes obtained from melanoma, NSCLC or renal cell cancer patients. The dScCas9-FokI system may be used to knock-out exon 1 of human CTLA4 gene. As shown in FIG. 9A, 9B, the human CTLA4 exon 1 as denoted by SEQ ID NO. 227, using the SCNAs of sett as denoted by SEQ ID NO. 228 and 229, or using the SCNAs of set2 as denoted by SEQ ID NOs. 230 and 231. Sequencing primers for NGS are SEQ ID NOs. 298 and 299.

Gene Knock-Out with Optimized dCasX-FokI System

Alternatively, dCasX-FokI system may be used to knock-out exon 1 of human CTLA4 gene. As shown in FIG. 12, the human CTLA4 exon 1 and 5′ untranslated sequence as denoted by SEQ ID NO. 285, using the SCNAs as denoted by SEQ ID NO. 286 and 287.

CasX are a separate class of Cas nucleases to Cas9 nucleases [11]. A major limitation to their function as effective nucleases is their PAM sequence of TTC. Alteration that reduce or remove the PAM sequence would significantly improve their utility.

Expected results are a naturally smaller protein fused to FokI, whereby CasX's natural PAM binding domain has been altered to allow greater flexibility in target choice. The Non-Target Strand Binding (NTSB) domain [11] is residues 101-191 and is involved in DNA cleavage and thus may be redundant for a FokI fusion. The Target Strand Loading (TSL) is residues 825-934 and is a domain of unknown function. Inventors' analysis suggests it may be dispensable for target site binding. The individual point mutations or their combinations below are expected to change the PAM binding domain to remove specific DNA-base-protein interaction replacing them with non-sequence specific interactions.

The following chimeric proteins are therefore prepared as described in the Experimental procedures section herein before. Constructs ordered as gene synthesis constructs from GeneArt: dCasX-FokI, as denoted by SEQ ID NO. 45, dCasX-FokI dNTSB, as denoted by SEQ ID NO. 46, dCasX-FokI dTSL, as denoted by SEQ ID NO. 47, dCasX-FokI dNTSB dTSL, as denoted by SEQ ID NO. 48, and variants thereof that comprise at least one substitution in at least one of K226, specifically, substituting lysine to A, K226, specifically, substituting lysine to Q, 5521, specifically, substituting serine to K, 5525, specifically, substituting serine to K and G577, specifically, substituting glycine to K. Gene-editing efficiency is determined by DNA sequencing of transformed lymphocytes. Sequencing primers for NGS are SEQ ID NO. 298 and 299.

### Example 12

The Gene Editing System of the Invention for Use in Tumor Associated Antigens (TAAs)

The inventors next establish the feasibility of using the gene editing systems of the invention in targeting TAAs, for cancer therapy.

Ex-Vivo Cell therapy for treatment of Ovarian and or triple-negative breast cancer by targeting Siglec-10

Many tumors overexpress CD24. Recently it has been shown (Barkal et al 2019, Nat Rev Cancer, 572:392-6) that CD24 can be the dominant innate immune checkpoint in ovarian cancer and breast cancer, and is a promising target for cancer immunotherapy. Expression of this surface protein is produced as a “self” anti-offensive or suppressive signal, which is involved in macrophage evasion of the cancer cell through its interaction with the inhibitory receptor sialic-acid-binding Ig-like lectin 10 (Siglec-10), which is expressed by tumor-associated macrophages. Genetic ablation and therapeutic blockade of either CD24 or Siglec-10, as well as blockade of the CD24-Siglec-10 interaction using monoclonal antibodies resulted in a macrophage-dependent reduction of tumor growth in vivo and an increase in survival time. Siglec-10 is expressed in cells of the innate immune system and cells transcribing and expressing Siglec-10 RNA and protein respectively can mainly be found in spleen, tonsil, lymph node, bone marrow and the appendix (human protein atlas). SIGLECs are members of the immunoglobulin superfamily that are expressed on the cell surface. Most SIGLECs have one or more cytoplasmic immune receptor tyrosine-based inhibitory motifs, or ITIMs. SIGLECs are typically expressed on cells of the innate immune system, with the exception of the B -cell expressed SIGLEC6.

Thus, targeting Siglec-10 by knocking it out for example by inducing a frameshift mutation, could be expected to produce Macrophages that are insusceptible to the tumor's CD24 suppressive signal.

The inventors therefore have designed an ex-vivo method to treat cancer in a human patient by targeting CD14+primary monocytes from the patient's bone-marrow or peripheral blood. Monocytes evolve into Macrophages and dendritic cells. Though protocols for CD34+gene-editing are much further developed, as in this example it was not intended to eliminate Siglec-10 expression from B-cells (which also evolve from CD34+HSCs), and as Siglec-10 is constitutively expressed on B cell surfaces and acts as an inhibitory coreceptor of the BCR it is probably not advisable to use targeted CD34+HSCs in this case. Alternatively, as CD34+cells are progenitors of CD14+ and easier to transfect, it may be preferable to differentiate CD14+ monocytes from this genome-edited precursor after a revival and/or expansion period.

Monocytes typically only live a few days in culture. In-vivo survival of HSC derived monocytes may also be short, though there are multiple pathways for the generation and maintenance of distinct mononuclear phagocyte subpopulations. For example, Kupffer cells, as well as lung, peritoneal and splenic macrophages are established before birth and remain in adulthood uncoupled from the steady state monocyte pool (Yona S. et al. Immunity, 2013, 38:79-91). Next, the inventors knockout the Siglec-10 gene in the monocytes (or CD34+) using an RNA-guided nuclease, optionally, expanding and selecting cells not expressing Siglec-10 and re-infusing the cells into the patient.

A protocol for CD14+monocyte genome editing using CRISPR-Cas9 has recently been developed (Wang et al, 2018, Molecular Therapy: Nucleic Acids, 11:P130-41). In this paper they achieved up to 12% cleavage of their target in primary monocytes using plasmid transfection. Here, pre-assembled (ribonucleo-protein) RNPs are used for enhanced efficiency. CRISPR-Cas9 RNPs can achieve more than 90% NHEJ erroneous repair in CD34+ HSCs using electroporation (Wu et al, 2019, Nature Medicine, 25:776-83).

The use of the gene editing systems of the invention that is a precise, dual sgRNA system, significantly diminish the danger of off-target mutations relatively to single RNA guided CRISPR-Cas9.

Delivery of the gene-editing reagents is made using nucleic acids encoding the protein and sgRNAs (SCNAs) or by producing the protein and a synthetic RNA, combining them in-vitro and supplying them to the monocyte cells in culture, or by different combination of nucleic acids and proteins. Use of a preassembled ribonucleic-acid — protein has several advantages when no foreign gene needs to be introduced as this enhances the safety as no foreign DNA is present to integrate into genomic breaks.

As shown by FIG. 10, the inventors next target exon 1 of the human Siglec-10 gene as denoted by SEQ ID NO. 232, using the SCNAs of sett as denoted by SEQ ID NO. 233 and 234, or using the SCNAs of set2 as denoted by SEQ ID NO. 235 and 236, or using the SCNAs of set3 as denoted by SEQ ID NO. 237 and 238, or using the SCNAs of set4 as denoted by SEQ ID NO. 239 and 240, or using the SCNAs of sets as denoted by SEQ ID NO. 241 and 242.

### Example 13

### dCas-Fok Variants with Activity Enhanced by Chromatin Binding Domains

Chromatin binding domains may enhance the interaction of nucleases with genomic DNA and/or allow greater accessibility into the genomic DNA. HMG (high mobility group proteins) are one such example of chromatin binding domains that may be fused with dCas-Fok variants. An example of this is shown in the Table 17 below where dScCas9Fok is compared to HMGN-dScCas9Fok, which is a fusion protein between the HMGN box chromatin opening domain (SEQ ID NO:324) and dScCas9Fok. SCNAs targeting the human MPO gene exon 1 were encoded by plasmid GeneMsgRNA15E1. MPO gene NHEJ erroneous repair was tested in HEK293 cells, harvested 72 hours after transfection with plasmids encoding the proteins and the SCNAs.

TABLE 17
Gene editing efficiencies of HMGN-dScCasFok construct.

| | Construct | |
|---|---|---|
| | (SEQ ID) | Editing % |
| dScCasFok, 2NLS variant | 13185 (330) | 10.88 |
| HMGN-dScCasFok, 2NLS variant | 13213 (323) | 14.20 |

Editing efficiency was quantified by ddPCR (ddPCR#63 results are shown). NLS = Nuclear localization sequence.

Addition of a chromatin binding domain, HMGN (SEQ ID NO:324) to the N-terminus of dScCas-Fok caused a 30.5% improvement of NHEJ-ER compared to a control dScCasFok variant with no chromatin binding domains. This shows that fusing dScCas-Fok to a chromatin binding domain may improve activity, possibly through improved chromatin binding or accessibility.

### Example 14

### dCas14-Fok1 and Derivatives

Alternative dCas variants may be used in the dCasFok system. Cas14 is a class of CRISPR-Cas nucleases that are approximately half the size of Cas9 nucleases. Cas14 has been reported to carry out targeted dsDNA cleavage at sites containing a PAM with 5′ TTTN sequence (Karvelis et al., 2019, Biorxiv, doi:10.1101/654897). Nuclease-deficient Cas14 proteins have been reported. dCas14-FokI is predicted to have significantly higher specificity than Cas14 enzymes due to the presence of two guide RNA sequences. Fusion with protein domains promoting chromatin binding and opening is predicted to increase cleavage efficiency without compromising specificity.

dCas14-FOKI is shown in SEQ ID NO. 271. dCas14-FOKI fused to chromatin binding StkC domain is shown in SEQ ID NO. 272. dCas14-FokI fused to HIV integrase DNA-binding domain is shown in SEQ ID NO. 273. dCas14-FOKI fused to Sso7d DNA-binding domain is shown in SEQ ID NO. 274. dCas14-FOKI with N-terminal HMGN box chromatin opening domain is shown in SEQ ID NO. 275. dCas14-FOKI with C-terminal HMGB1 chromatin opening domain is shown in SEQ ID NO. 279. dCas14-FOKI with C-terminal HMGB3 chromatin opening domain is shown in SEQ ID NO. 280. dCas14-FOKI with C-terminal HMGB4 chromatin opening domain is shown in SEQ ID NO. 281. dCas14-FOKI with N-terminal HMGN box chromatin opening domain and C-terminal HMGB1 chromatin opening domain is shown in SEQ ID NO. 276. dCas14-FOKI with N-terminal HMGN box chromatin opening domain and C-terminal HMGB3 chromatin opening domain is shown in SEQ ID NO. 277. dCas14-FOKI with N-terminal HMGN box chromatin opening domain and C-terminal HMGB4 chromatin opening domain is shown in SEQ ID NO. 278.

The dCas14-FokI system may be used to knock-out Siglec-10. SCNAs targeting exon 2 of Siglec 10 are SEQ ID NO. 282 and SEQ ID NO. 283. All constructs ordered as gene synthesis constructs from various suppliers. Treatment efficiency is detected by Cy5 labeled antibodies against each of the targeted immune checkpoint receptor proteins or ligands using FACS analysis. A Cy5 labeled secondary antibody (Goat Anti-Mouse IgM μ chain Antibody, Cy5 conjugate AP500S Merck) is used for ELISA or FACS analysis. Next-generation sequencing is also used to evaluate gene-editing efficiency, NGS primers that may be used for the dCas14-Fok system include SEQ ID NO. 300 and 301.

### Example 15

### The Gene Editing System of the Invention for Use in Receptors for Viral Antigens

Targeting genes encoding avr33 integrin in cows to form Foot and Mouth Disease resistance. Similar to CCR5 and HIV in human, the epithelial Integrin avr36 is a receptor for the foot-and-mouth disease virus. Therefore, knock-out of genes encoding subunits of the avr33 integrin may reduce susceptibility to foot-and-mouth disease. Since the alpha subunit may interact with many beta subunits, the ITGB3 gene encoding specifically the beta subunit of avr33 integrin was selected. FIG. 11 illustrates the targeted exon 1. The following SCNAs sets were designed to knockout exon 1 of the ITGB3 gene, as denoted by SEQ ID NO. 243, using the SCNAs of sett as denoted by SEQ ID NO. 244 and 245, or using the SCNAs of set2 as denoted by SEQ ID NO. 246 and 247, or using the SCNAs of set3 as denoted by SEQ ID NO. 248 and 249, or using the SCNAs of set4 as denoted by SEQ ID NO. 250 and 251, or using the SCNAs of sets as denoted by SEQ ID NO. 252 and 253.

A microinjection of the RNPs of the invention are performed to calf oocytes. More stable and active ancestral dCas9-FokI RNPs may be used for this purpose, described below. Gene-editing efficiency may be evaluated by PCR, using primers SEQ ID NO. 302 and SEQ ID NO. 303.

### Example 16

### Highly Stable and Active Ancestral dCas9-FokI Nucleases

Ancestral reconstruction is a method of inferring the ancestors of a protein of interest by comparing related proteins and using statistical methods to rewind their evolutionary history. Ancestral proteins often have increased stability, expression, and activity (Trudeau et al, 2016, Mol Biol Evol, 33:2633-41). For that reason they have been proposed as ideal starting points for further optimization (Trudeau & Tawfik, 2019, Curr Opin Biotechnol, 60:46-52). In the context of genome editing proteins, they may assist with expression and in vivo activity.

A multiple sequence alignment of 200 homologues of Cas9 with 25-90% identity was created by the inventors from the UniRef90 database (Suzek et al. 2015, Bioinformatics, 31:926-32). Maximum likelihood statistical methods were applied to infer the ancestors of these proteins (Ashkenazy et al., 2012, Nucleic Acids Research, 40:W580-4). An ancestral Cas9 is shown in SEQ ID NO. 268. A nuclease deficient Ancestral Cas9 fused to FokI nuclease is shown in SEQ ID NO. 284. Constructs ordered as gene synthesis constructs from GeneArt.

A multiple sequence alignment of 67 homologues of FokI with 25-90% identity was created by the inventors from the UniRef90 database (Suzek et al. 2015, Bioinformatics, 31:926-32). Analysis of the resulting sequence alignment was used to find consensus positions (where over 50% of the amino acids at a particular site in the alignment are the same residue and differ from that of wild-type FokI (Trudeau et al, 2016, Mol Biol Evol, 33:2633-41)). This consensus FokI (“FokI consensus”, also referred to as “ancestral FokI”) is shown in SEQ ID NO. 439. This variant also included two activity-enhancing mutations present in the “Sharkey” FokI mutant (Guo et al, 2010, J Mol Biol, 400(1):96-107), which were also tested independently in a separate FokI mutant (“enhanced FokI”, also dented by SEQ ID NO. 486). These FokI variants were used to construct several dCasFok variants with and without Cas9 ancestral mutations, specifically in the RuvC and REC1/2 domains comprising residues 1-738 of the Cas9 component of dCasFok (residues 227-965 of SEQ ID NO:2). Constructs were ordered as gene synthesis constructs from GeneArt.

As shown in Table 18 and in FIG. 14, ddPCR was used to evaluate gene editing activity of constructs containing ancestral mutations, at time points of 76 h and 120 h. Ancestral mutations in the RuvC and REC1/2 domains of the Cas9 component of dCasFok led to a moderate improvement in gene editing activity in the 120 h time point, from 20-24% (SEQ ID NO: 375) to 28% (SEQ ID NO:441), although these improvements were not present in the 76 h time point. When Fold consensus and activity-enhancing mutations were added to dCasFok, gene editing activity was increased in the 120 h time point to a maximum of 35% (SEQ ID NO: 444), and in the 76 h time point to a maximum of 38% in a construct with a C-terminal His tag construct (SEQ ID NO:445). When FokI consensus mutations were added to the HNH deletion variant (EXAMPLE 3), gene editing activity was increased to 12% at 120 h and 18.6% at 76 h (SEQ ID NO:448). These results demonstrate improvements to overall gene editing activity using ancestral/consensus mutations, as well as previously described activity-enhancing mutations. These results also show that gene editing efficiency may vary based on editing times (120 h vs 76 h), which may have different effects on different variants, potentially due to differences in stability, activity, or expression.

TABLE 18
Gene editing efficiencies of ancestral dCasFok variants.

| | SEQ ID | TG | editing %, | Editing %, |
|---|---|---|---|---|
| Description | NO | number | 120 h | 76 h |
| Control | | | | |
| Cas9 | 257 | 7665 | 50.38% | 41.98% |
| Cas9 | 257 | 7665 | 44.99% | 23.21% |
| dScCasFok, SV40 NLS | 2 | 11241 | 11.49% | 17.85% |
| dScCasFok, SV40 NLS | 2 | 11241 | 14.39% | 19.89% |
| dScCasFok, SV40 NLS | 2 | 11241 | 13.64% | 20.03% |
| dScCasFok, SV40 + SV40 bipartite NLS | 375 | 14280 | 19.66% | 27.41% |
| dScCasFok, SV40 + SV40 bipartite NLS | 375 | 14280 | 23.89% | 33.67% |
| dScCasFok, SV40 + SV40 bipartite NLS | 375 | 14280 | 23.59% | 32.73% |
| Ancestral Mutations in RuvC + Rec1/2 domains | | | | |
| dCasFok, SV40 + bipartiteSV40, ancestral mutations | 440 | 14607 | 0.27% | 0.27% |
| in RuvC + REC1/2 domains | | | | |
| dCasFok, SV40 + bipartiteSV40, ancestral mutations | 441 | 14608 | 28.08% | 22.14% |
| in RuvC + REC1/2 domains, 6His tag | | | | |
| dCasFok, SV40 + bipartiteSV40, ancestral mutations | 442 | 14609 | 6.46% | 6.90% |
| in RuvC + REC1/2 domains, HNH deletion | | | | |
| dCasFok, SV40 + bipartiteSV40, ancestral mutations | 443 | 14610 | 4.96% | 6.45% |
| in RuvC + REC1/2 domains, HNH deletion, 6His tag | | | | |
| Enhanced FokI | | | | |
| dCasFok, FokI enhanced, SV40 + bipartiteSV40 | 487 | TG14657 | 27.37% | 19.45% |
| dCasFok, FokI enhanced, SV40 + bipartiteSV40, | 488 | TG14658 | 25.72% | 23.30% |
| 6His tag | | | | |
| dCasFok, FokI enhanced, SV40 + bipartiteSV40, | 489 | TG14661 | 33.55% | 29.31% |
| ancestral mutations in RuvC + REC1/2 domains | | | | |
| dCasFok, FokI enhanced, SV40 + bipartiteSV40, | 490 | TG14662 | 24.46% | 17.75% |
| ancestral mutations in RuvC + REC1/2 domains, | | | | |
| 6His tag | | | | |
| dCasFok, FokI enhanced, SV40 + bipartiteSV40, | 491 | TG14665 | 10.24% | 10.39% |
| ancestral mutations in RuvC + REC1/2 domains, | | | | |
| HNH deletion longer linker | | | | |
| dCasFok, FokI enhanced SV40 + bipartiteSV40, | 492 | TG14666 | 0.04% | 0.29% |
| ancestral mutations in RuvC + REC1/2 domains, | | | | |
| HNH deletion longer linker, 6His tag | | | | |
| Consensus FokI | | | | |
| dCasFok, FokI consensus, SV40 + bipartiteSV40 | 444 | 14659 | 35.16% | 24.85% |
| dCasFok, FokI consensus, SV40 + bipartiteSV40, | 445 | 14660 | 29.22% | 38.11% |
| 6His tag | | | | |
| dCasFok, FokI consensus, SV40 + bipartiteSV40, | 446 | 14663 | 31.48% | 25.77% |
| ancestral mutations in RuvC + REC1/2 domains | | | | |
| dCasFok, FokI consensus, SV40 + bipartiteSV40, | 447 | 14664 | 33.05% | 21.95% |
| ancestral mutations in RuvC + REC1/2 domains, | | | | |
| 6His tag | | | | |
| dCasFok, FokI consensus, SV40 + bipartiteSV40, | 448 | 14667 | 12.10% | 18.60% |
| ancestral mutations in RuvC + REC1/2 domains, | | | | |
| HNH deletion longer linker | | | | |
| dCasFok, FokI consensus SV40 + bipartiteSV40, | 449 | 14668 | 6.85% | 8.12% |
| ancestral mutations in RuvC + REC1/2 domains, | | | | |
| HNH deletion longer linker, 6His tag | | | | |

Editing efficiency was quantified by ddPCR (ddPCR#86 results are shown for 120 h and 76 h of editing). NLS = Nuclear localization sequence.

### Example 17

### Gene-Editing with Alternative Nucleases

Nuclease domains play an important role in gene editing efficiency: catalytic rate can influence activity and specificity (Miller et al., 2019, Nature Biotechnology 37:945-52), and cellular stability can influence activity in vivo.

Type IIs restriction endonucleases contain both a recognition domain that binds to a DNA target sequence, and a cleavage domain that cuts at or near the target site. This cleavage might be dimerization-dependent, and require the association of the cleavage domain of two monomeric restriction endonucleases. Flavobacterium okeanokoites Type IIS restriction endonuclease FokI is known to require cleavage domain dimerization for DNA cleavage (Bitinaite et al, 1998, PNAS, 95:10570-5).

Alternative Type IIS nuclease cleavage domains for use in a dCas9-Fok system, instead of FokI, may be obtained from experimentally characterized Type IIS restriction enzymes (Roberts et al., 2015, Nucleic Acid Research, 43:D298-9). These may include cleavage domains from Type IIS restriction endonucleases: AarI, Acc36I, AceIII, AclWI, AcuI, AjuI, Alol, AlwI, Alw261, AmaCSI, ApyPI, AquII, AquIII, AquIV, ArsI, AsuHPI, BaeI, BarI, Bbr7I, BbsI, BbvI, BbvII, Bbv16II, BccI, BccI, Bce83I, BceAI, BceSIII, BceSIV, BcefI, BcgI, BciVI, Bco5I, Bcol 16I, BcoDI, BcoKI, BfiI, BfuI, BfuAI, BinI, Bli736I, Bme585I, BmrI, BmsI, BmuI, BpiI, BpmI, BpuAI, BpuEI, BpuSI, BsaI, BsaXI, BsbI, Bsc91I, BscAI, BseKI, BseMI, BseMII, BseRI, BseXI, BseZI, BsgI, BslFI, BsmAI, BsmBI, BsmFI, Bso31I, BsoMAI, Bsp423I, BspCNI, BspD6I, BspIS4I, BspKT5I, BspLU11III, BspQI, BspST5I, BspTNI, BspTS514I, Bst6I, Bst12I, Bst19I, Bst71I, BstBS32I, BstFZ438I, BstGZ53I, BstH9I, BstMAI, BstOZ616I, Bst31TI, BstTS5I, BstV1I, BstV2I, BsuI, Bsu6I, Bsu537I, BtgZI, BtsI, BtsIMutI, BtsCI, Bvel, Bve1B23I, CatHI, CchII, CchIII, Cco14983III, CdpI, CjeI, CjeF38011III, CjeIAIII, CjeNII, CjeNIII, CjePI, CjeP659IV, CjeYH002IV, CjuII, CseI, CspCI, CstMI, DraRI, DrdIV, EacI, Eam1104I, EarI, EciI, Eco31I, Eco57I, FaqI, FauI, Fph2801I, GeoICI, GsuI, HgaI, Hin4I, Hin4II, HphI, Hpy99XXII, HpyAV, HpyC1I, Ksp632I, LguI, Lsp1109I, LweI, MaqI, MboII, Mcr10I, MlyI, MmeI, MnlI, NcuI, NgoAVII, NgoAVIII, NlaCI, NmeAIII, NmeA6CIII, PciSI, PcoI, PhaI, PlaDI, PleI, PpiI, PpsI, PspOMII, PspPRI, PsrI, RceI, RdeGBII, RlaII, RleAI, RpaI, RpaBI, RpaB5I, Rtr1953I, SapI, SchI, SdeAI, SdeOSI, SfaNI, SmuI, SspD5I, SstE37I, Sth132I, StsI, TaqII, TaqIII, TsoI, TspDTI, TspGWI, TstI, Tth111II, TthHB27I, UbaF9I, UbaF11I, UbaF12I, UbaF13I, UbaF14I, Vga43942II, VpaK32I, WviI.

Alternatives to dScCas9-FokI include dScCas9-CspCI (SEQ ID NO. 288), dScCas9-BsgI (SEQ ID NO. 289), and dScCas9-BbvI (SEQ ID NO. 290). Constructs are ordered as gene synthesis constructs from GeneArt.

1. A clustered regularly interspaced short palindromic repeats (CRISPR)-Cas protein or cas protein derived domain having reduced or abolished Protospacer Adjacent Motif (PAM) constraint or any variant, mutant, fusion/chimeric protein, complex or conjugate thereof or composition thereof, wherein at least one of: the PAM binding domain (PBD) and/or the PAM recognition motif, and/or the HNH-nuclease domain of said Cas protein, any fragment of said PBD, and/or of said PAM recognition motif, and/or of said HNH-nuclease domain, and at least one amino acid residue adjacent to said PBD, and/or to said PAM recognition motif, and/or to said HNH-nuclease domain of said Cas protein is deleted, replaced or substituted.

2. The CRISPR-Cas protein according to claim 1, wherein said Cas protein is at least one of Cas9, CasX, Cas14a1, Cas14b5, CasF, an ancestral Cas9, and Cas12a, optionally, wherein said Cas protein is at least one of Streptococcus canis Cas9 (ScCas9), Streptococcus pyogenes Cas9 (SpCas9), an ancestral Cas9, deltaproteobacteria CasX, Cas12a, CasF-1, CasF-2, CasF-3, Cas14a1, or Cas14b5, and wherein at least one PAM-interacting Arginine and/or Lysine residue of the PBD of said Cas protein is deleted, substituted or replaced.

3. (canceled)

4. The CRISPR-Cas protein according to claim 1, wherein at least one of:
(a) said Cas protein is ScCas9, and wherein at least one of: residues Thr1330 to Arg1342, residues Ile367 to Ala376, residues Glu1228 to Tyr1343, residues Glu1108 to Asp1375, residue Lys1337 and residue Gln1338, or any fragment or at least one amino acid residue thereof, are replaced, substituted or deleted in said ScCas9;
(b) said Cas protein further comprises at least one Non-Specific DNA Binding Domain (NSBD), said NSBD is at least one of: (i) added to said Cas; and/or (ii) replaces at least one of: said PAM binding domain, and/or said PAM recognition motif, and/or said HNH-nuclease domain, and/or any fragment thereof, and/or at least one adjacent amino acid residue, optionally, said NSBD is at least one Double-Stranded DNA (dsDBP) binding domain or protein, and any variant and fragments thereof, optionally, said at least one dsDBP is at least one of: at least one Zinc finger (ZF), Non-specific RVD from AvrBS3 protein family, Helix-turn-helix (HTH), SRC Homology 3 (SH3) domain, chromatin-binding domain (CBD) protein and Sticky-C (StkC), domain or protein, and any variant and fragments thereof;
(c) wherein said Cas protein is a Cas mutant or variant, and wherein said Cas protein mutant or variant further comprises at least one of: (a) at least one point mutation substituting aspartic acid residue at position 10 to alanine (D10A) and/or at least one point mutation substituting histidine residue 849 to alanine (H849A); and (b) at least one deletion, substitution or replacement of at least one of: (i) the HNH-nuclease domain or any fragment thereof, and/or at least one amino acid residue thereof; (ii) the REC2 domain or any fragments thereof, and/or at least one amino acid residue thereof: (iii) the FLEX domain, or any fragments thereof, and/or at least one amino acid residue thereof; (iv) the RUVC domain or any fragments thereof, and/or at least one amino acid residue thereof; and (v) any combinations of (i), (ii), (iii), and (iv); and
(d) wherein said Cas protein or any variant, mutant, fusion or chimeric protein, complex or conjugate thereof, is capable of binding at least one target recognition element.

5-10. (canceled)

11. A nucleic acid guided genome modifier chimeric or fusion protein, complex or conjugate comprising:
(a) the at least one Cas protein or any Cas protein derived domain, having reduced or abolished PAM constraint according to claim 1, or any fragment, variant, or mutant thereof; and
(b) at least one nucleic acid modifier component.

12. The nucleic acid guided genome modifier chimeric protein, complex or conjugate according to claim 11, wherein said Cas protein is at least one of Cas9, CasX, Cas12a1, CasF, Cas14a1, an ancestral Cas9, and Cas14b5, optionally, said Cas protein is at least one of ScCas9, SpCas9, an ancestral Cas9, deltaproteobacteria CasX, Cas12a, CasF-1, CasF-2, CasF-3, Cas14a1, or Cas14b5, and wherein at least one PAM interacting Arginine and/or lysine residue of the PBD of said Cas protein is deleted or replaced.

13. (canceled)

14. The nucleic acid guided genome modifier chimeric protein, complex or conjugate according to claim 11, wherein at least one of:
(a) said Cas is ScCas9, and wherein at least one of: residues Thr1330 to Arg1342, residues Ile367 to Ala376, residue Lys1337, and residue Gln1338, or any fragments thereof, are replaced, substituted or deleted,
(b) said nucleic acid guided genome modifier chimeric protein, complex or conjugate further comprises at least one NSBD, said NSBD is at least one of: (i) added to said nucleic acid guided genome modifier chimeric protein, complex or conjugate; and/or (ii) replaces at least one of: said PAM binding domain, and/or said PAM recognition motif, and/or said HNH-nuclease domain, and/or any fragment thereof, and/or at least one adjacent amino acid residue, in said Cas protein of said nucleic acid guided genome modifier chimeric protein, complex or conjugate, optionally, said NSBD is at least one dsDBP binding domain or protein, and any variant and fragments thereof, optionally, said at least one dsDBP is at least one of: at least one ZF, HTH, SH3 domain, Non-specific RVD from AvrBS3 protein family, a CBD protein and StkC, domain or protein, and any variant and fragments thereof;
(c) said Cas protein is a Cas mutant or variant, said mutant or variant further comprises at least one of: (a) at least one point mutation substituting aspartic acid residue at position 10 to alanine (D10A), and/or at least one point mutation substituting histidine residue 849 to alanine (H849A); and (b) at least one deletion, substitution or replacement of at least one of: (i) the HNH-nuclease domain or any fragment thereof, and/or at least one amino acid residue thereof; (ii) the REC2 domain or any fragments thereof, and/or at least one amino acid residue thereof; (iii) the FLEX domain or any fragments thereof, and/or at least one amino acid residue thereof; (iv) the RUVC domain or any fragments thereof, and/or at least one amino acid residue thereof; and (v) any combinations of (i), (ii), (iii), and (iv); optionally, said Cas mutant is a defective CRISPR-Cas protein devoid of a nucleolytic activity; and
(d) said Cas protein or any variant, mutant, fusion protein, complex or conjugate thereof, is capable of binding at least one target recognition element, optionally, said at least one target recognition element is at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof.

15-22. (canceled)

23. The nucleic acid guided genome modifier chimeric protein, complex or conjugate according to claim 11, wherein said at least one nucleic acid modifier component is a protein-based modifier, a nucleic acid-based modifier or any combinations thereof, and wherein said protein-based modifier is at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, and any combinations thereof.

24. The nucleic acid guided genome modifier chimeric protein, complex or conjugate according to claim 23, wherein said nucleic acid modifier component is at least one nuclease, optionally, said nuclease is a Type IIS restriction endonuclease or any fragment, variant, mutant, fusion protein or conjugate thereof.

25. (canceled)

26. The nucleic acid guided genome modifier chimeric protein, complex or conjugate according to claim 25, wherein said Type IIS restriction endonuclease is Fold or any fragment, variant, mutant, fusion protein or conjugate thereof, optionally, said nucleic acid guided genome modifier is a chimeric protein, said chimeric protein is any one of: dScCasFok, SV40 NLS; dScCasFok, SV40+SV40 bipartite NLS; dCasFok, Fold consensus, SV40+bipartiteSV40; dCasFok, Fold consensus, SV40+bipartiteSV40, ancestral mutations in RuvC+REC domain, HNH deletion; dScCasFok, SV40+nucleoplasmin, ancestral mutations in RuvC+REC domain, Scloop QQmutation HNH deletion, whole PAMBD replaced with LacI DNA binding domain; dScCasFok, SV40+nucleoplasmin, ancestral mutations in RuvC+REC domain, Scloop SpReplacement, HNH deletion, PAMBD loop replaced with Zinc finger; dScCasFok, SV40+nucleoplasmin, ancestral mutations in RuvC+REC domain, Scloop SpReplacement HNH deletion, whole PAMBD replaced with SSO7D; dScCasFok, SV40+nucleoplasmin, ancestral mutations in RuvC+REC domain, Scloop SpReplacement HNH deletion, PAMBD loop replaced with HMGN; and dScCasFok, SV40+nucleoplasmin, ancestral mutations in RuvC+REC domain, Scloop SpReplacement HNH deletion, whole PAMBD replaced with STO7.

27. (canceled)

28. A nucleic acid molecule comprising a nucleic acid sequence encoding the at least one Cas protein or any Cas protein derived domain, according to claim 1, t or any fragment, variant, mutant, fusion protein, complex or conjugate thereof.

29. (canceled)

30. A nucleic acid guided genome modifier system or a composition thereof comprising:
(a) at least one Cas protein or Cas protein derived domain, having reduced or abolished PAM constraint, or any fragment, variant, mutant, fusion/chimeric protein, complex or conjugate thereof, or at least one nucleic acid sequence encoding said Cas protein or any fragment, variant, mutant, fusion/chimeric protein, complex or conjugate thereof, optionally, wherein at least one of the PBD and/or the PAM recognition motif, and/or the HNH-nuclease domain, any fragment of said PBD, and/or of said PAM recognition motif, and/or of said HNH-nuclease domain, and at least one amino acid residue adjacent to said PBD, and/or to said PAM recognition motif, and/or to said HNH-nuclease domain of said Cas protein, is deleted, substituted or replaced; and
(b) at least one target recognition element, or any nucleic acid sequence encoding said target recognition element.

31. The system according to claim 30, wherein said Cas protein is at least one of Cas9, CasX, Cas14a1, Cas14b5, Cas F, ancestral Cas9, and Cas12a or any variant, mutant, fusion/chimeric protein, complex or conjugate thereof, and wherein said chimeric or fusion protein thereof further comprises at least one nucleic acid modifier component, optionally, said Cas protein is at least one of ScCas9, SpCas9, an ancestral Cas9, deltaproteobacteria CasX, Cas12a, CasF-1, CasF-2, CasF-3, Cas14a1, or Cas14b5, and wherein at least one PAM interacting Arginine residue, and/or lysine residue of the PBD of said Cas protein is deleted, substituted or replaced.

32. (canceled)

33. The system according to claim 30, wherein at least one of:
(a) said Cas is ScCas9, with a replacement or deletion of at least one of: residues Thr1330 to Arg1342, residues Ile367 to Ala376, residues Glu1228 to Tyr1343, residues Glu1108 to Asp1375, residue Lys1337 and residue Gln1338;
(b) said Cas protein, nucleic acid guided genome modifier chimeric protein, complex or conjugate, further comprises at least one NSBD, said NSBD is at least one of: (i) added to said nucleic acid guided genome modifier chimeric protein, complex or conjugate; and/or (ii) replaces at least one of said PAM binding domain, and/or said PAM recognition motif, and/or said HNH-nuclease domain, and/or any fragment thereof, and/or at least one adjacent amino acid residue in said Cas protein of said nucleic acid guided genome modifier chimeric protein, complex or conjugate; optionally, said NSBD is at least one dsDBP binding domain or protein, and any variant and fragments thereof and wherein said at least one dsDBP is at least one of: at least one ZF, HTH, SH3 domain, Non-specific RVD of AvrBS3 protein family, CBD protein and StkC, domain or protein, and any variant and fragments thereof;
(c) said Cas protein is a Cas mutant or variant, said mutant or variant further comprises at least one of: (a) at least one point mutation substituting aspartic acid residue corresponding to position 10 of ScCas9 to alanine (D10A) and/or at least one point mutation substituting histidine residue corresponding to position 849 of ScCas9 to alanine (H849A); and (b) at least one deletion, substitution or replacement of at least one of: (i) the HNH-nuclease domain or any fragment thereof and/or at least one amino acid residue thereof; (ii) the REC2 domain or any fragments thereof, and/or at least one amino acid residue thereof; (iii) the FLEX domain or any fragments thereof, and/or at least one amino acid residue thereof; (iv) the RUVC domain or any fragments thereof, and/or at least one amino acid residue thereof; and (v) any combinations of (i), (ii), (iii), and (iv).

34-38. (canceled)

39. The system according to claim 30, wherein said Cas protein or any variant, mutant, fusion protein, complex or conjugate thereof, is capable of binding at least one target recognition element, and wherein said at least one target recognition element is at least one nucleic acid target recognition element, said target recognition element is at least one of: a single strand RNA molecule, a double strand RNA molecule, a single strand DNA, a double strand DNA, a modified DNA molecule, a modified RNA molecule, a LNA, a PNA and any hybrid or combinations thereof.

40. A host cell modified by, and/or comprising
(a) the at least one Cas protein or any Cas derived domain having reduced or abolished PAM constraint according to claim 1, or any variant, mutant, fusion/chimeric protein, complex or conjugate thereof, or at least one nucleic acid sequence encoding said Cas protein or any fragment, variant, mutant, fusion/chimeric protein or conjugate thereof; and
(b) at least one target recognition element or any nucleic acid sequence encoding said target recognition element;
(c) at least one nucleic acid cassette or any vector or vehicle comprising the nucleic acid sequence of (a), the nucleic acid sequence of (b) or the nucleic acid sequence of (a) and (b); or
(d) at least one system comprising (a) and (b) or a composition comprising said cell.

41-42. (canceled)

43. A composition comprising at least one of:
(a) at least one Cas protein or any Cas derived domain having reduced or abolished PAM constraint according to claim 1, or any fragment, variant, mutant, fusion/chimeric protein, complex or conjugate thereof, or at least one nucleic acid sequence encoding said Cas protein or any fragment, variant, mutant, fusion/chimeric protein or conjugate thereof;
(b) at least one target recognition element or any nucleic acid sequence encoding said target recognition element;
(c) at least one nucleic acid cassette or any vector or vehicle comprising the nucleic acid sequence of (a), the nucleic acid sequence of (b) or the nucleic acid sequence of (a) and (b);
(d) at least one system comprising (a) and (b); and
(e) at least one host cell comprising and/or modified by at least one of: the nucleic acid cassette or any vector or vehicle of (c) and the at least one system of (d); or any matrix, nano- or micro-particle comprising at least one of (a), (b), (c), (d) and (e), said composition optionally further comprises at least one of pharmaceutically acceptable carrier/s, diluent/s, excipient/s and additive/s.

44. (canceled)

45. A method of modifying at least one target nucleic acid sequence of interest in at least one cell or biochemical reaction, said method comprising the steps of contacting said cell or biochemical reaction with at least one of:
(a) at least one Cas protein having reduced or abolished PAM constraint or any fragment, variant, mutant, fusion/chimeric protein, complex or conjugate thereof, or at least one nucleic acid sequence encoding said Cas protein or any fragment, variant, mutant, fusion/chimeric protein, complex or conjugate thereof, optionally, wherein at least one of: the PBD and/or the PAM recognition motif, and/or the HNH-nuclease domain, any fragment of said PBD, and/or of said PAM recognition motif, and/or of said HNH-nuclease domain, and at least one amino acid residue adjacent to said PBD, and/or to said PAM recognition motif, and/or to said HNH-nuclease domain of said Cas protein, is deleted, substituted or replaced;
(b) at least one target recognition element or any nucleic acid sequence encoding said target recognition element;
(c) at least one nucleic acid cassette or any vector or vehicle comprising the nucleic acid sequence of (a), the nucleic acid sequence of (b) or the nucleic acid sequence of (a) and (b); and
(d) at least one system or composition comprising at least one of (a) and (b).

46. (canceled)

47. The method according to claim 45, wherein at least one of:
(a) said cell is of at least one organism of the biological kingdom Animalia;
(b) said target nucleic acid sequence of interest is and/or is comprised within at least one of: at least one gene encoding at least one tumor associated antigen (TAA), at least one gene encoding at least one immune checkpoint receptor proteins or ligand, at least one gene encoding a protein involved in at least one congenital disorder, at least one gene encoding receptors for at least one viral antigen, at least one gene associated with at least one inborn error of metabolism (IEM) disorder, Immunoglobulin locus, T cell receptor (TCR) locus, safe harbor site/s (SHS), and any coding sequence or non-coding sequence involved with at least one pathologic disorder;
(c) wherein said cell is of at least one organism of the biological kingdom Plantae; and
(d) said modification of at least one target nucleic acid sequence of interest in at least one cell is performed in at least one organism of at least one of: the biological kingdom Plantae and the biological kingdom Animalia.

48-50. (canceled)

51. A method according to claim 45, for curing or treating, preventing, inhibiting, reducing, eliminating, protecting or delaying the onset of a pathologic disorder or condition in a subject in need thereof, said method comprising the steps of administering to said subject an effective amount of at least one of:
(a) at least one Cas protein or any Cas protein derived domain, having reduced or abolished PAM constraint, or any variant, mutant, fusion/chimeric protein, complex or conjugate thereof, or at least one nucleic acid sequence encoding said Cas protein or any fragment, variant, mutant, fusion/chimeric protein, complex or conjugate thereof, optionally, wherein at least one of the PBD and/or the PAM recognition motif, and/or the HNH-nuclease domain, any fragment of said PBD, and/or of said PAM recognition motif, and/or of said HNH-nuclease domain, and at least one amino acid residue adjacent to said PBD, and/or to said PAM recognition motif, and/or to said HNH-nuclease domain of said Cas protein, is deleted, substituted or replaced;
(b) at least one target recognition element or any nucleic acid sequence encoding said target recognition element;
(c) at least one nucleic acid cassette or any vector or vehicle comprising the nucleic acid sequence of (a), the nucleic acid sequence of (b) or the nucleic acid sequence of (a) and (b);
(d) at least one system comprising (a) and (b);
(e) at least one host cell modified by and/or comprising at least one of: (a), (b), (c) and (d); and
(f) at least one composition comprising at least one of (a), (b), (c), (d) and (e)); optionally, wherein said subject is of the biological kingdom Animalia or of the biological kingdom Plantae.

52-53. (canceled)

54. The method according to claim 53, wherein said subject of the biological kingdom Animalia is a mammalian subject, optionally, said pathologic disorder is any one of a proliferative disorder, a congenital disorder, an immune-related condition, an inflammatory condition, a metabolic disorder, a disorder caused by a pathogen, an autoimmune disorder and an IEM disorder, optionally, said congenital disorder is any one of adRP and PSACH, said proliferative disorder is at least one of non-small cell lung cancer (NSCLC) melanoma, renal cell cancer, ovarian carcinoma and breast carcinoma, and wherein said a disorder caused by a pathogen is a viral disorder, said viral disorder is a Foot and Mouth Disease.

55-56. (canceled)

57. The method according to claim 51, said method comprising the step of administering to said subject a therapeutically effective amount of at least one host cell modified by, and/or comprising: at least one Cas protein or any Cas derived domain having reduced or abolished PAM constraint, or any variant, mutant, fusion/chimeric protein, complex or conjugate thereof, or at least one nucleic acid sequence encoding said Cas protein or any fragment, variant, mutant, fusion/chimeric protein or conjugate thereof or of any composition comprising said cells, wherein said cell is of an autologous or allogeneic source.

58-60. (canceled)
